(19) United States
(12) Patent Application Publication
(10) Pub. No.: US 2008/0306069 A1
(43) Pub. Date: Dec. 11, 2008

(54) Pyrazole Derivatives for the Inhibition of CDK'S and GSK'S

(75) Inventors: Paul Graham Wyatt, Perth (GB); Valerio Berdini, Cambridge (GB); Adrian Liam Gill, Buxton (GB); Gary Trewartha, Stevenage (GB); Andrew James Woodhead, Cambridge (GB); Eva Figueroa Navarro, Cambridge (GB); Michael Alistair O'Brien, Herts (GB); Theresa Rachael Phillips, Macclesfield (GB)

Correspondence Address: HESLIN ROTHENBERG FARLEY & MESITI PC, 5 COLUMBIA CIRCLE, ALBANY, NY, 12203, US

(73) Assignee: ASTEX THERAPEUTICS LIMITED, Cambridge (UK)

(21) Appl. No.: 11/814,443

(22) PCT Filed: Jan. 20, 2006
(86) PCT No.: PCT/GB2006/000191

§ 371 Date: Feb. 5, 2008

Related U.S. Application Data
(60) Provisional application No. 60/646,217, filed on Jan. 21, 2005.
(60) Provisional application No. 60/651,339, filed on Feb. 9, 2005.

(30) Foreign Application Priority Data
Jan. 22, 2005 (GB) 0501480.8
Jan. 27, 2005 (GB) 0501748.8

(51) Int. Cl. A61K 31/415; A61P 35/00; C07D 401/12; C07D 401/14; C07D 413/14; A61K 31/4439; C07D 231/38; A61K 31/454; A61K 31/4545; A61K 31/5377

(52) U.S. Cl. 514/236.5; 546/275.4; 546/194; 546/211; 544/130; 548/371.7; 514/326; 514/318; 514/407; 514/341

(57) ABSTRACT

The invention provides compounds of the formula (I), or salts, tautomers, N-oxides or solvates thereof wherein: R1 is selected from: (a) 2,6-dichlorophenyl; (b) 2,6-difluorophenyl; (c) a 2,3,6-trisubstituted phenyl group wherein the substituents for the phenyl group are selected from fluorine, chlorine, methyl and methoxy; (d) a group RO; (e) a group R a; (f) a group RIb; (g) a group RIc; (h) a group RId; and 0) 2,6-difluorophenylamino; wherein R) 0υ, r R>llaa, T Rj HbD, T R) HcC, r R>Iidα, r R>>2zaa, r R>22bD and RJ are as defined in the claims. The compounds have activity as inhibitors of cdk kinase (such as cdk1 or cdk2) and glycogen synthase kinase-3 activity.

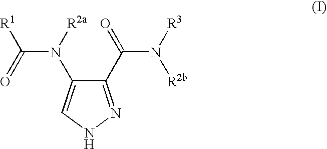

This invention relates to pyrazole compounds that inhibit or modulate the activity of Cyclin Dependent Kinases (CDK) and Glycogen Synthase Kinases (GSK) kinases, to the use of the compounds in the treatment or prophylaxis of disease states or conditions mediated by the kinases, and to novel compounds having kinase inhibitory or modulating activity. Also provided are pharmaceutical compositions containing the compounds and novel chemical intermediates.

### BACKGROUND OF THE INVENTION

Protein kinases constitute a large family of structurally related enzymes that are responsible for the control of a wide variety of signal transduction processes within the cell (Hardie, G. and Hanks, S. (1995) The Protein Kinase Facts Book. I and II, Academic Press, San Diego, Calif.). The kinases may be categorized into families by the substrates they phosphorylate (e.g., protein-tyrosine, protein-serine/threonine, lipids, etc.). Sequence motifs have been identified that generally correspond to each of these kinase families (e.g., Hanks, S. K., Hunter, T., FASEB J, 9:576-596 (1995); Knighton, et al, Science, 253:407-414 (1991); Hiles, et al, Cell, 70:419-429 (1992); Kunz, et al, Cell, 73:585-596 (1993); Garcia-Bustos, et al, EMBO J., 13:2352-2361 (1994)).

Protein kinases may be characterized by their regulation mechanisms. These mechanisms include, for example, autophosphorylation, transphosphorylation by other kinases, protein-protein interactions, protein-lipid interactions, and protein-polynucleotide interactions. An individual protein kinase may be regulated by more than one mechanism.

Kinases regulate many different cell processes including, but not limited to, proliferation, differentiation, apoptosis, motility, transcription, translation and other signalling processes, by adding phosphate groups to target proteins. These phosphorylation events act as molecular on/off switches that can modulate or regulate the target protein biological function. Phosphorylation of target proteins occurs in response to a variety of extracellular signals (hormones, neurotransmitters, growth and differentiation factors, etc.), cell cycle events, environmental or nutritional stresses, etc. The appropriate protein kinase functions in signalling pathways to activate or inactivate (either directly or indirectly), for example, a metabolic enzyme, regulatory protein, receptor, cytoskeletal protein, ion channel or pump, or transcription factor. Uncontrolled signalling due to defective control of protein phosphorylation has been implicated in a number of diseases, including, for example, inflammation, cancer, allergy/asthma, disease and conditions of the immune system, disease and conditions of the central nervous system, and angiogenesis.

### Cyclin Dependent Kinases

The process of eukaryotic cell division may be broadly divided into a series of sequential phases termed G1, S, G2 and M. Correct progression through the various phases of the cell cycle has been shown to be critically dependent upon the spatial and temporal regulation of a family of proteins known as cyclin dependent kinases (cdks) and a diverse set of their cognate protein partners termed cyclins. Cdks are cdc2 (also known as cdk1) homologous serine-threonine kinase proteins that are able to utilise ATP as a substrate in the phosphorylation of diverse polypeptides in a sequence dependent context. Cyclins are a family of proteins characterised by a homology region, containing approximately 100 amino acids, termed the “cyclin box” which is used in binding to, and defining selectivity for, specific cdk partner proteins.

Modulation of the expression levels, degradation rates, and activation levels of various cdks and cyclins throughout the cell cycle leads to the cyclical formation of a series of cdk/cyclin complexes, in which the cdks are enzymatically active. The formation of these complexes controls passage through discrete cell cycle checkpoints and thereby enables the process of cell division to continue. Failure to satisfy the pre-requisite biochemical criteria at a given cell cycle checkpoint, i.e. failure to form a required cdk/cyclin complex, can lead to cell cycle arrest and/or cellular apoptosis. Aberrant cellular proliferation, as manifested in cancer, can often be attributed to loss of correct cell cycle control. Inhibition of cdk enzymatic activity therefore provides a means by which abnormally dividing cells can have their division arrested and/or be killed. The diversity of cdks, and cdk complexes, and their critical roles in mediating the cell cycle, provides a broad spectrum of potential therapeutic targets selected on the basis of a defined biochemical rationale.

Progression from the G1 phase to the S phase of the cell cycle is primarily regulated by cdk2, cdk3, cdk4 and cdkó via association with members of the D and E type cyclins. The D-type cyclins appear instrumental in enabling passage beyond the G1 restriction point, where as the cdk2/cyclin E complex is key to the transition from the G1 to S phase. Subsequent progression through S phase and entry into G2 is thought to require the cdk2/cyclin A complex. Both mitosis, and the G2 to M phase transition which triggers it, are regulated by complexes of cdk1 and the A and B type cyclins.

During G1 phase Retinoblastoma protein (R^{b}), and related pocket proteins such as pi30, are substrates for cdk(2, 4, & 6)/cyclin complexes. Progression through G1 is in part facilitated by hyperphosphorylation, and thus inactivation, of R^{b} and pi30 by the cdk(4/6)/cyclin-D complexes. Hyperphosphorylation of R^{b} and pi30 causes the release of transcription factors, such as E2F, and thus the expression of genes necessary for progression through G1 and for entry into S-phase, such as the gene for cyclin E. Expression of cyclin E facilitates formation of the cdk2/cyclin E complex which amplifies, or maintains, E2F levels via further phosphorylation of Rb. The cdk2/cyclin E complex also phosphorylates other proteins necessary for DNA replication, such as NPAT, which has been implicated in histone biosynthesis. G1 progression and the G1/S transition are also regulated via the mitogen stimulated Myc pathway, which feeds into the cdk2/cyclin E pathway. Cdk2 is also connected to the p53 mediated DNA damage response pathway via p53 regulation of p21 levels. p21 is a protein inhibitor of cdk2/cyclin E and is thus capable of blocking, or delaying, the G1/S transition. The cdk2/cyclin E complex may thus represent a point at which biochemical stimuli from the R^{b}, Myc and p53 pathways are to some degree integrated. Cdk2 and/or the cdk2/cyclin E complex therefore represent good targets for therapeutics designed at arresting, or recovering control of, the cell cycle in aberrantly dividing cells.

The exact role of cdk3 in the cell cycle is not clear. As yet no cognate cyclin partner has been identified, but a dominant negative form of cdk3 delayed cells in G1, thereby suggesting that cdk3 has a role in regulating the G1/S transition.

Although most cdks have been implicated in regulation of the cell cycle there is evidence that certain members of the cdk family are involved in other biochemical processes. This is exemplified by cdk5 which is necessary for correct neuronal development and which has also been implicated in the phosphorylation of several neuronal proteins such as Tau, NUDE-I, synapsin1, DARPP32 and the Munc1S/Syntaxin1A complex. Neuronal cdk5 is conventionally activated by binding to the p35/p39 proteins. Cdk5 activity can, however, be deregulated by the binding of p25, a truncated version of p35. Conversion of p35 to p25, and subsequent deregulation of cdk5 activity, can be induced by ischemia, excitotoxicity, and β-amyloid peptide. Consequently p25 has been implicated in the pathogenesis of neurodegenerative diseases, such as Alzheimer's, and is therefore of interest as a target for therapeutics directed against these diseases.

Cdk7 is a nuclear protein that has cdc2 CAK activity and binds to cyclin H. Cdk7 has been identified as component of the TFIIH transcriptional complex which has RNA polymerase II C-terminal domain (CTD) activity. This has been associated with the regulation of HIV-I transcription via a Tat-mediated biochemical pathway. Cdk8 binds cyclin C and has been implicated in the phosphorylation of the CTD of RNA polymerase II. Similarly the cdk9/cyclin-Tl complex (P-TEFb complex) has been implicated in elongation control of RNA polymerase II. PTEF-b is also required for activation of transcription of the HIV-I genome by the viral transactivator Tat through its interaction with cyclin Tl. Cdk7, cdk8, cdk9 and the P-TEFb complex are therefore potential targets for anti-viral therapeutics.

At a molecular level mediation of cdk/cyclin complex activity requires a series of stimulatory and inhibitory phosphorylation, or dephosphorylation, events. Cdk phosphorylation is performed by a group of cdk activating kinases (CAKs) and/or kinases such as weel, Myt1 and Mik1. Dephosphorylation is performed by phosphatases such as cdc25(a & c), pp 2a, or KAP.

Cdk/cyclin complex activity may be further regulated by two families of endogenous cellular proteinaceous inhibitors: the Kip/Cip family, or the INK family. The INK proteins specifically bind cdk4 and cdk6. p16^{ink4} (also known as MTS1) is a potential tumour suppressor gene that is mutated, or deleted, in a large number of primary cancers. The Kip/Cip family contains proteins such as p21^{cip1 ′Waf1}, p27^{KiP1} and p57^{kip2}. As discussed previously p21 is induced by p53 and is able to inactivate the cdk2/cyclin(E/A) and cdk4/cyclin(D1/D2/D3) complexes. Atypically low levels of p27 expression have been observed in breast, colon and prostate cancers. Conversely over expression of cyclin E in solid tumours has been shown to correlate with poor patient prognosis. Over expression of cyclin D 1 has been associated with oesophageal, breast, squamous, and non-small cell lung carcinomas.

The pivotal roles of cdks, and their associated proteins, in coordinating and driving the cell cycle in proliferating cells have been outlined above. Some of the biochemical pathways in which cdks play a key role have also been described. The development of monotherapies for the treatment of proliferative disorders, such as cancers, using therapeutics targeted generically at cdks, or at specific cdks, is therefore potentially highly desirable. Cdk inhibitors could conceivably also be used to treat other conditions such as viral infections, autoimmune diseases and neuro-degenerative diseases, amongst others. Cdk targeted therapeutics may also provide clinical benefits in the treatment of the previously described diseases when used in combination therapy with either existing, or new, therapeutic agents. Cdk targeted anticancer therapies could potentially have advantages over many current antitumour agents as they would not directly interact with DNA and should therefore reduce the risk of secondary tumour development.

### Glycogen Synthase Kinase

Glycogen Synthase Kinase-3 (GSK3) is a serine-threonine kinase that occurs as two ubiquitously expressed isoforms in humans (GSK3α & beta GSK3β). GSK3 has been implicated as having roles in embryonic development, protein synthesis, cell proliferation, cell differentiation, microtubule dynamics, cell motility and cellular apoptosis. As such GSK3 has been implicated in the progression of disease states such as diabetes, cancer, Alzheimer's disease, stroke, epilepsy, motor neuron disease and/or head trauma. Phylogenetically GSK3 is most closely related to the cyclin dependent kinases (CDKs).

The consensus peptide substrate sequence recognised by GSK3 is (Ser/Thr)-X-X-X-(pSer/pThr), where X is any amino acid (at positions (n+1), (n+2), (n+3)) and pSer and pThr are phospho-serine and phospho-threonine respectively (n+4). GSK3 phosphorylates the first serine, or threonine, at position (n). Phospho-serine, or phospho-threonine, at the (n+4) position appear necessary for priming GSK3 to give maximal substrate turnover. Phosphorylation of GSK3α at Ser21, or GSK3β at Ser9, leads to inhibition of GSK3. Mutagenesis and peptide competition studies have led to the model that the phosphorylated N-terminus of GSK3 is able to compete with phospho-peptide substrate (S/TXXXpS/pT) via an autoinhibitory mechanism. There are also data suggesting that GSK3α and GSKβ may be subtly regulated by phosphorylation of tyrosines 279 and 216 respectively. Mutation of these residues to a Phe caused a reduction in in vivo kinase activity. The X-ray crystallographic structure of GSK3β has helped to shed light on all aspects of GSK3 activation and regulation.

GSK3 forms part of the mammalian insulin response pathway and is able to phosphorylate, and thereby inactivate, glycogen synthase. Upregulation of glycogen synthase activity, and thereby glycogen synthesis, through inhibition of GSK3, has thus been considered a potential means of combating type II, or non-insulin-dependent diabetes mellitus (NIDDM): a condition in which body tissues become resistant to insulin stimulation. The cellular insulin response in liver, adipose, or muscle tissues, is triggered by insulin binding to an extracellular insulin receptor. This causes the phosphorylation, and subsequent recruitment to the plasma membrane, of the insulin receptor substrate (IRS) proteins. Further phosphorylation of the IRS proteins initiates recruitment of phosphoinositide-3 kinase (PBK) to the plasma membrane where it is able to liberate the second messenger phosphatidylinosityl 3,4,5-trisphosphate (PIP3). This facilitates co-localisation of 3-phosphoinositide-dedependent protein kinase 1 (PDK1) and protein kinase B (PKB or Akt) to the membrane, where PDK1 activates PKB. PKB is able to phosphorylate, and thereby inhibit, GSK3α and/or GSKβ through phosphorylation of Ser9, or ser21, respectively. The inhibition of GSK3 then triggers upregulation of glycogen synthase activity. Therapeutic agents able to inhibit GSK3 may thus be able to induce cellular responses akin to those seen on insulin stimulation. A further in vivo substrate of GSK3 is the eukaryotic protein synthesis initiation factor 2B (eIF2B). eIF2B is inactivated via phosphorylation and is thus able to suppress protein biosynthesis. Inhibition of GSK3, e.g. by inactivation of the “mammalian target of rapamycin” protein (mTOR), can thus upregulate protein biosynthesis. Finally there is some evidence for regulation of GSK3 activity via the mitogen activated protein kinase (MAPK) pathway through phosphorylation of GSK3 by kinases such as mitogen activated protein kinase activated protein kinase 1 (MAPKAP-K1 or RSK). These data suggest that GSK3 activity may be modulated by mitogenic, insulin and/or amino acid stimuli.

It has also been shown that GSK3β is a key component in the vertebrate Wnt signalling pathway. This biochemical pathway has been shown to be critical for normal embryonic development and regulates cell proliferation in normal tissues. GSK3 becomes inhibited in response to Wnt stimulii. This can lead to the de-phosphorylation of GSK3 substrates such as Axin, the adenomatous polyposis coli (APC) gene product and β-catenin. Aberrant regulation of the Wnt pathway has been associated with many cancers. Mutations in APC, and/or β-catenin, are common in colorectal cancer and other tumours, β-catenin has also been shown to be of importance in cell adhesion. Thus GSK3 may also modulate cellular adhesion processes to some degree. Apart from the biochemical pathways already described there are also data implicating GSK3 in the regulation of cell division via phosphorylation of cyclin-D1, in the phosphorylation of transcription factors such as c-Jun, CCAAT/enhancer binding protein α (C/EBPα), c-Myc and/or other substrates such as Nuclear Factor of Activated T-cells (NFATc), Heat Shock Factor-1 (HSF-I) and the c-AMP response element binding protein (CREB). GSK3 also appears to play a role, albeit tissue specific, in regulating cellular apoptosis. The role of GSK3 in modulating cellular apoptosis, via a pro-apoptotic mechanism, may be of particular relevance to medical conditions in which neuronal apoptosis can occur. Examples of these are head trauma, stroke, epilepsy, Alzheimer's and motor neuron diseases, progressive supranuclear palsy, corticobasal degeneration, and Pick's disease. In vitro it has been shown that GSK3 is able to hyper-phosphorylate the microtubule associated protein Tau. Hyperphosphorylation of Tau disrupts its normal binding to microtubules and may also lead to the formation of intra-cellular Tau filaments. It is believed that the progressive accumulation of these filaments leads to eventual neuronal dysfunction and degeneration. Inhibition of Tau phosphorylation, through inhibition of GSK3, may thus provide a means of limiting and/or preventing neurodegenerative effects.

### Diffuse Large B-Cell Lymphomas (DLBCL)

Cell cycle progression is regulated by the combined action of cyclins, cyclin-dependent kinases (CDKs), and CDK-inhibitors (CDKi), which are negative cell cycle regulators. p27KIP1 is a CDKi key in cell cycle regulation, whose degradation is required for G1/S transition. In spite of the absence of p27KIP1 expression in proliferating lymphocytes, some aggressive B-cell lymphomas have been reported to show an anomalous p27KIP1 staining. An abnormally high expression of p27KIP1 was found in lymphomas of this type. Analysis of the clinical relevance of these findings showed that a high level of p27KIP1 expression in this type of tumour is an adverse prognostic marker, in both univariate and multivariate analysis. These results show that there is abnormal p27KIP1 expression in Diffuse Large B-cell Lymphomas (DLBCL), with adverse clinical significance, suggesting that this anomalous p27KIP1 protein may be rendered none functional through interaction with other cell cycle regulator proteins. (Br. J. Cancer. 1999 July; 80(9): 1427-34. p27KIP 1 is abnormally expressed in Diffuse Large B-cell Lymphomas and is associated with an adverse clinical outcome. Saez A, Sanchez E, Sanchez-Beato M, Cruz M A, Chacon I, Munoz E, Camacho F T, Martinez-Montero J C, Mollejo M, Garcia J F, Piris M A. Department of Pathology, Virgen de Ia Salud Hospital, Toledo, Spain.)

### Chronic Lymphocytic Leukemia

B-Cell chronic lymphocytic leukaemia (CLL) is the most common leukaemia in the Western hemisphere, with approximately 10,000 new cases diagnosed each year (Parker S L, Tong T, Bolden S, Wingo P A: Cancer statistics, 1997. Ca. Cancer. J. Clin. 47:5, (1997)). Relative to other forms of leukaemia, the overall prognosis of CLL is good, with even the most advanced stage patients having a median survival of 3 years.

The addition of fludarabine as initial therapy for symptomatic CLL patients has led to a higher rate of complete responses (27% v 3%) and duration of progression-free survival (33 v 17 months) as compared with previously used alkylator-based therapies. Although attaining a complete clinical response after therapy is the initial step toward improving survival in CLL, the majority of patients either do not attain complete remission or fail to respond to fludarabine. Furthermore, all patients with CLL treated with fludarabine eventually relapse, making its role as a single agent purely palliative (Rai K R, Peterson B, Elias L, Shepherd L, Hines J, Nelson D, Cheson B, Kolitz J, Schiffer C A: A randomized comparison of fludarabine and chlorambucil for patients with previously untreated chronic lymphocytic leukemia. A CALGB SWOG, CTG/NCI-C and ECOG Inter-Group Study. Blood 88:141a, 1996 (abstr 552, suppl 1). Therefore, identifying new agents with novel mechanisms of action that complement fludarabine's cytotoxicity and abrogate the resistance induced by intrinsic CLL drug-resistance factors will be necessary if further advances in the therapy of this disease are to be realized.

The most extensively studied, uniformly predictive factor for poor response to therapy and inferior survival in CLL patients is aberrant p53 function, as characterized by point mutations or chromosome 17p 3 deletions. Indeed, virtually no responses to either alkylator or purine analog therapy have been documented in multiple single institution case series for those CLL patients with abnormal p53 function. Introduction of a therapeutic agent that has the ability to overcome the drug resistance associated with p53 mutation in CLL would potentially be a major advance for the treatment of the disease.

Flavopiridol and CYC 202, inhibitors of cyclin-dependent kinases induce in vitro apoptosis of malignant cells from B-cell chronic lymphocytic leukemia (B-CLL).

Flavopiridol exposure results in the stimulation of caspase 3 activity and in caspase-dependent cleavage of p27(kip1), a negative regulator of the cell cycle, which is overexpressed99 in B-CLL (Blood. 1998 Nov. 15; 92(10):3804-16 Flavopiridol induces apoptosis in chronic lymphocytic leukemia cells via activation of caspase-3 without evidence of bc1-2 modulation or dependence on functional p53. Byrd J C, Shinn C, Waselenko J K, Fuchs E J, Lehman T A, Nguyen P L, Flinn I W, Diehl L F, Sausville E, Grever M R).

### Prior Art

WO 02/34721 from Du Pont discloses a class of indeno[1,2-c]pyrazol-4-ones as inhibitors of cyclin dependent kinases.

WO 01/81348 from Bristol Myers Squibb describes the use of 5-thio-, sulphinyl- and sulphonylpyrazolo[3,4-b]-pyridines as cyclin dependent kinase inhibitors.

WO 00/62778 also from Bristol Myers Squibb discloses a class of protein tyrosine kinase inhibitors.

WO 01/72745A1 from Cyclacel describes 2-substituted 4-heteroaryl-pyrimidines and their preparation, pharmaceutical compositions containing them and their use as inhibitors of cyclin-dependant kinases (CDKs) and hence their use in the treatment of proliferative disorders such as cancer, leukaemia, psoriasis and the like.

WO 99/21845 from Agouron describes 4-aminothiazole derivatives for inhibiting cyclin-dependent kinases (CDKs), such as CDK1, CDK2, CDK4, and CDK6. The invention is also directed to the therapeutic or prophylactic use of pharmaceutical compositions containing such compounds and to methods of treating malignancies and other disorders by administering effective amounts of such compounds.

WO 01/53274 from Agouron discloses as CDK kinase inhibitors a class of compounds which can comprise an amide-substituted benzene ring linked to an N-containing heterocyclic group.

WO 01/98290 (Pharmacia & Upjohn) discloses a class of 3-aminocarbonyl-2-carboxamido thiophene derivatives as protein kinase inhibitors.

WO 01/53268 and WO 01/02369 from Agouron disclose compounds that mediate or inhibit cell proliferation through the inhibition of protein kinases such as cyclin dependent kinase or tyrosine kinase. The Agouron compounds have an aryl or heteroaryl ring attached directly or though a CH═CH or CH═N group to the 3-position of an indazole ring.

WO 00/39108 and WO 02/00651 (both to Du Pont Pharmaceuticals) describe heterocyclic compounds that are inhibitors of trypsin-like serine protease enzymes, especially factor Xa and thrombin. The compounds are stated to be useful as anticoagulants or for the prevention of thromboembolic disorders.

US 2002/0091116 (Zhu et al\WO 01/19798 and WO 01/64642 each disclose diverse groups of heterocyclic compounds as inhibitors of Factor Xa. Some 1-substituted pyrazole carboxamides are disclosed and exemplified.

U.S. Pat. No. 6,127,382, WO 01/70668, WO 00/68191, WO 97/48672, WO 97/19052 and WO 97/19062 (all to Allergan) each describe compounds having retinoid-like activity for use in the treatment of various hyperproliferative diseases including cancers.

WO 02/070510 (Bayer) describes a class of amino-dicarboxylic acid compounds for use in the treatment of cardiovascular diseases. Although pyrazoles are mentioned generically, there are no specific examples of pyrazoles in this document.

WO 97/03071 (Knoll A G) discloses a class of heterocyclyl-carboxamide derivatives for use in the treatment of central nervous system disorders. Pyrazoles are mentioned generally as examples of heterocyclic groups but no specific pyrazole compounds are disclosed or exemplified.

WO 97/40017 (Novo Nordisk) describes compounds that are modulators of protein tyrosine phosphatases.

WO 03/020217 (Univ. Connecticut) discloses a class of pyrazole 3-carboxamides as cannabinoid receptor modulators for treating neurological conditions. It is stated (page 15) that the compounds can be used in cancer chemotherapy but it is not made clear whether the compounds are active as anti-cancer agents or whether they are administered for other purposes.

WO 01/58869 (Bristol Myers Squibb) discloses cannabinoid receptor modulators that can be used inter alia to treat a variety of diseases. The main use envisaged is the treatment of respiratory diseases, although reference is made to the treatment of cancer.

WO 01/02385 (Aventis Crop Science) discloses 1-(quinoline-4-yl)-1H-pyrazole derivatives as fungicides. 1-Unsubstituted pyrazoles are disclosed as synthetic intermediates.

WO 2004/039795 (Fujisawa) discloses amides containing a 1-substituted pyrazole group as inhibitors of apolipoprotein B secretion. The compounds are stated to be useful in treating such conditions as hyperlipidemia.

WO 2004/000318 (Cellular Genomics) discloses various amino-substituted monocycles as kinase modulators. None of the exemplified compounds are pyrazoles.

### SUMMARY OF THE INVENTION

The invention provides compounds that have cyclin dependent kinase inhibiting or modulating activity and glycogen synthase kinase-3 (GSK3) inhibiting or modulating activity, and which it is envisaged will be useful in preventing or treating disease states or conditions mediated by the kinases.

Thus, for example, it is envisaged that the compounds of the invention will be useful in alleviating or reducing the incidence of cancer.

In a first aspect, the invention provides a compound of the formula (I):

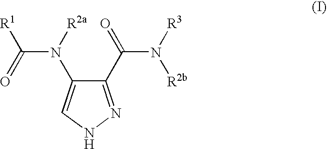

wherein:
R¹ is selected from:
(a) 2,6-dichlorophenyl;
(b) 2,6-difluorophenyl;
(c) a 2,3,6-trisubstituted phenyl group wherein the substituents for the phenyl group are selected from fluorine, chlorine, methyl and methoxy;
(d) a group R⁰;
(e) a group R^{1a};
(f) a group R^{1b};
(g) a group R^{1c};
(h) a group R^{1d}; and
(j) 2,6-difluorophenylamino;

R⁰ is a carbocyclic or heterocyclic group having from 3 to 12 ring members; or a C₁₋₈ hydrocarbyl group optionally substituted by one or more substituents selected from fluorine, hydroxy, cyano; C₁₋₄ hydrocarbyloxy, amino, mono- or di-C₁₋₄ hydrocarbylamino, and carbocyclic or heterocyclic groups having from 3 to 12 ring members, and wherein 1 or 2 of the carbon atoms of the hydrocarbyl group may optionally be replaced by an atom or group selected from O, S, NH, SO, SO₂;

R^{1a} is selected from cyclopropyl-cyano-methyl; furyl; benzoisoxazolyl; methylisoxazolyl; 2-monosubstituted phenyl and 2,6-disubstituted phenyl wherein the substituents on the phenyl moiety are selected from methoxy, ethoxy, fluorine, chlorine, and difluoromethoxy; provided that R^{1a} is not 2,6-difluorophenyl or 2,6-dichlorophenyl;

R^{1b} is selected from tetrahydrofuryl; and mono-substituted and disubstituted phenyl wherein the substituents on the phenyl moiety are selected from fluorine; chlorine; methoxy; ethoxy and methylsulphonyl;

R^{1c} is selected from; benzoisoxazolyl; five membered heteroaryl rings containing one or two heteroatoms selected from O and N and six-membered heteroaryl rings containing one or two nitrogen heteroatom ring members, the heteroaryl rings in each case being optionally substituted by methyl, fluorine, chlorine or trifluoromethyl; and phenyl substituted by one, two or three substituents selected from bromine, chlorine, fluorine, methyl, trifluoromethyl, ethoxy, methoxy, methoxyethoxy, methoxymethyl, dimethylaminomethyl and difluoromethoxy; provided that R^{1a} is not 2,6-difluorophenyl;

R^{1d} is a group R^{1e}—(CH₂)ₙCH(CN)— where n is 0-2 and R^{1e} is a carbocylic or heterocyclic group having from 3 to 12 ring members;

R^{2a} and R^{2b} are each hydrogen or methyl;

and wherein:
A. when R¹ is (a) 2,6-dichlorophenyl and R^{2a} and R^{2b} are both hydrogen; then R³ can be selected from:
(i) a group

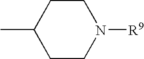

where R⁹ is selected from C(O)NR⁵R⁶; C(O)—R¹⁰ and 2-pyrimidinyl where R¹⁰ is a C₁₋₄ alkyl group optionally substituted by one or more substituents chosen from fluorine, chlorine, cyano and methoxy; and R¹¹ where R¹¹ is a C₁₋₄ alkyl group substituted by one or more substituents chosen from fluorine, chlorine and cyano;
(ii) a group

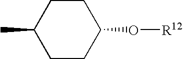

where R¹² is C₂₋₄ alkyl;
(iii) a group

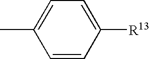

wherein R¹³ is selected from methylsulphonyl, 4-morpholino, 4-thiomorpholino, 1-piperidino, 1-methyl-4-piperazino and 1-pyrrolidino;
(iv) a substituted 3-pyridyl or 4-pyridyl group of the formula

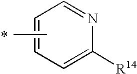

wherein the group R¹⁴ is meta or para with respect to the bond labelled with an asterisk and is selected from methyl, methylsulphonyl, 4-morpholino, 4-thiomorpholino, 1-piperidino, 1-methyl-4-piperazino, 1-pyrrolidino, 4-piperidinyloxy, 1-C₁₋₄alkoxycarbonyl-piperidin-4-yloxy, 2-hydroxyethoxy and 2-methoxyethoxy; and
(v) a group selected from 2-pyrazinyl, 5-pyrimidinyl, cyclohexyl, 1,4-dioxa-spiro[4.5]decan-8-yl (4-cyclohexanone ethylene glycol ketal), 4-methylsulphonylamino-cyclohexyl, tetrahydrothiopyran-4-yl, 1,1-dioxo-tetrahydrothiopyran-4-yl, tetrahydropyran-4-yl, 4,4-difluorocyclohexyl and 3,5-dimethylisoxazol-4-yl; and
   B. when R¹ is (b) 2,6-difluorophenyl and R^{2a} and R^{2b} are both hydrogen; then R³ can be selected from:
(vi) 1-methyl-piperidin-3-yl; 4-(2-dimethylaminoethoxy)-cyclohexyl; and an N-substituted 4-piperidinyl group wherein the N-substituent is selected from cyanomethyl and cyanoethyl; and
(vii) a group

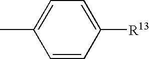

wherein R¹³ is as hereinbefore defined; and
   C. when R¹ is (c) a 2,3,6-trisubstituted phenyl group wherein the substituents for the phenyl group are selected from fluorine, chlorine, methyl and methoxy; and R^{2a} and R^{2b} are both hydrogen; then R³ can be selected from groups (ii), (xi), (xii) and (xiii) as defined herein; and
(viii) 4-piperidinyl and 1-methyl-4-piperidinyl;
(ix) tetrahydropyran-4-yl; and
(x) a group:

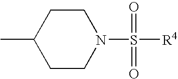

where R⁴ is C₁₋₄ alkyl;
   D. when R¹ is (d), a group R⁰, where R⁰ is a carbocyclic or heterocyclic group having from 3 to 12 ring members; or a C₁₋₈ hydrocarbyl group optionally substituted by one or more substituents selected from fluorine, hydroxy, cyano; C₁₋₄ hydrocarbyloxy, amino, mono- or di-C₁₋₄ hydrocarbylamino, and carbocyclic or heterocyclic groups having from 3 to 12 ring members, and wherein 1 or 2 of the carbon atoms of the hydrocarbyl group may optionally be replaced by an atom or group selected from O, S, NH, SO, SO₂; then R³ can be selected from:
(xi) a group:

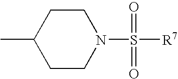

where R⁷ is:
   unsubstituted hydrocarbyl other than C₁₋₄ alkyl;
   substituted C₁₋₄ hydrocarbyl bearing one or more substituents chosen from fluorine, chlorine, hydroxy, methylsulphonyl, cyano, methoxy, NR⁵R⁶, and 4 to 7 membered saturated carbocyclic or heterocyclic rings containing up to two heteroatom ring members selected from O, N and S;
   a group NR⁵R⁶ where R⁵ and R⁶ are selected from hydrogen and C₁₋₄ alkyl, C₁₋₂ alkoxy and C₁₋₂ alkoxy-C₁₋₄ alkyl, provided that no more than one of R⁵ and R⁶ is C₁₋₂ alkoxy, or NR⁵R⁶ forms a five or six membered saturated heterocyclic ring containing one or two heteroatom ring members selected from O, N and S, the heterocyclic ring being optionally substituted by one or more methyl groups;
   a five or six membered heteroaryl group containing one or two heteroatom ring members selected from N, S and O and being optionally substituted by methyl, methoxy, fluorine, chlorine, or a group NR⁵R⁶;
   a phenyl group optionally substituted by methyl, methoxy, fluorine, chlorine, cyano or a group NR⁵R⁶;
   C₃₋₆ cycloalkyl; and
   a five or six membered saturated heterocyclic ring containing one or two heteroatom ring members selected from O, N and S, the heterocyclic ring being optionally substituted by one or more methyl groups;
(xii) a group:

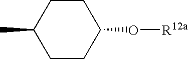

where R^{12a} is C₁₋₄ alkyl substituted by one or more substituents chosen from fluorine, chlorine, C₃₋₆ cycloalkyl, oxa-C₄₋₆ cycloalkyl, cyano, methoxy and NR⁵R⁶, provided that there are at least two carbon atoms between the oxygen atom to which R¹² is attached and a group NR⁵R⁶ when present; and
   E. when R¹ is (e) a group R^{1a} and R^{2a} and R^{2b} are both hydrogen, then R³ can be (xiii) a group

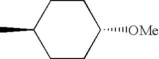

and
   F. when R¹ is (f) a group R^{1b}, and R^{2a} and R^{2b} are both hydrogen, then R³ can be (xiv) a methyl group; and
   G. when R¹ is (g) a group R^{1c} and R^{2a} and R^{2b} are both hydrogen, then R³ can be (xv) a group

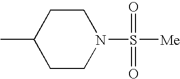

and:
H. when R¹ is (h), a group R^{1d}, then R³ is a group -Y-R^{3a} where Y is a bond or an alkylene chain of 1, 2 or 3 carbon atoms in length and R^{3a} is selected from hydrogen and carbocyclic and heterocyclic groups having from 3 to 12 ring members;
J. when R¹ is (j), 2,6-difluorophenylamino, and R^{2a} and R^{2b} are both hydrogen; then R³ can be methyl; and
K. when R¹ is 2,6-dichlorophenyl and either (k) R^{2a} is methyl and R^{2b} is hydrogen, or (1) R^{2a} is hydrogen and R^{2b} is methyl; then R³ can be a 4-piperidine group;
or salts, tautomers, solvates and N-oxides thereof.

The invention also provides inter alia:
A compound of the formula (I) or any sub-groups or examples thereof as defined herein for use in the prophylaxis or treatment of a disease state or condition mediated by a cyclin dependent kinase or glycogen synthase kinase-3.
A method for the prophylaxis or treatment of a disease state or condition mediated by a cyclin dependent kinase or glycogen synthase kinase-3, which method comprises administering to a subject in need thereof a compound of the formula (I) or any sub-groups or examples thereof as defined herein.
A method for alleviating or reducing the incidence of a disease state or condition mediated by a cyclin dependent kinase or glycogen synthase kinase-3, which method comprises administering to a subject in need thereof a compound of the formula (I) or any sub-groups or examples thereof as defined herein.
A method for treating a disease or condition comprising or arising from abnormal cell growth in a mammal, which method comprises administering to the mammal a compound of the formula (I) or any sub-groups or examples thereof as defined herein in an amount effective in inhibiting abnormal cell growth.
A method for alleviating or reducing the incidence of a disease or condition comprising or arising from abnormal cell growth in a mammal, which method comprises administering to the mammal a compound of the formula (I) or any sub-groups or examples thereof as defined herein in an amount effective in inhibiting abnormal cell growth.

A method for treating a disease or condition comprising or arising from abnormal cell growth in a mammal, the method comprising administering to the mammal a compound of the formula (I) or any sub-groups or examples thereof as defined herein in an amount effective to inhibit a cdk kinase (such as cdk1 or cdk2) or glycogen synthase kinase-3 activity.

A method for alleviating or reducing the incidence of a disease or condition comprising or arising from abnormal cell growth in a mammal, the method comprising administering to the mammal a compound of the formula (I) or any sub-groups or examples thereof as defined herein in an amount effective to inhibit a cdk kinase (such as cdk1 or cdk2) or glycogen synthase kinase-3 activity.

A method of inhibiting a cyclin dependent kinase or glycogen synthase kinase-3, which method comprises contacting the kinase with a kinase-inhibiting compound of the formula (I) or any sub-groups or examples thereof as defined herein.
A method of modulating a cellular process (for example cell division) by inhibiting the activity of a cyclin dependent kinase or glycogen synthase kinase-3 using a compound of the formula (I) or any sub-groups or examples thereof as defined herein.
A compound of the formula (I) or any sub-groups or examples thereof as defined herein for use in the prophylaxis or treatment of a disease state as described herein.
The use of a compound of the formula (I) or any sub-groups or examples thereof as defined herein for the manufacture of a medicament, wherein the medicament is for any one or more of the uses defined herein.
A pharmaceutical composition comprising a compound of the formula (I) or any sub-groups or examples thereof as defined herein and a pharmaceutically acceptable carrier.
A pharmaceutical composition comprising a compound of the formula (I) or any sub-groups or examples thereof as defined herein and a pharmaceutically acceptable carrier in a form suitable for oral administration.
A pharmaceutical composition for administration in an aqueous solution form, the pharmaceutical composition comprising a compound of the formula (I) or any sub-groups or examples thereof as defined herein in the form of a salt having a solubility in water of greater than 25 mg/ml, typically greater than 50 mg/ml and preferably greater than 100 mg/ml.
A compound of the formula (I) or any sub-groups or examples thereof as defined herein for use in medicine.
A method for the diagnosis and treatment of a disease state or condition mediated by a cyclin dependent kinase, which method comprises (i) screening a patient to determine whether a disease or condition from which the patient is or may be suffering is one which would be susceptible to treatment with a compound having activity against cyclin dependent kinases; and (ii) where it is indicated that the disease or condition from which the patient is thus susceptible, thereafter administering to the patient a compound of the formula (I) or any sub-groups or examples thereof as defined herein.
The use of a compound of the formula (I) or any sub-groups or examples thereof as defined herein for the manufacture of a medicament for the treatment or prophylaxis of a disease state or condition in a patient who has been screened and has been determined as suffering from, or being at risk of suffering from, a disease or condition which would be susceptible to treatment with a compound having activity against cyclin dependent kinase.
A compound of the formula (I) or any sub-groups or examples thereof as defined herein for use in inhibiting tumour growth in a mammal.
A compound of the formula (I) or any sub-groups or examples thereof as defined herein for use in inhibiting the growth of tumour cells (e.g. in a mammal).
A method of inhibiting tumour growth in a mammal (e.g. a human), which method comprises administering to the mammal (e.g. a human) an effective tumour growth-inhibiting amount of a compound of the formula (I) or any sub-groups or examples thereof as defined herein.
A method of inhibiting the growth of tumour cells (e.g. tumour cells present in a mammal such as a human), which method comprises contacting the tumour cells with an effective tumour cell growth-inhibiting amount of a compound of the formula (I) or any sub-groups or examples thereof as defined herein.
A compound as defined herein for any of the uses and methods set forth above, and as described elsewhere herein.

### General Preferences and Definitions

In this section, as in all other sections of this application, unless the context indicates otherwise, references to a compound of formula (I) includes all subgroups of formula (I) as defined herein and the term ‘subgroups’ includes all preferences, embodiments, examples and particular compounds defined herein.

Moreover, a reference to a compound of formula (I) and sub-groups thereof includes ionic forms, salts, solvates, isomers, tautomers, N-oxides, esters, prodrugs, isotopes and protected forms thereof, as discussed below:—preferably, the salts or tautomers or isomers or N-oxides or solvates thereof:—and more preferably, the salts or tautomers or N-oxides or solvates thereof.

The following general preferences and definitions shall apply to each of R¹ to R¹⁴, and their various sub-groups, sub-definitions, examples and embodiments unless the context indicates otherwise.

Any references to formula (I) herein shall also be taken to refer to and any subs group of compounds within formula (I) and any preferences and examples thereof unless the context requires otherwise.

References to “carbocyclic” and “heterocyclic” groups as used herein shall, unless the context indicates otherwise, include both aromatic and non-aromatic ring systems. Thus, for example, the term “carbocyclic and heterocyclic groups” includes within its scope aromatic, non-aromatic, unsaturated, partially saturated and fully saturated carbocyclic and heterocyclic ring systems. In general, such groups may be monocyclic or bicyclic and may contain, for example, 3 to 12 ring members, more usually 5 to 10 ring members. Examples of monocyclic groups are groups containing 3, 4, 5, 6, 7, and 8 ring members, more usually 3 to 7, and preferably 5 or 6 ring members. Examples of bicyclic groups are those containing 8, 9, 10, 11 and 12 ring members, and more usually 9 or 10 ring members.

The carbocyclic or heterocyclic groups can be aryl or heteroaryl groups having from 5 to 12 ring members, more usually from 5 to 10 ring members. The term “aryl” as used herein refers to a carbocyclic group having aromatic character and the term “heteroaryl” is used herein to denote a heterocyclic group having aromatic character. The terms “aryl” and “heteroaryl” embrace polycyclic (e.g. bicyclic) ring systems wherein one or more rings are non-aromatic, provided that at least one ring is aromatic. In such polycyclic systems, the group may be attached by the aromatic ring, or by a non-aromatic ring. The aryl or heteroaryl groups can be monocyclic or bicyclic groups and can be unsubstituted or substituted with one or more substituents, for example one or more groups R¹⁵ as defined herein.

The term “non-aromatic group” embraces unsaturated ring systems without aromatic character, partially saturated and fully saturated carbocyclic and heterocyclic ring systems. The terms “unsaturated” and “partially saturated” refer to rings wherein the ring structure(s) contains atoms sharing more than one valence bond i.e. the ring contains at least one multiple bond e.g. a C═C, C≡C or N═C bond. The terms “fully saturated” and “saturated” refer to rings where there are no multiple bonds between ring atoms. Saturated carbocyclic groups include cycloalkyl groups as defined below. Partially saturated carbocyclic groups include cycloalkenyl groups as defined below, for example cyclopentenyl, cycloheptenyl and cyclooctenyl. A further example of a cycloalkenyl group is cyclohexenyl.

Examples of heteroaryl groups are monocyclic and bicyclic groups containing from five to twelve ring members, and more usually from five to ten ring members. The heteroaryl group can be, for example, a five membered or six membered monocyclic ring or a bicyclic structure formed from fused five and six membered rings or two fused six membered rings or, by way of a further example, two fused five membered rings. Each ring may contain up to about four heteroatoms typically selected from nitrogen, sulphur and oxygen. Typically the heteroaryl ring will contain up to 4 heteroatoms, more typically up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general the number of basic nitrogen atoms present in the heteroaryl group, including any amino group substituents of the ring, will be less than five.

Examples of five membered heteroaryl groups include but are not limited to pyrrole, furan, thiophene, imidazole, furazan, oxazole, oxadiazole, oxatriazole, isoxazole, thiazole, isothiazole, pyrazole, triazole and tetrazole groups.

Examples of six membered heteroaryl groups include but are not limited to pyridine, pyrazine, pyridazine, pyrimidine and triazine.

A bicyclic heteroaryl group may be, for example, a group selected from:
a) a benzene ring fused to a 5- or 6-membered ring containing 1, 2 or 3 ring heteroatoms;
b) a pyridine ring fused to a 5- or 6-membered ring containing 1, 2 or 3 ring heteroatoms;
c) a pyrimidine ring fused to a 5- or 6-membered ring containing 1 or 2 ring heteroatoms;
d) a pyrrole ring fused to a a 5- or 6-membered ring containing 1, 2 or 3 ring heteroatoms;
e) a pyrazole ring fused to a a 5- or 6-membered ring containing 1 or 2 ring heteroatoms;
f) a pyrazine ring fused to a 5- or 6-membered ring containing 1 or 2 ring heteroatoms;
g) an imidazole ring fused to a 5- or 6-membered ring containing 1 or 2 ring heteroatoms;
h) an oxazole ring fused to a 5- or 6-membered ring containing 1 or 2 ring heteroatoms;
i) an isoxazole ring fused to a 5- or 6-membered ring containing 1 or 2 ring heteroatoms;
j) a thiazole ring fused to a 5- or 6-membered ring containing 1 or 2 ring heteroatoms;
k) an isothiazole ring fused to a 5- or 6-membered ring containing 1 or 2 ring heteroatoms;
l) a thiophene ring fused to a 5- or 6-membered ring containing 1, 2 or 3 ring heteroatoms;
m) a furan ring fused to a 5- or 6-membered ring containing 1, 2 or 3 ring heteroatoms;
n) a cyclohexyl ring fused to a 5- or 6-membered ring containing 1, 2 or 3 ring heteroatoms; and
o) a cyclopentyl ring fused to a 5- or 6-membered ring containing 1, 2 or 3 ring heteroatoms.

One sub-group of bicyclic heteroaryl groups consists of groups (a) to (e) and (g) to (o) above.

Particular examples of bicyclic heteroaryl groups containing a five membered ring fused to another five membered ring include but are not limited to imidazothiazole (e.g. imidazo[2,1-b]thiazole) and imidazoimidazole (e.g. imidazo[1,2-a]imidazole).

Particular examples of bicyclic heteroaryl groups containing a six membered ring fused to a five membered ring include but are not limited to benzfuran, benzthiophene, benzimidazole, benzoxazole, isobenzoxazole, benzisoxazole, benzthiazole, benzisothiazole, isobenzofuran, indole, isoindole, indolizine, indoline, isoindoline, purine (e.g., adenine, guanine), indazole, pyrazolopyrimidine (e.g. pyrazolo[1,5-a]pyrimidine), triazolopyrimidine (e.g. [1,2,4]triazolo[1,5-ajpyrimidine), benzodioxole and pyrazolopyridine (e.g. pyrazolo[1,5-a]pyridine) groups.

Particular examples of bicyclic heteroaryl groups containing two fused six membered rings include but are not limited to quinoline, isoquinoline, chroman, thiochroman, chromene, isochromene, chroman, isochroman, benzodioxan, quinolizine, benzoxazine, benzodiazine, pyridopyridine, quinoxaline, quinazoline, cinnoline, phthalazine, naphthyridine and pteridine groups.

One sub-group of heteroaryl groups comprises pyridyl, pyrrolyl, furanyl, thienyl, imidazolyl, oxazolyl, oxadiazolyl, oxatriazolyl, isoxazolyl, thiazolyl, isothiazolyl, pyrazolyl, pyrazinyl, pyridazinyl, pyrimidinyl, triazinyl, triazolyl, tetrazolyl, quinolinyl, isoquinolinyl, benzfuranyl, benzthienyl, chromanyl, thiochromanyl, benzimidazolyl, benzoxazolyl, benzisoxazole, benzthiazolyl and benzisothiazole, isobenzofuranyl, indolyl, isoindolyl, indolizinyl, indolinyl, isoindolinyl, purinyl (e.g., adenine, guanine), indazolyl, benzodioxolyl, chromenyl, isochromenyl, isochromanyl, benzodioxanyl, quinolizinyl, benzoxazinyl, benzodiazinyl, pyridopyridinyl, quinoxalinyl, quinazolinyl, cinnolinyl, phthalazinyl, naphthyridinyl and pteridinyl groups.

Examples of polycyclic aryl and heteroaryl groups containing an aromatic ring and a non-aromatic ring include tetrahydronaphthalene, tetrahydroisoquinoline, tetrahydroquinoline, dihydrobenzthiene, dihydrobenzfuran, 2,3-dihydro-benzo[1,4]dioxine, benzo[1,3]dioxole, 4,5,6,7-tetrahydrobenzofuran, indoline and indane groups.

Examples of carbocyclic aryl groups include phenyl, naphthyl, indenyl, and tetrahydronaphthyl groups.

Examples of non-aromatic heterocyclic groups include unsubstituted or substituted (by one or more groups R¹⁵) heterocyclic groups having from 3 to 12 ring members, typically 4 to 12 ring members, and more usually from 5 to 10 ring members. Such groups can be monocyclic or bicyclic, for example, and typically have from 1 to 5 heteroatom ring members (more usually 1, 2, 3 or 4 heteroatom ring members) typically selected from nitrogen, oxygen and sulphur.

When sulphur is present, it may, where the nature of the adjacent atoms and groups permits, exist as —S—, —S(O)— or —S(O)₂—.

The heterocylic groups can contain, for example, cyclic ether moieties (e.g. as in tetrahydrofuran and dioxane), cyclic thioether moieties (e.g. as in tetrahydrothiophene and dithiane), cyclic amine moieties (e.g. as in pyrrolidine), cyclic amide moieties (e.g. as in pyrrolidone), cyclic thioamides, cyclic thioesters, cyclic ester moieties (e.g. as in butyrolactone), cyclic sulphones (e.g. as in sulpholane and sulpholene), cyclic sulphoxides, cyclic sulphonamides and combinations thereof (e.g. morpholine and thiomorpholine and its S-oxide and S,S-dioxide). Further examples of heterocyclic groups are those containing a cyclic urea moiety (e.g. as in imidazolidin-2-one),

In one sub-set of heterocyclic groups, the heterocyclic groups contain cyclic ether moieties (e.g as in tetrahydrofuran and dioxane), cyclic thioether moieties (e.g. as in tetrahydrothiophene and dithiane), cyclic amine moieties (e.g. as in pyrrolidine), cyclic sulphones (e.g. as in sulpholane and sulpholene), cyclic sulphoxides, cyclic sulphonamides and combinations thereof (e.g. thiomorpholine).

Examples of monocyclic non-aromatic heterocyclic groups include 5-, 6- and 7-membered monocyclic heterocyclic groups. Particular examples include morpholine, piperidine (e.g. 1-piperidinyl, 2-piperidinyl, 3-piperidinyl and 4-piperidinyl), pyrrolidine (e.g. 1-pyrrolidinyl, 2-pyrrolidinyl and 3-pyrrolidinyl), pyrrolidone, pyran (2H-pyran or 4H-pyran), dihydrothiophene, dihydropyran, dihydrofuran, dihydrothiazole, tetrahydrofuran, tetrahydrothiophene, dioxane, tetrahydropyran (e.g. 4-tetrahydro pyranyl), imidazoline, imidazolidinone, oxazoline, thiazoline, 2-pyrazoline, pyrazolidine, piperazine, and N-alkyl piperazines such as N-methyl piperazine. Further examples include thiomorpholine and its S-oxide and S,S-dioxide (particularly thiomorpholine). Still further examples include azetidine, piperidone, piperazone, and N-alkyl piperidines such as N-methyl piperidine.

One preferred sub-set of non-aromatic heterocyclic groups consists of saturated groups such as azetidine, pyrrolidine, piperidine, morpholine, thiomorpholine, thiomorpholine S,S-dioxide, piperazine, N-alkyl piperazines, and N-alkyl piperidines.

Another sub-set of non-aromatic heterocyclic groups consists of pyrrolidine, piperidine, morpholine, thiomorpholine, thiomorpholine S,S-dioxide, piperazine and N-alkyl piperazines such as N-methyl piperazine.

One particular sub-set of heterocyclic groups consists of pyrrolidine, piperidine, morpholine and N-alkyl piperazines (e.g. N-methyl piperazine), and optionally thiomorpholine.

Examples of non-aromatic carbocyclic groups include cycloalkane groups such as cyclohexyl and cyclopentyl, cycloalkenyl groups such as cyclopentenyl, cyclohexenyl, cycloheptenyl and cyclooctenyl, as well as cyclohexadienyl, cyclooctatetraene, tetrahydronaphthenyl and decalinyl.

Preferred non-aromatic carbocyclic groups are monocyclic rings and most preferably saturated monocyclic rings.

Typical examples are three, four, five and six membered saturated carbocyclic rings, e.g. optionally substituted cyclopentyl and cyclohexyl rings.

One sub-set of non-aromatic carboyclic groups includes unsubstituted or substituted (by one or more groups R¹⁵) monocyclic groups and particularly saturated monocyclic groups, e.g. cycloalkyl groups. Examples of such cycloalkyl groups include cyclopropyl, cyclobutyl, cyclopentyl, cyclohexyl and cycloheptyl; more typically cyclopropyl, cyclobutyl, cyclopentyl and cyclohexyl, particularly cyclohexyl.

Further examples of non-aromatic cyclic groups include bridged ring systems such as bicycloalkanes and azabicycloalkanes although such bridged ring systems are generally less preferred. By “bridged ring systems” is meant ring systems in which two rings share more than two atoms, see for example Advanced Organic Chemistry, by Jerry March, 4^{th} Edition, Wiley Interscience, pages 131-133, 1992. Examples of bridged ring systems include bicyclo[2.2.1]heptane, aza-bicyclo[2.2.1]heptane, bicyclo[2.2.2]octane, aza-bicyclo[2.2.2]octane, bicyclo[3.2.1]octane and aza-bicyclo[3.2.1]octane. A particular example of a bridged ring system is the 1-aza-bicyclo[2.2.2]octan-3-yl group.

Where reference is made herein to carbocyclic and heterocyclic groups, the carbocyclic or heterocyclic ring can, unless the context indicates otherwise, be unsubstituted or substituted by one or more substituent groups R¹⁵ selected from halogen, hydroxy, trifluoromethyl, cyano, nitro, carboxy, amino, mono- or di-C₁₋₄ hydrocarbylamino, carbocyclic and heterocyclic groups having from 3 to 12 ring members; a group R^{a}—R^{b} wherein R^{a} is a bond, O, CO, X¹C(X²), C(X²)^{X}^{₁}, X¹C(X²)^{X}^{₁}, S, SO, SO₂, NR^{c}, SO₂NR ^{c̅} or NR ^{c̅}SO₂; and R^{b} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{C}, X¹C(X²), C(X²)X! or X¹C(X²)^{X}^{₁};
R^{o} is selected from hydrogen and C₁₋₄ hydrocarbyl; and
X¹ is O, S or NR^{o} and X² is ═O, ═S or ═NR^{o}.

Where the substituent group R¹⁵ comprises or includes a carbocyclic or heterocyclic group, the said carbocyclic or heterocyclic group may be unsubstituted or may itself be substituted with one or more further substituent groups R¹⁵. In one sub-group of compounds of the formula (I), such further substituent groups R¹⁵ may include carbocyclic or heterocyclic groups, which are typically not themselves further substituted. In another sub-group of compounds of the formula (I), the said further substituents do not include carbocyclic or heterocyclic groups but are otherwise selected from the groups listed above in the definition of R¹⁵.

The substituents R¹⁵ may be selected such that they contain no more than 20 non-hydrogen atoms, for example, no more than 15 non-hydrogen atoms, e.g. no more than 12, or 11, or 10, or 9, or 8, or 7, or 6, or 5 non-hydrogen atoms.

Where the carbocyclic and heterocyclic groups have a pair of substituents on the same or adjacent ring atoms, the two substituents may be linked so as to form a cyclic group. Thus, two adjacent groups R¹^{₅}, together with the carbon atoms or heteroatoms to which they are attached may form a 5-membered heteroaryl ring or a 5- or 6-membered non-aromatic carbocyclic or heterocyclic ring, wherein the said heteroaryl and heterocyclic groups contain up to 3 heteroatom ring members selected from N, O and S. For example, an adjacent pair of substituents on adjacent carbon atoms of a ring may be linked via one or more heteroatoms and optionally substituted alkylene groups to form a fused oxa-, dioxa-, aza-, diaza- or oxa-aza-cycloalkyl group.

Examples of such linked substituent groups include:

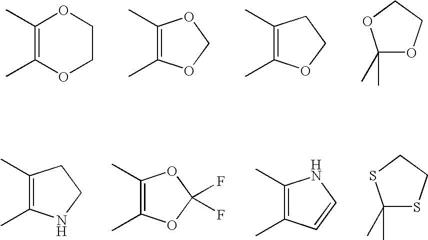

Examples of halogen substituents include fluorine, chlorine, bromine and iodine. Fluorine and chlorine are particularly preferred.

In the definition of the compounds of the formula (I) above and as used hereinafter, the term “hydrocarbyl” is a generic term encompassing aliphatic, alicyclic and aromatic groups having an all-carbon backbone and consisting of carbon and hydrogen atoms, except where otherwise stated.

In certain cases, as defined herein, one or more of the carbon atoms making up the carbon backbone may be replaced by a specified atom or group of atoms.

Examples of hydrocarbyl groups include alkyl, cycloalkyl, cycloalkenyl, carbocyclic aryl, alkenyl, alkynyl, cycloalkylalkyl, cycloalkenylalkyl, and carbocyclic aralkyl, aralkenyl and aralkynyl groups. Such groups can be unsubstituted or, where stated, substituted by one or more substituents as defined herein. The examples and preferences expressed below apply to each of the hydrocarbyl substituent groups or hydrocarbyl-containing substituent groups referred to in the various definitions of substituents for compounds of the formula (I) unless the context indicates otherwise.

The prefix “C_{x,y}” (where x and y are integers) as used herein refers to the number of carbon atoms in a given group. Thus, a C₁₋₄ hydrocarbyl group contains from 1 to 4 carbon atoms, and a C₃₋₆ cycloalkyl group contains from 3 to 6 carbon atoms, and so on.

Preferred non-aromatic hydrocarbyl groups are saturated groups such as alkyl and cycloalkyl groups.

Generally by way of example, the hydrocarbyl groups can have up to eight carbon atoms, unless the context requires otherwise. Within the sub-set of hydrocarbyl groups having 1 to 8 carbon atoms, particular examples are C₁₋₆ hydrocarbyl groups, such as C₁₋₄ hydrocarbyl groups (e.g. C₁₋₃ hydrocarbyl groups or C₁₋₂ hydrocarbyl groups or C₂₋₃ hydrocarbyl groups or C₂₋₄ hydrocarbyl groups), specific examples being any individual value or combination of values selected from C₁, C₂, C₃, C₄, C₅, C₆, C₇ and C₈ hydrocarbyl groups.

The term “alkyl” covers both straight chain and branched chain alkyl groups. Examples of alkyl groups include methyl, ethyl, propyl, isopropyl, n-butyl, isobutyl, tert-butyl, n-pentyl, 2-pentyl, 3-pentyl, 2-methyl butyl, 3-methyl butyl, and n-hexyl and its isomers. Within the sub-set of alkyl groups having 1 to 8 carbon atoms, particular examples are C₁₋₆ alkyl groups, such as C₁₋₄ alkyl groups (e.g. C₁₋₃ alkyl groups or C₁₋₂ alkyl groups or C₂₋₃ alkyl groups or C₂₋₄ alkyl groups).

Examples of cycloalkyl groups are those derived from cyclopropane, cyclobutane, cyclopentane, cyclohexane and cycloheptane. Within the sub-set of cycloalkyl groups the cycloalkyl group will have from 3 to 8 carbon atoms, particular examples being C₃₋₆ cycloalkyl groups.

Examples of alkenyl groups include, but are not limited to, ethenyl (vinyl), 1-propenyl, 2-propenyl (allyl), isopropenyl, butenyl, buta-1,4-dienyl, pentenyl, and hexenyl. Within the sub-set of alkenyl groups the alkenyl group will have 2 to 8 carbon atoms, particular examples being C₂₋₆ alkenyl groups, such as C₂₋₄ alkenyl groups.

Examples of cycloalkenyl groups include, but are not limited to, cyclopropenyl, cyclobutenyl, cyclopentenyl, cyclopentadienyl and cyclohexenyl. Within the sub-set of cycloalkenyl groups the cycloalkenyl groups have from 3 to 8 carbon atoms, and particular examples are C₃₋₆ cycloalkenyl groups.

Examples of alkynyl groups include, but are not limited to, ethynyl and 2-propynyl (propargyl) groups. Within the sub-set of alkynyl groups having 2 to 8 carbon atoms, particular examples are C₂₋₆ alkynyl groups, such as C₂₋₄ alkynyl groups.

Examples of carbocyclic aryl groups include substituted and unsubstituted phenyl groups.

Examples of cycloalkylalkyl, cycloalkenylalkyl, carbocyclic aralkyl, aralkenyl and aralkynyl groups include phenethyl, benzyl, styryl, phenylethynyl, cyclohexylmethyl, cyclopentylmethyl, cyclobutylmethyl, cyclopropylmethyl and cyclopentenylmethyl groups.

When present, and where stated, a hydrocarbyl group can be optionally substituted by one or more substituents selected from hydroxy, oxo, alkoxy, carboxy, halogen, cyano, nitro, amino, mono- or di-C₁₋₄ hydrocarbylamino, and monocyclic or bicyclic carbocyclic and heterocyclic groups having from 3 to 12 (typically 3 to 10 and more usually 5 to 10) ring members. Preferred substituents include halogen such as fluorine. Thus, for example, the substituted hydrocarbyl group can be a partially fluorinated or perfluorinated group such as difluoromethyl or trifluoromethyl. In one embodiment preferred substituents include monocyclic carbocyclic and heterocyclic groups having 3-7 ring members, more usually 3, 4, 5 or 6 ring members.

Where stated, one or more carbon atoms of a hydrocarbyl group may optionally be replaced by O, S, SO, SO₂, NR^{C}, X¹C(X²), C(X²)^{X}^{₁} or X¹C(X²)^{X}^{₁} (or a sub-group thereof) wherein X¹ and X² are as hereinbefore defined, provided that at least one carbon atom of the hydrocarbyl group remains. For example, 1, 2, 3 or 4 carbon atoms of the hydrocarbyl group may be replaced by one of the atoms or groups listed, and the replacing atoms or groups may be the same or different. In general, the number of linear or backbone carbon atoms replaced will correspond to the number of linear or backbone atoms in the group replacing them. Examples of groups in which one or more carbon atom of the hydrocarbyl group have been replaced by a replacement atom or group as defined above include ethers and thioethers (C replaced by O or S), amides, esters, thioamides and thioesters (C—C replaced by X¹C(X²) or C(X²)X¹), sulphones and sulphoxides (C replaced by SO or SO₂), amines (C replaced by NR ^{c̅}). Further examples include ureas, carbonates and carbamates (C—C—C replaced by X¹C(X²)^{X}^{₁}).

Where an amino group has two hydrocarbyl substituents, they may, together with the nitrogen atom to which they are attached, and optionally with another heteroatom such as nitrogen, sulphur, or oxygen, link to form a ring structure of 4 to 7 ring members, more usually 5 to 6 ring members.

The term “aza-cycloalkyl” as used herein refers to a cycloalkyl group in which one of the carbon ring members has been replaced by a nitrogen atom. Thus examples of aza-cycloalkyl groups include piperidine and pyrrolidine. The term “oxa-cycloalkyl” as used herein refers to a cycloalkyl group in which one of the carbon ring members has been replaced by an oxygen atom. Thus examples of oxa-cycloalkyl groups include tetrahydrofuran and tetrahydropyran. In an analogous manner, the terms “diaza-cycloalkyl”, “dioxa-cycloalkyl” and “aza-oxa-cycloalkyl” refer respectively to cycloalkyl groups in which two carbon ring members have been replaced by two nitrogen atoms, or by two oxygen atoms, or by one nitrogen atom and one oxygen atom. Thus, in an oxa-C₄₋₆ cycloalkyl group, there will be from 3 to 5 carbon ring members and an oxygen ring member. For example, an oxa-cyclohexyl group is a tetrahydropyranyl group.

The definition “R^{a}—R^{b}” as used herein, either with regard to substituents present on a carbocyclic or heterocyclic moiety, or with regard to other substituents present at other locations on the compounds of the formula (I), includes inter alia compounds wherein R^{a} is selected from a bond, O, CO, OC(O), SC(O), NR^{c}C(O), OC(S), SC(S), NR ^{c̅}C(S), OC(NR ^{c̅}), SC(NR^{c}), NRC^{c}(NR^{c}), C(O)O, C(O)S, C(O)NR ^{c̅}, C(S)O, C(S)S, C(S)NR ^{c̅}, C(NR^{o})0, C(NR^{o})S, C(NR^{o})NR^{C}, OC(O)O, SC(O)O, NRC^{c}(O)O, OC(S)O, SC(S)O, NR ^{c̅}C(S)O, 0C(NR^{o})0, SC(NR^{o})O, NR^{o}C(NR^{c})0, OC(O)S, SC(O)S, NR ^{c̅}C(O)S, OC(S)S, SC(S)S, NR ^{c̅}C(S)S, OC(NR^{o})S, SC(NR^{o})S, NR^{o}C(NR^{C})S, OC(O)NR ^{c̅}, SC(O)NR^{c}, NR^{c}C(O)NR^{c}, OC(S)NR ^{c̅}, SC(S)NR ^{c̅}, NR ^{c̅}C(S)NR ^{c̅}, OC(NR^{C})NR^{o}₅SC(NR^{o})NR^{C}, NR^{c}C(NR ^{c̅}NR ^{c̅}, S, SO, SO₂, NR ^{c̅}, SO₂NR ^{c̅} and NR ^{c̅}SO₂ wherein R^{o} is as hereinbefore defined.

The moiety R^{b} can be hydrogen or it can be a group selected from carbocyclic and heterocyclic groups having from 3 to 12 ring members (typically 3 to 10 and more usually from 5 to 10), and a C₁₋₈ hydrocarbyl group optionally substituted as hereinbefore defined. Examples of hydrocarbyl, carbocyclic and heterocyclic groups are as set out above.

When R^{a} is O and R^{b} is a C₁₋₈ hydrocarbyl group, R^{a} and R^{b} together form a hydrocarbyloxy group. Preferred hydrocarbyloxy groups include saturated hydrocarbyloxy such as alkoxy (e.g. C₁₋₆ alkoxy, more usually C₁₋₄ alkoxy such as ethoxy and methoxy, particularly methoxy), cycloalkoxy (e.g. C₃₋₆ cycloalkoxy such as cyclopropyloxy, cyclobutyloxy, cyclopentyloxy and cyclohexyloxy) and cycloalkyalkoxy (e.g. C₃₋₆ cycloalkyl-C₁₋₂ alkoxy such as cyclopropylmethoxy).

The hydrocarbyloxy groups can be substituted by various substituents as defined herein. For example, the alkoxy groups can be substituted by halogen (e.g. as in difluoromethoxy and trifluoromethoxy), hydroxy (e.g. as in hydroxyethoxy), C₁₋₂ alkoxy (e.g. as in methoxyethoxy), hydroxy-C₁₋₂ alkyl (as in hydroxyethoxyethoxy) or a cyclic group (e.g. a cycloalkyl group or non-aromatic heterocyclic group as hereinbefore defined). Examples of alkoxy groups bearing a non-aromatic heterocyclic group as a substituent are those in which the heterocyclic group is a saturated cyclic amine such as morpholine, piperidine, pyrrolidine, piperazine, C₁₋₄-alkyl-piperazines, C₃₋₇-cycloalkyl-piperazines, tetrahydropyran or tetrahydrofuran and the alkoxy group is a C₁₋₄ alkoxy group, more typically a C₁₋₃ alkoxy group such as methoxy, ethoxy or n-propoxy.

Alkoxy groups may be substituted by a monocyclic group such as pyrrolidine, piperidine, morpholine and piperazine and N-substituted derivatives thereof such as N-benzyl, N—C₁₋₄ acyl and N—C₁₋₄ alkoxycarbonyl. Particular examples include pyrrolidinoethoxy, piperidinoethoxy and piperazinoethoxy.

When R^{a} is a bond and R^{b} is a C₁₋₈ hydrocarbyl group, examples of hydrocarbyl groups R^{a}—R^{b} are as hereinbefore defined. The hydrocarbyl groups may be saturated groups such as cycloalkyl and alkyl and particular examples of such groups include methyl, ethyl and cyclopropyl. The hydrocarbyl (e.g. alkyl) groups can be substituted by various groups and atoms as defined herein. Examples of substituted alkyl groups include alkyl groups substituted by one or more halogen atoms such as fluorine and chlorine (particular examples including bromoethyl, chloroethyl and trifluoromethyl), or hydroxy (e.g. hydroxymethyl and hydroxyethyl), C₁₋₈ acyloxy (e.g. acetoxymethyl and benzyloxymethyl), amino and mono- and dialkylamino (e.g. aminoethyl, methylaminoethyl, dimethylaminomethyl, dimethylaminoethyl and tert-butylaminomethyl), alkoxy (e.g. C₁₋₂ alkoxy such as methoxy—as in methoxyethyl), and cyclic groups such as cycloalkyl groups, aryl groups, heteroaryl groups and non-aromatic heterocyclic groups as hereinbefore defined).

Particular examples of alkyl groups substituted by a cyclic group are those wherein the cyclic group is a saturated cyclic amine such as morpholine, piperidine, pyrrolidine, piperazine, Ĉ-alkyl-piperazines, C₃₋₇-cycloalkyl-piperazines, tetrahydropyran or tetrahydrofuran and the alkyl group is a C₁₋₄ alkyl group, more typically a C₁₋₃ alkyl group such as methyl, ethyl or n-propyl. Specific examples of alkyl groups substituted by a cyclic group include pyrrolidinomethyl, pyrrolidinopropyl, morpholinomethyl, morpholinoethyl, morpholinopropyl, piperidinylmethyl, piperazinomethyl and N-substituted forms thereof as defined herein.

Particular examples of alkyl groups substituted by aryl groups and heteroaryl groups include benzyl and pyridylmethyl groups.

When R^{a} is SO₂NR^{c}, R^{b} can be, for example, hydrogen or an optionally substituted C₁₋₈ hydrocarbyl group, or a carbocyclic or heterocyclic group. Examples of R^{a}—R^{b} where R^{a} is SO₂NR^{c} include aminosulphonyl, C₁₋₄ alkylaminosulphonyl and di-C₁₋₄ alkylaminosulphonyl groups, and sulphonamides formed from a cyclic amino group such as piperidine, morpholine, pyrrolidine, or an optionally N-substituted piperazine such as N-methyl piperazine.

Examples of groups R^{a}—R^{b} where R^{a} is SO₂ include alkylsulphonyl, heteroarylsulphonyl and arylsulphonyl groups, particularly monocyclic aryl and heteroaryl sulphonyl groups. Particular examples include methylsulphonyl, phenylsulphonyl and toluenesulphonyl.

When R^{a} is NR ^{c̅}, R^{b} can be, for example, hydrogen or an optionally substituted C₁₋₈ hydrocarbyl group, or a carbocyclic or heterocyclic group. Examples of R^{a}-R^{b} where R^{a} is NR^{c} include amino, C₁₋₄ alkylamino (e.g. methylamino, ethylamino, propylamino, isopropylamino, tert-butylamino), di-C₁₋₄ alkylamino (e.g. dimethylamino and diethylamino) and cycloalkylamino (e.g. cyclopropylamino, cyclopentylamino and cyclohexylamino).

### SPECIFIC EMBODIMENTS OF AND PREFERENCES FOR R¹ TO R¹⁵

In one embodiment, R¹ is 2,6-dichlorophenyl, R^{2a} and R^{2b} are both hydrogen and R³ is (i) a group:

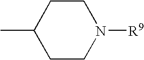

where R⁹ is selected from C(O)NR⁵R⁶; C(O)—R¹⁰ and 2-pyrimidinyl where R¹⁰ is a C₁₋₄ alkyl group optionally substituted by one or more substituents chosen from fluorine, chlorine, cyano and methoxy; and R¹¹ where R¹¹ is a C₁₋₄ alkyl group substituted by one or more substituents chosen from fluorine, chlorine and cyano.

In one sub-group of compounds within this embodiment, R⁹ is selected from C(O)NR⁵R⁶; C(O)—R¹⁰ where R¹⁰ is a C₁₋₄ alkyl group optionally substituted by one or more substituents chosen from fluorine, chlorine, cyano and methoxy; and R¹¹ where R¹¹ is a C₁₋₄ alkyl group substituted by one or more substituents chosen from fluorine, chlorine and cyano.

Within this embodiment, when R⁹ is C(O)NR⁵R⁶, the group NR⁵R⁶ can be, for example, dimethylamino and cyclic amines such as morpholine, piperidine, piperazine, N-methylpiperazine, pyrrolidine and thiazolidine. Particular heterocyclic rings include morpholinyl, 4-methylpiperazinyl and pyrrolidine

When R⁹ is C(O)—R¹⁰, particular examples of R¹⁰ include methyl, trifluoromethyl and methoxymethyl.

When R⁹ is a group R¹¹, examples of R¹¹ include substituted methyl groups and 2-substituted ethyl groups such as cyanomethyl, 2-cyanoethyl and 2-fluoroethyl.

In another embodiment of the invention, R¹ is 2,6-dichlorophenyl, R^{2a} and R^{2b} are both hydrogen and R³ is (ii) a group:

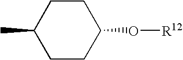

where R¹² is C₂₋₄ alkyl.

The C₂₋₄ alkyl group may be as set out in the General Preferences and Definitions section above. Thus, it can be a C₂-C₃ group or a C₂, C₃ or C₄ alkyl group. Particular C₂₋₄ alkyl groups are ethyl, i-propyl, n-butyl, i-butyl and tert-butyl groups; and more particular groups are i-propyl and i-butyl.

In another embodiment, R¹ is 2,6-dichlorophenyl, R^{2a} and R^{2b} are both hydrogen and R³ is (iii) a group:

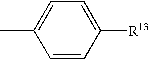

wherein R¹³ is selected from methylsulphonyl, 4-morpholino, 4-thiomorpholino, 1-piperidino, 1-methyl-4-piperazino and 1-pyrrolidino.

Particular groups R¹³ include 4-morpholino and 1-methyl-4-piperazino.

In another embodiment, R¹ is 2,6-dichlorophenyl, R^{2a} and R^{2b} are both hydrogen and R³ is (iv) a substituted 3-pyridyl or 4-pyridyl group of the formula

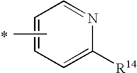

wherein the group R¹⁴ is meta oxpara with respect to the bond labelled with an asterisk and is selected from methyl, methylsulphonyl, 4-morpholino, Λ-thiomorpholino, 1-piperidino, 1-methyl-4-piperazino, 1-pyrrolidino, 4-piperidinyloxy, 1-C₁₋₄alkoxycarbonyl-piperidin-4-yloxy, 2-hydroxyethoxy and 2-methoxyethoxy.

More particularly, R¹⁴ is selected from methyl, methylsulphonyl, 4-morpholino, 1-methyl-4-piperazino, 4-piperidinyloxy, 1-C₁₋₄alkoxycarbonyl-piperidin-4-yloxy, 2-hydroxyethoxy and 2-methoxyethoxy.

In another embodiment, R¹ is 2,6-dichlorophenyl, R^{2a} and R^{2b} are both hydrogen and R³ is (v) a group selected from 2-pyrazinyl, 5-pyrimidinyl, cyclohexyl, 1,4-dioxa-spiro[4.5]decan-8-yl (4-cyclohexanone ethylene glycol ketal), A-methylsulphonylamino-cyclohexyl, tetrahydrothiopyran-4-yl, 1,1-dioxo-tetrahydrothiopyran-4-yl, tetrahydropyran-4-yl, 4,4-difluorocyclohexyl and 3,5-dimethylisoxazol-4-yl.

Within this embodiment, R³ may be selected from 2-pyrazinyl, 5-pyrimidinyl, cyclohexyl, 1,4-dioxa-spiro[4.5]decan-8-yl (4-cyclohexanone ethylene glycol ketal), 4-methylsulphonylamino-cyclohexyl, tetrahydrothiopyran-4-yl, 1,1-dioxo-tetrahydrothiopyran-4-yl and 3,5-dimethylisoxazol-4-yl.

In another embodiment, R¹ is (b) 2,6-difluorophenyl, R^{2a} and R^{2b} are both hydrogen and R³ is selected from:

(vi) 1-methyl-piperidin-3-yl; 4-(2-dimethylaminoethoxy)-cyclohexyl; and an N-substituted 4-piperidinyl group wherein the N-substituent is selected from cyanomethyl and cyanoethyl; and
(vii) a group:

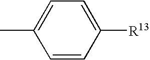

wherein R¹³ is an N-substituted 4-piperidinyl group wherein the N-substituent is C₁₋₄ alkoxycarbonyl, the C₁₋₄ alkoxy moiety in the C₁₋₄ alkoxycarbonyl group can be selected from methoxy, ethoxy, propyloxy, i-propyloxy, butyloxy, i-butyloxy and tert-butyloxy. A particular C₁₋₄ alkoxycarbonyl group is i-propyloxycarbonyl.

In one sub-group of compounds, R¹ is 2,6-difluorophenyl, R^{2a} and R^{2b} are both hydrogen and R³ is selected from 1-methyl-piperidin-3-yl; 4-(2-dimethylaminoethoxy)-cyclohexyl; and an N-substituted 4-piperidinyl group wherein the N-substituent is selected from cyanomethyl and cyanoethyl.

In another sub-group of compounds, R¹ is 2,6-difluorophenyl, R^{2a} and R^{2b} are both hydrogen and R³ is a group:

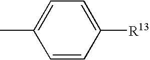

wherein R¹³ is selected from 4-morpholino, 4-thiomorpholino, 1-piperidino, 1-methyl-4-piperazino and 1-pyrrolidino.

Particular groups R¹³ include 4-morpholino and 1-methyl-4-piperazino.

In a further embodiment, R¹ is (c), a 2,3,6-trisubstituted phenyl group wherein the substituents for the phenyl group are selected from fluorine, chlorine, methyl and methoxy; and R^{2a} and R^{2b} are both hydrogen; and R³ is selected from (viii) 4-piperidinyl and 1-methyl-4-piperidinyl, (ix) tetrahydropyran-4-yl, groups (ii), (xi), (xii) and (xiii) as defined herein; and is further selected from: (x) a group:

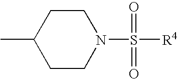

where R⁴ is C₁₋₄ alkyl.

Within this embodiment, R³ can be selected from (x) 4-piperidinyl and 1-methyl-4-piperidinyl, and groups (ii), (x), (xi), (xii) and (xiii) as defined herein.

Typically the 2,3,6-trisubstituted phenyl group has a fluorine, chlorine, methyl or methoxy group in the 2-position. The 2,3,6-trisubstituted phenyl group preferably has at least two substituents present that are chosen from fluorine and chlorine. A methoxy group, when present, is preferably located at the 2-position or 6-position, and more preferably the 2-position, of the phenyl group.

Particular examples of 2,3,6-trisubstituted phenyl groups are 2,3,6-trichlorophenyl, 2,3,6-trifluorophenyl, 2,3-difluoro-6-chlorophenyl, 2,3-difluoro-6-methoxyphenyl, 2,3-difluoro-6-methylphenyl, 3-chloro-2,6-difluorophenyl, 3-methyl-2,6-difluorophenyl, 2-chloro-3,6-difluorophenyl, 2-fluoro-3-methyl-6-chlorophenyl, 2-chloro-3-methyl-6-fluorophenyl, 2-chloro-3-methoxy-6-fluorophenyl and 2-methoxy-3-fluoro-6-chlorophenyl groups.

More particular examples are 2,3-difluoro-6-methoxyphenyl, 3-chloro-2,6-difluorophenyl, and 2-chloro-3,6-difluorophenyl groups.

In one sub-group of compounds wherein R¹ is a 2,3,6-trisubstituted phenyl group as defined herein, R³ is a 4-piperidinyl or 1-methyl-4-piperidinyl group.

In another sub-group of compounds wherein R¹ is a 2,3,6-trisubstituted phenyl group as defined herein, R³ is a group:

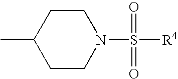

where R⁴ is a Ci₋₄ alkyl group as defined herein.

Examples of C₁₋₄ alkyl groups include methyl, ethyl, n-propyl, isopropyl, n-butyl, isobutyl and tert-butyl. One particular C₁₋₄ alkyl group is methyl.

In a further sub-group of compounds wherein R¹ is a 2,3,6-trisubstituted phenyl group as defined herein, R³ is a group:

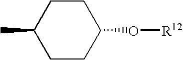

where R¹² is a C₂₋₄ alkyl group as defined herein. The C₂₋₄ alkyl group can be, for example, an ethyl, n-propyl, isopropyl, n-butyl, isobutyl or tert-butyl group Particular C₂₋₄ alkyl groups include ethyl, isopropyl and tert-butyl, and more particular C₁₋₄ alkyl groups R¹² are ethyl and isopropyl.

In another sub-group of compounds wherein R¹ is a 2,3,6-trisubstituted phenyl group as defined herein, R³ is a group:

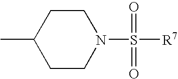

where R⁷ is as defined herein.

In one sub-group of compounds, R⁷ is unsubstituted hydrocarbyl other than C₁₋₄ alkyl. Examples of such hydrocarbyl groups include cyclopropyl and cyclopropylmethyl.

In another sub-group of compounds, R⁷ is substituted C₁₋₄ hydrocarbyl bearing one or more substituents chosen from fluorine, chlorine, hydroxy, methylsulphonyl, cyano, methoxy, NR⁵R⁶, and 4 to 7 membered saturated carbocyclic or heterocyclic rings containing up to two heteroatom ring members selected from O₅ N and S. Within this sub-group, particular examples include C₁₋₄ alkyl groups bearing one or more substituents (e.g. one, two or three substituents), and in particular substituted methyl and ethyl groups. More particularly, the C₁₋₄ hydrocarbyl group may be selected from trifluoromethyl, 2,2,2-trifluoroethyl, 2-methoxyethyl, 2-cyanoethyl, chloromethyl, 2-hydroxyethyl, tetrahydropyran-4-ylmethyl and groups of the formula —CH₂—CH₂—NR⁵R⁶. Particular examples of groups —CH₂—CH₂—NR⁵R⁶ include 2-(4-morpholinyl)ethyl, 2-(1-methyl-4-piperazinyl)ethyl, 2-(1-pyrrolidinyl)ethyl, 2-(3-thiazolidinyl)ethyl, 2-dimethylaminoethyl, 2-(N-methyl-N-methoxyamino)ethyl and 2-(N-methoxyamino)ethyl.

In another sub-group of compounds, R⁷ is a group NR⁵R⁶ where R⁵ and R⁶ are selected from hydrogen and C₁₋₄ alkyl, C₁₋₂ alkoxy and C₁₋₂ alkoxy-C₁₋₄ alkyl, provided that no more than one of R⁵ and R⁶ is C₁₋₂ alkoxy, or NR⁵R⁶ forms a five or six membered saturated heterocyclic ring containing one or two heteroatom ring members selected from O, N and S, the heterocyclic ring being optionally substituted by one or more methyl groups. Particular non-cyclic groups NR⁵R⁶ include amino, methylamino, ethylamino, dimethylamino, diethylamino, methoxyamino and N-methyl-N-methoxyamino, one preferred group being dimethylamino. Particular cyclic groups NR⁵R⁶ include morpholine, piperidine, piperazine, N-methylpiperazine, pyrrolidine and thiazolidine.

In another sub-group of compounds R⁷ is a five or six membered heteroaryl group containing one or two heteroatom ring members selected from N, S and O and being optionally substituted by methyl, methoxy, fluorine, chlorine, or a group NR⁵R⁶. Examples of five and six membered heteroaryl groups include imidazole, pyrazole and pyridyl, and particular examples of substituents include methyl and NR⁵R⁶.

In another sub-group of compounds, R⁷ is a phenyl group optionally substituted by methyl, methoxy, fluorine, chlorine, cyano or a group NR⁵R⁶ and particular examples of such groups include 4-fluorophenyl, 4-methoxyphenyl and 4-cyanophenyl.

In another sub-group of compounds, R⁷ is C₃₋₆ cycloalkyl; and examples of cycloalkyl groups are cyclopropyl, cyclobutyl, cyclopentyl and cyclohexyl groups; particular examples being cyclopropyl and cyclohexyl.

In a further sub-group of compounds, R⁷ is a five or six membered saturated heterocyclic ring containing one or two heteroatom ring members selected from O, N and S, the heterocyclic ring being optionally substituted by one or more methyl groups. The five or six membered saturated ring may be selected from, for example, morpholine, piperidine, piperazine, N-methylpiperazine, pyrrolidine and thiazolidine, with one particular example being morpholine.

In another sub-group of compounds wherein R¹ is a 2,3,6-trisubstituted phenyl group as defined herein, R³ is (xii) a group:

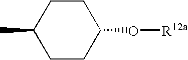

where R^{12a} is as defined herein.

In one sub-group of compounds, R¹^{₂ₐ} is C₁₋₄ alkyl substituted by one or more substituents chosen from fluorine, chlorine, C₃₋₆ cycloalkyl; oxa-C₄₋₆ cycloalkyl; cyano, and methoxy.

In another sub-group of compounds, R^{12a} is C₁₋₄ alkyl substituted by one or more substituents chosen from fluorine, C₃₋₆ cycloalkyl; oxa-C₄₋₆ cycloalkyl; cyano, and methoxy.

Examples of substituted alkyl groups are substituted methyl and substituted ethyl (e.g. 1-ethyl and 2-ethyl, preferably 2-ethyl) groups.

When R^{12a} is substituted methyl, particular examples include methoxymethyl, cyclopropylmethyl and tetrahydropyranylmethyl. A preferred R^{12a} is substituted methyl, in particular methoxymethyl.

When R^{12a} is substituted ethyl, particular examples include 2-dimethylaminoethyl, 2-methoxyethyl, and 2-(4-morpholino)ethyl groups.

In another embodiment, R¹ is (e) a group R^{1a}, R^{2a} and R^{2b} are both hydrogen, and

R³ is (xiü), a group

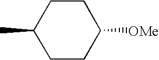

In this embodiment, R^{1a} is selected from cyclopropyl-cyano-methyl; furyl; benzoisoxazolyl; methylisoxazolyl; 2-monosubstituted phenyl and 2,6-disubstituted phenyl wherein the substituents on the phenyl moiety are selected from methoxy, ethoxy, fluorine, chlorine, and difluoromethoxy; provided that R^{1a} is not 2,6-difluorophenyl or 2,6-dichlorophenyl.

In one sub-group of compounds, R^{1a} is selected from furyl; benzoisoxazolyl; methylisoxazolyl; 2-monosubstituted phenyl and 2,6-disubstituted phenyl wherein the substituents on the phenyl moiety are selected from methoxy, ethoxy, fluorine, chlorine, and difluoromethoxy; provided that R^{1a} is not 2,6-difluorophenyl or 2,6-dichlorophenyl.

In another sub-groups of compounds, R^{1a} is selected from 2-monosubstituted phenyl and 2,6-disubstituted phenyl wherein the substituents on the phenyl moiety are selected from methoxy, ethoxy, fluorine, chlorine, and difluoromethoxy; provided that R^{1a} is not 2,6-difluorophenyl or 2,6-dichlorophenyl. Within this sub-group, particular examples of mono-substituted and di-substituted phenyl groups include 2-fluoro-6-methoxyphenyl, 2-fluoro-6-chlorophenyl, 2-difluoromethoxyphenyl and 2-chloro-6-methoxyphenyl.

In a further sub-group of compounds, R^{1a} is selected from furyl; benzoisoxazolyl and methylisoxazolyl.

In another sub-group of compounds, R^{1a} is cyclopropyl-cyano-methyl.

In another embodiment, R¹ is (f) a group R^{1b}, R^{2a} and R^{2b} are both hydrogen, and R³ is (xiv) a methyl group.

In another embodiment, R¹ is (g) a group R^{1c}, R^{2a} and R^{2b} are both hydrogen, and R³ is (xv), a group

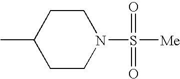

Within this embodiment, R^{1c} is selected from; benzoisoxazoyl; five membered heteroaryl rings containing one or two heteroatoms selected from O and N and six-membered heteroaryl rings containing one or two nitrogen heteroatom ring members, the heteroaryl rings in each case being optionally substituted by methyl, fluorine, chlorine or trifluoromethyl; and phenyl substituted by one, two or three substituents selected from bromine, chlorine, fluorine, methyl, trifluoromethyl, ethoxy, methoxy, methoxyethoxy, methoxymethyl, dimethylaminomethyl and difluoromethoxy; provided that R^{1a} is not 2,6-difluorophenyl; In one sub-group of compounds, R^{1c} is selected from benzoisoxazolyl; five membered heteroaryl rings containing one or two heteroatoms selected from O and N, the heteroaryl ring being optionally substituted by methyl, fluorine, chlorine or trifluoromethyl; and phenyl substituted by one, two or three substituents selected from bromine, chlorine, fluorine, methyl, trifluoromethyl, ethoxy, methoxy, methoxyethoxy and difluoromethoxy; provided that R^{1a} is not 2,6-difluorophenyl.

In another sub-group, R^{1c} is selected from benzoisoxazolyl and five membered heteroaryl rings containing one or two heteroatoms selected from O and N, wherein the heteroaryl ring is optionally substituted by methyl, fluorine, chlorine or trifluoromethyl. Examples of five membered heteroaryl rings include isoxazole, furyl and pyrazole rings, which rings may bear one or more substituents selected from, for example, methyl, chlorine and trifluoromethyl.

In another sub-group, R^{1c} is phenyl substituted by one, two or three substituents selected from bromine, chlorine, fluorine, methyl, trifluoromethyl, ethoxy, methoxy, methoxyethoxy, methoxymethyl, dimethylaminomethyl and difluoromethoxy; provided that R^{1a} is not 2,6-difluorophenyl. Within this subs group, R^{1c} may be, for example, phenyl substituted by one, two or three substituents selected from bromine, chlorine, fluorine, methyl, trifluoromethyl, ethoxy, methoxy, methoxyethoxy and difluoromethoxy; provided that R^{1a} is not 2,6-difluorophenyl. Examples of substituted phenyl groups include 2-monosubstituted, 3-monosubstituted, 4-monosubstituted, 2,3 disubstituted, 2,4-disubstituted, 2,5 disubstituted or 2,6 disubstituted, 2,3,5-trisubstituted, 2,4,5-trisubstituted and 2,3,6-trisubstituted phenyl groups; and more particularly 2-monosubstituted, 2,3-disubstituted, 2,6-disubstituted, and 2,3,6-trisubstituted phenyl groups. Particular examples of substituted phenyl groups include 2-ethoxyphenyl, 2-trifluoromethoxyphenyl, 2-fluoro-6-trifluoromethylphenyl, 2,6-dichlorophenyl, 2-chloro-6-methylphenyl, 2-fluoro-6-ethoxyphenyl, 2,6-dimethylphenyl, 2-methoxy-3-fluorophenyl, 2-fluoro-6-methoxyphenyl, 2-fluoro-3-methylphenyl, 2-chloro-6-bromophenyl, 2,3,6-trifluorophenyl, 2-chloro-3,6-difluorophenyl, 2-chloro-3-methyl-6-fluorophenyl, 2-fluoro-3-methyl-6-chlorophenyl, 2,3-difluoro-6-methoxyphenyl, 2,6-difluoro-3-chlorophenyl, 2-methoxy-3,6-dichlorophenyl, 2-methoxy-6-methylphenyl, 2,6-difluoro-3-methylphenyl and 2-chloro-3-methoxy-6-fluorophenyl. Further examples include 2-chloro-6-dimethylaminomethylphenyl and 2-choro-6-methoxymethylphenyl groups. Within this sub-group of compounds, in one particular group, the substituted phenyl group is 2,6-dichlorophenyl and in another particular group, the substituted phenyl group is other than 2,6-dichlorophenyl and/or other than a 2,3,6-trisubstituted phenyl group.

In another embodiment, R¹ is (j), 2,6-difluorophenylamino, R^{2a} and R^{2b} are both hydrogen; and R³ is methyl.

In a further embodiment, R¹ is 2,6-dichlorophenyl, R³ is a 4-piperidine group and either (k) R^{2a} is methyl and R^{2b} is hydrogen, or (1) R^{2a} is hydrogen and R^{2b} is methyl.

In another embodiment of the invention, R¹ is (d), a group R⁰, where R⁰ is a carbocyclic or heterocyclic group having from 3 to 12 ring members; or a C₁₋₈ hydrocarbyl group optionally substituted by one or more substituents selected from fluorine, hydroxy, cyano; C₁₋₄ hydrocarbyloxy, amino, mono- or di-C₁₋₄ hydrocarbylamino, and carbocyclic or heterocyclic groups having from 3 to 12 ring members, and wherein 1 or 2 of the carbon atoms of the hydrocarbyl group may optionally be replaced by an atom or group selected from O, S, NH, SO, SO₂; and R³ is selected from:
(xi) a group:

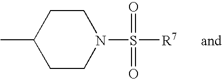

(xii) a group:

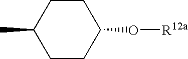

where R⁷ and R^{12a} are as defined herein.

In one group of compounds within this embodiment, R³ is a group:

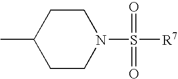

where R⁷ and its examples and preferences are as defined herein.

Thus, for example, in one sub-group of compounds, R⁷ is unsubstituted hydrocarbyl other than C₁₋₄ alkyl. Examples of such hydrocarbyl groups include cyclopropyl and cyclopropylmethyl.

In another sub-group of compounds, R⁷ is substituted C₁₋₄ hydrocarbyl bearing one or more substituents chosen from fluorine, chlorine, hydroxy, methylsulphonyl, cyano, methoxy, NR⁵R⁶, and 4 to 7 membered saturated carbocyclic or heterocyclic rings containing up to two heteroatom ring members selected from O, N and S. Within this sub-group, particular examples include C₁₋₄ alkyl groups bearing one or more substituents (e.g. one, two or three substituents), and in particular substituted methyl and ethyl groups. More particularly, the C₁₋₄ hydrocarbyl group may be selected from trifluoromethyl, 2,2,2-trifluoroethyl, 2-methoxyethyl, 2-cyanoethyl, chloromethyl, 2-hydroxyethyl, tetrahydropyran-4-ylmethyl and groups of the formula —CH₂—CH₂—NR⁵R⁶. Particular examples of groups —CH₂—CH₂—NR⁵R⁶ include 2-(4-morpholinyl)ethyl, 2-(1-methyl-4-piperazinyl)ethyl, 2-(1-pyrrolidinyl)ethyl, 2-(3-thiazolidinyl)ethyl, 2-dimethylaminoethyl, 2-(N-methyl-N-methoxyamino)ethyl and 2-(N-methoxyamino)ethyl.

In another sub-group of compounds, R⁷ is a group NR⁵R⁶ where R⁵ and R⁶ are selected from hydrogen and Ci₋₄ alkyl, C₁₋₂ alkoxy and C₁₋₂ alkoxy-C₁₋₄ alkyl, provided that no more than one of R⁵ and R⁶ is C₁₋₂ alkoxy, or NR⁵R⁶ forms a five or six membered saturated heterocyclic ring containing one or two heteroatom ring members selected from O, N and S₃ the heterocyclic ring being optionally substituted by one or more methyl groups. Particular non-cyclic groups NR⁵R⁶ include amino, methylamino, ethylamino, dimethylamino, diethylamino, methoxyamino and N-methyl-N-methoxyamino; one preferred group being dimethylamino. Particular cyclic groups NR⁵R⁶ include morpholine, piperidine, piperazine, N-methylpiperazine, pyrrolidine and thiazolidine.

In another sub-group of compounds R⁷ is a five or six membered heteroaryl group containing one or two heteroatom ring members selected from N, S and O and being optionally substituted by methyl, methoxy, fluorine, chlorine, or a group NR⁵R⁶. Examples of five and six membered heteroaryl groups include imidazole, prazole and pyridyl, and particular examples of substituents include methyl and NR⁵R⁶.

In another sub-group of compounds, R⁷ is a phenyl group optionally substituted by methyl, methoxy, fluorine, chlorine, cyano or a group NR⁵R⁵ and particular examples of such groups include 4-fluorophenyl, 4-methoxyphenyl and 4-cyanophenyl.

In another sub-group of compounds, R⁷ is C₃₋₆ cycloalkyl; and examples of cycloalkyl groups are cyclopropyl, cyclobutyl, cyclopentyl and cyclohexyl groups; particular examples being cyclopropyl and cyclohexyl.

In a further sub-group of compounds, R⁷ is a five or six membered saturated heterocyclic ring containing one or two heteroatom ring members selected from O₅ N and S, the heterocyclic ring being optionally substituted by one or more methyl groups. The five or six membered saturated ring may be selected from, for example, morpholine, piperidine, piperazine, N-methylpiperazine, pyrrolidine and thiazolidine, with one particular example being morpholine.

In another group of compounds wherein R¹ is R⁰, R³ is a group:

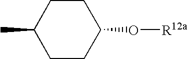

where R^{12a} and its preferences and examples are as defined herein.

In one sub-group of compounds, R^{12a} is C₁₋₄ alkyl substituted by one or more substituents chosen from fluorine, chlorine, C₃₋₆ cycloalkyl; OXa-C₄₋₆ cycloalkyl; cyano, and methoxy.

In another sub-group of compounds, R^{12a} is C₁₋₄ alkyl substituted by one or more substituents chosen from fluorine, C₃₋₆ cycloalkyl; oxa-C₄₋₆ cycloalkyl; cyano, and methoxy.

Examples of substituted alkyl groups are substituted methyl and substituted ethyl (e.g. 1-ethyl and 2-ethyl, preferably 2-ethyl) groups.

When R^{12a} is substituted methyl, particular examples include methoxymethyl, cyclopropylmethyl and tetrahydropyranylmethyl. A preferred group R^{12a} is substituted methyl, in particular methoxymethyl.

When R^{12a} is substituted ethyl, particular examples include 2-dimethylaminoethyl, 2-methoxyethyl, and 2-(4-morpholino)ethyl groups.

In the foregoing embodiments, examples, groups and sub-groups in which R¹ is R⁰, examples of carbocyclic or heterocyclic groups R⁰ having from 3 to 12 ring members; and optionally substituted C₁₋₈ hydrocarbyl groups are as set out above in the General Preferences and Definitions section.

More particularly, in one embodiment, R⁰ is an aryl or heteroaryl group.

When R⁰ is a heteroaryl group, particular heteroaryl groups include monocyclic heteroaryl groups containing up to three heteroatom ring members selected from O, S and N, and bicyclic heteroaryl groups containing up to 2 heteroatom ring members selected from O, S and N and wherein both rings are aromatic.

Examples of such groups include furanyl (e.g. 2-furanyl or 3-furanyl), indolyl (e.g. 3-indolyl, 6-indolyl), 2,3-dihydro-benzo[1,4]dioxinyl (e.g. 2,3-dihydro-benzo[1,4]dioxin-5-yl), pyrazolyl (e.g. pyrazole-5-yl), pyrazolo[1,5-a]pyridinyl (e.g. pyrazolo[1,5-a]pyridine-3-yl), oxazolyl (e.g.), isoxazolyl (e.g. isoxazol-4-yl), pyridyl (e.g. 2-pyridyl, 3-pyridyl, 4-pyridyl), quinolinyl (e.g. 2-quinolinyl), pyrrolyl (e.g. 3-pyrrolyl), imidazolyl and thienyl (e.g. 2-thienyl, 3-thienyl).

One sub-group of heteroaryl groups R⁰ consists of furanyl (e.g. 2-furanyl or 3-furanyl), indolyl, oxazolyl, isoxazolyl, pyridyl, quinolinyl, pyrrolyl, imidazolyl and thienyl.

A preferred sub-set of R⁰ heteroaryl groups includes 2-furanyl, 3-furanyl, pyrrolyl, imidazolyl and thienyl.

Preferred aryl groups R⁰ are phenyl groups.

The group R⁰ can be an unsubstituted or substituted carbocylic or heterocyclic group in which one or more substituents can be selected from the group R¹⁵ as hereinbefore defined. In one embodiment, the substituents on R⁰ may be selected from the group R^{15a} consisting of halogen, hydroxy, trifluoromethyl, cyano, nitro, carboxy, a group R^{a}-R^{b} wherein R^{a} is a bond, O, CO, X³C(X⁴), C(X⁴)X³, X³C(X⁴)X³, S, SO, or SO₂, and R^{b} is selected from hydrogen and a C₁₋₈ hydrocarbyl group optionally substituted by one or more substituents selected from hydroxy, oxo, halogen, cyano, nitro, carboxy and monocyclic non-aromatic carbocyclic or heterocyclic groups having from 3 to 6 ring members; wherein one or more carbon atoms of the C₁₋₈ hydrocarbyl group may optionally be replaced by O, S, SO₅ SO₂, X³C(X⁴), C(X⁴)X³ or X³C(X⁴)X³; X³ is O or S; and X⁴ is =0 or ═S.

Where the carbocyclic and heterocyclic groups have a pair of substituents on the same or adjacent ring atoms, the two substituents may be linked so as to form a cyclic group. Thus, two adjacent groups R¹⁵, together with the carbon atom(s) or heteroatom(s) to which they are attached may form a 5-membered heteroaryl ring or a 5- or 6-membered non-aromatic carbocyclic or heterocyclic ring, wherein the said heteroaryl and heterocyclic groups contain up to 3 heteroatom ring members selected from N, O and S. In particular the two adjacent groups R¹⁵, together with the carbon atoms or heteroatoms to which they are attached, may form a 6-membered non-aromatic heterocyclic ring, containing up to 3, in particular 2, heteroatom ring members selected from N, O and S. More particularly the two adjacent groups R¹⁵ may form a 6-membered non-aromatic heterocyclic ring, containing 2 heteroatom ring members selected from N, or O, such as dioxan e.g. [1,4 dioxan]. In one embodiment R¹ is a carbocyclic group e.g. phenyl having a pair of substituents on adjacent ring atoms linked so as to form a cyclic group e.g. to form 2,3-dihydro-benzo[1,4]dioxine.

More particularly, the substituents on R⁰ may be selected from halogen, hydroxy, trifluoromethyl, a group R^{a}—R^{b} wherein R^{a} is a bond or O, and R^{b} is selected from hydrogen and a C₁₋₄ hydrocarbyl group optionally substituted by one or more substituents selected from hydroxyl, halogen (preferably fluorine) and 5 and 6 membered saturated carbocyclic and heterocyclic groups (for example groups containing up to two heteroatoms selected from O, S and N, such as unsubstituted piperidine, pyrrolidine, morpholino, piperazino and N-methyl piperazino).

The group R⁰ may be substituted by more than one substituent. Thus, for example, there may be 1 or 2 or 3 or 4 substituents. In one embodiment, where R⁰ is a six membered ring (e.g. a carbocyclic ring such as a phenyl ring), there may be one, two or three substituents and these may be located at the 2-, 3-, 4- or 6-positions around the ring.

In one preferred group of compounds, R⁰ is a substituted phenyl group. By way of example, a substituted phenyl group R⁰ may be 2-monosubstituted, 3-monosubstituted, 2,6-disubstituted, 2,3-disubstituted, 2,4-disubstituted 2,5-disubstituted, 2,3,6-trisubstituted or 2,4,6-trisubstituted.

More particularly, in one particular group of compounds, a phenyl group R⁰ may be monosubstituted at the 2-position or disubstituted at positions 2- and 6- with substituents selected from fluorine, chlorine and R^{a}—R^{b}, where R^{a} is O and R^{b} is C₁₋₄ alkyl (e.g. methyl or ethyl). In one preferred embodiment, the phenyl group is 2,6-disubstituted, wherein the substituents are selected from, for example, fluorine, chlorine, methyl, ethyl, trifluoromethyl, difluoromethoxy and methoxy, and particular examples of such substituted phenyl groups include 2-fluoro-6-trifluoromethylphenyl, 2,6-dichlorophenyl, 2,6-difluorophenyl, 2-chloro-6-methylphenyl, 2-fluoro-6-ethoxyphenyl, 2,6-dimethylphenyl, 2-methoxy-3-fluorophenyl, 2-fluoro-6-methoxyphenyl, 2-fluoro-3-methylphenyl and 2-chloro-6-bromophenyl. One particularly preferred 2,6-disubstituted group is 2,6-dichlorophenyl.

In another particular group of compounds, a phenyl group R⁰ may be trisubstituted at the 2-, 3- and 6-positions.

Typically the 2,3,6-trisubstituted phenyl group R^{o} has a fluorine, chlorine, methyl or methoxy group in the 2-position. The 2,3,6-trisubstituted phenyl group preferably has at least two substituents present that are chosen from fluorine and chlorine. A methoxy group, when present, is preferably located at the 2-position or 6-position, and more preferably the 2-position, of the phenyl group.

Particular examples of 2,3,6-trisubstituted phenyl groups R⁰ are 2,3,6-trichlorophenyl, 2,3,6-trifluorophenyl, 2,3-difluoro-6-chlorophenyl, 2,3-difluoro-6-methoxyphenyl, 2,3-difluoro-6-methylphenyl, 3-chloro-2,6-difluorophenyl, 3-methyl-2,6-difluorophenyl, 2-chloro-3,6-difluorophenyl, 2-fluoro-3-methyl-6-chlorophenyl, 2-chloro-3-methyl-6-fluorophenyl, 2-chloro-3-methoxy-6-fluorophenyl and 2-methoxy-3-fluoro-6-chlorophenyl groups.

More particular examples are 2,3-difluoro-6-methoxyphenyl, 3-chloro-2,6-difluorophenyl, and 2-chloro-3,6-difluorophenyl groups.

Particular examples of non-aromatic groups R⁰ include unsubstituted or substituted (by one or more groups R¹⁵) monocyclic cycloalkyl groups. Examples of such cycloalkyl groups include cyclopropyl, cyclobutyl, cyclopentyl, cyclohexyl and cycloheptyl; more typically cyclopropyl, cyclobutyl, cyclopentyl and cyclohexyl, particularly cyclohexyl.

Further examples of non-aromatic groups R⁰ include unsubstituted or substituted (by one or more groups R¹⁵) heterocyclic groups having from 3 to 12 ring members, typically 4 to 12 ring members, and more usually from 5 to 10 ring members. Such groups can be monocyclic or bicyclic, for example, and typically have from 1 to 5 heteroatom ring members (more usually 1, 2, 3 or 4 heteroatom ring members) typically selected from nitrogen, oxygen and sulphur.

When sulphur is present, it may, where the nature of the adjacent atoms and groups permits, exist as —S—, —S(O)— or —S(O)₂—.

The heterocylic groups can contain, for example, cyclic ether moieties (e.g as in tetrahydrofuran and dioxane), cyclic thioether moieties (e.g. as in tetrahydrothiophene and dithiane), cyclic amine moieties (e.g. as in pyrrolidine), cyclic amides (e.g. as in pyrrolidone), cyclic esters (e.g. as in butyrolactone), cyclic thioamides and thioesters, cyclic sulphones (e.g. as in sulpholane and sulpholene), cyclic sulphoxides, cyclic sulphonamides and combinations thereof (e.g. morpholine and thiomorpholine and its S-oxide and S,S-dioxide).

In one sub-set of heterocyclic groups R⁰, the heterocyclic groups contain cyclic ether moieties (e.g. as in tetrahydrofuran and dioxane), cyclic thioether moieties (e.g. as in tetrahydrothiophene and dithiane), cyclic amine moieties (e.g. as in pyrrolidine), cyclic sulphones (e.g. as in sulpholane and sulpholene), cyclic sulphoxides, cyclic sulphonamides and combinations thereof (e.g. thiomorpholine).

Examples of monocyclic non-aromatic heterocyclic groups R⁰ include 5-, 6- and 7-membered monocyclic heterocyclic groups such as morpholine, piperidine (e.g. 1-piperidinyl, 2-piperidinyl 3-piperidinyl and 4-piperidinyl), pyrrolidine (e.g. 1-pyrrolidinyl, 2-pyrrolidinyl and 3-pyrrolidinyl), pyrrolidone, pyran (2H-pyran or 4H-pyran), dihydrothiophene, dihydropyran, dihydrofuran, dihydrothiazole, tetrahydrofuran, tetrahydrothiophene, dioxane, tetrahydropyran (e.g. 4-tetrahydro pyranyl), imidazoline, imidazolidinone, oxazoline, thiazoline, 2-pyrazoline, pyrazolidine, piperazine, and N-alkyl piperazines such as N-methyl piperazine. Further examples include thiomorpholine and its S-oxide and S,S-dioxide (particularly thiomorpholine). Still further examples include N-alkyl piperidines such as N-methyl piperidine.

One sub-group of non-aromatic heterocyclic groups R⁰ includes unsubstituted or substituted (by one or more groups R¹^{₅}) 5-, 6- and 7-membered monocyclic heterocyclic groups such as morpholine, piperidine (e.g. 1-piperidinyl, 2-piperidinyl 3-piperidinyl and 4-piperidinyl), pyrrolidine (e.g. 1-pyrrolidinyl, 2-pyrrolidinyl and 3-pyrrolidinyl), pyrrolidone, piperazine, and N-alkyl piperazines such as N-methyl piperazine, wherein a particular sub-set consists of pyrrolidine, piperidine, morpholine, thiomorpholine and N-methyl piperazine.

In general, preferred non-aromatic heterocyclic groups include pyrrolidine, piperidine, morpholine, thiomorpholine, thiomorpholine S,S-dioxide, piperazine, N-alkyl piperazines, and N-alkyl piperidines.

Another particular sub-set of heterocyclic groups consists of pyrrolidine, piperidine, morpholine and N-alkyl piperazines, and optionally, N-methyl piperazine and thiomorpholine.

When R⁰ is a C₁₋₈ hydrocarbyl group substituted by a carbocyclic or heterocyclic group, the carbocyclic and heterocyclic groups can be aromatic or non-aromatic and can be selected from the examples of such groups set out hereinabove. The substituted hydrocarbyl group is typically a saturated C₁₋₄ hydrocarbyl group such as an alkyl group, preferably a CH₂ or CH₂CH₂ group. Where the substituted hydrocarbyl group is a C₂₋₄ hydrocarbyl group, one of the carbon atoms and its associated hydrogen atoms may be replaced by a sulphonyl group, for example as in the moiety SO₂CH₂.

When the carbocyclic or heterocylic group attached to the a C₁₋₈ hydrocarbyl group is aromatic, examples of such groups include monocyclic aryl groups and monocyclic heteroaryl groups containing up to four heteroatom ring members selected from O, S and N, and bicyclic heteroaryl groups containing up to 2 heteroatom ring members selected from O, S and N and wherein both rings are aromatic.

Examples of such groups are set out in the “General Preferences and Definitions” section above.

Particular examples of such groups include furanyl (e.g. 2-furanyl or 3-furanyl), indolyl, oxazolyl, isoxazolyl, pyridyl, quinolinyl, pyrrolyl, imidazolyl and thienyl.

Particular examples of aryl and heteroaryl groups as substituents for a C₁₋₈ hydrocarbyl group include phenyl, imidazolyl, tetrazolyl, triazolyl, indolyl, 2-furanyl, 3-furanyl, pyrrolyl and thienyl. Such groups may be substituted by one or more substituents R¹ or R^{15a} as defined herein.

When R⁰ is a C₁₋₈ hydrocarbyl group substituted by a non-aromatic carbocyclic or heterocyclic group, the non-aromatic or heterocyclic group may be a group selected from the lists of such groups set out hereinabove. For example, the non-aromatic group can be a monocyclic group having from 4 to 7 ring members, e.g. 5 to 7 ring members, and typically containing from 0 to 3, more typically 0, 1 or 2, heteroatom ring members selected from O, S and N. When the cyclic group is a carbocyclic group, it may additionally be selected from monocyclic groups having 3 ring members. Particular examples include monocyclic cycloalkyl groups such as cyclopropyl, cyclobutyl, cyclopentyl, cyclohexyl and cycloheptyl, and 5-, 6- and 7-membered monocyclic heterocyclic groups such as morpholine, piperidine (e.g. 1-piperidinyl, 2-piperidinyl, 3-piperidinyl and 4-piperidinyl), pyrrolidine (e.g. 1-pyrrolidinyl, 2-pyrrolidinyl and 3-pyrrolidinyl), pyrrolidone, piperazine, and N-alkyl piperazines such as N-methyl piperazine. In general, preferred non-aromatic heterocyclic groups include pyrrolidine, piperidine, morpholine, thiomorpholine and N-methyl piperazine.

When R⁰ is an optionally substituted C₁₋₈ hydrocarbyl group, the hydrocarbyl group may be as hereinbefore defined, and is preferably up to four carbon atoms in length, more usually up to three carbon atoms in length for example one or two carbon atoms in length.

In one embodiment, the hydrocarbyl group is saturated and may be acyclic or cyclic, for example acyclic. An acyclic saturated hydrocarbyl group (i.e. an alkyl group) may be a straight chain or branched alkyl group.

Examples of straight chain alkyl groups R⁰ include methyl, ethyl, propyl and butyl.

Examples of branched chain alkyl groups R⁰ include isopropyl, isobutyl, tert-butyl and 2,2-dimethylpropyl.

In one embodiment, the hydrocarbyl group is a linear saturated group having from 1-6 carbon atoms, more usually 1-4 carbon atoms, for example 1-3 carbon atoms, e.g. 1, 2 or 3 carbon atoms. When the hydrocarbyl group is substituted, particular examples of such groups are substituted (e.g. by a carbocyclic or heterocyclic group) methyl and ethyl groups.

A C₁₋₈ hydrocarbyl group R⁰ can be optionally substituted by one or more substituents selected from halogen (e.g. fluorine), hydroxy, C₁₋₄ hydrocarbyloxy, amino, mono- or di-C₁₋₄ hydrocarbylamino, and carbocyclic or heterocyclic groups having from 3 to 12 ring members, and wherein 1 or 2 of the carbon atoms of the hydrocarbyl group may optionally be replaced by an atom or group selected from O, S, NH, SO₅ SO₂. Particular substituents for the hydrocarbyl group include hydroxy, chlorine, fluorine (e.g. as in trifluoromethyl), methoxy, ethoxy, amino, methylamino and dimethylamino, preferred substituents being hydroxy and fluorine.

Particular groups R⁰—CO are the groups set out in Table 1 below.

In Table 1, the point of attachment of the group to the nitrogen atom of the pyrazole-4-amino group is represented by the terminal single bond extending from the carbonyl group. Thus, by way of illustration, group B in the table is the trifluoroacetyl group, group D in the table is the phenylacetyl group and group I in the table is the 3-(4-chlorophenyl)propionyl group.

TABLE 1
Examples of the group R⁰—CO

| | |
|---|---|
| CH₃—C(═O)— | CF₃—C(═O)— |
| A | B |
| 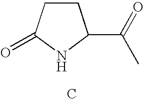 | 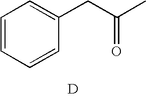 |
| 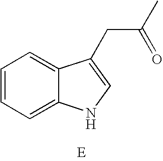 | 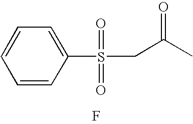 |
| 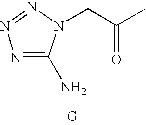 | 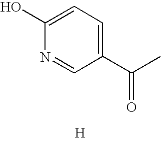 |
| 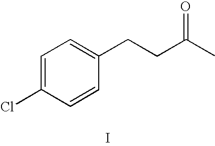 | 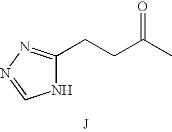 |
| 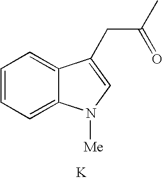 | 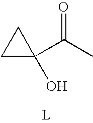 |
| 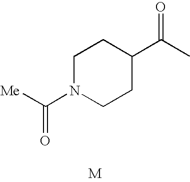 | 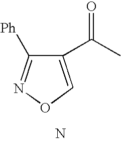 |
| 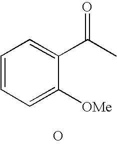 | 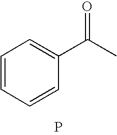 |
| 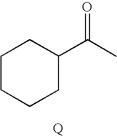 | 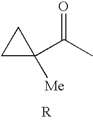 |
| 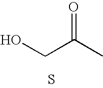 | 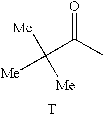 |
| 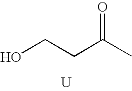 | 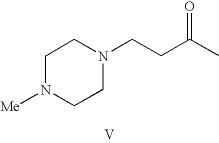 |
| 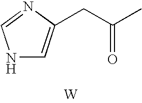 | 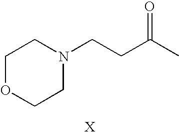 |
| 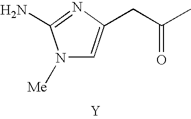 | 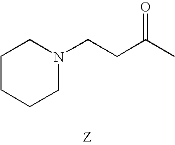 |
| 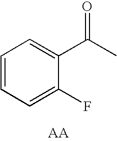 | 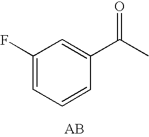 |
| 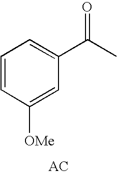 | 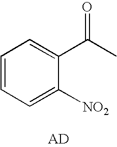 |
| 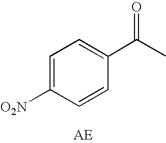 | 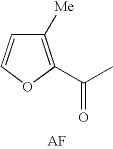 |
| 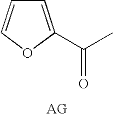 | 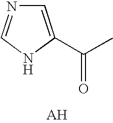 |
| 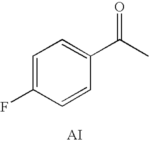 | 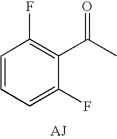 |
| 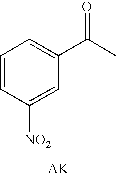 | 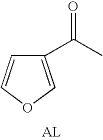 |
| 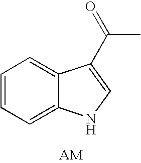 | 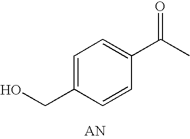 |
| 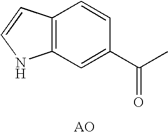 | 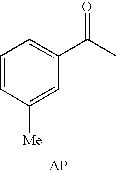 |
| 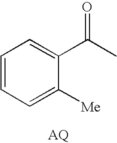 | 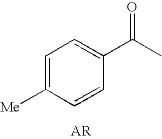 |
| 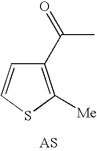 | 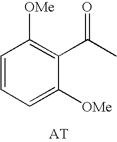 |
| 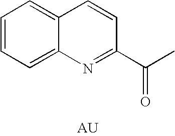 | 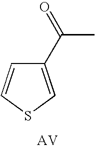 |
| 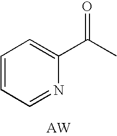 | 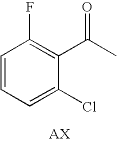 |
| 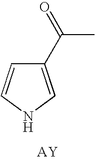 | 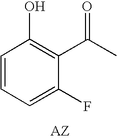 |
| 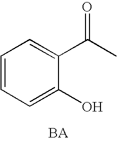 | 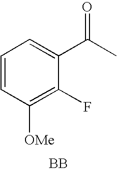 |
| 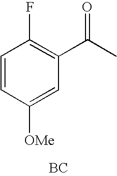 | 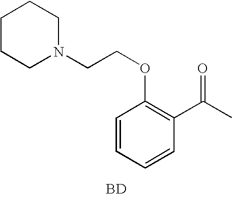 |
| 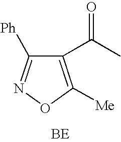 | 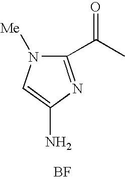 |
| 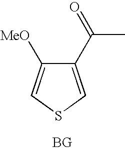 | 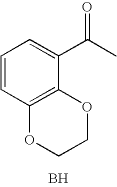 |
| 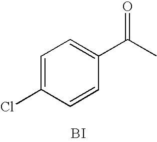 | 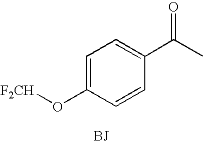 |
| 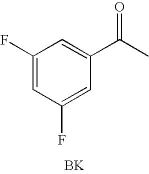 | 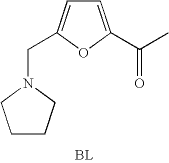 |
| 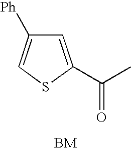 | 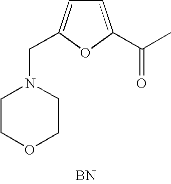 |
| 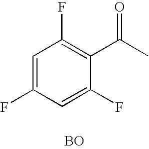 | 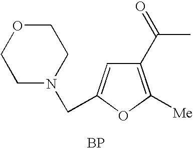 |
| 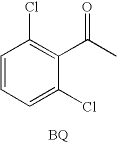 | 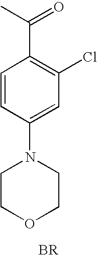 |
| 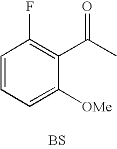 | 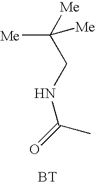 |
| 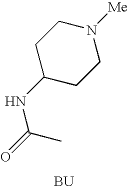 | 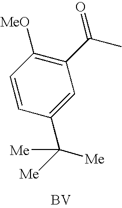 |
| 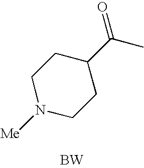 | 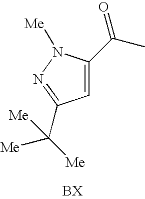 |
| 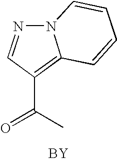 | 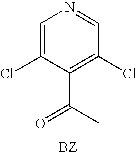 |
| 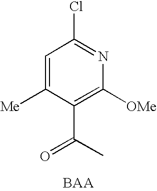 | 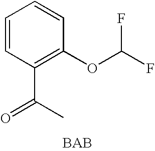 |
| 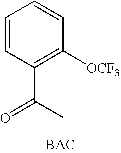 | 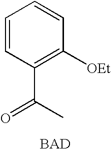 |
| 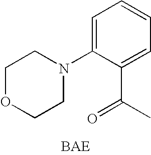 | 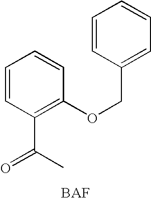 |
| 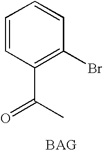 | 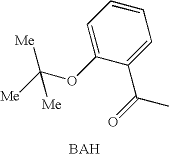 |
| 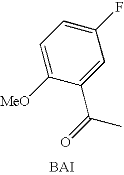 | 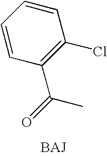 |
| 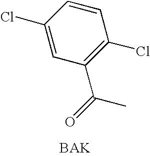 | 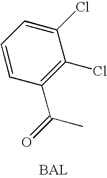 |
| 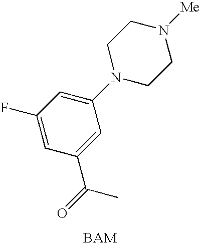 | 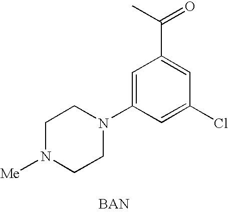 |
| 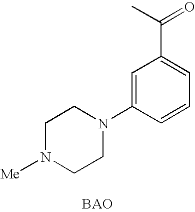 | 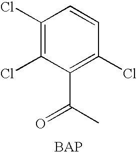 |
| 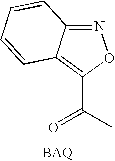 | 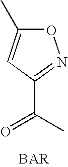 |
| 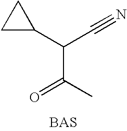 | 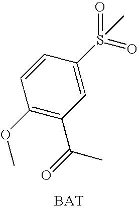 |
| 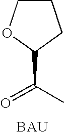 | 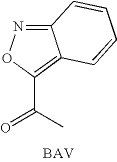 |
| 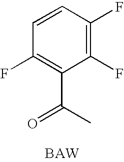 | 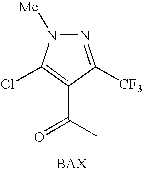 |
| 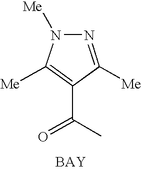 | 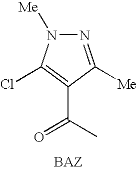 |
| 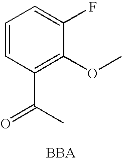 | 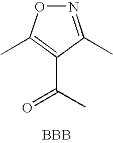 |
| 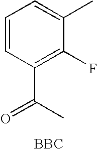 | 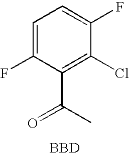 |
| 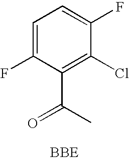 | 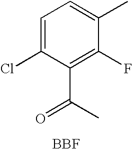 |
| 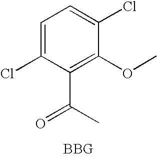 | 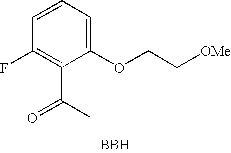 |
| 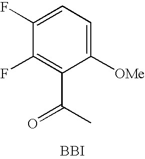 | 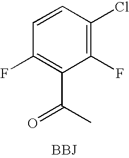 |
| 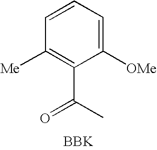 | 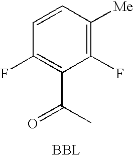 |
| 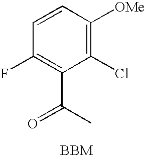 | 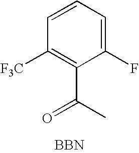 |
| 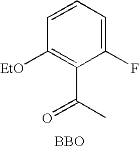 | 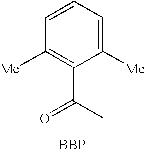 |
| 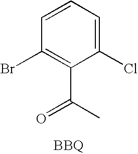 | 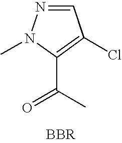 |
| 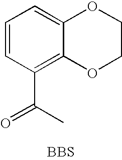 | |

Preferred groups R^{o}—C0 include groups A to B S in Table 1 above.

More preferred groups R^{o}—C0- are AJ₅ AX, BQ, BS and BAI.

One particularly preferred sub-set of groups R^{o}—CO— consists of AJ, BQ and BS.

Another particularly preferred sub-set of groups R^{o}—C0- consists of AJ and BQ.

A further set of preferred groups includes BBD, BBI and BBJ.

In embodiment (H) of the invention, R¹ is (h), a group R^{1d}, and R³ is a group —Y—R^{3a} where Y is a bond or an alkylene chain of 1, 2 or 3 carbon atoms in length and R^{3a} is selected from hydrogen and carbocyclic and heterocyclic groups having from 3 to 12 ring members.

The term “alkylene” has its usual meaning and refers to a divalent saturated acyclic hydrocarbon chain. The hydrocarbon chain may be branched or unbranched.

Where an alkylene chain is branched, it may have one or more methyl group side chains. Examples of alkylene groups include —CH₂—, —CH₂—CH₂—, —CH₂—CH₂—CH₂—, CH(CH₃)—, —C(CHs)₂—, —CH₂—CH(CH₃)—, —CH₂—C(CH₃)₂— and —CH(CH₃)—CH(CH₃)—.

In one embodiment, Y is a bond.

In another embodiment, Y is an alkylene chain.

When Y is an alkylene chain, preferably it is unbranched and more particularly contains 1 or 2 carbon atoms, preferably 1 carbon atom. Thus preferred groups Y are —CH₂— and —CH₂—CH₂—, a most preferred group being (CH₂)—.

Where Y is a branched chain, preferably it has no more than two methyl side chains. For example, it may have a single methyl side chain. In one embodiment,

Y is a group —CH(Me)—.

In one sub-group of compounds, Y is a bond, CH₂, CH₂CH₂ or CH₂CH(CH₃).

The group R^{3a} is selected from hydrogen and carbocyclic and heterocyclic groups having from 3 to 12 ring members.

In one sub-group of compounds, Y is a bond and R^{3a} is hydrogen.

In another sub-group of compounds Y is an alkylene chain as hereinbefore defined and R^{3a} is hydrogen.

In a another sub-group of compounds, Y is a bond or an alkylene chain (e.g. a group —(CH₂)—) and R^{3a} is a carbocyclic or heterocyclic group.

In a further sub-group of compounds, Y is a bond and R^{3a} is a carbocyclic or heterocyclic group.

In a still further sub-group of compounds, Y is an alkylene chain (e.g. a group —(CH₂)—) and R^{3a} is a carbocyclic or heterocyclic group.

The carbocyclic and heterocyclic groups R^{3a} can be aryl, heteroaryl, non-aromatic carbocyclic or non-aromatic heterocyclic and examples of such groups are as set out in detail above in the General Preferences and Definitions section, and as set out below.

Preferred aryl groups R^{3a} are unsubstituted and substituted phenyl groups.

Examples of heteroaryl groups R^{3a} include monocyclic heteroaryl groups containing up to three (and more preferably up to two) heteroatom ring members selected from O, S and N. Preferred heteroaryl groups include five membered rings containing one or two heteroatom ring members and six membered rings containing a single heteroatom ring member, most preferably nitrogen. Particular examples of heteroaryl groups include unsubstituted or substituted pyridyl, imidazole, pyrazole, thiazole, isothiazole, isoxazole, oxazole, furyl and thiophene groups.

Particular heteroaryl groups are unsubstituted and substituted pyridyl groups, e.g. 2-pyridyl, 3-pyridyl and 4-pyridyl groups, especially 3- and 4-pyridyl groups. When the pyridyl groups are substituted, they can bear one or more substituents, typically no more than two, and more usually one substituent selected, for example, from C₁₋₄ alkyl (e.g. methyl), halogen (e.g. fluorine or chlorine, preferably chlorine), and C₁₋₄ alkoxy (e.g. methoxy). Substituents on the pyridyl group may further be selected from amino, mono-C₁₋₄ alkylamino and di-C₁₋₄ alkylamino, particularly amino.

In one embodiment, when R^{3a} is an aryl (e.g. phenyl) or heteroaryl group, the substituents on the carbocyclic or heterocyclic group may be selected from the group R^{1Oa} consisting of halogen, hydroxy, trifluoromethyl, cyano, monocyclic carbocyclic and heterocyclic groups having from 3 to 7 (typically 5 or 6) ring members, and a group R^{a}—R^{b} wherein R^{a} is a bond, O, CO, X¹C(X²), C(X²)X¹, X¹C(X²)^{X}^{₁}, S, SO, SO₂, NR ^{c̅}, SO₁NR^{c} or NR ^{c̅SO}₂; and R^{b} is selected from hydrogen, a carbocyclic or heterocyclic group with 3-7 ring members and a C₁₋₈ hydrocarbyl group optionally substituted by one or more substituents selected from hydroxy, oxo, halogen, cyano, nitro, carboxy, amino, mono- or di-Ci₋₄ hydrocarbylamino, a carbocyclic or heterocyclic group with 3-7 ring members and wherein one or more carbon atoms of the C₁₋₈ hydrocarbyl group may optionally be replaced by O, S, SO, SO₂, NR^{C}, X¹C(X²), C(X²)^{X}^{₁} or X¹C(X²)^{X}^{₁}; and R^{c}, X¹ and X² are as hereinbefore defined.

Examples of non-aromatic groups R^{3a} include optionally substituted (by R¹⁰ or R^{1Oa}) cycloalkyl, oxa-cycloalkyl, aza-cycloalkyl, diaza-cycloalkyl, dioxa-cycloalkyl and aza-oxa-cycloalkyl groups. Further examples include C₇₋₁₀ aza-bicycloalkyl groups such as 1-aza-bicyclo[2.2.2]octan-3-yl.

Particular examples of such groups include unsubstituted or substituted cyclopropyl, cyclobutyl, cyclopentyl, cyclohexyl, tetrahydropyran, moφ holine, tetrahydrofuran, piperidine and pyrrolidine groups.

One sub-set of non-aromatic groups R^{3a} consists of cyclopropyl, cyclobutyl, cyclopentyl, cyclohexyl, tetrahydropyran, tetrahydrofuran, piperidine and pyrrolidine groups.

Preferred non-aromatic groups R^{3a} include unsubstituted or substituted cyclopentyl, cyclohexyl, tetrahydropyran, tetrahydrofuran, piperidine and pyrrolidine groups,

The non-aromatic groups may be unsubstituted or substituted with one or more groups R¹⁵ or R^{15a} as hereinbefore defined.

Particular substituents for R^{3a} (e.g. (1) when R^{3a} is an aryl or heteroaryl group or (2) when R^{3a} is a non-aromatic group) are selected from the group R^{15a} consisting of halogen; hydroxy; monocyclic carbocyclic and heterocyclic groups having from 3 to 6 ring members and containing up to 2 heteroataom ring members selected from O, N and S; and a group R^{a}—R^{b} wherein R^{a} is a bond, O, CO, CO₂, SO₂, NH, SO₂NH or NHSO₂; and R^{b} is selected from hydrogen, a carbocyclic or heterocyclic group with 3-6 ring members and containing up to 2 heteroatom ring members selected from O, N and S; and a C₁₋₆ hydrocarbyl group optionally substituted by one or more substituents selected from hydroxy, oxo, halogen, carboxy, amino, mono- or di-C₁₋₄hydrocarbylamino, a carbocyclic or heterocyclic group with 3-6 ring members and containing up to 2 heteroatom ring members selected from O, N and S; and wherein one or two carbon atoms of the C₁₋₆ hydrocarbyl group may optionally be replaced by O, S, SO, SO₂ or NH.

In one embodiment, preferred R^{1Oa} substituent groups on R³ (e.g. (1) when R³ is an aryl or heteroaryl group or (2) when R^{3a} is a non-aromatic group) include halogen, a group R^{a}-R^{b} wherein R^{a} is a bond, O, CO, C(X²)X¹, and R^{b} is selected from hydrogen, heterocyclic groups having 3-7 ring members and a C₁₋₄ hydrocarbyl group optionally substituted by one or more substituents selected from hydroxy, carboxy, amino, mono- or di-C₁₋₄ hydrocarbylamino, and heterocyclic groups having 3-7 ring members.

Particularly preferred substituent groups R^{15a} on R^{3a} (e.g. (1) when R^{3a} is an aryl or heteroaryl group or (2) when R^{3a} is a non-aromatic group) include halogen, especially fluorine, C₁₋₃ alkoxy such as methoxy, and C₁₋₃ hydrocarbyl optionally substituted by fluorine, hydroxy (e.g. hydroxymethyl), C₁₋₂ alkoxy or a 5- or 6-membered saturated heterocyclic ring such as piperidino, morpholino, piperazino and N-methylpiperazino.

In another embodiment, the substituents for R^{3a} (whether aromatic or non-aromatic) are selected from:
halogen (e.g. fluorine and chlorine)
C₁₋₄ alkoxy (e.g. methoxy and ethoxy) optionally substituted by one or substituents selected from halogen, hydroxy, C₁₋₂ alkoxy and five and six membered saturated heterocyclic rings containing 1 or 2 heteroatoms selected from O, N and S, the heterocyclic rings being optionally further substituted by one or more C₁₋₄ groups (e.g. methyl) and wherein the S, when present, may be present as S, SO or SO₂;
C₁₋₄ alkyl optionally substituted by one or substituents selected from halogen, hydroxy, C₁₋₄ alkoxy, amino, C₁₋₄ alkylsulphonylamino, 3 to 6 membered cycloalkyl groups (e.g. cyclopropyl), phenyl (optionally substituted by one or more substituents selected from halogen, methyl, methoxy and amino) and five and six membered saturated heterocyclic rings containing 1 or 2 heteroatoms selected from O, N and S, the heterocyclic rings being optionally further substituted by one or more Ci₋₄ groups (e.g. methyl) and wherein the S, when present, may be present as S, SO or SO₂;
hydroxy;
amino, mono-Ci₋₄ alkylamino, di-C₁₋₄ alkylamino, benzyloxycarbonylamino and Ci₋₄ alkoxycarbonylamino;
carboxy and Ci₋₄ alkoxycarbonyl;
Ci₋₄ alkylaminosulphonyl and Ci₋₄ alkylsulphonylamino;
C₁₋₄ alkylsulphonyl;
a group O-Het^{s} or NH-Het^{s} where Het^{s} is a five or six membered saturated heterocyclic ring containing 1 or 2 heteroatoms selected from O, N and S, the heterocyclic rings being optionally further substituted by one or more C₁₋₄ groups (e.g. methyl) and wherein the S, when present, may be present as S, SO or SO₂;
five and six membered saturated heterocyclic rings containing 1 or 2 heteroatoms selected from O, N and S, the heterocyclic rings being optionally further substituted by one or more C₁₋₄ groups (e.g. methyl) and wherein the S, when present, may be present as S, SO or SO₂;
oxo; and
six membered aryl and heteroaryl rings containing up to two nitrogen ring members and being optionally substituted by one or substituents selected from halogen, methyl and methoxy.

In one preferred sub-group of compounds, R^{3a} is a carbocyclic or heterocyclic group R^{3b} selected from phenyl; C₃₋₆ cycloalkyl; five and six membered saturated non-aromatic heterocyclic rings containing up to two heteroatom ring members selected from N, O, S and SO₂; six membered heteroaryl rings containing one, two or three nitrogen ring members; and five membered heteroaryl rings having up to three heteroatom ring members selected from N, O and S;

wherein each carbocyclic or heterocyclic group R^{3b} is optionally substituted by up to four, preferably up to three, and more preferably up to two (e.g. one) substituents selected from amino; hydroxy; oxo; fluorine; chlorine; Ci₋₄ alkyl-(O)_{q}— wherein q is 0 or 1 and the C₁₋₄ alkyl moiety is optionally substituted by fluorine, hydroxy or Ci₋₂ alkoxy; mono-C₁₋₄ alkylamino; di-Ci-4 alkylamino; C₁₋₄ alkoxycarbonyl; carboxy; a group R^{e}—R¹⁶ where R^{e} is a bond or a C₁₋₃ alkylene chain and R¹⁶ is selected from C₁₋₄ alkylsulphonyl; C₁₋₄ alkylaminosulphonyl; C₁₋₄ alkylsulphonylamino-; amino; mono-Ci-4 alkylamino; di-C₁₋₄ alkylamino; C₁₋₇-hydrocarbyloxycarbonylamino; six membered aromatic groups containing up to three nitrogen ring members; C₃₋₆ cycloalkyl; five or six membered saturated non-aromatic heterocyclic groups containing one or two heteroatom ring members selected from N, O, S and SO₂, the group R¹⁶ when a saturated non-aromatic group being optionally substituted by one or more methyl groups, and the group R¹⁶ when aromatic being optionally substituted by one or more groups selected from fluorine, chlorine, hydroxy, C₁₋₂ alkoxy and C₁₋₂ alkyl.

In a further embodiment, R^{3a} is selected from:
monocyclic aryl groups optionally substituted by 1-4 (for example 1-2, e.g. 1) substituents R¹⁵ or R^{15a};
C₃-C₇ cycloalkyl groups optionally substituted by 1-4 (for example 1-2, e.g. 1) substituents R¹⁵ or R^{15a};

saturated five membered heterocyclic rings containing 1 ring heteroatom selected from O, N and S and being optionally substituted by an oxo group and/or by 1-4 (for example 1-2, e.g. 1) substituents R¹⁰ or R^{1Oa};
saturated six membered heterocyclic rings containing 1 or 2 ring heteroatoms selected from O, N and S and being optionally substituted by an oxo group and/or by 1-4 (for example 1-2, e.g. 1) substituents R¹⁰ or R^{1Oa};
five membered heteroaryl rings containing 1 or 2 ring heteroatoms selected from O, N and S and being optionally substituted by 1-4 (for example 1-2, e.g. 1) substituents R¹⁵ or R^{15a};
six membered heteroaryl rings containing 1 or 2 nitrogen ring members (preferably 1 nitrogen ring member) and being optionally substituted by 1-4 (for example 1-2, e.g. 1) substituents R¹⁵ or R^{15a};
mono-azabicycloalkyl and diazabicycloalkyl groups each having 7 to 9 ring members and being optionally substituted by 1-4 (for example 1-2, e.g. 1) substituents R¹⁵ or R^{15a}.

The group Y—R^{3a} can be a group R³ of any one of formulae (i), (ii), (iii), (iv), (v), (vi), (vii), (x), (xi), (xii), (xiii), (xiv) and (xv) as defined herein.

In addition, the group Y-R^{3a} can be further selected from:

a group (xvi):

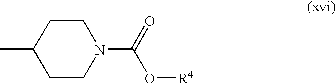

where R⁴ is C₁₋₄ alkyl; and
a group (xvii):

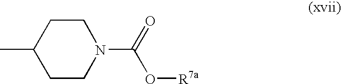

where R^{7a} is selected from:
unsubstituted C₁₋₄ hydrocarbyl other than C₁₋₄ alkyl;
C₁₋₄ hydrocarbyl substituted by one or more substituents chosen from C₃₋₆ cycloalkyl, fluorine, chlorine, methylsulphonyl, acetoxy, cyano, methoxy; and a group NR⁵R⁶; and
a group —(CH₂)ₙ—R⁸ where n is O or 1 and R⁸ is selected from C₃₋₆ cycloalkyl; oxa-C₄₋₆ cycloalkyl; phenyl optionally substituted by one or more substituents selected from fluorine, chlorine, methoxy, cyano, methyl and trifluoromethyl; an aza-bicycloalkyl group; and a 5-membered heteroaryl group containing one or two heteroatom ring members selected from O, N and S and being optionally substituted by methyl, methoxy, fluorine, chlorine, or a group NR⁵R⁶.

In group (xvii), R⁴ is C₁₋₄ alkyl.

The C₁₋₄ alkyl group may be as set out in the General Preferences and Definitions section above. Thus, it can be a C₁, C₂, C₃ or C₄ alkyl group. Particular C₁₋₄ alkyl groups are methyl, ethyl, i-propyl, n-butyl, i-butyl and tert-butyl groups.

One particular group is a methyl group.

Other particular groups R⁴ are ethyl and isopropyl.

In group (xvii), when R^{7a} is unsubstituted C₁₋₄ hydrocarbyl other than C₁₋₄ alkyl, particular hydrocarbyl groups are unsubstituted C₂₋₄ alkenyl groups such as vinyl and 2-propenyl. A preferred group R^{7a} is vinyl.

Examples of substituted C₁₋₄ hydrocarbyl groups are C₁₋₄ hydrocarbyl groups substituted by one or more substituents chosen from C₃₋₆ cycloalkyl, fluorine, chlorine, methylsulphonyl, acetoxy, cyano, methoxy; and a group NR⁵R⁶. The C₁₋₄ hydrocarbyl groups can be, for example, substituted methyl groups, 1-substituted ethyl groups and 2-substituted ethyl groups. Preferred groups R^{7a} include 2-substituted ethyl groups, for example 2-substituted ethyl groups wherein the 2-substituent is a single substituent such as methoxy.

When the substituted C₁₋₄ hydrocarbyl groups are substituted by NR⁵R⁶, examples of NR⁵R⁶ include dimethylamino and heterocyclic rings selected from morpholine, piperidine, piperazine, N-methylpiperazine, pyrrolidine and thiazolidine. Particular heterocyclic rings include morpholinyl, 4-methylpiperazinyl and pyrrolidine.

When R^{7a} is a group —(CH₂)ₙ—R⁸ where n is 0 or 1, R⁸ can be a C₃₋₆ cycloalkyl group such as cyclopropyl, cyclopentyl, or an oxa-C₄₋₆ cycloalkyl group such as tetrahydrofuranyl and tetrahydropyranyl. In one sub-group of compounds, n is 0 and in another sub-group of compounds, n is 1.

Alternatively, when R^{7a} is a group —(CH₂)ₙ—R⁸ where n is 0 or 1, R⁸ can be phenyl optionally substituted by one or more substituents selected from fluorine, chlorine, methoxy, cyano, methyl and trifluoromethyl. In one sub-group of compounds, n is 0 and the optionally substituted phenyl group is attached directly to the oxygen atom of the carbamate. In another sub-group of compounds, n is 1 and hence the optionally substituted phenyl group forms part of a benzyl group. Particular examples of a group —(CH₂)ₙ—R⁸ where R⁸ is a phenyl group are unsubstituted phenyl, 4-fluorophenyl and benzyl.

In another alternative, when R^{7a} is a group —(CHa)ₙ—R⁸ where n is 0 or 1, R⁸ can be a 5-membered heteroaryl group containing one or two heteroatom ring members selected from O, N and S and being optionally substituted by methyl, methoxy, fluorine, chlorine, or a group NR⁵R⁶. Examples of heteroaryl groups are as set out above in the General Preferences and Definitions section. One particular heteroaryl group is a thiazole group, more particularly a 5-thiazole group, preferably when n is 1.

Specific examples of the group Y—R^{3a} are set out in Table 2. In Table 2, the point of attachment of the group to the nitrogen atom of the pyrazole-3-carboxamide group is represented by the terminal single bond extending from the group. Thus, by way of illustration, group CA in the table is the 4-fluorophenyl, group CB in the table is the 4-methoxybenzyl group and group CC in the table is the 4-(4-methylpiperazino)-phenylmethyl group.

TABLE 2
Example of the Group Y—R^{3a}

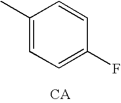

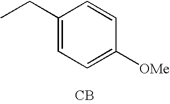

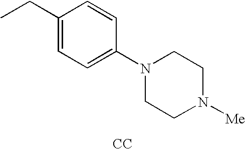

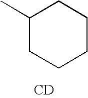

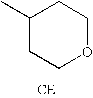

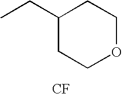

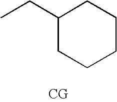

H
CH

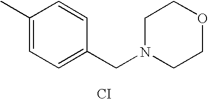

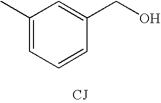

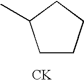

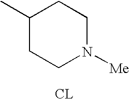

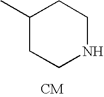

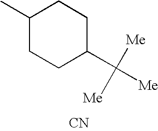

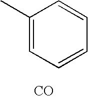

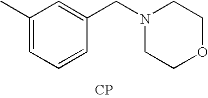

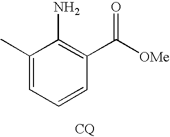

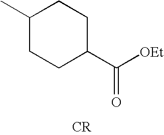

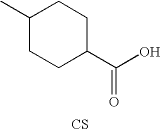

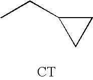

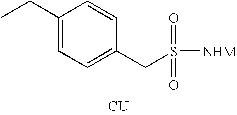

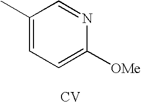

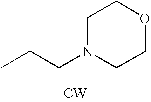

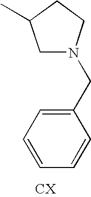

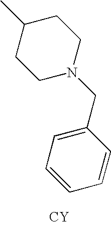

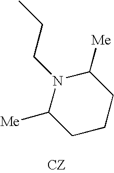

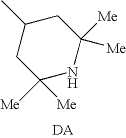

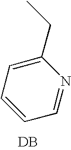

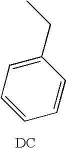

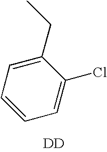

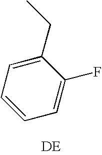

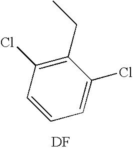

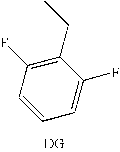

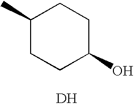

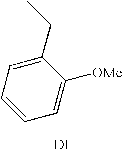

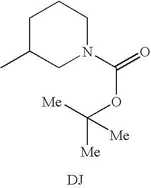

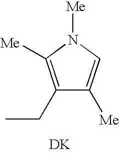

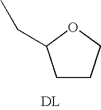

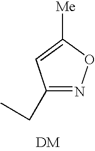

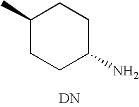

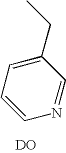

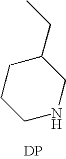

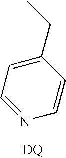

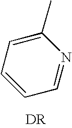

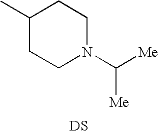

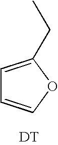

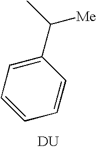

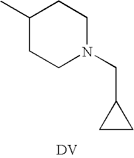

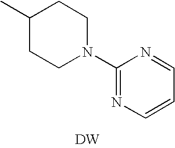

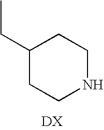

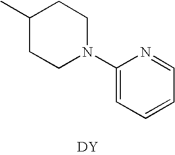

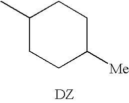

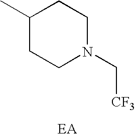

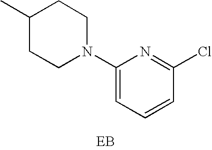

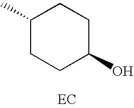

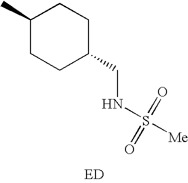

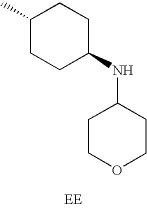

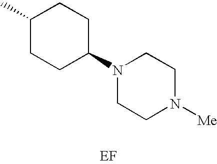

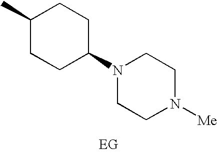

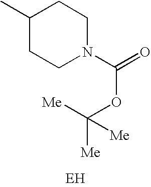

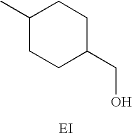

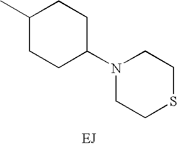

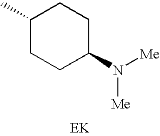

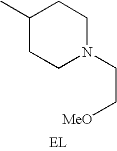

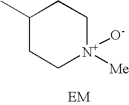

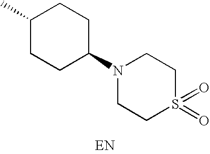

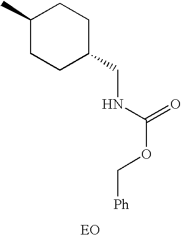

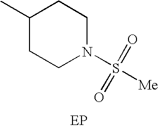

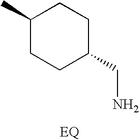

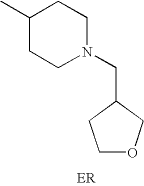

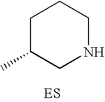

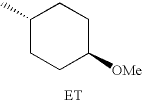

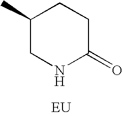

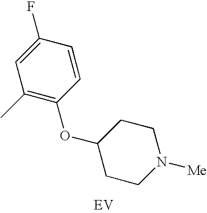

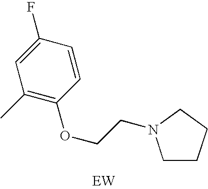

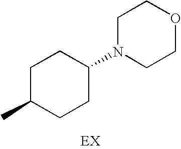

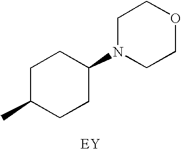

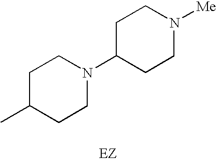

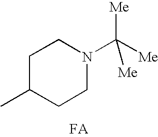

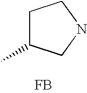

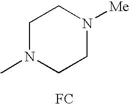

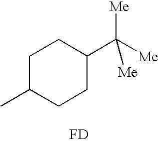

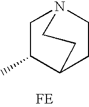

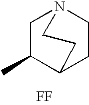

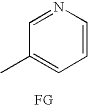

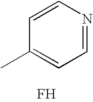

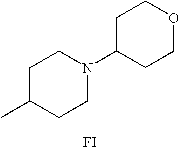

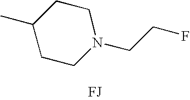

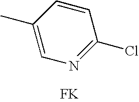

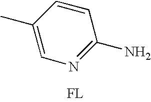

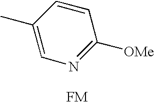

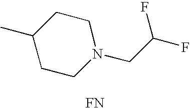

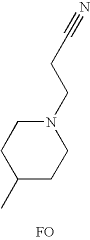

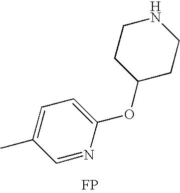

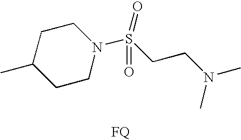

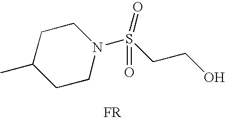

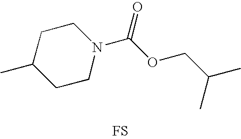

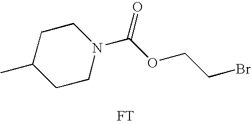

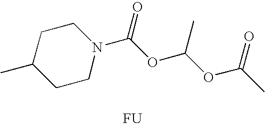

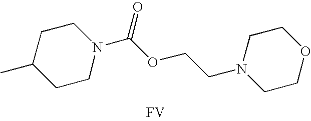

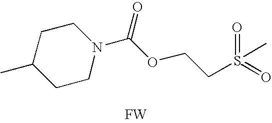

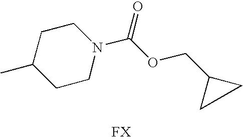

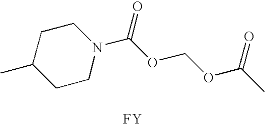

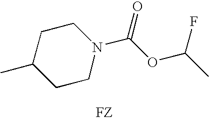

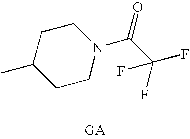

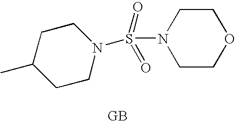

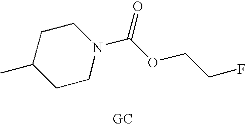

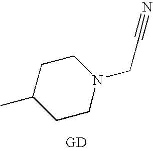

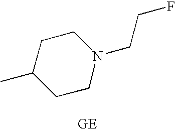

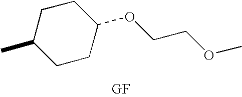

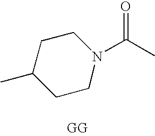

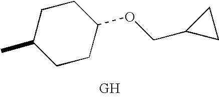

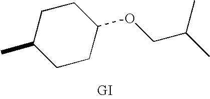

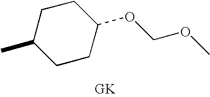

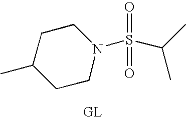

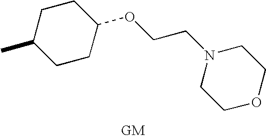

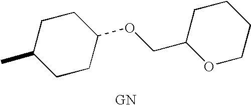

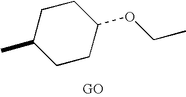

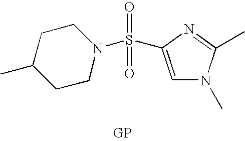

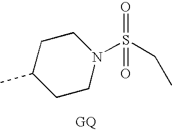

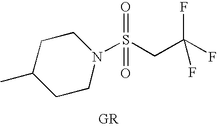

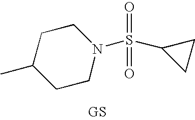

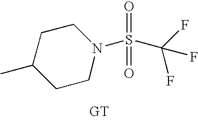

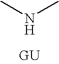

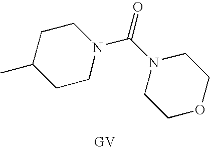

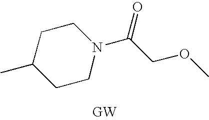

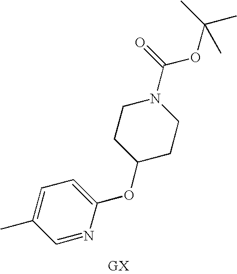

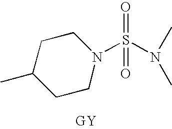

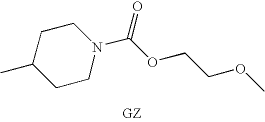

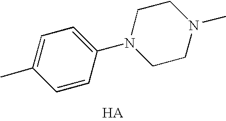

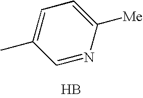

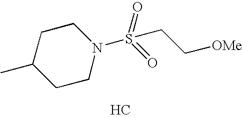

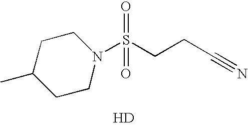

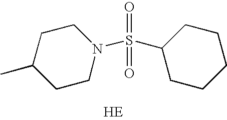

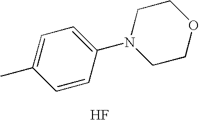

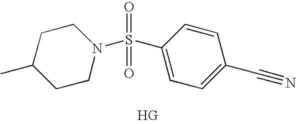

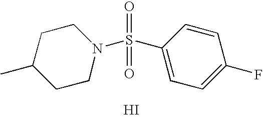

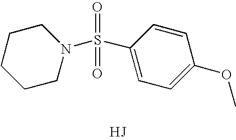

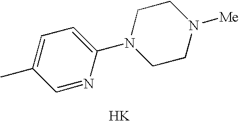

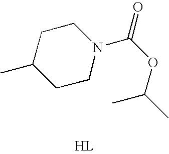

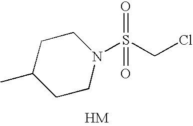

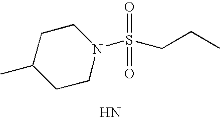

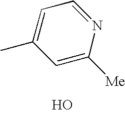

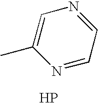

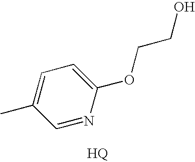

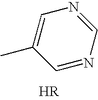

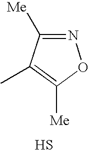

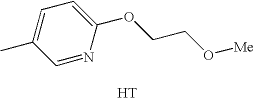

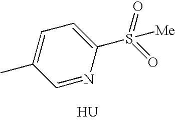

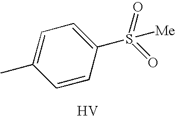

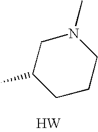

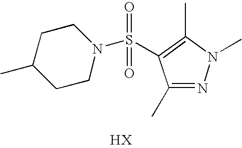

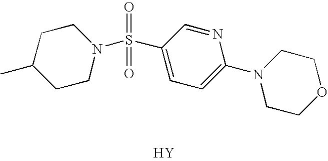

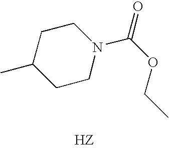

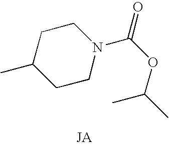

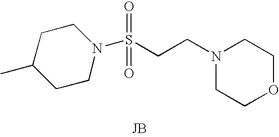

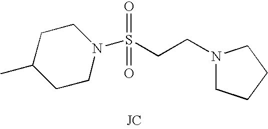

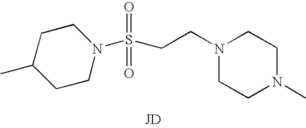

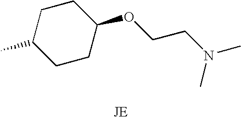

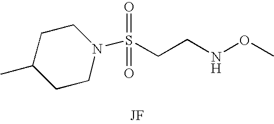

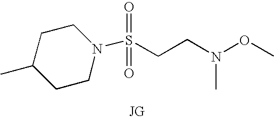

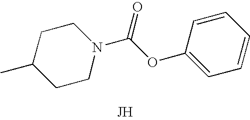

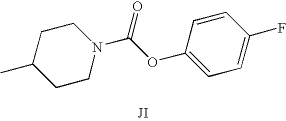

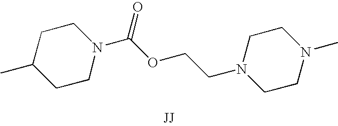

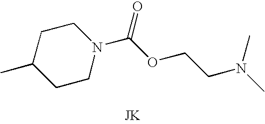

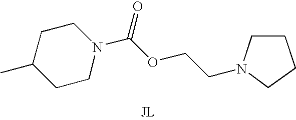

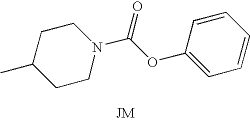

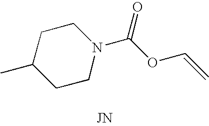

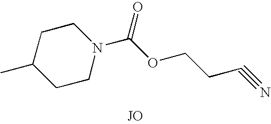

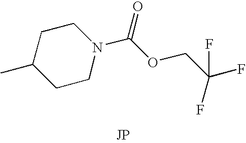

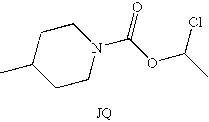

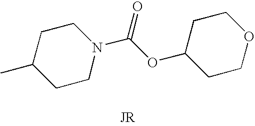

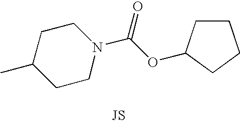

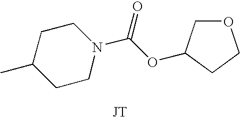

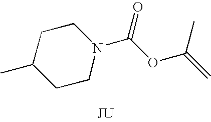

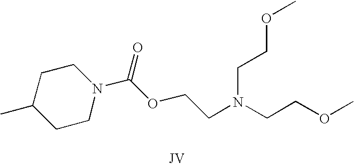

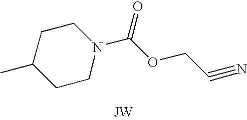

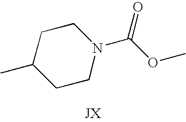

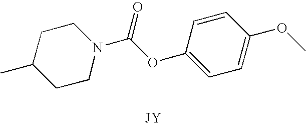

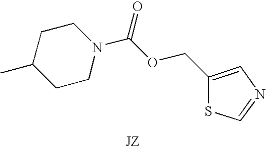

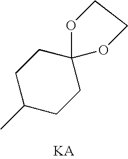

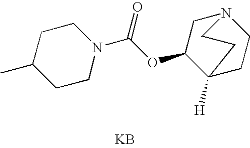

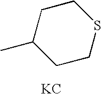

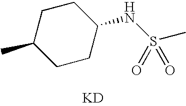

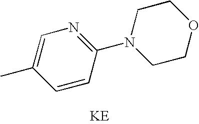

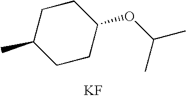

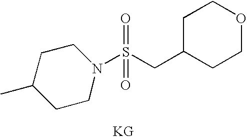

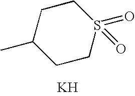

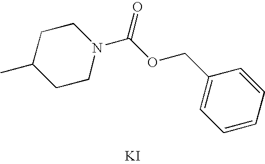

Preferred groups selected from table 2 include groups CA to CV.

One sub-set of preferred groups in table 2 consists of groups CL₅ CM, ES, ET, FC, FG and FH.

Another preferred set of groups selected from Table 2 includes groups CL, CM and ES, and most preferably CL and CM.

Another preferred group is EP.

Within embodiment (H), one sub-group of compounds of the formula (I) can be represented by the formula (IV):

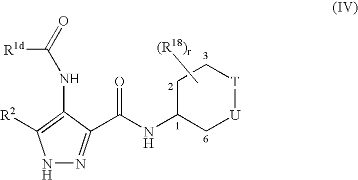

or salts or tautomers or N-oxides or solvates thereof;
wherein R^{1d} and R² are as defined herein;
an optional second bond may be present between carbon atoms numbered 1 and 2; one of U and T is selected from CH₂, CHR²⁰, CR¹⁸R²⁰, NR²¹, N(O)R²², O and S(O)ₜ; and the other of U and T is selected from, NR²¹, O, CH₂, CHR¹⁸, C(R¹⁸)₂, and C═O; r is 0, 1, 2, 3 or 4; t is 0, 1 or 2;
R¹⁸ is selected from hydrogen, halogen (particularly fluorine), C₁₋₃ alkyl (e.g. methyl) and C₁₋₃ alkoxy (e.g. methoxy);
R²⁰ is selected from hydrogen, NHR²¹, NOH, NOR²¹ and R^{a}—R^{b};
R²¹ is selected from hydrogen and R^{d}—R^{b};
R^{d} is selected from a bond, CO, C(X²)^{X}^{₁}, SO₂ and SO₂NR ^{c̅;}
R^{a}, R^{b} and R^{o} are as hereinbefore defined; and
R²² is selected from C₁₋₄ saturated hydrocarbyl optionally substituted by hydroxy, C₁₋₂ alkoxy, halogen or a monocyclic 5- or 6-membered carbocyclic or heterocyclic group, provided that U and T cannot be O simultaneously.

Within formula (IV), r can be 0, 1, 2, 3 or 4. In one embodiment, r is 0. In another embodiment, r is 2, and in a further embodiment r is 4.

Within formula (IV), one sub-set of preferred compounds is the set of compounds where there is only a single bond between the carbon atoms numbered 1 and 2.

However, in another sub-set of compounds, there is a double bond between the carbon atoms numbered 1 and 2.

Another sub-set of compounds is characterised by gem disubstitution at the 2-carbon (when there is a single bond between carbon atoms numbers 1 and 2) and/or the 6-carbon. Preferred gem disubstituents include difluoro and dimethyl.

A further sub-set of compounds is characterised by the presence of an alkoxy group, for example a methoxy group at the carbon atom numbered 3, i.e. at a position α with respect to the group T.

Within formula (IV) are compounds wherein, for example, R^{3a} is selected from any of the following ring systems:

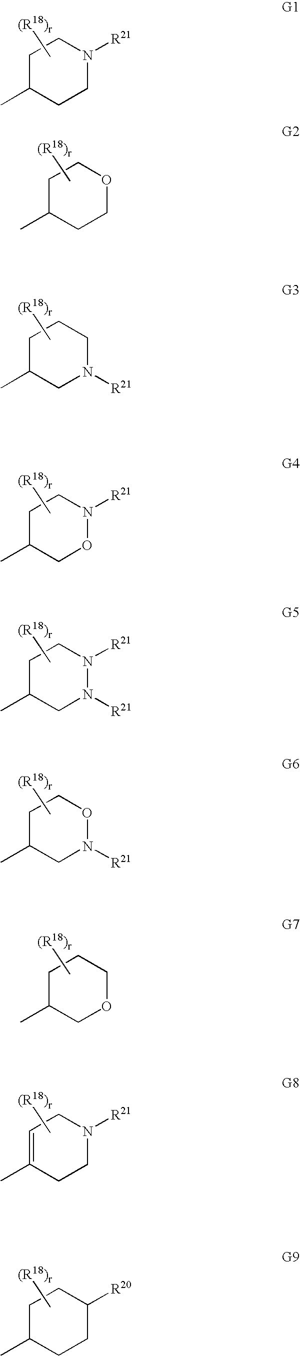

Preferred ring systems include G1 and G3.

A preferred sub-group of compounds within formula (IV) can be represented by the formula (IVa):

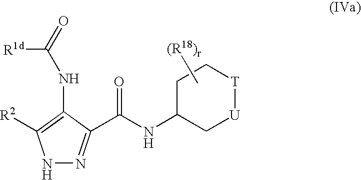

or salts or tautomers or N-oxides or solvates thereof;
wherein R^{1d} and R² are as hereinbefore defined;
one of U and T is selected from CH₂, CHR²⁰, CR¹⁸R²⁰, NR²¹, N(O)R²², O and S(O)ₜ; and the other of U and T is selected from CH₂, CHR¹⁸, C(R¹⁸)₂, and C═O; r is 0, 1 or 2; t is o, 1 or 2;
R¹⁸ is selected from hydrogen and C₁₋₃ alkyl;
R²⁰ is selected from hydrogen and R^{a}—R^{b};
R²¹ is selected from hydrogen and R^{d}—R^{b};
R^{d} is selected from a bond, CO, C(X²)X¹, SO₂ and SO₂R^{c};
R^{a}, R^{b} and R^{c} are as hereinbefore defined; and
R²² is selected from C₁₋₄ saturated hydrocarbyl optionally substituted by hydroxy, C₁₋₂ alkoxy, halogen or a monocyclic 5- or 6-membered carbocyclic or heterocyclic group.

In formula (IVa), T is preferably selected from CH₂, CHR²⁰, CR¹⁸R²⁰, NR²¹, N(O)R²², O and S(O)ₜ; and U is preferably selected from CH₂, CHR¹⁸, C(R¹⁸)₂, and C═O.

In the definitions for substituents R¹⁸ and R²¹, R^{b} is preferably selected from hydrogen; monocyclic carbocyclic and heterocyclic groups having from 3 to 7 ring members; and C₁₋₄ hydrocarbyl (more preferably acyclic saturated C₁₋₄ groups) optionally substituted by one or more substituents selected from hydroxy, oxo, halogen, amino, mono- or di-C₁₋₄hydrocarbylamino, and monocyclic carbocyclic and heterocyclic groups having from 3 to 7 ring members (more preferably 3 to 6 ring members) and wherein one or more carbon atoms of the C₁₋₄ hydrocarbyl group may optionally be replaced by O, S, SO, SO₂, NR^{c}, X¹C(X²), C(X²)^{X}^{₁};

R^{c} is selected from hydrogen and C₁₋₄ hydrocarbyl; and
X¹ is O, S or NR^{c} and X² is =0, ═S or ═NR^{c}.

R¹⁸ is preferably selected from hydrogen and methyl and most preferably is hydrogen.

R²⁰ is preferably selected from hydrogen; hydroxy; halogen; cyano; amino; mono-C₁₋₄ saturated hydrocarbylamino; di-C₁₋₄ saturated hydrocarbylamino; monocyclic 5- or 6-membered carbocyclic and heterocyclic groups; C₁₋₄ saturated hydrocarbyl optionally substituted by hydroxy, C₁₋₂ alkoxy, halogen or a monocyclic 5- or 6-membered carbocyclic or heterocyclic group.

Particular examples of R²⁰ are hydrogen, hydroxy, amino, C₁₋₂ alkylamino (e.g. methylamino) C₁₋₄ alkyl (e.g. methyl, ethyl, propyl and butyl), C₁₋₂ alkoxy (e.g. methoxy), C₁₋₂ alkylsulphonamido (e.g. methanesulphonamido), hydroxy-C₁₋₂ alkyl (e.g. hydroxymethyl), C₁₋₂-alkoxy-C₁₋₂ alkyl (e.g. methoxymethyl and methoxyethyl), carboxy, C₁₋₄ alkoxycarbonyl (e.g. ethoxycarbonyl) and amino-C₁₋₂-alkyl (e.g. aminomethyl).

Particular examples of R²⁰ are hydrogen; C₁₋₄ alkyl optionally substituted by fluoro or a five or six membered saturated heterocyclic group (e.g. a group selected from (i) methyl, ethyl, n-propyl, i-propyl, butyl, 2,2,2-trifluoroethyl and tetrahydrofuranylmethyl; and/or (ii) 2-fluoroethyl and 2,2-difluoroethyl); cyclopropylmethyl; substituted or unsubstituted pyridyl-C₁₋₂ alkyl (e.g. 2-pyridylmethyl); substituted or unsubstituted phenyl-C₁₋₂ alkyl (e.g. benzyl); C₁₋₄ alkoxycarbonyl (e.g. ethoxycarbonyl and t-butyloxycarbonyl); substituted and unsubstituted phenyl-C₁₋₂ alkoxycarbonyl (e.g. benzyloxycarbonyl); substituted and unsubstituted 5- and 6-membered heteroaryl groups such as pyridyl (e.g. 2-pyridyl and 6-chloro-2-pyridyl) and pyrimidinyl (e.g. 2-pyrimidinyl); C₁₋₂-alkoxy-C₁₋₂ alkyl (e.g. methoxymethyl and methoxyethyl); C₁₋₄ alkylsulphonyl (e.g. methanesulphonyl).

In each of the above of the examples and preferences for embodiment (H), R^{1d} is a group R^{1e}—(CH₂)ₙCH(CN)— where n is 0, 1 or 2 and R^{1e} is a carbocyclic or heterocyclic group having from 3 to 12 ring members.

The carbocyclic and heterocyclic groups can be as set out in the General Preferences and Definitions section.

Preferably n is 0.

Particular carbocyclic and heterocyclic groups are saturated monocyclic groups having from 3 to 7 ring members, such as cycloalkyl groups.

One particular cycloalkyl group is a cyclopropyl group.

The various functional groups and substituents making up the compounds of the formula (I) are typically chosen such that the molecular weight of the compound of the formula (I) does not exceed 1000. More usually, the molecular weight of the compound will be less than 750, for example less than 700, or less than 650, or less than 600, or less than 550. More preferably, the molecular weight is less than 525 and, for example, is 500 or less.

Particular compounds of the invention are as illustrated in the examples below.

One set of specific compounds of the invention is the set of compounds of Examples 1 to 132. Within this set of compounds, one sub-set consists of the compounds of Examples 1 to 114. Another sub-set consists of the compounds of Examples 115 to 132. A further sub-set consists of the compounds of Examples 133 to 137.

Preferred compounds of the invention include:
4-(2,3-difluoro-6-methoxy-benzoylamino)-1H-pyrazole-3-carboxylic acid (1-methanesulphonyl-piperidin-4-yl)-amide;
4-(3-chloro-2,6-difluoro-benzoylamino)-1H-pyrazole-3-carboxylic acid (1-methanesulphonyl-piperidin-4-yl)-amide;
4-(2-chloro-3,6-difluoro-benzoylamino)-1H-pyrazole-3-carboxylic acid (1-methanesulfonyl-piperidin-4-yl)-amide; and salts, solvates, tautomers and N-oxides thereof.

### Salts, Solvates, Tautomers, Isomers, N-Oxides, Esters, Prodrugs and Isotopes

A reference to a compound of the formulae (I) and sub-groups thereof also includes ionic forms, salts, solvates, isomers, tautomers, N-oxides, esters, prodrugs, isotopes and protected forms thereof, for example, as discussed below; preferably, the salts or tautomers or isomers or N-oxides or solvates thereof; and more preferably, the salts or tautomers or N-oxides or solvates thereof.

Many compounds of the formula (I) can exist in the form of salts, for example acid addition salts or, in certain cases salts of organic and inorganic bases such as carboxylate, sulphonate and phosphate salts. AU such salts are within the scope of this invention, and references to compounds of the formula (I) include the salt forms of the compounds.

The salts of the present invention can be synthesized from the parent compound that contains a basic or acidic moiety by conventional chemical methods such as methods described in Pharmaceutical Salts: Properties, Selection, and Use, P. Heinrich Stahl (Editor), Camille G. Wermuth (Editor), ISBN: 3-90639-026-8, Hardcover, 388 pages, August 2002. Generally, such salts can be prepared by reacting the free acid or base forms of these compounds with the appropriate base or acid in water or in an organic solvent, or in a mixture of the two; generally, nonaqueous media such as ether, ethyl acetate, ethanol, isopropanol, or acetonitrile are used.

Acid addition salts may be formed with a wide variety of acids, both inorganic and organic. Examples of acid addition salts include salts formed with an acid selected from the group consisting of acetic, 2,2-dichloroacetic, adipic, alginic, ascorbic (e.g. L-ascorbic), L-aspartic, benzenesulphonic, benzoic, 4-acetamidobenzoic, butanoic, (+) camphoric, camphor-sulphonic, (+)-(15)-camphor-10-sulphonic, capric, caproic, caprylic, cinnamic, citric, cyclamic, dodecylsulphuric, ethane-1,2-disulphonic, ethanesulphonic, 2-hydroxyethanesulphonic, formic, fumaric, galactaric, gentisic, glucoheptonic, D-gluconic, glucuronic (e.g. D-glucuronic), glutamic (e.g. L-glutamic), α-oxoglutaric, glycolic, hippuric, hydrobromic, hydrochloric, hydriodic, isethionic, (+)-L-lactic, (±)-DL-lactic, lactobionic, maleic, malic, (−)-L-malic, malonic, (±)-DL-mandelic, methanesulphonic, naphthalene-2-sulphonic, naphthalene-1,5-disulphonic, 1-hydroxy-2-naphthoic, nicotinic, nitric, oleic, orotic, oxalic, palmitic, pamoic, phosphoric, propionic, L-pyroglutamic, salicylic, 4-amino-salicylic, sebacic, stearic, succinic, sulphuric, tannic, (+)-L-tartaric, thiocyanic, />-toluenesulphonic, undecylenic and valeric acids, as well as acylated amino acids and cation exchange resins.

One particular group of salts consists of salts formed from acetic, hydrochloric, hydriodic, phosphoric, nitric, sulphuric, citric, lactic, succinic, maleic, malic, isethionic, fumaric, benzenesulphonic, toluenesulphonic, methanesulphonic (mesylate), ethanesulphonic, naphthalenesulphonic, valeric, acetic, propanoic, butanoic, malonic, glucuronic and lactobionic acids.

One sub-group of salts consists of salts formed from hydrochloric, acetic, methanesulphonic, adipic, L-aspartic and DL-lactic acids.

Another sub-group of salts consists of the acetate, mesylate, ethanesulphonate, DL-lactate, adipate, D-glucuronate, D-gluconate and hydrochloride salts.

Preferred salts for use in the preparation of liquid (e.g. aqueous) compositions of the compounds of formulae (I) and sub-groups and examples thereof as described herein are salts having a solubility in a given liquid carrier (e.g. water) of greater than 10 mg/ml of the liquid carrier (e.g. water), more typically greater than 15 mg/ml and preferably greater than 20 mg/ml.

In one embodiment of the invention, there is provided a pharmaceutical composition comprising an aqueous solution containing a compound of the formula (I) and sub-groups and examples thereof as described herein in the form of a salt in a concentration of greater than 10 mg/ml, typically greater than 15 mg/ml and preferably greater than 20 mg/ml.

If the compound is anionic, or has a functional group which may be anionic (e.g., —COOH may be —COO⁻), then a salt may be formed with a suitable cation. Examples of suitable inorganic cations include, but are not limited to, alkali metal ions such as Na⁺ and K⁺, alkaline earth metal cations such as Ca²⁺ and Mg²⁺, and other cations such as Al³⁺. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

Where the compounds of the formula (I) contain an amine function, these may form quaternary ammonium salts, for example by reaction with an alkylating agent according to methods well known to the skilled person. Such quaternary ammonium compounds are within the scope of formula (I).

The salt forms of the compounds of the invention are typically pharmaceutically acceptable salts, and examples of pharmaceutically acceptable salts are discussed in Berge et ah, 1977, “Pharmaceutically Acceptable Salts,” J. Pharm. ScL, Vol. 66, pp. 1-19. However, salts that are not pharmaceutically acceptable may also be prepared as intermediate forms which may then be converted into pharmaceutically acceptable salts. Such non-pharmaceutically acceptable salts forms, which may be useful, for example, in the purification or separation of the compounds of the invention, also form part of the invention.

Compounds of the formula (I) containing an amine function may also form N-oxides. A reference herein to a compound of the formula (I) that contains an amine function also includes the N-oxide.

Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle.

N-Oxides can be formed by treatment of the corresponding amine with an oxidizing agent such as hydrogen peroxide or a per-acid (e.g. a peroxycarboxylic acid), see for example Advanced Organic Chemistry, by Jerry March, 4^{th} Edition, Wiley Interscience, pages. More particularly, N-oxides can be made by the procedure of L. W. Deady (Syn. Comm. 1977, 7, 509-514) in which the amine compound is reacted with m-chloroperoxybenzoic acid (MCPBA), for example, in an inert solvent such as dichloromethane.

Compounds of the formula (I) may exist in a number of different geometric isomeric, and tautomeric forms and references to compounds of the formula (I) include all such forms. For the avoidance of doubt, where a compound can exist in one of several geometric isomeric or tautomeric forms and only one is specifically described or shown, all others are nevertheless embraced by formula (I).

For example, in compounds of the formula (I) the pyrazole ring can exist in the two tautomeric forms A and B below. For simplicity, the general formula (I) illustrates form A but the formula is to be taken as embracing both tautomeric forms.

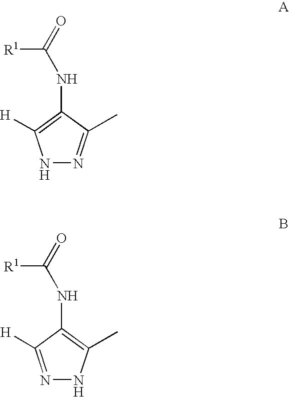

Other examples of tautomeric forms include, for example, keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

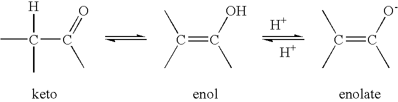

Where compounds of the formula (I) contain one or more chiral centres, and can exist in the form of two or more optical isomers, references to compounds of the formula (I) include all optical isomeric forms thereof (e.g. enantiomers, epimers and diastereoisomers), either as individual optical isomers, or mixtures (e.g. racemic mixtures) or two or more optical isomers, unless the context requires otherwise.

The optical isomers may be characterised and identified by their optical activity (i.e. as + and − isomers, or d and l isomers) or they may be characterised in terms of their absolute stereochemistry using the “R and S” nomenclature developed by Cahn, Ingold and Prelog, see Advanced Organic Chemistry by Jerry March, 4^{th} Edition, John Wiley & Sons, New York, 1992, pages 109-114, and see also Cahn, Ingold & Prelog, Angew. Chem. Int. Ed. Engl, 1966, 5, 385-415.

Optical isomers can be separated by a number of techniques including chiral chromatography (chromatography on a chiral support) and such techniques are well known to the person skilled in the art.

As an alternative to chiral chromatography, optical isomers can be separated by forming diastereoisomeric salts with chiral acids such as (+)-tartaric acid, (−)-pyroglutamic acid, (−)-di-toluoyl-L-tartaric acid, (+)-mandelic acid, (−)-malic acid, and (−)-camphorsulphonic, separating the diastereoisomers by preferential crystallisation, and then dissociating the salts to give the individual enantiomer of the free base.

Where compounds of the formula (I) exist as two or more optical isomeric forms, one enantiomer in a pair of enantiomers may exhibit advantages over the other enantiomer, for example, in terms of biological activity. Thus, in certain circumstances, it may be desirable to use as a therapeutic agent only one of a pair of enantiomers, or only one of a plurality of diastereoisomers. Accordingly, the invention provides compositions containing a compound of the formula (I) having one or more chiral centres, wherein at least 55% (e.g. at least 60%, 65%, 70%, 75%, 80%, 85%, 90% or 95%) of the compound of the formula (I) is present as a single optical isomer (e.g. enantiomer or diastereoisomer). In one general embodiment, 99% or more (e.g. substantially all) of the total amount of the compound of the formula (I) may be present as a single optical isomer (e.g. enantiomer or diastereoisomer).

The compounds of the invention include compounds with one or more isotopic substitutions, and a reference to a particular element includes within its scope all isotopes of the element. For example, a reference to hydrogen includes within its scope ¹H, ²H (D), and ³H (T). Similarly, references to carbon and oxygen include within their scope respectively ¹²C, ¹³C and ¹⁴C and ¹⁶O and ¹⁸O.

The isotopes may be radioactive or non-radioactive. In one embodiment of the invention, the compounds contain no radioactive isotopes. Such compounds are preferred for therapeutic use. In another embodiment, however, the compound may contain one or more radioisotopes. Compounds containing such radioisotopes may be useful in a diagnostic context.

Esters such as carboxylic acid esters and acyloxy esters of the compounds of formula (I) bearing a carboxylic acid group or a hydroxyl group are also embraced by Formula (I). Examples of esters are compounds containing the group —C(═O)OR, wherein R is an ester substituent, for example, a C₁₋₇ alkyl group, a C₃₋₂₀ heterocyclyl group, or a C₅₋₂₀ aryl group, preferably a C₁₋₇ alkyl group. Particular examples of ester groups include, but are not limited to, —C(O)OCH₃, —C(═O)OCH₂CH₃, —C(═O)OC(CH₃)₃, and —C(O)OPh. Examples of acyloxy (reverse ester) groups are represented by −0C(=0)R, wherein R is an acyloxy substituent, for example, a C₁₋₇ alkyl group, a C₃₋₂₀heterocyclyl group, or a C₅₋₂₀ aryl group, preferably a C₁₋₇ alkyl group. Particular examples of acyloxy groups include, but are not limited to, —0C(=0)CH₃ (acetoxy), —OC(═O)CH₂CH₃, —OC(═O)C(CH₃)₃, —OC(O)Ph, and —OC(O)CH₂Ph.

Also encompassed by formula (I) are any polymorphic forms of the compounds, solvates (e.g. hydrates), complexes (e.g. inclusion complexes or clathrates with compounds such as cyclodextrins, or complexes with metals) of the compounds, and pro-drugs of the compounds. By “prodrugs” is meant for example any compound that is converted in vivo into a biologically active compound of the formula (I).

For example, some prodrugs are esters of the active compound (e.g., a physiologically acceptable metabolically labile ester). During metabolism, the ester group (—C(═O)OR) is cleaved to yield the active drug. Such esters may be formed by esterification, for example, of any of the carboxylic acid groups (—C(O)OH) in the parent compound, with, where appropriate, prior protection of any other reactive groups present in the parent compound, followed by deprotection if required.

Examples of such metabolically labile esters include those of the formula —C(O)OR wherein R is:
C₁₋₇alkyl
(e.g., -Me, -Et, -nPr, -iPr, -nBu, -sBu, -iBu, -tBu);
C₁₋₇aminoalkyl
(e.g., aminoethyl; 2-(N,N-diethylamino)ethyl; 2-(4-morpholino)ethyl); and
acyloxy-C₁₋₇alkyl
(e.g., acyloxymethyl;
acyloxyethyl;
pivaloyloxymethyl;
acetoxymethyl;
1-acetoxyethyl;
1-(1-methoxy-1-methyl)ethyl-carbonxyloxyethyl;
1-(benzoyloxy)ethyl; isopropoxy-carbonyloxymethyl;
1-isopropoxy-carbonyloxyethyl; cyclohexyl-carbonyloxymethyl;
1-cyclohexyl-carbonyloxyethyl;
cyclohexyloxy-carbonyloxymethyl;
1-cyclohexyloxy-carbonyloxyethyl;
(4-tetrahydropyranyloxy) carbonyloxymethyl;
1-(4-tetrahydropyranyloxy)carbonyloxyethyl;
(4-tetrahydropyranyl)carbonyloxymethyl; and
1-(4-tetrahydropyranyl)carbonyloxyethyl).

Also, some prodrugs are activated enzymatically to yield the active compound, or a compound which, upon further chemical reaction, yields the active compound (for example, as in ADEPT, GDEPT, LIDEPT, etc.). For example, the prodrug may be a sugar derivative or other glycoside conjugate, or may be an amino acid ester derivative.

### Biological Activity

The compounds of the formulae (I) and sub-groups thereof are inhibitors of cyclin dependent kinases. For example, compounds of the invention are inhibitors of cyclin dependent kinases, and in particular cyclin dependent kinases selected from CDK1, CDK2, CDK3, CDK4, CDK5, CDK6 and CDK9, and more particularly selected from CDK1, CDK2, CDK3, CDK4, CDK5 and CDK9.

Preferred compounds are compounds that inhibit one or more CDK kinases selected from CPK1, CDK2, CDK4 and CDK9, for example CDK1 and/or CDK2.

Compounds of the invention also have activity against glycogen synthase kinase-3 (GSK-3).

As a consequence of their activity in modulating or inhibiting CDK and glycogen synthase kinase, they are expected to be useful in providing a means of arresting, or recovering control of, the cell cycle in abnormally dividing cells. It is therefore anticipated that the compounds will prove useful in treating or preventing proliferative disorders such as cancers. It is also envisaged that the compounds of the invention will be useful in treating conditions such as viral infections, type II or non-insulin dependent diabetes mellitus, autoimmune diseases, head trauma, stroke, epilepsy, neurodegenerative diseases such as Alzheimer's, motor neurone disease, progressive supranuclear palsy, corticobasal degeneration and Pick's disease for example autoimmune diseases and neurodegenerative diseases.

One sub-group of disease states and conditions where it is envisaged that the compounds of the invention will be useful consists of viral infections, autoimmune diseases and neurodegenerative diseases.

CDKs play a role in the regulation of the cell cycle, apoptosis, transcription, differentiation and CNS function. Therefore, CDK inhibitors could be useful in the treatment of diseases in which there is a disorder of proliferation, apoptosis or differentiation such as cancer. In particular RB+ve tumours may be particularly sensitive to CDK inhibitors. RB−ve tumours may also be sensitive to CDK inhibitors.

Examples of cancers which may be inhibited include, but are not limited to, a carcinoma, for example a carcinoma of the bladder, breast, colon (e.g. colorectal carcinomas such as colon adenocarcinoma and colon adenoma), kidney, epidermis, liver, lung, for example adenocarcinoma, small cell lung cancer and non-small cell lung carcinomas, oesophagus, gall bladder, ovary, pancreas e.g. exocrine pancreatic carcinoma, stomach, cervix, thyroid, prostate, or skin, for example squamous cell carcinoma; a hematopoietic tumour of lymphoid lineage, for example leukemia, acute lymphocytic leukemia, chronic lymphocytic leukaemia, B-cell lymphoma (such as diffuse large B cell lymphoma), T-cell lymphoma, Hodgkin's lymphoma, non-Hodgkin's lymphoma, hairy cell lymphoma, or Burkett's lymphoma; a hematopoietic tumour of myeloid lineage, for example acute and chronic myelogenous leukemias, myelodysplastic syndrome, or promyelocytic leukemia; thyroid follicular cancer; a tumour of mesenchymal origin, for example fibrosarcoma or habdomyosarcoma; a tumour of the central or peripheral nervous system, for example astrocytoma, neuroblastoma, glioma or schwannoma; melanoma; seminoma; teratocarcinoma; osteosarcoma; xeroderma pigmentosum; keratoctanthoma; thyroid follicular cancer; or Kaposi's sarcoma.

The cancers may be cancers which are sensitive to inhibition of any one or more cyclin dependent kinases selected from CDK1, CDK2, CDK3, CDK4, CDK5 and CDK6, for example, one or more CDK kinases selected from CDK1, CDK2, CDK4 and CDK5, e.g. CDK1 and/or CDK2.

Whether or not a particular cancer is one which is sensitive to inhibition by a cyclin dependent kinase may be determined by means of a cell growth assay as set out in the examples below or by a method as set out in the section headed “Methods of Diagnosis”.

CDKs are also known to play a role in apoptosis, proliferation, differentiation and transcription and therefore CDK inhibitors could also be useful in the treatment of the following diseases other than cancer; viral infections, for example herpes virus, pox virus, Epstein-Barr virus, Sindbis virus, adenovirus, HIV, HPV, HCV and HCMV; prevention of AIDS development in HIV-infected individuals; chronic inflammatory diseases, for example systemic lupus erythematosus, autoimmune mediated glomerulonephritis, rheumatoid arthritis, psoriasis, inflammatory bowel disease, and autoimmune diabetes mellitus; cardiovascular diseases for example cardiac hypertrophy, restenosis, atherosclerosis; neurodegenerative disorders, for example Alzheimer's disease, AIDS-related dementia, Parkinson's disease, amyotropic lateral sclerosis, retinitis pigmentosa, spinal muscular atrophy and cerebellar degeneration; glomerulonephritis; myelodysplastic syndromes, ischemic injury associated myocardial infarctions, stroke and reperfusion injury, arrhythmia, atherosclerosis, toxin-induced or alcohol related liver diseases, haematological diseases, for example, chronic anemia and aplastic anemia; degenerative diseases of the musculoskeletal system, for example, osteoporosis and arthritis, aspirin-sensitive rhinosinusitis, cystic fibrosis, multiple sclerosis, kidney diseases and cancer pain.

It has also been discovered that some cyclin-dependent kinase inhibitors can be used in combination with other anticancer agents. For example, the cyclin-dependent kinase inhibitor flavopiridol has been used with other anticancer agents in combination therapy.

Thus, in the pharmaceutical compositions, uses or methods of this invention for treating a disease or condition comprising abnormal cell growth, the disease or condition comprising abnormal cell growth in one embodiment is a cancer.

One group of cancers includes human breast cancers (e.g. primary breast tumours, node-negative breast cancer, invasive duct adenocarcinomas of the breast, non-endometrioid breast cancers); and mantle cell lymphomas. In addition, other cancers are colorectal and endometrial cancers.

Another sub-set of cancers includes hematopoietic tumours of lymphoid lineage, for example leukemia, chronic lymphocytic leukaemia, mantle cell lymphoma and B-cell lymphoma (such as diffuse large B cell lymphoma).

One particular cancer is chronic lymphocytic leukaemia.

Another particular cancer is mantle cell lymphoma.

Another particular cancer is diffuse large B cell lymphoma

Another sub-set of cancers includes breast cancer, ovarian cancer, colon cancer, prostate cancer, oesophageal cancer, squamous cancer and non-small cell lung carcinomas.

The activity of the compounds of the invention as inhibitors of cyclin dependent kinases and glycogen synthase kinase-3 can be measured using the assays set forth in the examples below and the level of activity exhibited by a given compound can be defined in terms of the IC₅₀ value. Preferred compounds of the present invention are compounds having an IC₅₀ value of less than 1 micromolar, more preferably less than 0.1 micromolar.

### Advantages of the Compounds of the Invention

Compounds of the formulae (I) and sub-groups thereof as defined herein have advantages over prior art compounds.

Potentially the compounds of the invention have physiochemical properties suitable for oral exposure.

Compounds of the invention have a higher IC₅₀ for transcription than IC₅₀ for proliferation in HCT-1 16 cells for example is ˜100-fold higher. This is advantageous as the compound could be better tolerated thus allowing it to be dosed at higher and for longer doses.

In particular, compounds of the formula (I) exhibit improved oral bioavailability relative to prior art compounds. Oral bioavailability can be defined as the ratio (F) of the plasma exposure of a compound when dosed by the oral route to the plasma exposure of the compound when dosed by the intravenous (i.v.) route, expressed as a percentage.

Compounds having an oral bioavailability (F value) of greater than 30%, more preferably greater than 40%, are particularly advantageous in that they may be adminstered orally rather than, or as well as, by parenteral administration.

### Methods for the Preparation of Compounds of the Formula (I)

In this section, as in all other sections of this application unless the context indicates otherwise, references to Formula (I) also include all sub-groups and examples thereof as defined herein. Where a reference is made to a group R¹, R³, R⁴, R⁷ or any other “R” group, the definition of the group in question is as set out above and as set out in the following sections of this application unless the context requires otherwise.

Compounds of the formula (I) can be prepared in accordance with synthetic methods well known to the skilled person, and by methods set out below and as described in our application PCT/GB2004/003179, the contents of which are incorporated herein by reference.

For example, compounds of the formula (I) can be prepared by the sequence of reactions shown in Scheme 1.

The starting material for the synthetic route shown in Scheme 1 is the 4-nitro-pyrazole-3-carboxylic acid (X) which can either be obtained commercially or can be prepared by nitration of the corresponding 4-unsubstituted pyrazole carboxy compound.

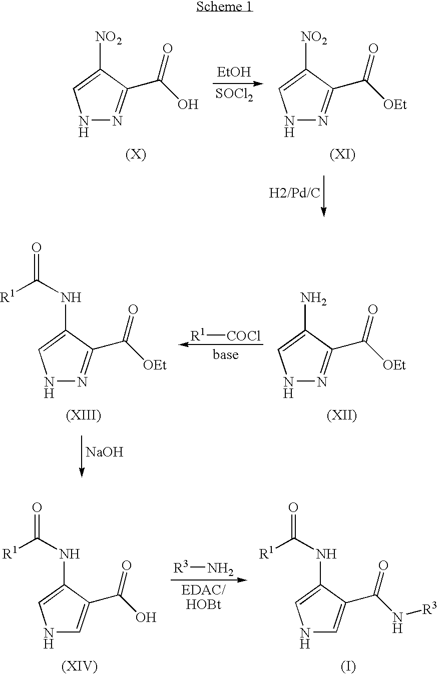

The nitro-pyrazole carboxylic acid (X) is converted to the corresponding ester (XI), for example the methyl or ethyl ester (of which the ethyl ester is shown), by reaction with the appropriate alcohol such as ethanol in the presence of an acid catalyst or thionyl chloride. The reaction may be carried out at ambient temperature using the esterifying alcohol as the solvent.

The nitro-ester (XI) can be reduced to the corresponding amine (XII) by standard methods for converting a nitro group to an amino group. Thus, for example, the nitro group can be reduced to the amine by hydrogenation over a palladium on charcoal catalyst. The hydrogenation reaction can be carried out in a solvent such as ethanol at ambient temperature.

The resulting amine (XII) can be converted to the amide (XIII) by reaction with an acid chloride of the formula R¹COCl in the presence of a non-interfering base such as triethylamine. The reaction may be carried out at around room temperature in a polar solvent such as dioxan. The acid chloride can be prepared by treatment of the carboxylic acid R¹CO₂H with thionyl chloride, or by reaction with oxalyl chloride in the presence of a catalytic amount of dimethyl formamide, or by reaction of a potassium salt of the acid with oxalyl chloride.

As an alternative to using the acid chloride method described above, the amine (XII) can be converted to the amide (XIII) by reaction with the carboxylic acid R¹CO₂H in the presence of amide coupling reagents of the type commonly used in the formation of peptide linkages. Examples of such reagents include 1,3-dicyclohexylcarbodiimide (DCC) (Sheehan et al, J. Amer. Chem. Soc. 1955, 77, 1067), 1-ethyl-3-(3′-dimethylaminopropyl)-carbodiimide (referred to herein either as EDC or EDAC but also known in the art as EDCI and WSCDI) (Sheehan et al, J. Org. Chem., 1961, 26, 2525), uronium-based coupling agents such as O-(7-azabenzotriazol-1-ŷ-N,Λ^{t},iV′,iV′-tetramethyluronium hexafluorophosphate (HATU) and phosphonium-based coupling agents such as 1-benzo-triazolyloxytris-(pyrrolidino)phosphonium hexafluorophosphate (PyBOP) (Castro et al, Tetrahedron Letters, 1990, 31, 205). Carbodiimide-based coupling agents are advantageously used in combination with 1-hydroxy-7-azabenzotriazole (HOAt) (L. A. Carpino, J. Amer. Chem. Soc, 1993, 115, 4397) or 1-hydroxybenzotriazole (HOBt) (Konig et al, Chem. Ber., 103, 708, 2024-2034). Preferred coupling reagents include EDC (EDAC) and DCC in combination with HOAt or HOBt.

The coupling reaction is typically carried out in a non-aqueous, non-protic solvent such as acetonitrile, dioxan, dimethylsulphoxide, dichloromethane, dimethylformamide or N-methylpyrrolidine, or in an aqueous solvent optionally together with one or more miscible co-solvents. The reaction can be carried out at room temperature or, where the reactants are less reactive (for example in the case of electron-poor anilines bearing electron withdrawing groups such as sulphonamide groups) at an appropriately elevated temperature. The reaction may be carried out in the presence of a non-interfering base, for example a tertiary amine such as triethylamine or N,iV-diisopropylethylamine.

The amide (XIII) is subsequently hydrolysed to the carboxylic acid (XIV) by treatment with an aqueous alkali metal hydroxide such sodium hydroxide. The saponification reaction may be carried out using an organic co-solvent such as an alcohol (e.g. methanol) and the reaction mixture is typically heated to a non-extreme temperature, for example up to about 50-60° C.

The carboxylic acid (XIV) can then be converted to a compound of the formula (I) by reaction with an amine R³—NH₂ using the amide forming conditions described above. Thus, for example, the amide coupling reaction may be carried out in the presence of EDC and HOBt in a polar solvent such as DMF.

An alternative general route to compounds of the formula (I) is shown in Scheme 2.

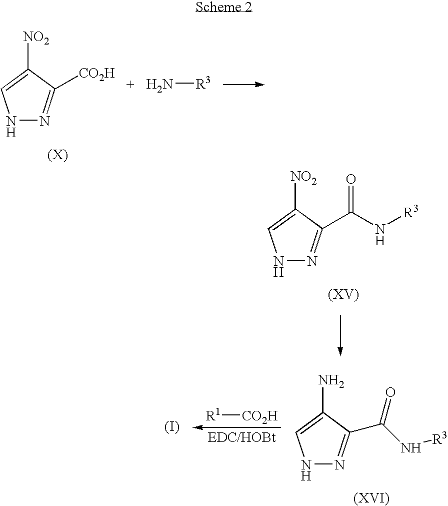

In Scheme 2, the nitro-pyrazole-carboxylic acid (X), or an activated derivative thereof such as an acid chloride, is reacted with amine R³—NH₂ using the amide forming conditions described above to give the nitro-pyrazole-amide (XV) which is then reduced to the corresponding amino compound (XVI) using a standard method of reducing nitro groups, for example the method involving hydrogenation over a Pd/C catalyst as described above.

The amine (XVI) is then coupled with a carboxylic acid of the formula RZ-CO₂H or an activated derivative thereof such as an acid chloride or anhydride under the amide-forming conditions described above in relation to Scheme 1. Thus, for example, as an alternative to using an acid chloride, the coupling reaction can be carried out in the presence of EDAC (EDC) and HOBt in a solvent such as DMF to give a compound of the formula (I).

Compounds of the formula (I) in which R³ is a sulphonyl piperidinyl group (i), or acyl piperidine group can be prepared by the methods described above or they can be prepared from a compound of the formula (XVII):

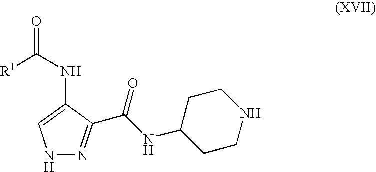

by reaction with an appropriate acylating or sulphonylating agent. Thus, for example, sulphonyl piperidinyl compounds can be prepared by reaction with the appropriate sulphonyl chloride such as methanesulphonyl chloride whereas acyl piperidine compounds and carbamate derivatives can be prepared by reacting a compound of the formula (XVII) with the appropriate acid chloride or chloroformate derivative respectively.

Illustrative reaction sequences showing the conversion of a compound of the formula (XVII) into sulphonyl and acyl and carbamate derivatives of the formula (I) are set out in Scheme 3.

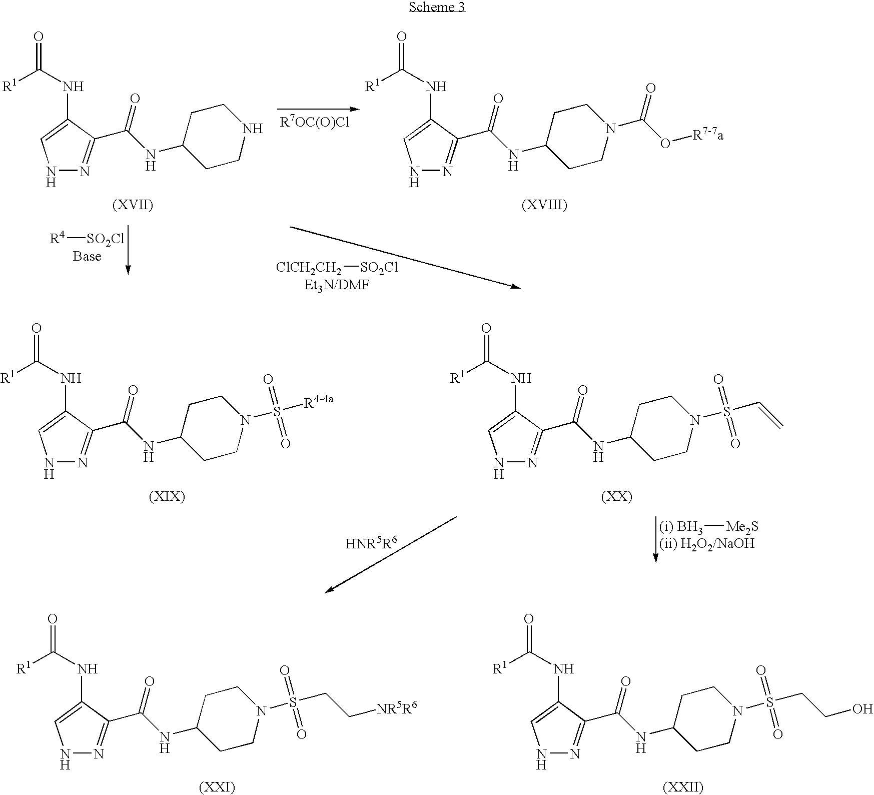

As shown in Scheme 3, a compound of the formula (I) in which R³ is a piperidine ring bearing a sulphonyl group —SO₂R⁴ (i.e. a compound of the formula (XIX)) can be prepared by reacting the compound of the formula (XVII) with a sulphonyl chloride R⁴SO₂Cl or R^{4a}SO₂Cl (such as methane sulphonyl chloride) in the presence of a non-interfering base such as diisopropylethylamine. The reaction is typically carried out at room temperature in a non-aqueous non-protic solvent such as dioxane and dichloromethane.

The sulphonyl chlorides of the formula R⁴SO₂Cl or R^{4a}SO₂Cl may be obtained from commercial sources, or can be prepared by a number of procedures. For example, alkylsulphonyl chlorides can be prepared by reacting an alkyl halide with sodium sulphite with heating in an aqueous organic solvent such as water/dioxane to form the corresponding sulphonic acid followed by treatment with thionyl chloride in the presence of DMF to give the sulphonyl chloride.

In an alternative preparation, a thiol R⁴SH/R^{4a}SH can be reacted with potassium nitrate and sulphuryl chloride to give the required sulphonyl chloride.

In a variation of this route, the piperidine compound of formula (XVII) can be reacted with 2-chloroethylsulphonyl chloride in the presence of a base such as triethylamine to give the vinylsulphonyl derivative (XX). The vinyl sulphonyl derivative may then be reacted with amines of the formula HNR⁵R⁶ in a Michael-type addition reaction to give compounds of the formula (XXI), in which the moiety NR⁵R⁶ is as defined elsewhere herein. The addition reaction is typically carried out at room temperature in a polar solvent such as an alcohol, e.g. ethanol. In a further variation, the amine HNR⁵R⁶ can be replaced by methoxylamine or methyl(methoxy)amine to give a methoxyl aminoethylsulphonyl or methyl(methoxy)aminosulphonyl analogue of the compound of formula (XXI).

The vinylsulphonyl compound (XX) may also be converted to the corresponding 2-hydroxyethyl compound by reaction with borane-dimethyl sulphide followed by alkaline hydrogen peroxide. The addition of the borane-dimethyl sulphide is typically carried out under the cover of an inert gas such as nitrogen in a polar non-protic solvent such as THF, for example at room temperature. The subsequent oxidation step with hydrogen peroxide may also be carried out at room temperature.

Compounds in which R³ is a piperidine ring bearing a carbamate group —C(O)OR⁷ or —C(O)OR^{7a} (i.e. compounds of the formula (XVIII) can be prepared by the reaction of a compound of the formula (XVII) with a chloroformate of the formula R⁷—O—C(O)—Cl or R^{7a}—O—C(O)—Cl in a polar solvent such as THF in the presence of a non-interfering base such as diisopropylethylamine, usually at or around room temperature. In a variation on this procedure, the compound of the formula (XVII) can be reacted with a chloroformate in which the group R⁷/R^{7a} contains a bromoalkyl moiety, for example a bromoethyl group. The resulting bromoalkylcarbamate can then be reacted with nucleophiles such as HNR⁵R⁶ or methoxylamine or methyl(methoxy)amine to give a compound in which R⁷/R^{7a} contains a group NR⁵R⁶ or a methoxylamino or methyl(methoxy)amino group.

In a further variation of the synthetic route shown in Scheme 3, the piperidine compound of formula (XVII) can be reacted with chloromethyl chloroformate and the resulting chloromethylcarbamate intermediate (not shown) treated with potassium acetate to form the acetoxymethyl carbamate compound. The reaction with potassium acetate is typically carried out in a polar solvent such as DMF with heating, for example to an elevated temperature in excess of 100° C. (e.g. up to about 110° C. Further variations on the synthetic route shown in Scheme 3 can be found in the Examples below.

In many of the reactions described above, it may be necessary to protect one or more groups to prevent reaction from taking place at an undesirable location on the molecule. Examples of protecting groups, and methods of protecting and deprotecting functional groups, can be found in Protective Groups in Organic Synthesis (T. Green and P. Wuts; 3rd Edition; John Wiley and Sons, 1999).

A hydroxy group may be protected, for example, as an ether (—OR) or an ester (—OC(═O)R), for example, as: a t-butyl ether; a benzyl, benzhydryl (diphenylmethyl), or trityl (triphenylmethyl)ether; a trimethylsilyl or t-butyldimethylsilyl ether; or an acetyl ester (—OC(═O)CH₃, —OAc). An aldehyde or ketone group may be protected, for example, as an acetal (R—CH(OR)₂) or ketal (R₂C(OR)₂), respectively, in which the carbonyl group (>C═O) is converted to a diether (>C(OR)₂), by reaction with, for example, a primary alcohol. The aldehyde or ketone group is readily regenerated by hydrolysis using a large excess of water in the presence of acid. An amine group may be protected, for example, as an amide (—NRCO—R) or a urethane (—NRCO—OR), for example, as: a methyl amide (—NHCO—CH ₃); a benzyloxy amide (—NHCO—OCH₂C₆H₅, —NH-Cbz); as a t-butoxy amide (—NHCO—OC(CH ₃)₃, —NH-Boc); a 2-biphenyl-2-propoxy amide (—NHCO—OC(CH₃)₂C₆H₄C₆H₅, —NH-Bpoc), as a 9-fluorenylmethoxy amide (—NH-Fmoc), as a 6-nitroveratryloxy amide (—NH-Nvoc), as a 2-trimethylsilylethyloxy amide (—NH-Teoc), as a 2,2,2-trichloroethyloxy amide (—NH-Troc), as an allyloxy amide (—NH-Alloc), or as a 2(-phenylsulphonyl)ethyloxy amide (—NH-Psec). Other protecting groups for amines, such as cyclic amines and heterocyclic N—H groups, include toluenesulphonyl (tosyl) and methanesulphonyl (mesyl) groups and benzyl groups such as apara-methoxybenzyl (PMB) group. A carboxylic acid group may be protected as an ester for example, as: an Ci₋₇ alkyl ester (e.g., a methyl ester; a t-butyl ester); a Ci₋₇ haloalkyl ester (e.g., a Ci₋₇ trihaloalkyl ester); atriCîalkylsilyl-Ĉalkyl ester; or a C₅₋₂₀ aryl-C₁₋₇ alkyl ester (e.g., a benzyl ester; a nitrobenzyl ester); or as an amide, for example, as a methyl amide. A thiol group may be protected, for example, as a thioether (—SR), for example, as: a benzyl thioether; an acetamidomethyl ether (—S—CH₂NHC(═O)CH₃).

Many of the intermediate compounds described above are novel. Accordingly, in a further aspect, the invention provides novel chemical intermediates, for example a novel compound of the formula (XIII), (XIV), (XV), (XVI) or (XVII) wherein R¹ and R³ are as defined herein.

### Methods of Purification

The compounds may be isolated and purified by a number of methods well known to those skilled in the art and examples of such methods include chromatographic techniques such as column chromatography (e.g. flash chromatography) and HPLC. Preparative LC-MS is a standard and effective method used for the purification of small organic molecules such as the compounds described herein. The methods for the liquid chromatography (LC) and mass spectrometry (MS) can be varied to provide better separation of the crude materials and improved detection of the samples by MS. Optimisation of the preparative gradient LC method will involve varying columns, volatile eluents and modifiers, and gradients. Methods are well known in the art for optimising preparative LC-MS methods and then using them to purify compounds. Such methods are described in Rosentreter U, Huber U.; Optimal fraction collecting in preparative LC/MS; J Comb Chem.; 2004; 6(2), 159-64 and Leister W, Strauss K, Wisnoski D, Zhao Z, Lindsley C, Development of a custom high-throughput preparative liquid chromatography/mass spectrometer platform for the preparative purification and analytical analysis of compound libraries; J Comb Chem.; 2003; 5(3); 322-9.

One such system for purifying compounds via preparative LC-MS is described in the experimental section below although a person skilled in the art will appreciate that alternative systems and methods to those described could be used. In particular, normal phase preparative LC based methods might be used in place of the reverse phase methods described here. Most preparative LC-MS systems utilise reverse phase LC and volatile acidic modifiers, since the approach is very effective for the purification of small molecules and because the eluents are compatible with positive ion electrospray mass spectrometry. Employing other chromatographic solutions e.g. normal phase LC, alternatively buffered mobile phase, basic modifiers etc as outlined in the analytical methods described above could alternatively be used to purify the compounds.

### Pharmaceutical Formulations

While it is possible for the active compound to be administered alone, it is preferable to present it as a pharmaceutical composition (e.g. formulation) comprising at least one active compound of the invention together with one or more pharmaceutically acceptable carriers, adjuvants, excipients, diluents, fillers, buffers, stabilisers, preservatives, lubricants, or other materials well known to those skilled in the art and optionally other therapeutic or prophylactic agents; for example agents that reduce or alleviate some of the side effects associated with chemotherapy. Particular examples of such agents include anti-emetic agents and agents that prevent or decrease the duration of chemotherapy-associated neutropenia and prevent complications that arise from reduced levels of red blood cells or white blood cells, for example erythropoietin (EPO), granulocyte macrophage-colony stimulating factor (GM-CSF), and granulocyte-colony stimulating factor (G-CSF).

Thus, the present invention further provides pharmaceutical compositions, as defined above, and methods of making a pharmaceutical composition comprising admixing at least one active compound, as defined above, together with one or more pharmaceutically acceptable carriers, excipients, buffers, adjuvants, stabilizers, or other materials, as described herein.

The term “pharmaceutically acceptable” as used herein pertains to compounds, materials, compositions, and/or dosage forms which are, within the scope of sound medical judgment, suitable for use in contact with the tissues of a subject (e.g. human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

Accordingly, in a further aspect, the invention provides compounds of the formula (I) and sub-groups thereof as defined herein in the form of pharmaceutical compositions.

The pharmaceutical compositions can be in any form suitable for oral, parenteral, topical, intranasal, ophthalmic, otic, rectal, intra-vaginal, or transdermal administration. Where the compositions are intended for parenteral administration, they can be formulated for intravenous, intramuscular, intraperitoneal, subcutaneous administration or for direct delivery into a target organ or tissue by injection, infusion or other means of delivery. The delivery can be by bolus injection, short term infusion or longer term infusion and can be via passive delivery or through the utilisation of a suitable infusion pump.

Pharmaceutical formulations adapted for parenteral administration include aqueous and non-aqueous sterile injection solutions which may contain anti-oxidants, buffers, bacteriostats, co-solvents, organic solvent mixtures, cyclodextrin complexation agents, emulsifying agents (for forming and stabilizing emulsion formulations), liposome components for forming liposomes, gellable polymers for forming polymeric gels, lyophilisation protectants and combinations of agents for, inter alia, stabilising the active ingredient in a soluble form and rendering the formulation isotonic with the blood of the intended recipient. Pharmaceutical formulations for parenteral administration may also take the form of aqueous and non-aqueous sterile suspensions which may include suspending agents and thickening agents (R. G. Strickly, Solubilizing Excipients in oral and injectable formulations, Pharmaceutical Research, Vol 21(2) 2004, p 201-230).

A drug molecule that is ionizable can be solubilized to the desired concentration by pH adjustment if the drug's pKₐ is sufficiently away from the formulation pH value. The acceptable range is pH 2-12 for intravenous and intramuscular administration, but subcutaneously the range is pH 2.7-9.0. The solution pH is controlled by either the salt form of the drug, strong acids/bases such as hydrochloric acid or sodium hydroxide, or by solutions of buffers which include but are not limited to buffering solutions formed from glycine, citrate, acetate, maleate, succinate, histidine, phosphate, tris(hydroxymethyl)aminomethane (TRIS), or carbonate.

The combination of an aqueous solution and a water-soluble organic solvent/surfactant (i.e., a cosolvent) is often used in injectable formulations. The water-soluble organic solvents and surfactants used in injectable formulations include but are not limited to propylene glycol, ethanol, polyethylene glycol 300, polyethylene glycol 400, glycerin, dimethylacetamide (DMA), N-methyl-2-pyrrolidone (NMP; Pharmasolve), dimethylsulphoxide (DMSO), Solutol HS 15, Cremophor EL, Cremophor RH 60, and polysorbate 80. Such formulations can usually be, but are not always, diluted prior to injection.

Propylene glycol, PEG 300, ethanol, Cremophor EL, Cremophor RH 60, and polysorbate 80 are the entirely organic water-miscible solvents and surfactants used in commercially available injectable formulations and can be used in combinations with each other. The resulting organic formulations are usually diluted at least 2-fold prior to rV bolus or IV infusion.

Alternatively increased water solubility can be achieved through molecular complexation with cyclodextrins

Liposomes are closed spherical vesicles composed of outer lipid bilayer membranes and an inner aqueous core and with an overall diameter of <100 μm. Depending on the level of hydrophobicity, moderately hydrophobic drugs can be solubilized by liposomes if the drug becomes encapsulated or intercalated within the liposome. Hydrophobic drugs can also be solubilized by liposomes if the drug molecule becomes an integral part of the lipid bilayer membrane, and in this case, the hydrophobic drug is dissolved in the lipid portion of the lipid bilayer. A typical liposome formulation contains water with phospholipid at −5-20 mg/ml, an isotonicifier, a pH 5-8 buffer, and optionally cholesterol.

The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use.

The pharmaceutical formulation can be prepared by lyophilising a compound of Formula (I) or acid addition salt thereof. Lyophilisation refers to the procedure of freeze-drying a composition. Freeze-drying and lyophilisation are therefore used herein as synonyms. A typical process is to solubilised the compound and the resulting formulation is clarified, sterile filtered and aseptically transferred to containers appropriate for lyophilisation (e.g. vials). In the case of vials, they are partially stoppered with lyo-stoppers. The formulation can be cooled to freezing and subjected to lyophilisation under standard conditions and then hermetically capped forming a stable, dry lyophile formulation. The composition will typically have a low residual water content, e.g. less than 5% e.g. less than 1% by weight based on weight of the lyophile.

The lyophilisation formulation may contain other excipients for example, thickening agents, dispersing agents, buffers, antioxidants, preservatives, and tonicity adjusters. Typical buffers include phosphate, acetate, citrate and glycine. Examples of antioxidants include ascorbic acid, sodium bisulphite, sodium metabisulphite, monothioglycerol, thiourea, butylated hydroxytoluene, butylated hydroxy! anisole, and ethylenediamietetraacetic acid salts. Preservatives may include benzoic acid and its salts, sorbic acid and its salts, alkyl esters of para-hydroxybenzoic acid, phenol, chlorobutanol, benzyl alcohol, thimerosal, benzalkonium chloride and cetylpyridinium chloride. The buffers mentioned previously, as well as dextrose and sodium chloride, can be used for tonicity adjustment if necessary.

Bulking agents are generally used in lyophilisation technology for facilitating the process and/or providing bulk and/or mechanical integrity to the lyophilized cake. Bulking agent means a freely water soluble, solid particulate diluent that when co-lyophilised with the compound or salt thereof, provides a physically stable lyophilized cake, a more optimal freeze-drying process and rapid and complete reconstitution. The bulking agent may also be utilised to make the solution isotonic.

The water-soluble bulking agent can be any of the pharmaceutically acceptable inert solid materials typically used for lyophilisation. Such bulking agents include, for example, sugars such as glucose, maltose, sucrose, and lactose; polyalcohols such as sorbitol or mannitol; amino acids such as glycine; polymers such as polyvinylpyrrolidine; and polysaccharides such as dextran.

The ratio of the weight of the bulking agent to the weight of active compound is typically within the range from about 1 to about 5, for example of about 1 to about 3, e.g. in the range of about 1 to 2.

Alternatively they can be provided in a solution form which may be concentrated and sealed in a suitable vial. Sterilisation of dosage forms may be via filtration or by autoclaving of the vials and their contents at appropriate stages of the formulation process. The supplied formulation may require further dilution or preparation before delivery for example dilution into suitable sterile infusion packs.

Extemporaneous injection solutions and suspensions may be prepared from sterile powders, granules and tablets.

In one preferred embodiment of the invention, the pharmaceutical composition is in a form suitable for i.v. administration, for example by injection or infusion.

Pharmaceutical compositions of the present invention for parenteral injection can also comprise pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol, and the like), carboxymethylcellulose and suitable mixtures thereof, vegetable oils (such as olive oil), and injectable organic esters such as ethyl oleate. Proper fluidity can be maintained, for example, by the use of coating materials such as lecithin, by the maintenance of the required particle size in the case of dispersions, and by the use of surfactants.

The compositions of the present invention may also contain adjuvants such as preservatives, wetting agents, emulsifying agents, and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents such as sugars, sodium chloride, and the like. Prolonged absorption of the injectable pharmaceutical form may be brought about by the inclusion of agents which delay absorption such as aluminum monostearate and gelatin.

If a compound is not stable in aqueous media or has low solubility in aqueous media, it can be formulated as a concentrate in organic solvents. The concentrate can then be diluted to a lower concentration in an aqueous system, and can be sufficiently stable for the short period of time during dosing. Therefore in another aspect, there is provided a pharmaceutical composition comprising a non aqueous solution composed entirely of one or more organic solvents, which can be dosed as is or more commonly diluted with a suitable IV excipient (saline, dextrose; buffered or not buffered) before administration (Solubilizing excipients in oral and injectable formulations, Pharmaceutical Research, 21(2), 2004, p 201-230). Examples of solvents and surfactants are propylene glycol, PEG300, PEG40Q, ethanol, dimethylacetamide (DMA), N-methyl-2-pyrrolidone (NMP, Pharmasolve), Glycerin, Cremophor EL, Cremophor RH 60 and polysorbate. Particular non aqueous solutions are composed of 70-80% propylene glycol, and 20-30% ethanol. One particular non aqueous solution is composed of 70% propylene glycol, and 30% ethanol. Another is 80% propylene glycol and 20% ethanol. Normally these solvents are used in combination and usually diluted at least 2-fold before IV bolus or IV infusion. The typical amounts for bolus IV formulations are −50% for Glycerin, propylene glycol, PEG300, PEG400, and −20% for ethanol. The typical amounts for IV infusion formulations are −15% for Glycerin, 3% for DMA, and −10% for propylene glycol, PEG300, PEG400 and ethanol.

In one preferred embodiment of the invention, the pharmaceutical composition is in a form suitable for i.v. administration, for example by injection or infusion. For intravenous administration, the solution can be dosed as is, or can be injected into an infusion bag (containing a pharmaceutically acceptable excipient, such as 0.9% saline or 5% dextrose), before administration.

In another preferred embodiment, the pharmaceutical composition is in a form suitable for sub-cutaneous (s.c.) administration.

Pharmaceutical dosage forms suitable for oral administration include tablets, capsules, caplets, pills, lozenges, syrups, solutions, powders, granules, elixirs and suspensions, sublingual tablets, wafers or patches and buccal patches.

Pharmaceutical compositions containing compounds of the formula (I) can be formulated in accordance with known techniques, see for example, Remington's Pharmaceutical Sciences, Mack Publishing Company, Easton, Pa., USA.

Thus, tablet compositions can contain a unit dosage of active compound together with an inert diluent or carrier such as a sugar or sugar alcohol, eg; lactose, sucrose, sorbitol or mannitol; and/or a non-sugar derived diluent such as sodium carbonate, calcium phosphate, calcium carbonate, or a cellulose or derivative thereof such as methyl cellulose, ethyl cellulose, hydroxypropyl methyl cellulose, and starches such as corn starch. Tablets may also contain such standard ingredients as binding and granulating agents such as polyvinylpyrrolidone, disintegrants (e.g. swellable crosslinked polymers such as crosslinked carboxymethylcellulose), lubricating agents (e.g. stearates), preservatives (e.g. parabens), antioxidants (e.g. BHT), buffering agents (for example phosphate or citrate buffers), and effervescent agents such as citrate/bicarbonate mixtures. Such excipients are well known and do not need to be discussed in detail here.

Capsule formulations may be of the hard gelatin or soft gelatin variety and can contain the active component in solid, semi-solid, or liquid form. Gelatin capsules can be formed from animal gelatin or synthetic or plant derived equivalents thereof.

The solid dosage forms (eg; tablets, capsules etc.) can be coated or un-coated, but typically have a coating, for example a protective film coating (e.g. a wax or varnish) or a release controlling coating. The coating (e.g. a Eudragit™ type polymer) can be designed to release the active component at a desired location within the gastrointestinal tract. Thus, the coating can be selected so as to degrade under certain pH conditions within the gastrointestinal tract, thereby selectively release the compound in the stomach or in the ileum or duodenum.

Instead of, or in addition to, a coating, the drug can be presented in a solid matrix comprising a release controlling agent, for example a release delaying agent which may be adapted to selectively release the compound under conditions of varying acidity or alkalinity in the gastrointestinal tract. Alternatively, the matrix material or release retarding coating can take the form of an erodible polymer (e.g. a maleic anhydride polymer) which is substantially continuously eroded as the dosage form passes through the gastrointestinal tract. As a further alternative, the active compound can be formulated in a delivery system that provides osmotic control of the release of the compound. Osmotic release and other delayed release or sustained release formulations may be prepared in accordance with methods well known to those skilled in the art.

The pharmaceutical compositions comprise from approximately 1% to approximately 95%, preferably from approximately 20% to approximately 90%, active ingredient. Pharmaceutical compositions according to the invention may be, for example, in unit dose form, such as in the form of ampoules, vials, suppositories, dragees, tablets or capsules.

Pharmaceutical compositions for oral administration can be obtained by combining the active ingredient with solid carriers, if desired granulating a resulting mixture, and processing the mixture, if desired or necessary, after the addition of appropriate excipients, into tablets, dragee cores or capsules. It is also possible for them to be incorporated into plastics carriers that allow the active ingredients to diffuse or be released in measured amounts.

The compounds of the invention can also be formulated as solid dispersions. Solid dispersions are homogeneous extremely fine disperse phases of two or more solids. Solid solutions (molecularly disperse systems), one type of solid dispersion, are well known for use in pharmaceutical technology (see (Chiou and Riegelman, J. Pharm. ScL, 60, 1281-1300 (1971)) and are useful in increasing dissolution rates and increasing the bioavailability of poorly water-soluble drugs.

Solid dispersions of drugs are generally produced by melt or solvent evaporation methods. For melt processing, the materials (excipients) which are usually semisolid and waxy in nature, are heated to cause melting and dissolution of the drug substance, followed by hardening by cooling to very low temperatures. The solid dispersion can then be pulverized, sieved, mixed with excipients, and encapsulated into hard gelatin capsules or compressed into tablets. Alternatively the use of surface-active and self-emulsifying carriers allows the encapsulation of solid dispersions directly into hard gelatin capsules as melts. Solid plugs are formed inside the capsules when the melts are cooled to room temperature.

Solid solutions can also be manufactured by dissolving the drug and the required excipient in either an aqueous solution or a pharmaceutically acceptable organic solvent, followed by removal of the solvent, using a pharmaceutically acceptable method, such as spray drying. The resulting solid can be particle sized if required, optionally mixed with excipients and either made into tablets or filled into capsules.

A particularly suitable polymeric auxiliary for producing such solid dispersions or solid solutions is polyvinylpyrrolidone (PVP).

The present invention provides a pharmaceutical composition comprising a substantially amorphous solid solution, said solid solution comprising

(a) a compound of the formula (I), for example the compound of Example 1; and
(b) a polymer selected from the group consisting of:
polyvinylpyrrolidone (povidone), crosslinked polyvinylpyrrolidone (crospovidone), hydroxypropyl methylcellulose, hydroxypropylcellulose, polyethylene oxide, gelatin, crosslinked polyacrylic acid (carbomer), carboxymethylcellulose, crosslinked carboxymethylcellulose (croscarmellose), methylcellulose, methacrylic acid copolymer, methacrylate copolymer, and water soluble salts such as sodium and ammonium salts of methacrylic acid and methacrylate copolymers, cellulose acetate phthalate, hydroxypropylmethylcellulose phthalate and propylene glycol alginate;
wherein the ratio of said compound to said polymer is about 1:1 to about 1:6, for example a 1:3 ratio, spray dried from a mixture of one of chloroform or dichloromethane and one of methanol or ethanol, preferably dichloromethane/ethanol in a 1:1 ratio.

This invention also provides solid dosage forms comprising the solid solution described above. Solid dosage forms include tablets, capsules and chewable tablets. Known excipients can be blended with the solid solution to provide the desired dosage form. For example, a capsule can contain the solid solution blended with (a) a disintegrant and a lubricant, or (b) a disintegrant, a lubricant and a surfactant. A tablet can contain the solid solution blended with at least one disintegrant, a lubricant, a surfactant, and a glidant. The chewable tablet can contain the solid solution blended with a bulking agent, a lubricant, and if desired an additional sweetening agent (such as an artificial sweetener), and suitable flavours.

The pharmaceutical formulations may be presented to a patient in “patient packs” containing an entire course of treatment in a single package, usually a blister pack. Patient packs have an advantage over traditional prescriptions, where a pharmacist divides a patient's supply of a pharmaceutical from a bulk supply, in that the patient always has access to the package insert contained in the patient pack, normally missing in patient prescriptions. The inclusion of a package insert has been shown to improve patient compliance with the physician's instructions.

Compositions for topical use include ointments, creams, sprays, patches, gels, liquid drops and inserts (for example intraocular inserts). Such compositions can be formulated in accordance with known methods.

Compositions for parenteral administration are typically presented as sterile aqueous or oily solutions or fine suspensions, or may be provided in finely divided sterile powder form for making up extemporaneously with sterile water for injection.

Examples of formulations for rectal or intra-vaginal administration include pessaries and suppositories which may be, for example, formed from a shaped moldable or waxy material containing the active compound.

Compositions for administration by inhalation may take the form of inhalable powder compositions or liquid or powder sprays, and can be administrated in standard form using powder inhaler devices or aerosol dispensing devices. Such devices are well known. For administration by inhalation, the powdered formulations typically comprise the active compound together with an inert solid powdered diluent such as lactose.

The compounds of the formula (I) will generally be presented in unit dosage form and, as such, will typically contain sufficient compound to provide a desired level of biological activity. For example, a formulation may contain from 1 nanogram to 2 grams of active ingredient, e.g. from 1 nanogram to 2 milligrams of active ingredient. Within this range, particular sub-ranges of compound are 0.1 milligrams to 2 grams of active ingredient (more usually from 10 milligrams to 1 gram, e.g. 50 milligrams to 500 milligrams), or 1 microgram to 20 milligrams (for example 1 microgram to 10 milligrams, e.g. 0.1 milligrams to 2 milligrams of active ingredient).

For oral compositions, a unit dosage form may contain from 1 milligram to 2 grams, more typically 10 milligrams to 1 gram, for example 50 milligrams to 1 gram, e.g. 100 miligrams to 1 gram, of active compound.

The active compound will be administered to a patient in need thereof (for example a human or animal patient) in an amount sufficient to achieve the desired therapeutic effect.

### Methods of Treatment

It is envisaged that the compounds of the formulae (I), (II), (III) and sub-groups as defined herein will be useful in the prophylaxis or treatment of a range of disease states or conditions mediated by cyclin dependent kinases and glycogen synthase kinase-3. Examples of such disease states and conditions are set out above.

The compounds are generally administered to a subject in need of such administration, for example a human or animal patient, preferably a human.

The compounds will typically be administered in amounts that are therapeutically or prophylactically useful and which generally are non-toxic. However, in certain situations (for example in the case of life threatening diseases), the benefits of administering a compound of the formula (I) may outweigh the disadvantages of any toxic effects or side effects, in which case it may be considered desirable to administer compounds in amounts that are associated with a degree of toxicity.

The compounds may be administered over a prolonged term to maintain beneficial therapeutic effects or may be administered for a short period only. Alternatively they may be administered in a pulsatile or continuous manner.

A typical daily dose of the compound of formula (I) can be in the range from 100 picograms to 100 milligrams per kilogram of body weight, more typically 5 nanograms to 25 milligrams per kilogram of bodyweight, and more usually 10 nanograms to 15 milligrams per kilogram (e.g. 10 nanograms to 10 milligrams, and more typically 1 microgram per kilogram to 20 milligrams per kilogram, for example 1 microgram to 10 milligrams per kilogram) per kilogram of bodyweight although higher or lower doses may be administered where required. The compound of the formula (I) can be administered on a daily basis or on a repeat basis every 2, or 3, or 4, or 5, or 6, or 7, or 10 or 14, or 21, or 28 days for example.

The compounds of the invention may be administered orally in a range of doses, for example 1 to 1500 mg, 2 to 800 mg, or 5 to 500 mg, e.g. 2 to 200 mg or 10 to 1000 mg, particular examples of doses including 10, 20, 50 and 80 mg. The compound may be administered once or more than once each day. The compound can be administered continuously (i.e. taken every day without a break for the duration of the treatment regimen). Alternatively, the compound can be administered intermittently (i.e. taken continuously for a given period such as a week, then discontinued for a period such as a week and then taken continuously for another period such as a week and so on throughout the duration of the treatment regimen. Examples of treatment regimens involving intermittent administration include regimens wherein administration is in cycles of one week on, one week off; or two weeks on, one week off; or three weeks on, one week off; or two weeks on, two weeks off; or four weeks on two weeks off; or one week on three weeks off—for one or more cycles, e.g. 2, 3, 4, 5, 6, 7, 8, 9 or 10 or more cycles.

An example of a dosage for a 60 kilogram person comprises administering a compound of the formula (I) as defined herein at a starting dosage of 4.5-10.8 mg/60 kg/day (equivalent to 75-180 μg/kg/day) and subsequently by an efficacious dose of 44-97 mg/60 kg/day (equivalent to 0.7-1.6 mg/kg/day) or an efficacious dose of 72-27.4 mg/60 kg/day (equivalent to 1.2-4.6 mg/kg/day) although higher or lower doses may be administered where required. The mg/kg dose would scale pro-rata for any given body weight.

In one particular dosing schedule, a patient will be given an infusion of a compound of the formula (I) for periods of one hour daily for up to ten days in particular up to five days for one week, and the treatment repeated at a desired interval such as two to four weeks, in particular every three weeks.

More particularly, a patient may be given an infusion of a compound of the formula (I) for periods of one hour daily for 5 days and the treatment repeated every three weeks.

In another particular dosing schedule, a patient is given an infusion over 30 minutes to 1 hour followed by maintenance infusions of variable duration, for example 1 to 5 hours, e.g. 3 hours.

In a further particular dosing schedule, a patient is given a continuous infusion for a period of 12 hours to 5 days, an in particular a continuous infusion of 24 hours to 72 hours.

Ultimately, however, the quantity of compound administered and the type of composition used will be commensurate with the nature of the disease or physiological condition being treated and will be at the discretion of the physician.

The compounds of formula (I) and sub-groups as defined herein can be administered as the sole therapeutic agent or they can be administered in combination therapy with one of more other compounds for treatment of a particular disease state, for example a neoplastic disease such as a cancer as hereinbefore defined. Examples of other therapeutic agents or therapies that may be administered or used together (whether concurrently or at different time intervals) with the compounds of the invention include but are not limited to topoisomerase inhibitors, alkylating agents, antimetabolites, DNA binders, microtubule inhibitors (tubulin targeting agents), monoclonal antibodies and signal transduction inhibitors, particular examples being cisplatin, cyclophosphamide, doxorubicin, irinotecan, fludarabine, 5FU, taxanes, mitomycin C and radiotherapy.

For the case of CDK inhibitors combined with other therapies, the two or more treatments may be given in individually varying dose schedules and via different routes.

Where the compound of the formula (I) is administered in combination therapy with one, two, three, four or more other therapeutic agents (preferably one or two, more preferably one), the compounds can be administered simultaneously or sequentially. When administered sequentially, they can be administered at closely spaced intervals (for example over a period of 5-10 minutes) or at longer intervals (for example 1, 2, 3, 4 or more hours apart, or even longer periods apart where required), the precise dosage regimen being commensurate with the properties of the therapeutic agent(s).

The compounds of the invention may also be administered in conjunction with non-chemotherapeutic treatments such as radiotherapy, photodynamic therapy, gene therapy; surgery and controlled diets.

For use in combination therapy with another chemotherapeutic agent, the compound of the formula (I) and one, two, three, four or more other therapeutic agents can be, for example, formulated together in a dosage form containing two, three, four or more therapeutic agents. In an alternative, the individual therapeutic agents may be formulated separately and presented together in the form of a kit, optionally with instructions for their use.

A person skilled in the art would know through his or her common general knowledge the dosing regimes and combination therapies to use.

### Methods of Diagnosis

Prior to administration of a compound of the formula (I), a patient may be screened to determine whether a disease or condition from which the patient is or may be suffering is one which would be susceptible to treatment with a compound having activity against cyclin dependent kinases.

For example, a biological sample taken from a patient may be analysed to determine whether a condition or disease, such as cancer, that the patient is or may be suffering from is one which is characterised by a genetic abnormality or abnormal protein expression which leads to over-activation of CDKs or to sensitisation of a pathway to normal CDK activity. Examples of such abnormalities that result in activation or sensitisation of the CDK2 signal include up-regulation of cyclin E, (Harwell R M, Mull B B, Porter D C, Keyomarsi K.; J Biol. Chem. 2004 Mar. 26; 279(13):12695-705) or loss of p21 or p27, or presence of CDC4 variants (Rajagopalan H, Jallepalli P V, Rago C, Velculescu V E, Kinzler K W, Vogelstein B, Lengauer C; Nature. 2004 Mar. 4; 428(6978):77-81). Tumours with mutants of CDC4 or up-regulation, in particular over-expression, of cyclin E or loss of p21 or p27 may be particularly sensitive to CDK inhibitors. The term up-regulation includes elevated expression or over-expression, including gene amplification (i.e. multiple gene copies) and increased expression by a transcriptional effect, and hyperactivity and activation, including activation by mutations.

Thus, the patient may be subjected to a diagnostic test to detect a marker characteristic of up-regulation of cyclin E, or loss of p21 or p27, or presence of CDC4 variants. The term diagnosis includes screening. By marker we include genetic markers including, for example, the measurement of DNA composition to identify mutations of CDC4. The term marker also includes markers which are characteristic of up regulation of cyclin E, including enzyme activity, enzyme levels, enzyme state (e.g. phosphorylated or not) and mRNA levels of the aforementioned proteins. Tumours with upregulation of cyclin E, or loss of p21 or p27 may be particularly sensitive to CDK inhibitors. Tumours may preferentially be screened for upregulation of cyclin E, or loss of p21 or p27 prior to treatment. Thus, the patient may be subjected to a diagnostic test to detect a marker characteristic of up-regulation of cyclin E, or loss of p21 or p27.

The diagnostic tests are typically conducted on a biological sample selected from tumour biopsy samples, blood samples (isolation and enrichment of shed tumour cells), stool biopsies, sputum, chromosome analysis, pleural fluid, peritoneal fluid, or urine.

It has been found, Rajagopalan et al (Nature. 2004 Mar. 4; 428(6978):77-81), that there were mutations present in CDC4 (also known as Fbw7 or Archipelago) in human colorectal cancers and endometrial cancers (Spruck et al, Cancer Res. 2002 Aug. 15; 62(16):4535-9). Identification of individual carrying a mutation in CDC4 may mean that the patient would be particularly suitable for treatment with a CDK inhibitor. Tumours may preferentially be screened for presence of a CDC4 variant prior to treatment. The screening process will typically involve direct sequencing, oligonucleotide microarray analysis, or a mutant specific antibody.

Methods of identification and analysis of mutations and up-regulation of proteins are well known to a person skilled in the art. Screening methods could include, but are not limited to, standard methods such as reverse-transcriptase polymerase chain reaction (RT-PCR) or in-situ hybridisation.

In screening by RT-PCR, the level of mRNA in the tumour is assessed by creating a cDNA copy of the mRNA followed by amplification of the cDNA by PCR. Methods of PCR amplification, the selection of primers, and conditions for amplification, are known to a person skilled in the art. Nucleic acid manipulations and PCR are carried out by standard methods, as described for example in Ausubel, F. M. et al., eds. Current Protocols in Molecular Biology, 2004, John Wiley & Sons Inc., or Innis, M. A. et-al., eds. PCR Protocols: a guide to methods and applications, 1990, Academic Press, San Diego. Reactions and manipulations involving nucleic acid techniques are also described in Sambrook et al., 2001, 3^{rd} Ed, Molecular Cloning: A Laboratory Manual, Cold Spring Harbor Laboratory Press. Alternatively a commercially available kit for RT-PCR (for example Roche Molecular Biochemicals) may be used, or methodology as set forth in U.S. Pat. Nos. 4,666,828; 4,683,202; 4,801,531; 5,192,659, 5,272,057, 5,882,864, and 6,218,529 and incorporated herein by reference.

An example of an in-situ hybridisation technique for assessing mRNA expression would be fluorescence in-situ hybridisation (FISH) (see Angerer, 1987 Meth. Enzymol., 152: 649).

Generally, in situ hybridization comprises the following major steps: (1) fixation of tissue to be analyzed; (2) prehybridization treatment of the sample to increase accessibility of target nucleic acid, and to reduce nonspecific binding; (3) hybridization of the mixture of nucleic acids to the nucleic acid in the biological structure or tissue; (4) post-hybridization washes to remove nucleic acid fragments not bound in the hybridization, and (5) detection of the hybridized nucleic acid fragments. The probes used in such applications are typically labeled, for example, with radioisotopes or fluorescent reporters. Preferred probes are sufficiently long, for example, from about 50, 100, or 200 nucleotides to about 1000 or more nucleotides, to enable specific hybridization with the target nucleic acid(s) under stringent conditions. Standard methods for carrying out FISH are described in Ausubel, F. M. et al, eds. Current Protocols in Molecular Biology, 2004, John Wiley & Sons Inc and Fluorescence In Situ Hybridization: Technical Overview by John M. S. Bartlett in Molecular Diagnosis of Cancer, Methods and Protocols, 2nd ed.; ISBN: 1-59259-760-2; March 2004, pps. 077-088; Series: Methods in Molecular Medicine.

Alternatively, the protein products expressed from the mRNAs may be assayed by immunohistochemistry of tumour samples, solid phase immunoassay with microtiter plates, Western blotting, 2-dimensional SDS-polyacrylamide gel electrophoresis, ELISA, flow cytometry and other methods known in the art for detection of specific proteins. Detection methods would include the use of site specific antibodies. The skilled person will recognize that all such well-known techniques for detection of upregulation of cyclin E, or loss of p21 or p27, or detection of CDC4 variants could be applicable in the present case.

Therefore, all of these techniques could also be used to identify tumours particularly suitable for treatment with the compounds of the invention.

Tumours with mutants of CDC4 or up-regulation, in particular over-expression, of cyclin E or loss of p21 or p27 may be particularly sensitive to CDK inhibitors. Tumours may preferentially be screened for up-regulation, in particular over-expression, of cyclin E (Harwell R M, Mull B B, Porter D C, Keyomarsi K.; J Biol. Chem. 2004 Mar. 26; 279(13): 12695-705) or loss of p21 or p27 or for CDC4 variants prior to treatment (Rajagopalan H, Jallepalli P V, Rago C, Velculescu V E, Kinzler K W, Vogelstein B, Lengauer C; Nature. 2004 Mar. 4; 428(6978):77-81).

Patients with mantle cell lymphoma (MCL) could be selected for treatment with a compound of the invention using diagnostic tests outlined herein. MCL is a distinct clinicopathologic entity of non-Hodgkin's lymphoma, characterized by proliferation of small to medium-sized lymphocytes with co-expression of CD5 and CD20, an aggressive and incurable clinical course, and frequent t(1 I; 14)(q13; q32) translocation. Over-expression of cyclin D1 mRNA, found in mantle cell lymphoma (MCL), is a critical diagnostic marker. Yatabe et al (Blood. 2000 Apr. 1; 95(7):2253-61) proposed that cyclin D1-positively should be included as one of the standard criteria for MCL, and that innovative therapies for this incurable disease should be explored on the basis of the new criteria. Jones et al (J Mol Diagn. 2004 May; 6(2):84-9) developed a real-time, quantitative, reverse transcription PCR assay for cyclin D1 (CCND1) expression to aid in the diagnosis of mantle cell lymphoma (MCL). Howe et al (Clin Chem. 2004 January; 50(1):80-7) used real-time quantitative RT-PCR to evaluate cyclin D1 mRNA expression and found that quantitative RT-PCR for cyclin D1 mRNA normalized to CD19 mRNA can be used in the diagnosis of MCL in blood, marrow, and tissue. Alternatively, patients with breast cancer could be selected for treatment with a CDK inhibitor using diagnostic tests outline above. Tumour cells commonly overexpress cyclin E and it has been shown that cyclin E is over-expressed in breast cancer (Harwell et al, Cancer Res, 2000, 60, 481-489). Therefore breast cancer may in particular be treated with a CDK inhibitor as provided herein.

### Antifungal Use

In a further aspect, the invention provides the use of the compounds of the formula (I) and sub-groups thereof as defined herein as antifungal agents.

The compounds of the formula (I) and sub-groups thereof as defined herein may be used in animal medicine (for example in the treatment of mammals such as humans), or in the treatment of plants (e.g. in agriculture and horticulture), or as general antifungal agents, for example as preservatives and disinfectants.

In one embodiment, the invention provides a compound of the formula (I) and sub-groups thereof as defined herein for use in the prophylaxis or treatment of a fungal infection in a mammal such as a human.

Also provided is the use of a compound of the formula (I) and sub-groups thereof as defined herein for the manufacture of a medicament for use in the prophylaxis or treatment of a fungal infection in a mammal such as a human.

For example, compounds of the invention may be administered to human patients suffering from, or at risk of infection by, topical fungal infections caused by among other organisms, species of Candida, Trichophyton, Microsporum or Epidermophyton, or in mucosal infections caused by Candida albicans (e.g. thrush and vaginal candidiasis). The compounds of the invention can also be administered for the treatment or prophylaxis of systemic fungal infections caused by, for example, Candida albicans, Cryptococcus neoformans, Aspergillus flavus, Aspergillus fumigatus, Coccidiodies, Paracoccidioides, Histoplasma or Blastomyces.

In another aspect, the invention provides an antifungal composition for agricultural (including horticultural) use, comprising a compound of the formulae (I) and sub-groups thereof as defined herein together with an agriculturally acceptable diluent or carrier.

The invention further provides a method of treating an animal (including a mammal such as a human), plant or seed having a fungal infection, which comprises treating said animal, plant or seed, or the locus of said plant or seed, with an effective amount of a compound of the formula (I) and sub-groups thereof as defined herein.

The invention also provides a method of treating a fungal infection in a plant or seed which comprises treating the plant or seed with an antifungally effective amount of a fungicidal composition containing a compound of the formula (I) and sub-groups thereof as defined herein.

Differential screening assays may be used to select for those compounds of the present invention with specificity for non-human CDK enzymes. Compounds which act specifically on the CDK enzymes of eukaryotic pathogens can be used as anti-fungal or anti-parasitic agents. Inhibitors of the Candida CDK kinase, CKSI, can be used in the treatment of candidiasis. Antifungal agents can be used against infections of the type hereinbefore defined, or opportunistic infections that commonly occur in debilitated and immunosuppressed patients such as patients with leukemias and lymphomas, people who are receiving immunosuppressive therapy, and patients with predisposing conditions such as diabetes mellitus or AIDS, as well as for non-immunosuppressed patients.

Assays described in the art can be used to screen for agents which may be useful for inhibiting at least one fungus implicated in mycosis such as candidiasis, aspergillosis, mucormycosis, blastomycosis, geotrichosis, cryptococcosis, chromoblastomycosis, coccidiodomycosis, conidiosporosis, histoplasmosis, maduromycosis, rhinosporidosis, nocardiosis, para-actinomycosis, penicilliosis, monoliasis, or sporotrichosis. The differential screening assays can be used to identify anti-fungal agents which may have therapeutic value in the treatment of aspergillosis by making use of the CDK genes cloned from yeast such as Aspergillus fumigatus, Aspergillus flavus, Aspergillus niger, Aspergillus nidulans, or Aspergillus terreus, or where the mycotic infection is mucon-nycosis, the CDK assay can be derived from yeast such as Rhizopus arrhizus, Rhizopus oryzae, Absidia corymbifera, Absidia ramosa, or Mucor pusillus. Sources of other CDK enzymes include the pathogen Pneumocystis carinii.

By way of example, in vitro evaluation of the antifungal activity of the compounds can be performed by determining the minimum inhibitory concentration (M.I.C.) which is the concentration of the test compounds, in a suitable medium, at which growth of the particular microorganism fails to occur. In practice, a series of agar plates, each having the test compound incorporated at a particular concentration is inoculated with a standard culture of, for example, Candida albicans and each plate is then incubated for an appropriate period at 37° C. The plates are then examined for the presence or absence of growth of the fungus and the appropriate M.I.C. value is noted. Alternatively, a turbidity assay in liquid cultures can be performed and a protocol outlining an example of this assay can be found in the Examples below.

The in vivo evaluation of the compounds can be carried out at a series of dose levels by intraperitoneal or intravenous injection or by oral administration, to mice that have been inoculated with a fungus, e.g., a strain of Candida albicans or Aspergillus flavus. The activity of the compounds can be assessed by monitoring the growth of the fungal infection in groups of treated and untreated mice (by histology or by retrieving fungi from the infection). The activity may be measured in terms of the dose level at which the compound provides 50% protection against the lethal effect of the infection (PD₅₀).

For human antifungal use, the compounds of the formula (I) and sub-groups thereof as defined herein can be administered alone or in admixture with a pharmaceutical carrier selected in accordance with the intended route of administration and standard pharmaceutical practice. Thus, for example, they may be administered orally, parenterally, intravenously, intramuscularly or subcutaneously by means of the formulations described above in the section headed “Pharmaceutical Formulations”.

For oral and parenteral administration to human patients, the daily dosage level of the antifungal compounds of the invention can be from 0.01 to 10 mg/kg (in divided doses), depending on inter alia the potency of the compounds when administered by either the oral or parenteral route. Tablets or capsules of the compounds may contain, for example, from 5 mg to 0.5 g of active compound for administration singly or two or more at a time as appropriate. The physician in any event will determine the actual dosage (effective amount) which will be most suitable for an individual patient and it will vary with the age, weight and response of the particular patient.

Alternatively, the antifungal compounds can be administered in the form of a suppository or pessary, or they may be applied topically in the form of a lotion, solution, cream, ointment or dusting powder. For example, they can be incorporated into a cream consisting of an aqueous emulsion of polyethylene glycols or liquid paraffin; or they can be incorporated, at a concentration between 1 and 10%, into an ointment consisting of a white wax or white soft paraffin base together with such stabilizers and preservatives as may be required.

In addition to the therapeutic uses described above, anti-fungal agents developed with such differential screening assays can be used, for example, as preservatives in foodstuff, feed supplement for promoting weight gain in livestock, or in disinfectant formulations for treatment of non-living matter, e.g., for decontaminating hospital equipment and rooms. In similar fashion, side by side comparison of inhibition of a mammalian CDK and an insect CDK, such as the Drosophilia CDK5 gene (Hellmich et al. (1994) FEBS Lett 356:317-21), will permit selection amongst the compounds herein of inhibitors which discriminate between the human/mammalian and insect enzymes. Accordingly, the present invention expressly contemplates the use and formulation of the compounds of the invention in insecticides, such as for use in management of insects like the fruit fly.

In yet another embodiment, certain of the subject CDK inhibitors can be selected on the basis of inhibitory specificity for plant CDK's relative to the mammalian enzyme. For example, a plant CDK can be disposed in a differential screen with one or more of the human enzymes to select those compounds of greatest selectivity for inhibiting the plant enzyme. Thus, the present invention specifically contemplates formulations of the subject CDK inhibitors for agricultural applications, such as in the form of a defoliant or the like.

For agricultural and horticultural purposes the compounds of the invention may be used in the form of a composition formulated as appropriate to the particular use and intended purpose. Thus the compounds may be applied in the form of dusting powders, or granules, seed dressings, aqueous solutions, dispersions or emulsions, dips, sprays, aerosols or smokes. Compositions may also be supplied in the form of dispersible powders, granules or grains, or concentrates for dilution prior to use. Such compositions may contain such conventional carriers, diluents or adjuvants as are known and acceptable in agriculture and horticulture and they can be manufactured in accordance with conventional procedures. The compositions may also incorporate other active ingredients, for example, compounds having herbicidal or insecticidal activity or a further fungicide. The compounds and compositions can be applied in a number of ways, for example they can be applied directly to the plant foliage, stems, branches, seeds or roots or to the soil or other growing medium, and they may be used not only to eradicate disease, but also prophylactically to protect the plants or seeds from attack. By way of example, the compositions may contain from 0.01 to 1 wt. % of the active ingredient. For field use, likely application rates of the active ingredient may be from 50 to 5000 g/hectare.

The invention also contemplates the use of the compounds of the formula (I) and sub-groups thereof as defined herein in the control of wood decaying fungi and in the treatment of soil where plants grow, paddy fields for seedlings, or water for perfusion. Also contemplated by the invention is the use of the compounds of the formula (I) and sub-groups thereof as defined herein to protect stored grain and other non-plant loci from fungal infestation.

### EXAMPLES

The invention will now be illustrated, but not limited, by reference to the specific embodiments described in the following examples.

In the examples, the following abbreviations are used.
AcOH acetic acid
BOC tert-butyloxycarbonyl
CDI 1,1-carbonyldiimidazole
DMAW90 Solvent mixture: DCM: MeOH, AcOH, H₂O (90:18:3:2)
DMAW120 Solvent mixture: DCM: MeOH, AcOH₅H₂O (120:18:3:2)
DMAW240 Solvent mixture: DCM: MeOH, AcOH, H₂O (240:20:3:2)
DCM dichloromethane
DMF dimethylformamide
DMSO dimethyl sulphoxide
EDC 1-ethyl-3-(3′-dimethylaminopropyl)-carbodiimide
Et₃N triethylamine
EtOAc ethyl acetate
Et₂O diethyl ether
HOAt 1-hydroxyazabenzotriazole
HOBt 1-hydroxybenzotriazole
MeCN acetonitrile
MeOH methanol
P.E. petroleum ether
SiO₂ silica
TBTU N,N,N′,N′-tetramethyl-O-(benzotriazol-1-yl)uronium tetrafluoroborate
THF tetrahydrofuran

### Analytical LC-MS System and Method Description

In the examples, the compounds prepared were characterised by liquid chromatography and mass spectroscopy using the systems and operating conditions set out below. Where atoms with different isotopes are present, and a single mass quoted, the mass quoted for the compound is the monoisotopic mass (i.e. ³⁵CT; ⁷⁹Br etc.). Several systems were used, as described below, and these were equipped with, and were set up to run under, closely similar operating conditions. The operating conditions used are also described below.

Waters Platform LC-MS system:

| | |
|---|---|
| HPLC System: | Waters 2795 |
| Mass Spec Detector: | Micromass Platform LC |
| PDA Detector: | Waters 2996 PDA |

Analytical Acidic conditions:

| | |
|---|---|
| Eluent A: | H₂O (0.1% Formic Acid) |
| Eluent B: | CH₃CN (0.1% Formic Acid) |
| Gradient: | 5-95% eluent B over 3.5 minutes |
| Flow: | 0.8 ml/min |
| Column: | Phenomenex Synergi 4μ MAX-RP 8OA, 2.0 × 50 mm |

Analytical Basic conditions:

| | |
|---|---|
| Eluent A: | H₂O (1O mM NH₄HCO₃ buffer adjusted to pH = 9.2 |
| | with NH₄OH) |
| Eluent B: | CH₃CN |
| Gradient: | 05-95% eluent B over 3.5 minutes |
| Flow: | 0.8 ml/min |
| Column: | Phenomenex Luna C18(2) 5 μm 2.0 × 50 mm |

Analytical Polar conditions:

| | |
|---|---|
| Eluent A: | H₂O (0.1% Formic Acid) |
| Eluent B: | CH₃CN (0.1% Formic Acid) |
| Gradient: | 00-50% eluent B over 3 minutes |
| Flow: | 0.8 ml/min |
| Column: | Phenomenex Synergi 4μ MAX-RP 8OA, 2.0 × 50 mm |

Analytical Lipophilic conditions:

| | |
|---|---|
| Eluent A: | H₂O (0.1% Formic Acid) |
| Eluent B: | CH₃CN (0.1% Formic Acid) |
| Gradient: | 55-95% eluent B over 3.5 minutes |
| Flow: | 0.8 ml/min |
| Column: | Phenomenex Synergi 4μ MAX-RP 80A, 2.0 × 50 mm |

Analytical Long Acidic conditions:

| | |
|---|---|
| Eluent A: | H₂O (0.1% Formic Acid) |
| Eluent B: | CH₃CN (0.1% Formic Acid) |
| Gradient: | 05-95% eluent B over 15 minutes |
| Flow: | 0.4 ml/min |
| Column: | Phenomenex Synergi 4μ MAX-RP 8OA, 2.0 × 150 mm |

Analytical Long Basic Conditions:

| | |
|---|---|
| Eluent A: | H₂O (1OmM NH₄HCO₃ buffer adjusted to ρH = 9.2 |
| | with NH₄OH) |
| Eluent B: | CH₃CN |
| Gradient: | 05-95% eluent B over 15 minutes |
| Flow: | 0.8 ml/min |
| Column: | Phenomenex Luna C18(2) 5 μm 2.0 × 50 mm |

Platform MS conditions:

| | |
|---|---|
| Capillary voltage: | 3.6 kV (3.40 kV on ES negative) |
| Cone voltage: | 25 V |
| Source Temperature: | 120° C. |
| Scan Range: | 100-800 amu |
| Ionisation Mode: | ElectroSpray Positive or |
| | ElectroSpray Negative or |
| | ElectroSpray Positive & Negative |

Waters Fractionlynx LC-MS system:

| | |
|---|---|
| HPLC System: | 2767 autosampler - 2525 binary gradient pump |
| Mass Spec Detector: | Waters ZQ |
| PDA Detector: | Waters 2996 PDA |

Analytical Acidic conditions:

| | |
|---|---|
| Eluent A: | H₂O (0.1% Formic Acid) |
| Eluent B: | CH₃CN (0.1% Formic Acid) |
| Gradient: | 5-95% eluent B over 4 minutes |
| Flow: | 2.0 ml/min |
| Column: | Phenomenex Synergi 4μ MAX-RP 8OA, 4.6 × 50 mm |

Analytical Polar conditions:

| | |
|---|---|
| Eluent A: | H₂O (0.1% Formic Acid) |
| Eluent B: | CH₃CN (0.1% Formic Acid) |
| Gradient: | 00-50% eluent B over 4 minutes |
| Flow: | 2.0 ml/min |
| Column: | Phenomenex Synergi 4μ MAX-RP 8OA, 4.6 × 50 mm |

Analytical Lipophilic conditions:

| | |
|---|---|
| Eluent A: | H₂O (0.1% Formic Acid) |
| Eluent B: | CH₃CN (0.1% Formic Acid) |
| Gradient: | 55-95% eluent B over 4 minutes |
| Flow: | 2.0 ml/min |
| Column: | Phenomenex Synergi 4μ MAX-RP 80A, 4.6 × 50 mm |

Fractionlynx MS conditions:

| | |
|---|---|
| Capillary voltage: | 3.5 kV (3.2 kV on ES negative) |
| Cone voltage: | 25 V (30 V on ES negative) |
| Source Temperature: | 120° C. |
| Scan Range: | 100-800 amu |
| Ionisation Mode: | ElectroSpray Positive or |
| | Electro Spray Negative or |
| | ElectroSpray Positive & Negative |

### Mass Directed Purification LC-MS System

Preparative LC-MS is a standard and effective method used for the purification of small organic molecules such as the compounds described herein. The methods for the liquid chromatography (LC) and mass spectrometry (MS) can be varied to provide better separation of the crude materials and improved detection of the samples by MS. Optimisation of the preparative gradient LC method will involve varying columns, volatile eluents and modifiers, and gradients. Methods are well known in the art for optimising preparative LC-MS methods and then using them to purify compounds. Such methods are described in Rosentreter U, Huber U.; Optimal fraction collecting in preparative LC/MS; J Comb Chem.; 2004; 6(2), 159-64 and Leister W, Strauss K, Wisnoski D, Zhao Z, Lindsley C, Development of a custom high-throughput preparative liquid chromatography/mass spectrometer platform for the preparative purification and analytical analysis of compound libraries; J Comb Chem.; 2003; 5(3); 322-9.

One such system for purifying compounds via preparative LC-MS is described below although a person skilled in the art will appreciate that alternative systems and methods to those described could be used. In particular, normal phase preparative LC based methods might be used in place of the reverse phase methods described here. Most preparative LC-MS systems utilise reverse phase LC and volatile acidic modifiers, since the approach is very effective for the purification of small molecules and because the eluents are compatible with positive ion electrospray mass spectrometry. Employing other chromatographic solutions e.g. normal phase LC, alternatively buffered mobile phase, basic modifiers etc as outlined in the analytical methods described above could alternatively be used to purify the compounds.

### Preparative LC-MS Systems:

### Waters Fractionlynx System:

Hardware:

### 2767 Dual Loop Autosampler/Fraction Collector

2525 preparative pump
CFO (column fluidic organiser) for column selection
RMA (Waters reagent manager) as make up pump

### Waters ZQ Mass Spectrometer

Waters 2996 Photo Diode Array detector

### Waters ZQ Mass Spectrometer

Software:

### Masslynx 4.0

Waters MS running conditions:

| | |
|---|---|
| Capillary voltage: | 3.5 kV (3.2 kV on ES Negative) |
| Cone voltage: | 25 V |
| Source Temperature: | 120° C. |
| Multiplier: | 500 V |
| Scan Range: | 125-800 amu |
| Ionisation Mode: | ElectroSpray Positive or |
| | ElectroSpray Negative |

### Agilent 1100 LC-MS Preparative System:

Hardware:

Autosampler: 1100 series “prepALS”
Pump: 1100 series “PrepPump” for preparative flow gradient and 1100 series “QuatPump” for pumping modifier in prep flow
UV detector: 1100 series “MWD” Multi Wavelength Detector
MS detector: 1100 series “LC-MSD VL”

### Fraction Collector: 2×“Prep-FC”

Make Up pump: “Waters RMA”

### Agilent Active Splitter

Software:

### Chemstation: Chem32

Agilent MS running conditions:

| | |
|---|---|
| Capillary voltage: | 4000 V (3500 V on ES Negative) |
| Fragmentor/Gain: | 150/1 |
| Drying gas flow: | 13.0 L/min |
| Gas Temperature: | 350° C. |
| Nebuliser Pressure: | 50 psig |
| Scan Range: | 125-800 amu |
| Ionisation Mode: | ElectroSpray Positive or |
| | ElectroSpray Negative |

### Chromatographic Conditions:

Columns:

1. Low pH chromatography:

### Phenomenex Synergy MAX-RP, 10μ, 100×21.2 mm

(alternatively used Thermo Hypersil-Keystone HyPurity Aquastar, 5μ, 100×21.2 mm for more polar compounds)
2. High pH chromatography:
Phenomenex Luna C18 (2), 10μ, 100×21.2 mm (alternatively used Phenomenex Gemini, 5μ, 100×21.2 mm)

Eluents:

1. Low pH chromatography:

Solvent A: H₂O+0.1% Formic Acid, pH˜1.5

### Solvent B: CH₃CN+0.1% Formic Acid

2. High pH chromatography:
Solvent A: H₂O+10 mM NH₄HCO₃+NH₄OH, pH=9.2

### Solvent B: CH₃CN

3. Make up solvent:
MeOH+0.2% Formic Acid (for both chromatography type)

Methods:

According to the analytical trace the most appropriate preparative chromatography type was chosen. A typical routine was to run an analytical LC-MS using the type of chromatography (low or high pH) most suited for compound structure. Once the analytical trace showed good chromatography a suitable preparative method of the same type was chosen. Typical running condition for both low and high pH chromatography methods were:

Flow rate: 24 ml/min
Gradient: Generally all gradients had an initial 0.4 min step with 95% A+5% B. Then according to analytical trace a 3.6 min gradient was chosen in order to achieve good separation (e.g. from 5% to 50% B for early retaining compounds; from 35% to 80% B for middle retaining compounds and so on)
Wash: 1.2 minute wash step was performed at the end of the gradient
Re-equilibration: 2.1 minutes re-equilibration step was ran to prepare the system for the next run
Make Up flow rate: 1 ml/min

Solvent:

AU compounds were usually dissolved in 100% MeOH or 100% DMSO

From the information provided someone skilled in the art could purify the compounds described herein by preparative LC-MS.

The starting materials for each of the Examples are commercially available unless otherwise specified.

### Preparation of Starting Materials

### Preparation I

### Synthesis of trans-4-(2-methoxy-ethoxy)-cyclohexylamine

### Step 1. trans-4-dibenzylamino-cyclohexanol

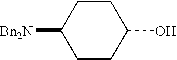

Benzyl bromide (12.0 g, 70 mmol), trans-4-aminocyclohexanol (4.0 g, 35 mmol), sodium hydrogen carbonate (7.8 g, 93 mmol) and ethanol (100 ml) were combined and stirred at reflux for 16 hours. The reaction mixture was reduced in vacuo, diluted with dichloromethane, washed (1 M NaOH, brine), dried (MgSO₄) and reduced in vacuo. The residue was purified by column chromatography (SP4-biotage), eluting with 0-50% ethyl acetate in petroleum ether to give trans-4-dibenzylamino-cyclohexanol as a white solid (3.83 g, 37%). (LC/MS: Rₜ 1.78, [M+H]⁺ 296.39).

### Step 2. Dibenzyl-[trans-4-(2-methoxy-ethoxy)-cyclohexyl-amine

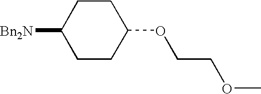

Sodium hydride (60% in mineral oil) (0.240 g, 6 mmol) was washed twice with petroleum ether under nitrogen. Dioxane (5 ml) and trans-4-dibenzylamino-cyclohexanol (0.590 g, 2 mmol) were added and the mixture heated to 95° C. for 30 minutes. After cooling to ambient temperature 2-chloroethyl methyl ether (0.73 ml, 8 mmol) was added and the whole stirred at 95° C. for 18 hours. The reaction mixture was allowed to cool to ambient temperature then was diluted with dichloromethane, washed (1 M NaOH, brine), dried (MgSO₄) and reduced in vacuo. The residue was purified by column chromatography (SP4-biotage), eluting with 0-50% ethyl acetate in petroleum ether to give dibenzyl-[trans-4-(2-methoxy-ethoxy)-cyclohexyl]-amine as a yellow oil (0.275 g, 39%). (LC/MS: Rₜ 2.08, [M+H]⁺ 354.37).

### Step 3. Trans-4-(2-methoxy-ethoxy)-cyclohexylamine

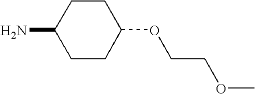

Dibenzyl-[trans-4-(2-methoxy-ethoxy)-cyclohexyl]-amine (0.275 g, 0.77 mmol) was dissolved in ethanol (10 ml). Palladium hydroxide on carbon (20%, 0.120 mg) was added under a flow of nitrogen and the reaction mixture was shaken for 4 hours under 40 psi of hydrogen in a Parr hydrogenator. The reaction mixture was diluted with further ethanol, filtered through Celite™, washing with ethanol and the filtrate reduced in vacuo to give trans-4-(2-methoxy-ethoxy)-cyclohexylamine as a clear colourless oil (0.123 g, 92%).

### Preparation II

### Preparation of 2-(5-amino-pyridin-2-yloxy)-ethanol

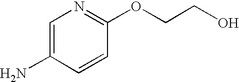

To a solution of 2-[(5-nitro-2-pyridyl)oxy]ethan-1-ol (0.5 g, 2.72 mmoles) in ethanol (10 ml) under nitrogen was added 10% palladium on carbon (50 mg), and the resultant suspension was hydrogenated at room temperature and pressure (RTP) for 3 hours. The reaction mixture was filtered through Celite™. The filtrate was evaporated in vacuo to give 2-(5-amino-pyridin-2-yloxy)-ethanol as a colourless oil (410 mg, 98%). (LC/MS: Rₜ 0.36, [M+H]⁺ 155.10).

### Preparation III

### Preparation of 6-(2-methoxy-ethoxy)-pyridin-3-yl amine

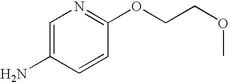

A suspension of 2-chloro-5-nitropyridine (Ig, 6.31 mmoles), 2-methoxyethanol (0.55 ml, 6.94 mmoles) and potassium tert-butoxide (850 mg, 7.57 mmoles) in DMF (10 ml) was stirred at ambient temperature for 2 hours. The reaction mixture was diluted with EtOAc (100 ml), washed with water (x3), dried (MgSO₄), filtered and evaporated in vacuo to give 2-(2-methoxy-ethoxy)-5-nitro-pyridine as a yellow solid (1.0 g, 80%). (LC/MS: Rₜ 2.55, [M+H]⁺ 199.19).

To a solution of 2-(2-methoxy-ethoxy)-5-nitro-pyridine (1 g, 5.05 mmoles) in methanol (10 ml) under nitrogen was added 10% palladium on carbon (100 mg) and the resultant suspension hydrogenated at RTP for 2 hours. The reaction mixture was filtered through Celite. The filtrate was evaporated in vacuo to give 6-(2-methoxy-ethoxy)-pyridin-3-ylamine as a light brown oil (0.9 g, 100%). (LC/MS: Rₜ 0.74, [M+H]⁺ 169.13).

### Preparation IV

### Synthesis of 1-methyl-piperidin-3-(S)-ylamine

### Step 1. Synthesis of (S)-(1-methyl-piperidin-3-yl)-carbamic acid tert-butyl ester

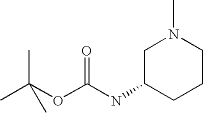

A mixture of (S)-3-BOC-aminopiperidine (600 mg, 3.0 mmol), potassium carbonate (470 mg, 3.4 mmol) and methyl iodide (188 μl. 3.0 mmol) was heated at reflux for 12 hours. The mixture was reduced in vacuo, partitioned between EtOAc and water and the organic portion washed with brine, dried (MgSO₄) and reduced in vacuo to give the title compound as a yellow solid (450 mg).

### Step 2. Synthesis of 1-methyl-piperidin-3-(S) ylamine

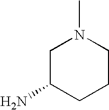

A mixture of (S)-(1-methyl-piperidin-3-yl)-carbamic acid tert-butyl ester (440 mg) in trifluoroacetic acid (5 ml) and DCM (5 ml) was stirred at ambient temperature for 1 hour then reduced in vacuo azeotroping with toluene (x3) to give the title compound as an orange oil.

### Preparation V

### Synthesis of 1-methyl-piperidin-3-(R) vlamine

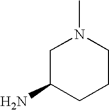

This compound was prepared in a manner analogous to that described for 1-methyl-piperidin-3-(S)-ylamine, except using (R)-3-BOC-aminopiperidine as the starting material.

### Preparation VII

### Synthesis of trans-4-(2-dimethylamino-ethoxy)-cyclohexylamine

### Step 1. Synthesis of trans-4-dibenzylamino-cyclohexanol

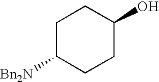

A mixture of trans-4-aminocyclohexanol (3.80 g, 33 mmol), benzyl chloride (11.5 ml, 100 mmol) and sodium hydrogen carbonate (11.2 g, 133 mmol) in ethanol (100 ml) was heated at reflux for 14 hours, then reduced in vacuo. The residue was partitioned between DCM and water, the layers separated and the organic portion washed with 1 M aqueous NaOH solution and brine, dried (MgSO₄) and reduced in vacuo. Residue purified by column chromatography using P.E.-EtOAc (1:2) to give the title compound as a white solid (4.38 g).

### Step 2. Synthesis of trans-diberizyl-[4-(2-dimethylamino-ethoxy)-cyclohexyl]-amine

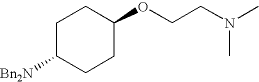

To a mixture of NaH, 60% dispersion in mineral oil (167 mg, 2.5 mmol) in dry dioxane (5 ml) stirring under a nitrogen atmosphere at ambient temperature was added trans-4-dibenzylamino-cyclohexanol (590 mg, 2 mmol). The mixture was stirred for 5 minutes, then (2-chloro-ethyl)-dimethyl-amine (753 mg, 7 mmol) added. The mixture was heated at 95° C. for 2 hours, cooled to ambient temperature and diluted with DCM. IM Aqueous NaOH solution was cautiously added, the layers separated and the organic portion washed with brine, dried (MgSO₄) and reduced in vacuo to give an orange oil (739 mg). On analysis, it was noted that the product was an approximately 1:1 mixture of the title compound and starting material.

### Step 3. Synthesis of trans-4-(2-dimethylamino-ethoxy)-cyclohexylamine

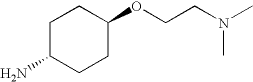

A mixture of the VIIb product (400 mg) and Pd(OH)₂/C (200 mg) in methanol (15 ml) was shaken under an atmosphere of hydrogen (40 psi) for 3 hours, filtered through a plug of Celite and reduced in vacuo to give the title compound alongside trans-4-aminocyclohexanol in an approximately 1:1 mixture (184 mg).

### Preparation VIII

### Synthesis of 4-ammo-1H-pyrazole-3-carboxylic acid ethyl ester

### Step 1. 4-Nitro-1H-pyrazole-3-carboxylic acid ethyl ester

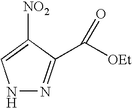

Thionyl chloride (2.90 ml, 39.8 mmol) was slowly added to a mixture of 4-nitro-3-pyrazolecarboxylic acid (5.68 g, 36.2 mmol) in EtOH (100 ml) at ambient temperature and the mixture stirred for 48 hours. The mixture was reduced in vacuo and dried through azeotrope with toluene to afford 4-nitro-1H-pyrazole-3-carboxylic acid ethyl ester as a white solid (6.42 g, 96%). (¹H NMR (400 MHz, DMSO-d₆) δ 14.4 (s, IH), 9.0 (s, IH), 4.4 (q, 2H), 1.3 (t, 3H)).

### Step 2. 4-Amino-1H-pyrazole-3-carboxylic acid ethyl ester

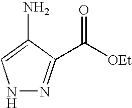

A mixture of 4-nitro-1H-pyrazole-3-carboxylic acid ethyl ester (6.40 g, 34.6 mmol) and 10% Pd/C (650 mg) in EtOH (150 ml) was stirred under an atmosphere of hydrogen for 20 hours. The mixture was filtered through a plug of Celite, reduced in vacuo and dried through azeotrope with toluene to afford 4-amino-1H-pyrazole-3-carboxylic acid ethyl ester as a pink solid (5.28 g, 98%). (¹H NMR (400 MHz, DMSO-d₆) δ 12.7 (s, 1H), 7.1 (s, 1H)₅ 4.8 (s, 2H), 4.3 (q, 2H), 1.3 (t, 3H)).

### Preparation IX

### Synthesis of 4-C2,6-dichloro-benzoylamino)-1H-pyrazole-3-carboxylic acid

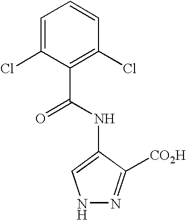

2,6-dichlorobenzoyl chloride (8.2 g; 39.05 mmol) was added cautiously to a solution of 4-amino-1H-pyrazole-3-carboxylic acid methyl ester (prepared in a manner analogous to Preparation VIII) (5 g; 35.5 mmol) and triethylamine (5.95 ml; 42.6 mmol) in dioxane (50 ml) then stirred at room temperature for 5 hours. The reaction mixture was filtered and the filtrate treated with methanol (50 ml) and 2M sodium hydroxide solution (100 ml), heated at 50° C. for 4 hours, and then evaporated. 100 ml of water was added to the residue then acidified with concentrated hydrochloric acid. The solid was collected by filtration, washed with water (100 ml) and sucked dry to give 10.05 g of 4-(2,6-dichloro-benzoylamino)-1H-pyrazole-3-carboxylic acid as a pale violet solid. (LC/MS: Rₜ 2.26, [M+H]⁺ 300/302).

### Preparation X

### Preparation of 4-(2,6-dichloro-benzoylaminoV1II-pyrazole-3-carboxylic acid piperidin-4-ylamide hydrochloride

### Step 1. Preparation of 4-{[4-(2,6-dichloro-benzoylaminoV1H-pyrazole-3-carbonyl]-amino1-piperidine-1-carboxylic acid tert-butyl ester

A mixture of 4-(2,6-dichloro-benzoylamino)-1H-pyrazole-3-carboxylic acid (6.5 g, 21.6 mmol) (Preparation IX), 4-amino-1-BOC-piperidine (4.76 g, 23.8 mmol), EDC (5.0 g, 25.9 mmol) and HOBt (3.5 g, 25.9 mmol) in DMF (75 ml) was stirred at room temperature for 20 hours. The reaction mixture was reduced in vacuo and the residue partitioned between ethyl acetate (100 ml) and saturated aqueous sodium bicarbonate solution (100 ml). The organic layer was washed with brine, dried (MgSO₄) and reduced in vacuo. The residue was taken upin 5% MeOH-DCM (−30 ml). The insoluble material was collected by filtration and, washed with DCM and dried in vacuo to give 4-{[4-(2,6-dichloro-benzoylamino)-1H-pyrazole-3-carbonyl]-amino}-piperidine-1-carboxy lie acid tert-butyl ester (5.38 g) as a white solid. The filtrate was reduced in vacuo and the residue purified by column chromatography using gradient elution 1:2 EtOAc/hexane to EtOAc to give further 4-{[4-(2,6-dichloro-benzoylamino)-1H-pyrazole-3-carbonyl]-amino}-piperidine-1-carboxylic acid tert-butyl ester (2.54 g) as a white solid.

### Step 2. 4-C2,6-dichloro-benzoylamino)-1H-pyrazole-3-carboxylic acid piperidin-4-ylamide hydrochloride

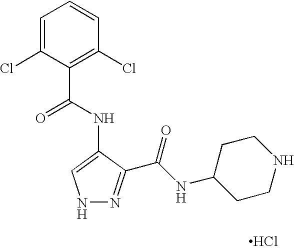

A solution of 4-{[4-(2,6-dichloro-benzoylamino)-1H-pyrazole-3-carbonyl]-amino}-piperidine-1-carboxylic acid tert-butyl ester (7.9 g) in MeOH (50 mL) and EtOAc (50 ml) was treated with sat. HCl-EtOAc (40 mL) then stirred at r.t. overnight. The product did not crystallise due to the presence of methanol, and therefore the reaction mixture was evaporated and the residue triturated with EtOAc. The resulting off white solid was collected by filtration, washed with EtOAc and sucked dry on the sinter to give 6.3 g of 4-(2,6-dichloro-benzoylamino)-1H-pyrazole-3-carboxylic acid piperidin-4-ylamide as the hydrochloride salt. (LC/MS: Rₜ 5.89, [M+H]⁺ 382/384).

### Preparation XI

### Step 1. Synthesis of 4-C2,6-difluoro-benzoylaminoy1H-pyrazole-3-carboxylic acid ethyl ester

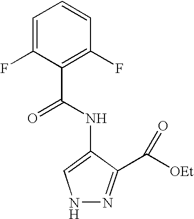

A mixture of 2,6-difluorobenzoic acid (6.32 g, 40.0 mmol), 4-amino-1H-pyrazole-3-carboxylic acid ethyl ester (5.96 g, 38.4 mmol), EDC (8.83 g, 46.1 mmol) and HOBt (6.23 g, 46.1 mmol) in DMF (100 ml) was stirred at ambient temperature for 6 h. The mixture was reduced in vacuo, water added and the solid formed collected by filtration and air-dried to give 4-(2,6-difluoro-benzoylamino)-1H-pyrazole-3-carboxylic acid ethyl ester as the major component of a mixture (15.3 g). (LC/MS: Rₜ 3.11, [M+H]⁺ 295.99).

### Step 2. Synthesis of 4-(2,6-difluoro-benzoylamino)-1H-pyrazole-3-carboxylic acid

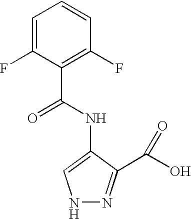

A mixture of 4-(2,6-difluoro-benzoylamino)-1H-pyrazole-3-carboxylic acid ethyl ester (10.2 g) in 2 M aqueous NaOH/MeOH (1:1, 250 ml) was stirred at ambient temperature for 14 h. Volatile materials were removed in vacuo, water (300 ml) added and the mixture taken to pH 5 using IM aqueous HCl. The resultant precipitate was collected by filtration and dried through azeotrope with toluene to afford 4-(2,6-difluoro-benzoylamino)-1H-pyrazole-3-carboxylic acid as a pink solid (5.70 g). (LC/MS: Rₜ 2.33, [M+H]⁺ 267.96).

### Preparation XII

### Synthesis of N-trans-(4-amino-cyclohexyl)-methanesulphonamide hydrochloride

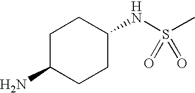

### Step 1: Synthesis of trans-(N-Boc-4-amino-cyclohexyl)-methanesulfonamide

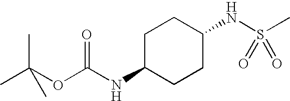

A mixture of N-Boc-trans-4-aminocyclohexane (860 mg; 4 mmol) and methane sulphonic anhydride (1.05 g; 6 mmol) in pyridine (10 ml) was stirred at room temperature overnight. The reaction was evaporated then partitioned between EtOAc and 2M hydrochloric acid. The undissolved solid was collected by filtration, washed with water, sucked dry then purified by flash column chromatography, eluting with 2% then 5% MeOH/DCM. 185 mg of trans-(N-Boc-4-amino-cyclohexyl)-methanesulphonamide was isolated as a white solid.

### Step 2: Synthesis of N-trans-(4-amino-cyclohexyl′)-methanesulfonamide hydrochloride

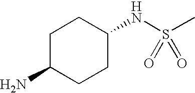

Trans-(N-Boc-4-amino-cyclohexyl)-methanesulphonamide (180 mg) was dissolved in a saturated HCl/ethyl acetate solution and stirred at room temperature for 4 hours. The solid was collected by filtration, washed with diethyl ether and dried under vacuum to give 85 mg of N-trans-(4-amino-cyclohexyl)-methanesulfonamide hydrochloride as a pale pink solid.

### Preparation XIII

### Synthesis of 2-fluoro-6-(2-methoxy-ethoxy)-benzoic acid

### Step 1: Synthesis of 2-fluoro-6-(2-methoxy-ethoxyVbenzoic acid methyl ester

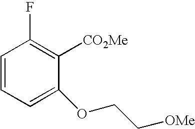

To a stirred solution of methyl-6-fluorosalicylic acid (1 g, 5.88 mmoles) in DMF (10 ml) under nitrogen was added sodium hydride (282 mg, 7.06 mmoles). The resultant solution was stirred at ambient temperature for 10 minutes. 2-Chloroethyl methyl ether (591 μl, 6.47 mmoles) was added to the reaction mixture and the resultant solution heated at 85° C. for 24 hours. The reaction mixture was diluted with ethyl acetate, and then washed sequentially with sodium hydroxide solution (2N, twice), water (twice) and then brine solution. The organic portion was dried (MgSO₄), filtered and evaporated in vacuo to give 2-fluoro-6-(2-methoxy-ethoxy)-benzoic acid methyl ester as a colourless oil (600 mg, 45%). (LC/MS: Rₜ 2.73, [M+H]⁺ 229.17).

### Step 2: Synthesis of 2-fluoro-6-(2-methoxy-ethoxyVbenzoic acid

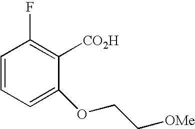

To a stirred solution of 2-fluoro-6-(2-methoxy-ethoxy)-benzoic acid methyl ester (600 mg, 2.63 mmoles) in methanol (10 ml) was added a solution of sodium hydroxide (2N, 10 ml) and the resultant solution was heated at 50° C. for 2 hours. The methanol was evaporated in vacuo. The residue was partitioned between EtOAc and water. The aqueous portion was acidified to pH 2 with HCl solution (2N) and then washed with EtOAc. This organic portion was dried (MgSO₄), filtered and evaporated in vacuo to give 2-fluoro-6-(2-methoxy-ethoxy)-benzoic acid as a colourless oil (400 mg, 71%). (LC/MS: Rₜ 2.13, [M+H]⁺ 215.17).

### Preparation XIV

### Synthesis of 2,3-difluoro-6-methoxy-benzoic acid

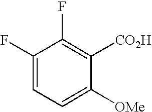

To a suspension of 2,3-difluoro-6-methoxybenzaldehyde (0.5 g, 2.91 mmoles) in potassium hydroxide solution (3 g of KOH in 20 ml of water) was added hydrogen peroxide solution (27.5% w/w, 4 ml) and then heated at 70° C. for 2 hours. The reaction mixture was acidified to pH 2 with concentrated HCl, and then washed with ethyl acetate. The organic portion was dried (MgSO₄), filtered, evaporated in vacuo and then azeotoped with toluene to give 2,3-difluoro-6-methoxy-benzoic acid as a white solid (500 mg, 91%). (LC/MS: Rₜ 2.08, no molecular ion observed).

### Preparation XV

### Synthesis of 2-methoxy-6-methyl-benzoic acid

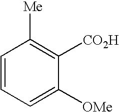

To a solution of ethyl-2-methoxy-6-methyl-benzoate (5 g, 25.77 mmoles) in ethanol (20 ml) was added a solution of sodium hydroxide (2N, 20 ml). The reaction mixture was heated at 70° C. for 24 hours. Sodium hydroxide (10 g, 0.25 mmoles) was added to the reaction mixture and the resultant solution heated at 70° C. for another 4 hours. The ethanol was removed in vacuo. The residue was partitioned between ethyl acetate and water. The aqueous portion was acidified with concentrated HCl to pH 2 and then washed with ethyl acetate. This organic portion was dried (MgSO₄), filtered and evaporated in vacuo to give 2-methoxy-6-methyl-benzoic acid as a pale yellow solid (3 g, 70%). (LC/MS: Rₜ 2.21, [M+H]⁺ 167.11).

### Preparation XVI

### Synthesis of 2-chloro-6-fluoro-3-methoxy-benzoic acid

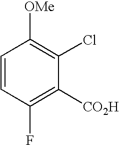

To a solution of 2-chloro-4-fluoroanisole (1.9 ml, 15 mmoles) in THF (50 ml) under nitrogen at −70° C. was added a solution of n-BuLi (1.6M, 13 ml, 21 mmoles) dropwise. After the addition the reaction mixture was stirred for a further 1.5 hours at −70° C. Several pellets of dry ice were added to the reaction mixture and stirred for 10 minutes. The reaction mixture was then poured into a 250 ml beaker half-filled with dry ice. The reaction mixture was then allowed to warm to room temperature and partitioned between ethyl acteate and sodium hydroxide solution (2N). The aqueous portion was acidified with concentrated HCl to pH 2 and then washed with ethyl acetate. This organic portion was dried (MgSO₄), filtered and evaporated in vacuo. The residue was azeotroped with toluene in vacuo to give 2-chloro-6-fluoro-3-methoxy-benzoic acid as a white solid (2.9 g, 95%). (LC/MS: Rₜ 1.91, no molecular ion observed).

### Preparation XVII

### 2-chloro-6-dimethylaminomethyl-benzoic acid

### Step 1. Synthesis of 2-bromomethyl-6-chloro-benzoic acid methyl ester

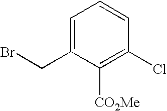

2-Chloro-6-methyl benzoic acid (5.8 g, 34.0 mmoles) was suspended in dichloromethane (100 ml). To the suspension was added DMF (250 mg, 3.4 mmoles) and then dropwise oxalyl chloride (3.9 ml, 44.2 mmoles). The resultant solution was stirred at ambient temperature for 24 hours. Further DMF (250 mg, 3.4 mmoles) and oxalyl chloride (3.9 ml, 44.2 mmoles) was added to the reaction mixture, and the resultant solution stirred for a further 24 hours at ambient temperature. The reaction mixture was concentrated in vacuo. The residue was dissolved in methanol (100 ml) and stirred at ambient temperature for 3 hours. The reaction mixture was concentrated in vacuo. The residue was partitioned between ethyl acetate and sodium hydroxide solution (2N). The organic portion was washed with sodium hydroxide solution (2N), and then brine, dried (MgSO₄), filtered and the concentrated in vacuo. The residue was purified by flash chromatography (eluent 3:5 EtOAc iPetrol to give 2-chloro-6-methyl-benzoic acid methyl ester as a yellow oil (4.5 g, 72%).

To a solution of 2-chloro-6-methyl-benzoic acid methyl ester (4.5 g, 24.4 mmoles) in CCl₄ (50 ml) was added N-bromosuccinimide (4.3 g, 24.4 mmoles) and benzoyl peroxide (50 mg, 0.2 mmoles), and the resultant suspension was heated at 70° C. for 24 hours. Further benzoyl peroxide (50 mg, 0.2 mmoles) was added to the reaction mixture and stirred at 70° C. for a further 3 hours. The reaction mixture was cooled to ambient temperature and filtered. The filtrate was concentrated in vacuo. The residue was purified by flash chromatography (Biotage SP4, 4OM, flow rate 40 ml/min, gradient Petrol to 2:3 EtOAc:Petrol) to give 2-bromomethyl-6-chloro-benzoic acid methyl ester as a yellow oil (6.2 g, 97%).

Step 2. Synthesis of 2-chloro-6-dimethylaminomethyl-benzoic acid methyl ester.

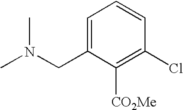

A solution of 2-bromomethyl-6-chloro-benzoic acid methyl ester (2 g, 7.6 mmoles) in an ethanolic solution of dimethylamine (5.6M, 13.6 ml) was stirred at ambient temperature for 24 hours. The reaction mixture was concentrated in vacuo. The residue was partitioned between ethyl acetate and hydrochloric acid solution (IN). The aqueous phase was basified with sodium hydroxide solution (2N) to pH 12 and then partitioned against ethyl acetate. The organic portion was dried (MgSO₄), filtered and concentrated in vacuo to give 2-chloro-6-dimethylaminomethyl-benzoic acid methyl ester as a colourless oil (300 mg, 17%). (LC/MS: Rₜ 1.55, [M+H]⁺ 228.10).

### Step 3. Synthesis of 2-chloro-6-dimethylaminomethyl-benzoic acid

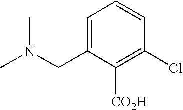

To a solution of 2-chloro-6-dimethylaminomethyl-benzoic acid methyl ester (300 mg, 1.32 mmoles) in methanol (10 ml) was added sodium hydroxide solution (2N, 10 ml), and the resultant solution was stirred at ambient temperature for 1 hour, and then at 50° C. for 72 hours. Methanol was evaporated in vacuo, the residue was acidified to pH 4 with hydrochloric acid (2N) and then concentrated in vacuo. The residue was co-evaporated in vacuo with methanol and toluene. The residue was triturated with methanol and filtered. The filtrate was evaporated in vacuo, triturated with 1:4 MeOHiEtOAc and then filtered. The filtrate was evaporated in vacuo to give 2-chloro-6-dimethylaminomethyl-benzoic acid as a white solid (200 mg, 71%).

### Preparation XVIII

### 2-chloro-6-methoxymethyl-benzoic acid

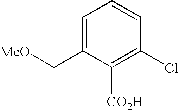

To a solution of 2-bromomethyl-6-chloro-benzoic acid methyl ester (2 g, 7.60 mmoles) in methanol (20 ml) under nitrogen was added sodium hydride (912 mg, 22.80 mmoles). The reaction mixture was heated at 50° C. for 2 hours. After cooling to ambient temperature the reaction mixture was partitioned between ethyl acetate and water. The organic portion was dried (MgSO₄), filtered and evaporated in vacuo. The residue was purified by flash chromatography (Biotage SP4, 40S, flow rate 40 ml/min, gradient 3:17 EtOAc:Petrol to 1:1 EtOAc rPetrol) to give 2-chloro-6-methoxymethyl-benzoic acid methyl ester as a colourless oil (400 mg, 25%).

To a solution of 2-chloro-6-methoxymethyl-benzoic acid methyl ester (400 mg, 1.86 mmoles) in methanol (10 ml) was added a solution of sodium hydroxide (2N, 10 ml) and the resultant solution stirred at 50° C. for 24 hours. Further sodium hydroxide solution (2N, 10 ml) was added and the reaction mixture heated at 50° C. for a further 24 hours. Methanol was removed by evaporation in vacuo. The residue was partitioned between ethyl acetate and water. The aqueous portion was acidified to pH 2 with concentrated hydrochloric acid and then partitioned against ethyl acetate. The organic portion was dried (MgSO₄), filtered and evaporated in vacuo to give 2-chloro-6-methoxymethyl-benzoic acid as a white solid (340 mg, 91%). (LC/MS: Rₜ 2.23, [M+Na]⁺223.11).

### Preparation XIX

### Synthesis of 4-Amino-1H-pyrazole-3-carboxylic acid (trans-4-methoxymethoxy-cyclohexyl)-amide

### Step 1. Synthesis of trans-4-methoxymethoxy-cyclohexyl amine

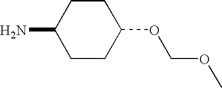

Sodium hydride (1.6 g, 40 mmol) and trans-4-dibenzylamino-cyclohexanol (Preparation I, Step 1) (4.0 g, 13.6 mmol) in dioxane (50 ml) were heated to 95° C. for 30 minutes. After cooling to ambient temperature chloromethyl methyl ether (3 ml, 40 mmol) was added and the reaction mixture was stirred at ambient temperature for 5 hours, then diluted with dichloromethane, washed (1 M NaOH, brine), dried (MgSO₄) and reduced in vacuo to give crude dibenzyl-(trans-4-methoxymethoxy-cyclohexyl)-amine as a yellow gel (4.84 g). (LC/MS: Rₜ 2.01, [M+H]⁺ 340.28).

The crude dibenzyl-(trans-4-methoxymethoxy-cyclohexyl)-amine was taken upin ethanol (100 ml). Palladium hydroxide on carbon (20%, 2.5 g) was added under a flow of nitrogen and the reaction mixture was shaken for 5 hours under 48 psi of hydrogen in a Parr hydrogenator. The reaction mixture was diluted with ethyl acetate, filtered through Celite™, washing with further ethyl acetate and the filtrate reduced in vacuo to give trans-4-methoxymethoxy-cyclohexylamine as a sticky white solid (2.95 g). (¹H NMR (400 MHz, MeOD-d₄) δ 4.6 (s, 2H), 3.5 (m, 1H), 3.35 (s, 3H), 2.7 (m, 1H), 1.9-2.1 (m, 4H), 1.2-1.4 (m, 4H).

### Step 2. Synthesis of 4-nitro-1H-pyrazole-3-carboxylic acid (trans-4-methoxymethoxy-cyclohexyl)-amide

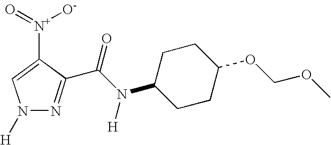

A mixture of 4-nitro-3-pyrazolecarboxylic acid (2.32 g, 14.8 mmol), trans 4-aminocyclohexanol (2.95 g, 18.5 mmol), EDAC (3.55 g, 18.5 mmol) and HOBt (2.50 g, 18.5 mmol) in DMF (75 ml) was stirred at ambient temperature for 16 hours. The mixture was reduced in vacuo, partitioned between saturated aqueous sodium bicarbonate and ethyl acetate. The organic layer was washed (water, brine) dried (MgSO₄), and reduced in vacuo to give a yellow oil (3.25 g), which was purified by column chromatography, eluting 0-100% EtOAc in petroleum ether, then 1-25% MeOH in EtOAc to give 4-nitro-1H-pyrazole-3-carboxylic acid (trans-4-methoxymethoxy-cyclohexyl)-amide as a pale yellow solid (1.25 g). (LC/MS: Rₜ 2.11 [M+H⁺] 297.25).

### Step 3. 4-Amino-1H-pyrazole-3-carboxylic acid ((trans-4-methoxymethoxy-cyclohexyl)-amide

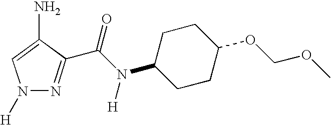

A solution of 4-nitro-1H-pyrazole-3-carboxylic acid (4-methoxymethoxy-cyclohexyl)-amide (1.25 g, 4.2 mmol) in DMF (100 ml), was treated with 10% palladium on carbon (0.125 g) then shaken under hydrogen at room temperature and pressure for 5 hours. The reaction mixture was diluted with ethyl acetate, filtered through Celite™, washing with further ethyl acetate and the filtrate reduced in vacuo to give crude 4-amino-1H-pyrazole-3-carboxylic acid (4-methoxymethoxy-cyclohexyl)-amide trans-4-methoxymethoxy-cyclohexylamine as a brown oil (1.45 g). (LC/MS: Rₜ 1.41 [M+H]⁺ 269.37).

### General Procedures

### General Procedure A

### Preparation of Amide from Pyrazole Carboxylic Acid

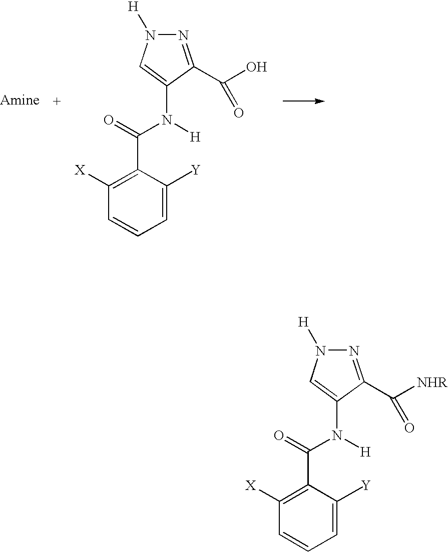

A mixture of the appropriate benzoylamino-1H-pyrazole-3-carboxylic acid (0.50 mmol), EDAC (104 mg, 0.54 mmol), HOBt (73.0 mg, 0.54 mmol) and the corresponding amine (0.45 mmol) in DMF (3 ml) was stirred at ambient temperature for 16 hours. The mixture was reduced in vacuo, the residue taken up in EtOAc and washed successively with saturated aqueous sodium bicarbonate, water and brine. The organic portion was dried (MgSO₄) and reduced in vacuo to give the desired product.

### General Procedure B

### Preparation of Amide from Amino-Pyrazole

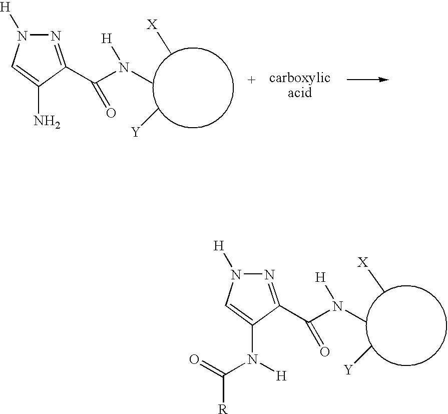

To a stirred solution of the appropriate 4-amino-1H-pyrazole-3-carboxylic acid amide (0.23 mmol), EDAC (52 mg; 0.27 mmol) and HOBt (37 mg; 0.27 mmol) in 5 ml of N,N-dimethylformamide was added the corresponding carboxylic acid (0.25 mmol), and the mixture was then left at room temperature overnight. The reaction mixture was evaporated and the residue purified by preparative LC/MS, to give the product.

### General Procedure C

### Synthesis of Amides of 4-(2,6-difluoro-benzoylammo)-1H-pyrazole-3-carboxylic acid

A mixture of 4-(2,6-difluoro-benzoylamino)-1H-pyrazole-3-carboxylic acid (134 mg, 0.50 mmol), an amine (0.45 mmol), EDAC (104 mg, 0.54 mmol) and HOBt (73.0 mg, 0.54 mmol) in DMF (3 ml) was stirred at ambient temperature for 16 hours. The mixture was reduced in vacuo, the residue taken upin EtOAc and washed successively with saturated aqueous sodium bicarbonate, water and brine. The organic portion was dried (MgSO₄) and reduced in vacuo to give the amide of 4-(2,6-difluoro-benzoylamino)-1H-pyrazole-3-carboxylic acid.

### General Procedure D

### Preparation of Protected 4-amino-pyrazol-3-yl carboxylic acid 4-hydroxy-cyclohexylamide

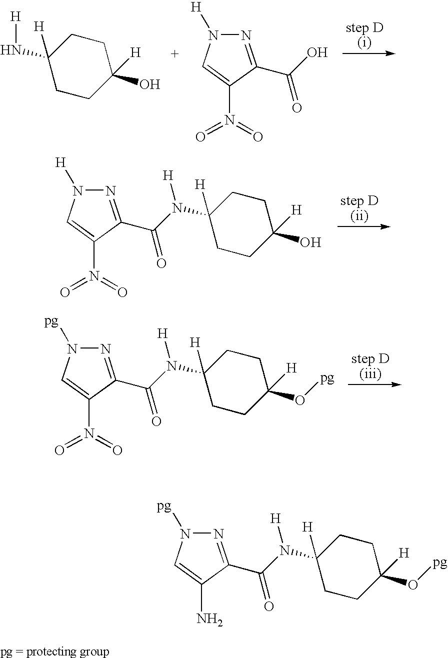

### Step D (i):

A mixture of 4-nitro-3-pyrazolecarboxylic acid (4.98 g, 31.7 mmol), trans 4-aminocyclohexanol (3.65 g, 31.7 mmol), EDAC (6.68 g, 34.8 mmol) and HOBt (4.7 g, 34.8 mmol) in DMF (120 ml) was stirred at ambient temperature for 16 hours. The mixture was reduced in vacuo, the residue taken upin CH₂Cl₂ and washed successively with 5% citric acid, saturated aqueous sodium bicarbonate, water and brine. The product was found to be mainly in the citric acid wash, which was basified and extracted with EtOAc. The organic layer was dried over MgSO₄, filtered and evaporated to give a white solid, which was triturated with CHCl₃ to give 1.95 g of 4-nitro-1H-pyrazole-3-carboxylic acid 4-hydroxy-cyclohexylamide. (LC/MS: Rₜ 1.62, [M+H]⁺ 255).

### Step D (ii):

### Introduction of Tetrahydro-pyran-2-yl Protecting Group

A solution of 4-nitro-1H-pyrazole-3-carboxylic acid 4-hydroxy-cyclohexylamide (1.95 g; 7.67 mmol) in a mix of THF (50 ml) and chloroform (100 ml), was treated with 3,4-dihydro-2H-pyran (1.54 ml, 15.34 mmol) and p-toluenesulphonic acid monohydrate (100 mg). The reaction mixture was stirred at room temperature overnight, and then excess pyran (0.9 ml) was added in total to bring the reaction to completion. The reaction mixture was diluted with DCM and washed successively with saturated aqueous sodium bicarbonate, water and brine. The resulting solution was reduced in vacuo and subject to Biotage column chromatography, eluting with hexane (2 column lengths) followed by 30% ethyl acetate:hexane (10 column lengths), 70% ethyl acetate:hexane (10 column lengths) to give 1.25 g of 4-nitro-1-(tetrahydro-pyran-2-yl-1H-pyrazole-3-carboxylic acid [4-(tetrahydro-pyran-2-yloxy)-cyclohexyl]-amide. (LC/MS: Rₜ 2.97, [M+H]⁺ 423).

### Step D Cm):

A solution of 4-nitro-1-(tetrahydro-pyran-2-yl)-1H-pyrazole-3-carboxylic acid [4-(tetrahydro-pyran-2-yloxy)-cyclohexyl]-amide (0.3 g; 0.71 mmol) in methanol (25 ml), was treated with 10% palladium on carbon (30 mg) then hydrogenated at room temperature and pressure overnight. The catalyst was removed by filtration and washed three times with methanol. The filtrate was evaporated to give 0.264 g of the required product. (LC/MS: Rₜ 2.39, [M+H]⁺ 393).

### General Procedure E

### Synthesis of an Amide of 4-(2,6-dichloro-benzoylammo′)-1H-pyrazole-3-carboxylic acid

A mixture of 4-(2,6-dichloro-benzoylamino)-1H-pyrazole-3-carboxylic acid (Preparation IX) (6.5 g, 21.6 mmol), an amine (23.8 mmol), EDC (5.0 g, 25.9 mmol) and HOBt (3.5 g, 25.9 mmol) in DMF (75 ml) was stirred at room temperature for 20 hours. The reaction mixture was reduced in vacuo and the residue partitioned between ethyl acetate (100 ml) and saturated aqueous sodium bicarbonate solution (100 ml). The organic layer was washed with brine, dried (MgSO₄) and reduced in vacuo. The residue was taken upin 5% MeOH-DCM (˜30 ml). The insoluble material was collected by filtration and, washed with DCM and dried in vacuo to give the amide of 4-(2,6-dichloro-benzoylamino)-1H-pyrazole-3-carboxylic acid. Where desired, the filtrate was reduced in vacuo and the residue purified by column chromatography using gradient elution 1:2 EtOAc/hexane to EtOAc to give further amide.

### General Procedure F

### Preparation of a Urea from a 4-Amino-pyrazole-3-carboxylic acid amide

To a solution of a 4-amino-pyrazole-3-carboxylic acid amide or protected derivative thereof (0.2 mmol) in toluene (2 ml) was added an appropriately substituted phenyl isocyanate (0.24 mmol). The reaction mixture was heated at 70° C. for 1 hour. The reaction mixture was diluted with EtOAc and washed successively with water and brine. The resulting solution was reduced in vacuo to give an oil or dried with magnesium sulphate to give the desired urea.

### General Procedure G

### Sulphonylation or Acylation of Piperidines

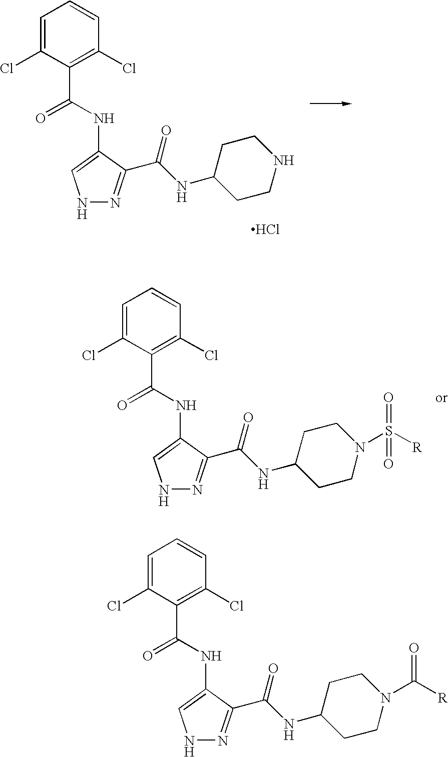

To a mixture of 4-(2,6-dichloro-benzoylamino)-1H-pyrazole-3-carboxylic acid piperidin-4-ylamide hydrochloride (Preparation X) (1 mmol) in acetonitrile (10 ml) was added diisopropylethylamine (2.2 mmol) followed by the appropriate sulphonyl or acid chloride (1 mmol). The mixture was stirred at ambient temperature for 16 hours then reduced in vacuo. The residue was partitioned between ethyl acetate and water, the layers separated and the organic portion washed with brine, dried (MgSO₄) and reduced in vacuo to give the desired sulphonamide or amide derivative.

### General Procedure H

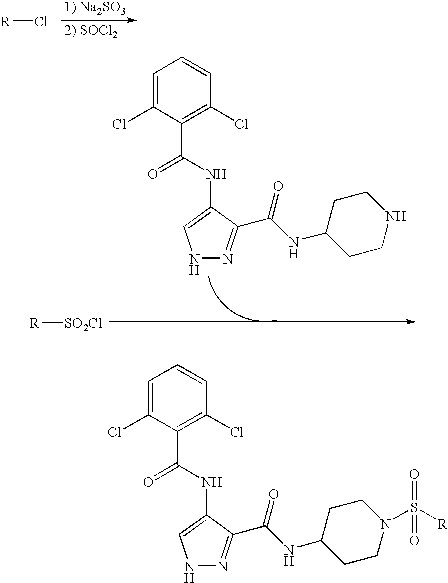

A mixture of alkyl chloride (10 mmol) and sodium sulphite (15 mmol) in 1,4-dioxane/water (1:1, 16 ml) was heated at reflux for 16 hours, allowed to cool to ambient temperature and then reduced in vacuo azeotroping with toluene (x3). To the residue was added thionyl chloride (10 ml) and 2 drops of DMF₅ the mixture was heated at reflux for 2 hours, allowed to cool to ambient temperature and then reduced in vacuo azeotroping with toluene. The residue was partitioned between EtOAc and water, the layers separated and the organic portion washed with brine, dried (MgSO₄) and reduced in vacuo to give the desired sulphonyl chloride derivative.

To a mixture of 4-(2,6-dichloro-benzoylamino)-1H-pyrazole-3-carboxylic acid piperidin-4-ylamide hydrochloride (Preparation X) (2 mmol) in acetonitrile (10 ml) was added diisopropylethylamine (4.2 mmol) followed by the appropriate sulphonyl chloride (approximately 2 mmol). The mixture was stirred at ambient temperature for 16 hours then reduced in vacuo. The residue was partitioned between ethyl acetate and water, the layers separated and the organic portion washed with brine, dried (MgSO₄) and reduced in vacuo to give the desired sulphonamide derivative.

### General Procedure I

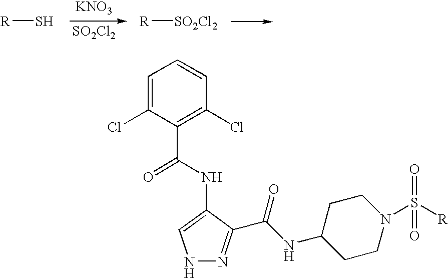

To a solution of thiol (5 mmol) in acetonitrile (50 ml) at 0° C. was added potassium nitrate (12.5 mmol) followed by the drop-wise addition of sulphuryl chloride (12.5 mmol). The mixture was stirred at 0° C. for 2 hours and the mixture neutralised through addition of saturated aqueous NaHCO₃. The mixture was extracted with EtOAc, the layers separated and the organic portion washed with brine, dried (MgSO₄) and reduced in vacuo to give the desired sulphonyl chloride.

To a mixture of 4-(2,6-dichloro-benzoylamino)-1H-pyrazole-3-carboxylic acid piperidin-4-ylamide hydrochloride (Preparation X) (2 mmol) in acetonitrile (10 ml) was added diisopropylethylamine (4.2 mmol) followed by the appropriate sulphonyl chloride (approximately 2 mmol). The mixture was stirred at ambient temperature for 16 hours then reduced in vacuo. The residue was partitioned between ethyl acetate and water, the layers separated and the organic portion washed with brine, dried (MgSO₄) and reduced in vacuo to give the desired sulphonamide derivative.

### General Procedure J

### Preparation of a 4-amino-1H-pyrazole-3-carboxylic acid amide

### Step J (T). Preparation of a 4-nitro-1H-pyrazole-3-carboxylic acid amide

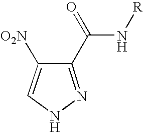

4-Nitropyrazole-3-carboxylic acid (10 g; 63.66 mmol, 1 equiv.) was added to a stirred solution of an amine RNH₂ (70 mmol, 1.1 equiv.), EDC (14.6 g; 76.4 mmol, 1.2 equiv.), and HOBt (10.3 g; 76.4 mmol, 1.2 equiv.) in DMF (250 ml), then stirred at room temperature overnight. The solvent was removed by evaporation under reduced pressure and the residue triturated with ethyl acetate/saturated brine solution. The resultant solid was collected by filtration, washed with 2M hydrochloric acid, then dried under vacuum to give 15.5 g of the amide compound.

### Step J (ii). 4-Amino-1H-pyrazole-3-carboxylic acid (4-fluoro-phenyl)-amide

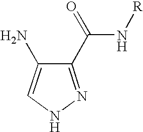

The 4-nitro-1H-pyrazole-3-carboxylic acid amide of Step J (i) (15 g) was dissolved in 200 ml of ethanol, treated with 1.5 g of 10% palladium on carbon under a nitrogen atmosphere, then hydrogenated at room temperature and pressure overnight. The catalyst was removed by filtration through Celite and the filtrate evaporated. The crude product was dissolved in acetone/water (100 ml: 100 ml) and, after slow evaporation of the acetone, the product was collected by filtration as a solid.

### EXAMPLE 1

### Synthesis of 4-(2,3,6-trichloro-benzoylamino)-1H-pyrazole-3-carboxylic acid (1-methyl-piperidin-4-vD-amide

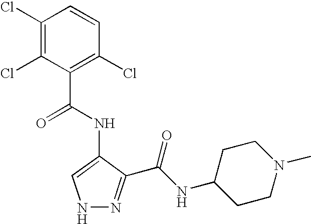

A mixture of 2,3,6-trichlorobenzoic acid (282 mg, 1.25 mmol) in thionyl chloride (4 mL) was heated at reflux for 3 hours, then reduced in vacuo azeotroping with toluene (x3). The residue was taken upin dioxane (8 ml) and 4-amino-1H-pyrazole-3-carboxylic acid (1-methyl-piperidin-4-yl)-amide (283 mg, 1 mmol) added, followed by triethylamine (280 μl, 2 mmol). The mixture was stirred at ambient temperature for 14 hours, reduced in vacuo and the residue partitioned between EtOAc and saturated aqueous NaHCO₃. The layers were separated and the organic portion washed with brine, dried (MgSO₄) and reduced in vacuo. Residue purified by preparative LC/MS to give the title compound as a white solid (60 mg). (LC/MS: r.t. 2.06 min; m/z 430).

### EXAMPLE 2

### Synthesis of 4-(2,6-difluoro-benzoylamino)-1H-pyrazole-3-carboxylic acid [1-(2-cyano-ethyl)-piperidin-4-yl]-amide

### 2A. ri-(2-cyano-ethyl)-piperidin-4-vn-carbamic acid tert-butyl ester

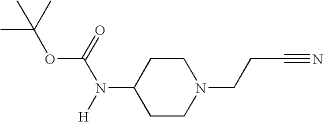

4-Boc-amino-piperidine (1.0 g, 5 mmol), 3-bromo-propionitrile (0.80 g, 6 mmol) and potassium carbonate (1.04 g, 7.5 mmol) in THF (15 ml) were heated at reflux for 16 hours. The reaction mixture was cooled to ambient temperature, poured into water and extracted three times with ethyl acetate. The combined organics were washed (brine) dried (MgSO₄) and reduced in vacuo to a cream solid. NMR revealed partial conversion to the desired product. The solid obtained was redissolved in THF (15 ml) and further 3-bromo-propionitrile (0.80 g, 6 mmol) was added, followed by potassium tert-butoxide (0.84 g, 7.5 mmol) The reaction mixture was heated at reflux for a further 16 hours, cooled to ambient temperature, poured into water and extracted three times with ethyl acetate. The combined organics were washed (brine) dried (MgSO₄) and reduced in vacuo to give [1-(2-cyano-ethyl)-piperidin-4-yl]-carbamic acid tert-butyl ester as a yellow solid (0.704 g, 56%).

### 2B. 4-(2,6-difluoro-benzoylamino)-1H-pyrazole-3-carboxylic acid[1-(2-cyano-ethyl)-piperidin-4-vH-amide

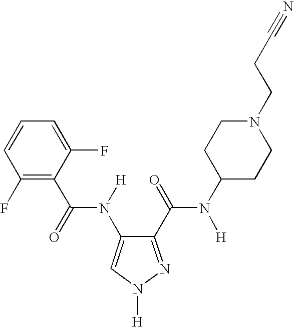

[1-(2-cyano-ethyl)-piperidin-4-yl]-carbamic acid tert-butyl ester (0.230 g, 0.9 mmol) was stirred for 20 minutes in a 1:5 mixture of TFA:DCM (3 ml). The reaction mixture was diluted with methanol, reduced in vacuo and the residue re-evaporated twice with methanol to give a yellow oil. To this was added 4-(2,6-difluoro-benzoylamino)-1H-pyrazole-3-carboxylic acid (Preparation XI) (200 mg, 0.75 mmol), EDC (173 mg, 0.9 mmol), HOBT (122 mg, 0.9 mmol) and DMF (4 ml). The reaction mixture was stirred for 16 hours at ambient temperature, reduced in vacuo and partitioned between ethyl acetate and saturated NaHCO₃ solution. The organic layer was washed (water, brine) dried (MgSO₄) and reduced in vacuo. The residue was purified by column chromatography (SP4-biotage) eluting with 100% ethyl acetate-5% methanol in ethyl acetate to give 4-(2,6-difluoro-benzoylamino)-1H-pyrazole-3-carboxylic acid [1-(2-cyano-ethyl)-piperidin-4-yl]-amide as an off-white solid (55 mg, 18%). (LC/MS: Rₜ 1.79, [M+H]⁺ 403.23).

### EXAMPLE 3

### 4-(2,6-Dichloro-benzo ylamino V 1H-pyrazole-3-carboxylic acid [6-(piperidin-4-yloxyVpyridin-3-yl1-amide

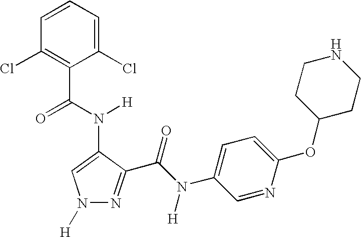

A solution of 4-(5-{4-(dichloro-benzoylamino)-1H-pyrazole-3-carbonyl]-amino}-pyridin-2-yloxy)-piperidine-1-carboxylic acid tert-butyl ester (see Example 45 for this starting material) (260 mg, 0.45 mmoles) in HCl in dioxane (4 M, 10 ml) was stirred at room temperature for 24 hours. The reaction mixture was evaporated in vacuo. The residue was azeotroped with a toluene methanol mixture (1:1). The residue was triturated with ether and filtered to give 4-(2,6-dichloro-benzoylamino)-1H-pyrazole-3-carboxylic acid [6-(piperidin-4-yloxy)-pyridin-3-yl]-amide as a white hydrochloride solid (213 mg, 93%). (LC/MS: Rₜ 2.10, [M+H]⁺ 475.22).

### EXAMPLE 4

### Preparation of 4-(2-chloro-6-fluoro-benzoylamino)-1H-pyrazole-3-carboxylic acid (1-methanesulphonyl-piperidin-4-yl)-amide

### 4A. 4-Amino-1H-pyrazole-3-carboxylic acid (1-methanesulphonyl-piperidin-4-yl) amide

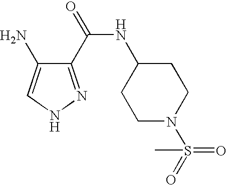

To a stirred solution of 4-(N-BOC amino)piperidine (2.5 g, 12.5 mmoles) in dichloromethane (30 ml) was added triethylamine (2.1 ml, 15.0 mmoles), and then dropwise methanesulphonyl chloride (1.06 ml, 13.8 mmoles). The solution formed was stirred at room temperature for one hour. The reaction mixture was partitioned between EtOAc and water. The organic portion was washed with water, 2N HCl, brine, dried (MgSO₄) filtered and evaporated in vacuo to give 4-(N-BOC-amino)-1-methanesulphonylpiperidine as a white solid (3.1 g, 89%).

A solution of 4-(N-BOC-amino)-1-methanesulphonylpiperidine (3.1 g, 11.15 mmoles) in HCl in dioxane (4 M, 40 ml) was stirred at room temperature for 24 hours. The reaction mixture was evaporated in vacuo. The residue was azeotroped with a toluene: methanol mixture (1:1) to give 1-methanesulphonyl-piperidin-4-ylamine as a white hydrochloride salt (2.4 g, 100%).

A solution of 1-methanesulphonyl-piperidin-4-ylamine hydrochloride (2.4 g, 111.1 mmoles), 4-nitro-1H-pyrazole-3-carboxylic acid (1.8 g, 11.1 mmoles), EDC (2.6 g (13.5 mmoles), HOBt (1.8 g, 13.3 mmoles) and triethylamine (3.4 ml, 24.6 mmoles) in DMF (30 ml) was stirred at room temperature for 24 hours. The reaction mixture was partitioned between EtOAc and a saturated solution of sodium hydrogen carbonate. The organic portion was dried (MgSO₄), filtered and evaporated in vacuo to give 4-nitro-1H-pyrazole-3-carboxylic acid (1-methanesulphonyl-piperidin-4-yl)-amide as a pale orange solid (1.7 g, 48%).

To a solution of 4-nitro-1H-pyrazole-3-carboxylic acid (1-methanesulphonyl-piperidin-4-yl)-amide (1 Jg, 5.36 mmoles) in ethanol (20 ml) under nitrogen was added 10% palladium on carbon (150 mg) and then hydrogenated at RTP for 2 hours. Further palladium on carbon (150 mg) was added and the resultant suspension hydrogenated at RTP for a further 2 hours. The reaction mixture was filtered through Celite. The filtrate was evaporated in vacuo to give 4-amino-1H-pyrazole-3-carboxylic acid (1-methanesulphonyl-piperidin-4-yl)amide as a yellow/brown oil (1.5 g, 98%) (LC/MS: Rₜ 0.33, [M+H]⁺ 288.21).

### 4B. 4-(2-Chloro-6-fluoro-benzoylamino)-1H-pyrazole-3-carboxyric acid (1-methanesulphonyl-piperidin-4-yl)-amide

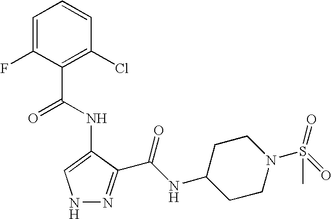

A solution of 4-amino-1H-pyrazole-3-carboxylic acid (1-methanesulphonyl-piperidin-4-yl) amide (150 mg, 5.23 mmoles), 2-chloro-6-fluorobenzoic acid (91 mg, 0.523 mmoles), HOBt (85 mg, 0.627 mmoles) and EDC (120 mg, 0.627 mmoles) in DMF (10 ml) was stirred at ambient temperature for 3 hours. The reaction mixture was partitioned between EtOAc and a saturated solution of sodium hydrogen carbonate. The organic portion was washed with water (x2), brine, dried (MgSO₄), filtered and evaporated in vacuo. The residue was purified by flash chromatography (Biotage SP4, 25S, flow rate 25 ml/min, gradient EtOAc/Petrol (1:1) to EtOAc) to give 4-(2-chloro-6-fluoro-benzoylamino)-1H-pyrazole-3-carboxylic acid (1-methanesulphonyl-piperidin-4-yl)-amide as a white solid (25 mg, 11%). (LC/MS: Rₜ 2.57, [M+H]⁺444.22)

### EXAMPLE 5

### Preparation of 4-(2-chloro-6-methoxy-benzoylamino)-1H-pyrazole-3-carboxylic acid (1-methanesulphonyl-piperidin-4-yl)-amide

### 5A. 4-{[4-(2-Chloro-6-methoxy-benzoylamino)-1H-pyrazole-3-carbonyl]-amino}-piperidine-1-carboxylic acid tert-butyl ester

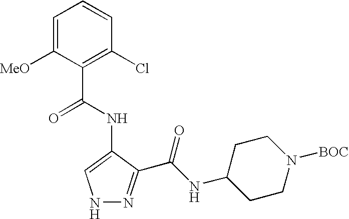

To a suspension of 2-methoxy-6-chlorobenzonitrile (1.0 g, 5.97 mmoles) in potassium hydroxide solution (3 g of KOH in 20 ml water) was added 4 ml of hydrogen peroxide solution (30% w/w). The reaction mixture was heated at 70° C. for 20 hours, then at 100° C. for 6 hours. The reaction mixture was cooled to ambient temperature to give a white suspension. The reaction mixture was filtered to give a white solid. The solid was dissolved in acetonitrile (2 ml), and to the solution formed was added cautiously concentrated sulphuric acid (10 ml). The reaction mixture was stirred below 30° C. for 30 minutes. Sodium nitrite (2.58 g, 37 mmoles) was added to the reaction mixture portionwise. The reaction mixture was stirred at ambient temperature for 16 hours and then poured onto ice. The ice mixture was then washed with EtOAc (x3). The organic portions were combined, dried (MgSO₄) filtered and evaporated in vacuo to give 2-chloro-6-methoxybenzoic acid (786 mg, 71%).

A stirred solution of 4-[4-amino-1H-pyrazole-3-carbonyl)-amino]-piperidine-1-carboxylic acid tert-butyl ester (100 mg, 0.324 mmoles), 2-chloro-6-methoxybenzoic acid (60 mg, 0.324 mmoles), EDC 75 mg (0.389 mmoles) and HOBt (53 mg, 0.389 mmoles) in DMF (5 ml) was stirred at 70° C. for 48 hours. The reaction mixture was diluted with EtOAc (50 ml) and washed with a saturated solution of sodium hydrogen carbonate, water (x3), brine, dried (MgSO₄), filtered and evaporated in vacuo. The residue was purified by flash chromatography (Biotage SP4, 25S, flow rate 25 ml/min, gradient EtOAc/Petrol, 1:1, to EtOAc) to give 4-{[4-(2-chloro-6-methoxy-benzoylamino)-1H-pyrazole-3-carbonyl]-amino}-piperidine-1-carboxylic acid tert-butyl ester as a pale yellow solid (100 mg, 65%). (LC/MS: Rₜ 3.18, [M+H]⁺ 478.29).

### 5B. 4-(2-Chloro-6-methoxy-benzoylamino)-1H-pyrazole-3-carboxylic acid Cl-methanesurphonyl-piperidin-4-ylV amide

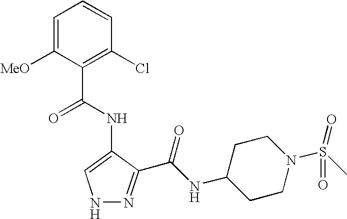

4-{[4-(2-Chloro-6-methoxy-benzoylamino)-1H-pyrazole-3-carbonyl]-amino}-piperidine-1-carboxylic acid tert-butyl ester (100 mg, 0.21 mmoles) was dissolved in HCl in dioxane (4M, 10 ml) and stirred at ambient temperature for 30 minutes. The reaction was evaporated in vacuo. The residue was azeotroped with a toluene:methanol mixture (1:1). The residue was dissolved in dichloromethane (10 ml) and DMF (1 ml). To the resultant solution was added diisopropylethylamine (84 μl, 0.46 mmoles) and methanesulphonyl chloride (17 μl, 0.21 mmoles). The reaction mixture was stirred at ambient temperature for 30 minutes, and then purified firstly by flash chromatography (Biotage SP4, 25S, flow rate 25 ml/min, gradient EtOAc/Petrol (1:1) to EtOAc) and then by trituration with ether to give 4-(2-chloro-6-methoxy-benzoylamino)-1H-pyrazole-3-carboxylic acid (1-methanesulphonyl-piperidin-4-yl)-amide as a white solid (34 mg, 36%). (LC/MS: Rₜ 2.56, [M+H]⁺ 456.23).

### EXAMPLE 6

### Preparation of 4-(2,6-dichloro-benzoylamino)-1H-pyrazole-3-carboxylic acid[1-(2-dimethylamino-ethanesulphonyl)-piperidin-4-yl]-amide

### 6A. 4-r2,6-dichloro-benzoylamino)-1H-pyrazole-3-carboxylic acid (1-ethenesulphonyl-piperidin-4-ylV amide

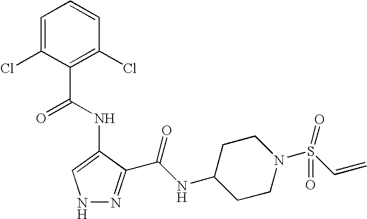

To a solution of 4-(2,6-dichloro-benzoylamino)-1H-pyrazole-3-carboxylic acid piperidine-4-ylamide hydrochloride (Preparation X) (2 g, 4.78 mmoles) in DMF (20 ml) was added triethylamine (2.7 ml, 19.12 mmoles) and then 2-chloro-1-ethanesulphonyl chloride (0.5 ml, 4.78 mmoles). The reaction mixture was stirred at ambient temperature for 30 minutes. Further 2-chloro-1-ethanesulphonyl chloride (175 μl, 1.67 mmoles) was added and the reaction mixture was stirred at ambient temperature for a further hour. The reaction mixture was diluted with EtOAc and washed with water (x3) and then brine. The organic portion was dried (MgSO₄), filtered and evaporated in vacuo. The residue was purified by flash chromatography (Biotage SP4, 40S, flow rate 40 ml/min, gradient 1:1 EtOAc/Petrol to EtOAc) to give 4-(2,6-dichloro-benzoylamino)-1H-pyrazole-3-carboxylic acid (1-ethenesulphonyl-piperidin-4-yl)-amide as a white solid (500 mg, 22%). (LC/MS: Rₜ2.94, [M+H]⁺ 472.15).

### 6B. 4-(2,6-dichloro-benzoylamino)-1H-pyrazole-3-carboxylic acid[1-(2-dimethylamino-ethanesulphonylVpiperidin-4-yll -amide

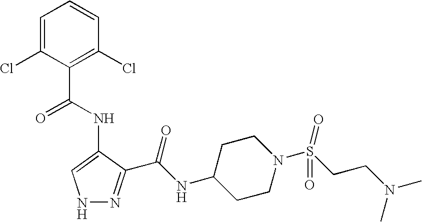

A solution of 4-(2,6-dichloro-benzoylamino)-1H-pyrazole-3-carboxylic acid (1-ethenesulphonyl-piperidin-4-yl)-amide (100 mg, 0.212 mmoles) in ethanolic dimethylamine (10 ml, 35% w/v) was stirred at ambient temperature for 10 minutes. The solvent was evaporated in vacuo. The residue was purified by flash chromatography (Biotage SP4, 25S, flow rate 25 ml/min, gradient 1:20 MeOH/DCM to 1:10 MeOH/DCM) to give 4-(2,6-dichloro-benzoylamino)-1H-pyrazole-3-carboxylic acid [1-(2-dimethylamino-ethanesulphonyl)-piperidin-4-yl]-amide as a white solid (30 mg, 27%). (LC/MS: Rₜ 2.16, [M+H]⁺ 517.22).

### EXAMPLE 7

### Preparation of 4-(2,6-dichloro-benzoylamino)-1H-pyrazole-3-carboχylic acid [1-(2-hydroxy-ethanesulphonyiypiperidin-4-yl]-amide

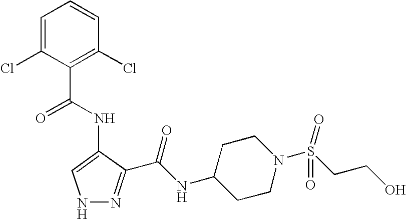

To a solution of 4-(2,6-dichloro-benzoylamino)-1H-pyrazole-3-carboxylic acid (1-ethenesulphonyl-piperidin-4-yl)-amide (Example 6A) (100 mg, 0.212 mmoles) in THF (10 ml) under nitrogen was added borane-dimethylsulphide in THF (2M, 106 μl, 0.212 mmoles). The resultant solution was stirred at ambient temperature for 30 minutes. Hydrogen peroxide solution (5 ml, 30% w/v) and sodium hydroxide solution (5 ml, 2N) was added to the reaction mixture. The reaction mixture was stirred at ambient temperature for 24 hours. The reaction mixture was partitioned between EtOAc and water. The organic portion was dried (MgSO₄), filtered and evaporated in vacuo. The residue was purified by flash chromatography (Biotage SP4, 25S, flow rate 25 ml/min, gradient 1:1 EtOAc/Petrol to EtOAc) to give 4-(2,6-dichloro-benzoylamino)-1H-pyrazole-3-carboxylic acid [1-(2-hydroxy-ethanesulphonyl)-piperidin-4-yl]-amide as a white solid (10 mg, 10%). (LC/MS: Rₜ 2.66, [M+H]⁺ 490.16).

### EXAMPLE 8

### Synthesis of 4-(2,6-dichloro-benzoylamino)-1-H-pyrazole-3-carboxylic acid[1-(2,2,2-trifluoro-acetyl)-piperidin-4—yl1-amide

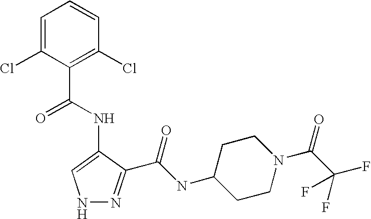

To a suspension of 4-(2,6-dichloro-benzoylamino)-1H-pyrazole-3-carboxylic acid piperidin-4-ylamide hydrochloride (PreparationX) (0.3 g, 0.71 mmol), triethylamine (0.213 ml, 1.42 mmol) in THF (5 ml) was added trifluoroacetic anhydride (0.1 ml, 0.71 mmol). The reaction mixture was stirred at room temperature for 15 hours. The crude product was partitioned between EtOAc and water, the organic phase was dried over MgSO₄, filtered and evaporated in vacuo. The residue was triturated with diethyl ether to afford the title compound as pale yellow solid (0.1 g, 30%) (LC/MS: Rₜ 2.96, [M+H]⁺ 478).

### EXAMPLE 9

### Synthesis of 4-(2,6-dichloro-benzoylaminoV1-H-pyrazole-3-carboxylic acid[1-Cmorpholine-4-sulphonyl)-piperidin-4vn-amide

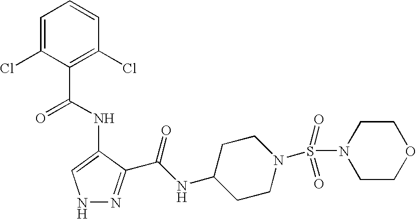

To morpholinium chloride (0.5 g, 4 mmol), was added triethylamine (6 ml, 40 mmol) and the mixture was stirred for 15 minutes at room temperature. Chloroform was added (10 ml), the mixture was cooled to −5° C. and chlorosulphonic acid (0.266 ml, 4 mmol) was added dropwise so as to maintain the temperature below 0° C. The chloroform was evaporated and the mixture was treated with 0.03 mol of NaOH in 16 ml of water. The solution was evaporated to dryness to afford morpholine-4-sodium sulphamate. The crude material was dissolved in 1,2-dichloroethane (5 ml) and POCl₃ (0.7 ml, 8 mmol) was added. The reaction mixture was heated at 80° C. for 18 hours. Petroleum ether and EtOAc were then added to the mixture and solids were removed by filtration. The filtrate was evaporated to dryness to afford morpholine sulphamoyl chloride. The resulting crude material was dissolved in DCM (30 ml), triethylamine (1 ml, 10 mmol) was added followed by the addition of 4-(2,6-dichloro-benzoylamino)-1H-pyrazole-3-carboxylic acid piperidin-4-ylamide hydrochloride (Preparation X) (1 g, 4 mmol) at 0° C. The reaction mixture was stirred at room temperature for 16 hours, then added dioxane (5 ml) and heated at 50° C. for 3 hours. The crude product was partitioned between EtOAc and water. The organic phase was dried over MgSO₄, filtered and evaporated in vacuo. The residue was purified by flash chromatography on silica eluting with EtOAc:hexane 1:2 to 100% EtOAc to afford the title compound as white solid (130 mg, 10% over 3 steps) (LC/MS: Rₜ 2.80, [M+H]⁺ 531).

### EXAMPLES 10-134

By using the methods set out above, the compounds of Examples 18 to 138 were prepared. In the Table below, the general synthetic route used in each case, together with any modifications (if any) to the reactants and conditions, are given for each example.

| Ex- | | | |
|---|---|---|---|
| ample | Structure | Method of Preparation | LCMS |
| 10 | 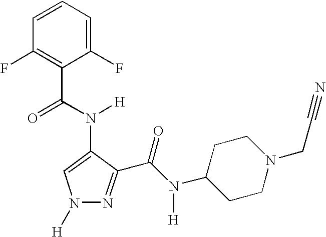 | Example 2Step A: bromoacetonitrile, KOtBu,heated at reflux for 1 h. | [M + H]⁺389.18Rₜ 2.26 |
| 11 | 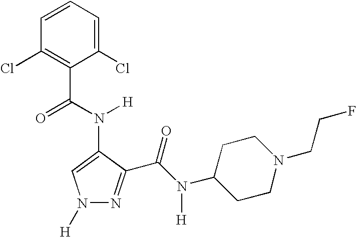 | Example 2Step A: 1-bromo-2-fluoroethane,K₂CO₃, heated at reflux for 16 h.Step B: 4-(2,6-dichloro-benzoylamino)-1H-pyrazole-3-carboxylic acid (Preparation IX, step1) used in coupling | [M + H]⁺428.30Rₜ 1.91 |
| 12 | 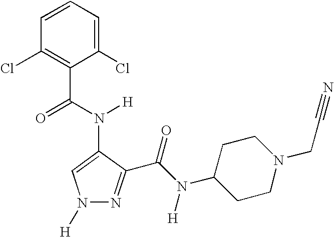 | Example 2Step A: bromoacetonitrile, K₂CO₃,heated at reflux for 16 h.Step B: 4-(2,6-dichloro-benzoylamino)-1H-pyrazole-3-carboxylic acid (Preparation IX, step1) used in coupling | [M + H]⁺421.25Rₜ 2.44 |
| 13 | 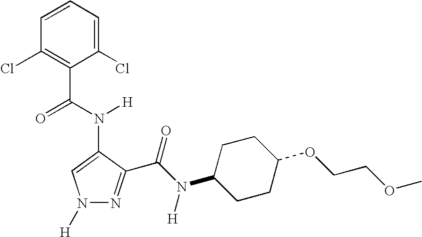 | General Procedure A using trans-4-(2-methoxy-ethoxy)-cyclohexylamine(Preparation I) | [M + H]⁺455.33Rₜ 2.74 |
| 14 | 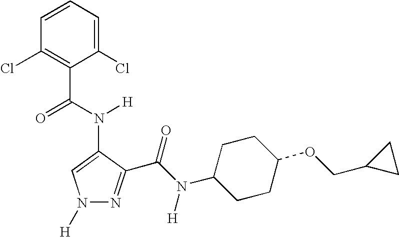 | Preparation I, except using(bromomethyl)cyclopropane in step2, then general Procedure A. | [M + H]⁺451.28Rₜ 3.12 |
| 15 | 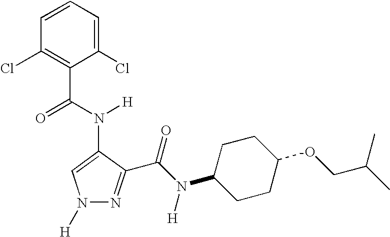 | Preparation I, except using 1-bromo-2-methylpropane in step 2 thenGeneral Procedure A | [M + H]⁺453.28Rₜ 3.45 |
| 16 | 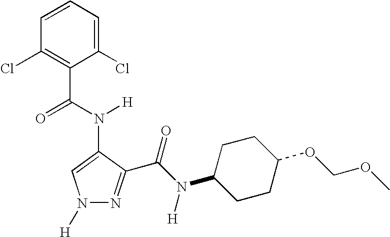 | Preparation I, except usingmethoxymethyl chloride in step 2then General Procedure A | [M + H]⁺441.21Rₜ 2.88 |
| 17 | 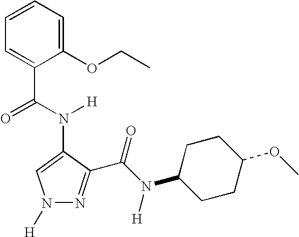 | Preparation I, except usingiodomethane in step 2 then Generalprocedure D (i) and (iii) then GeneralProcedure B using 2-ethoxybenzoicacid | [M + H]⁺387.27Rₜ 2.94 |
| 18 | 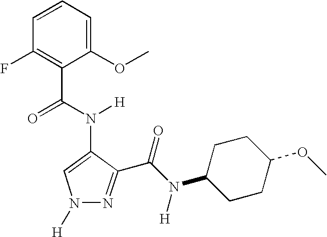 | Preparation I, except usingiodomethane in step 2 then Generalprocedure D (i) and (iii) then GeneralProcedure B using 2-fluoro-6-methoxybenzoic acid | [M + H]⁺391.32Rₜ 2.61 |
| 19 | 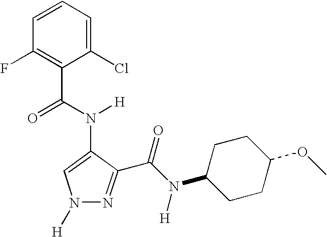 | Preparation I, except usingiosomethane in step 2 then Generalprocedure D (i) and (iii) then GeneralProcedure B using 2-chloro-6-fluorobenzoic acid | [M + H]⁺395.27Rₜ 2.71 |
| 20 | 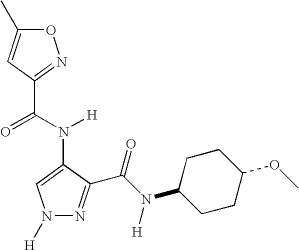 | Preparation I, except usingiodomethane in step 2 then Generalprocedure D (i) and (iii) then GeneralProcedure B using 5-methyl-isoxazole-3-carboxylic acid | [M + H]⁺384.29Rₜ 2.56 |
| 21 | 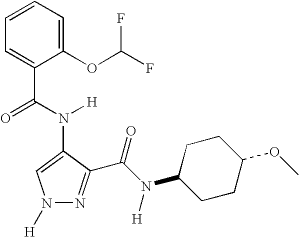 | Preparation I, except usingiodomethane in step 2 then Generalprocedure D (i) and (iii) then GeneralProcedure B using 2-difluoromethoxybenzoic acid | [M + H]⁺409.29Rₜ 2.84 |
| 22 | 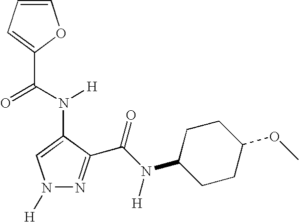 | Preparation I, except usingiodomethane in step 2 then Generalprocedure D (i) and (iii) then GeneralProcedure B using furan-2-carboxylicacid | [M + H]⁺333.25Rₜ 2.45 |
| 23 | 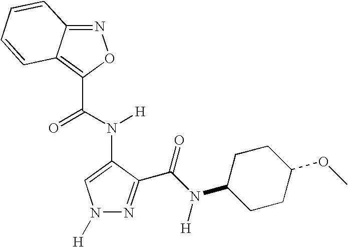 | Preparation I, except usingiodomethane in step 2 then Generalprocedure D (i) and (iii) then GeneralProcedure B using benzo[c]isoxazole-3-carboxylic acid | [M + H]⁺384.28Rₜ 2.81 |
| 24 | 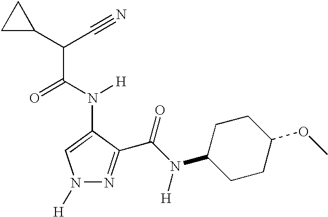 | Preparation I, except usingiosomethane in step 2 then Generalprocedure D (i) and (iii) then GeneralProcedure B using cyano-cyclopropyl-acetic acid | [M + H]⁺346.30Rₜ 2.51 |
| 25 | 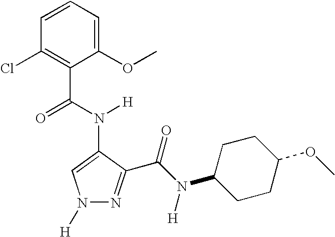 | Preparation I, except usingiodomethane in step 2 then Generalprocedure D (i) and (iii) then GeneralProcedure B using 2-chloro-6-methoxybenzoic acid. | [M + H]⁺407.19Rₜ 2.85 |
| 26 | 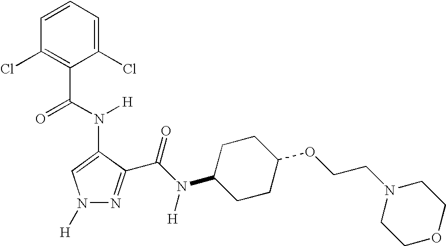 | Preparation I, except using N-(2-chloroethyl)morpholine, (preformedfrom the HCl salt by treatment withNEt₃ in dioxane) in step 2 thenGeneral Procedure A | [M + H]⁺510.23Rₜ 1.99 |
| 27 | 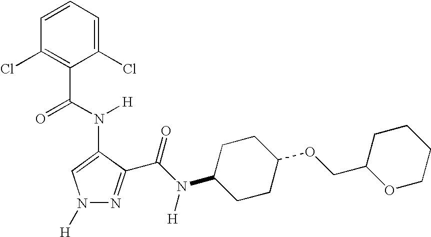 | Preparation I, except using 2-(bromomethyl)tetrahydro-2H-pyranin step 2, and DMF as solvent in step3, then General Procedure A | [M + H]⁺495.24Rₜ 3.06 |
| 28 | 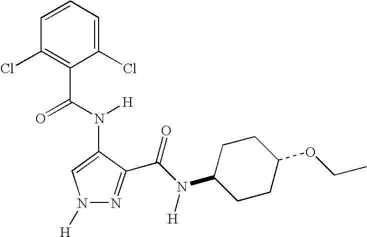 | Preparation I, except using ethyliodide in step 2, then GeneralProcedure A | [M + H]⁺425.15Rₜ 2.96 |
| 29 | 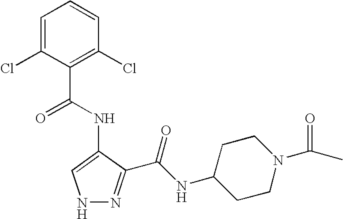 | General procedure G using acetylchloride. | [M + H]⁺ 424Rₜ 2.44 |
| 30 | 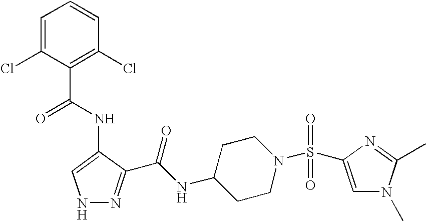 | General procedure G using 1,2-dimethyl-1H-imidazole-4-sulphonylchloride.Purification by preparative LC/MS | [M + H]⁺ 540Rₜ 2.51 |
| 31 | 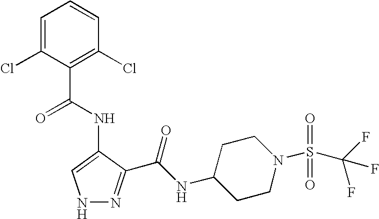 | General procedure G usingtrifluoromethylsulphonyl chloride.Purification by columnchromatography[P.E.-EtOAc (1:1-0:1)] | [M + H]⁺ 514Rₜ 3.21 |
| 32 | 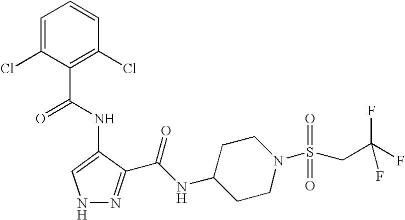 | General procedure G using 2,2,2-trifluoro-ethanesulphnyl chloride.Purification by columnchromatography[P.E.-EtOAc (1:1-0:1)] | [M + H]⁺ 528Rₜ 3.04 |
| 33 | 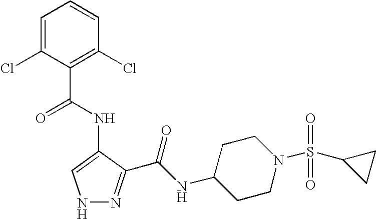 | General procedure G usingcyclopropylsulphonyl chloride.Purification by hot slurry[EtOAc-MeOH (4:1)] | [M + H]⁺ 486Rₜ 2.76 |
| 34 | 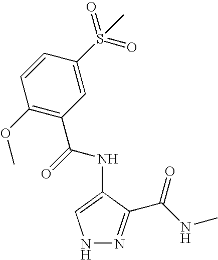 | General Procedure J (i) (usingmethylamine hydrochloride) and step(ii), followed by general procedure B(using 5-methanesulphonyl-2-methoxy-benzoic acid). | [M + H]⁺ 353Rₜ 2.1 |
| 35 | 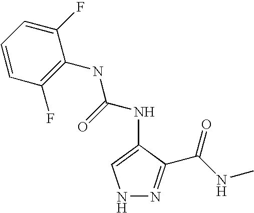 | General Procedure J (i) (usingmethylamine hydrochloride) and step(ii), followed by general procedure F(using 2,6-difluorophenylisocyanate). | [M + H]⁺ 296Rₜ 2.17 |
| 36 | 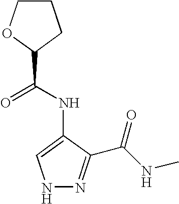 | General Procedure J (i) (usingmethylamine hydrochloride) and step(ii), followed by general procedure B(using (S)-(−)-2-tetrahydrofuroicacid) | [M + H]⁺ 239Rₜ 1.83 |
| 37 | 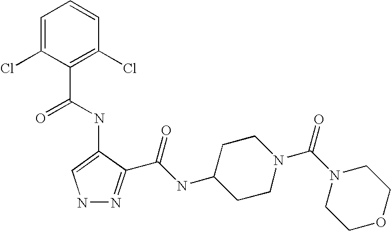 | General procedure G (using 4-morpholinecarbonyl chloride).Crystallisation from DCM/Et₂O | [M + H]⁺ 495Rₜ 2.56 |
| 38 | 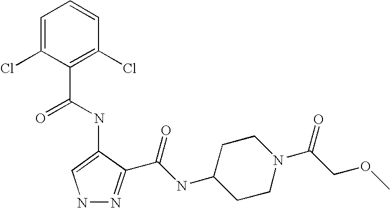 | General procedure G (usingmethoxyacetyl chloride).Crystallisation from DCM/Et₂O | [M + H]⁺ 454Rₜ 2.44 |
| 39 | 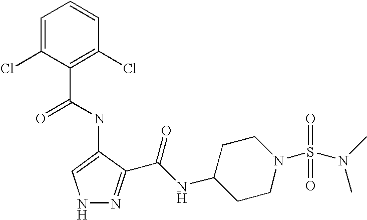 | General procedure G (usingdimethylsulphamoyl chloride).Purification by columnchromatographyMeOH/DCM (2% then 5%) | [M + H]⁺ 489Rₜ 2.86 |
| 40 | 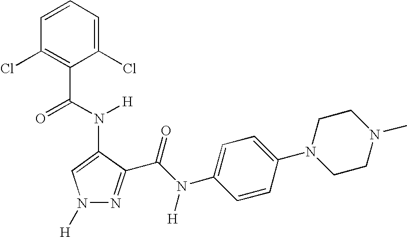 | General Procedure E, except productpurified by trituration with diethylether. 4-(4-methylpiperazino)-anilineused as the amine. | [M + H]⁺473.15Rₜ 2.15 |
| 41 | 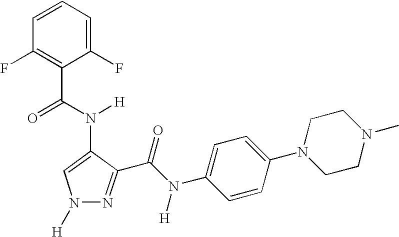 | General Procedure C except purifiedby trituration with ether. 4-(4-methylpiperazino)-aniline used as theamine. | [M + H]⁺441.23Rₜ 2.06 |
| 42 | 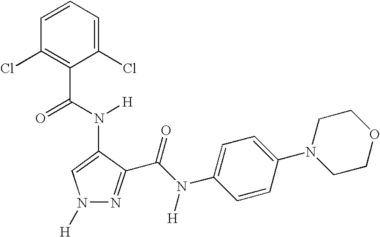 | General Procedure E. except productpurified by trituration with ether:petrol (1:1). 4-morpholinoanilineused as the amine. | [M + H]⁺460.09Rₜ 3.00 |
| 43 | 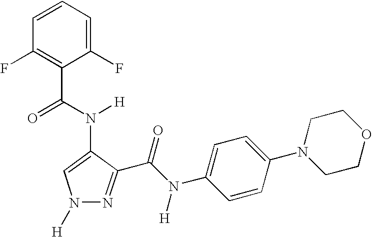 | General Procedure C, except purifiedby trituration with ether:petrol (1:1).4-morpholinoaniline used as theamine. | [M + H]⁺428.18Rₜ 2.94 |
| 44 | 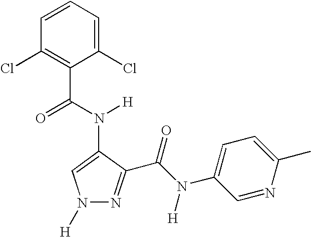 | General Procedure E, except productpurified by flash chromatography.3-amino-6-picoline used as the amine | [M + H]⁺390.11Rₜ 2.08 |
| 45 | 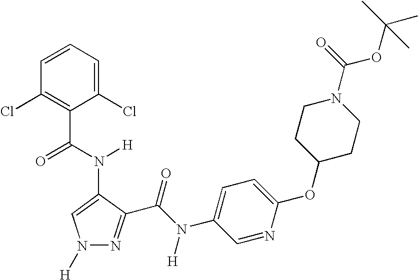 | General Procedure E, except productpurified by flash chromatography.4-(5-amino-pyridin-2-yloxy)-piperidine-1-carboxylic acid tert-butyl ester used as amine | [M + H]⁺575.31Rₜ 3.51 |
| 46 | 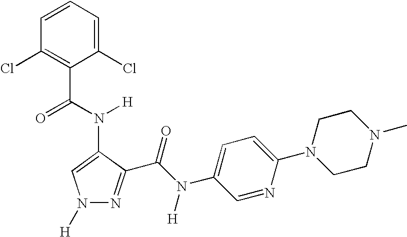 | General Procedure E, except productpurified by trituration with diethylether. 6-(4-methylpiperazino)-3-pyridamine used as amine | [M + H]⁺474.23Rₜ 2.08 |
| 47 | 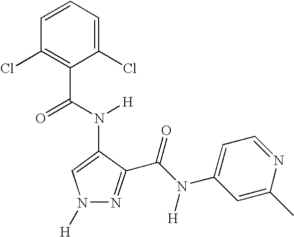 | General Procedure E, except productpurified by flash chromatography.4-amino-2-picoline used as amine | [M + H]⁺390.12Rₜ 1.97 |
| 48 | 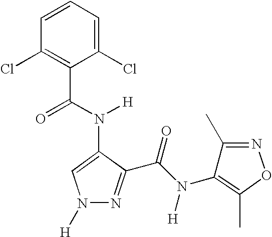 | General Procedure E, except productpurified by flash chromatography.3,5-dimethyl-4-aminoisoxazole usedas amine. | [M + H]⁺394.08Rₜ 2.76 |
| 49 | 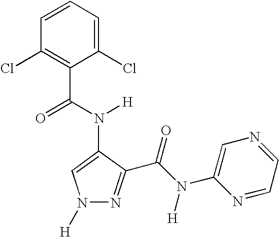 | General Procedure E, except productpurified by flash chromatography.Aminopyrazine used as amine | [M + H]⁺377.11Rₜ 2.70 |
| 50 | 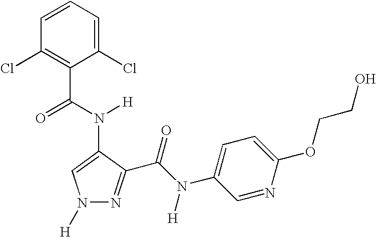 | General Procedure E, except productpurified by trituration with diethylether. 2-(5-amino-pyridin-2-yloxy)-ethanol (Preparation II) used asamine. | [M + H]⁺436.13Rₜ 2.54 |
| 51 | 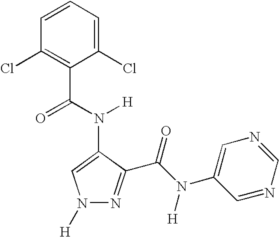 | General Procedure E, except productpurified by flash chromatography.5-aminopyrimidine used as amine. | [M + H]⁺377.21Rₜ 2.51 |
| 52 | 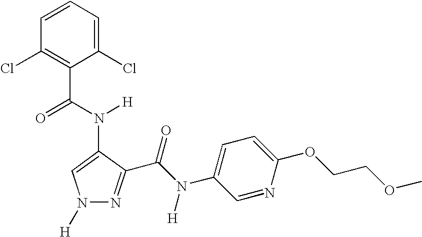 | General Procedure E, except productpurified by flash chromatography.6-(2-methoxy-ethoxy)-pyridin-3-ylamine (Preparation III) used asamine. | [M + H]⁺450.27Rₜ 2.85 |
| 53 | 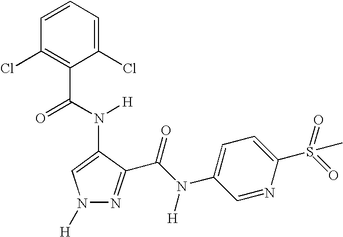 | General Procedure E, except productpurified by flash chromatography.6-methanesulphonyl-pyrid-3-ylamine(prepared by method described inEP1104745A1) used as amine. | [M + H]⁺454.16Rₜ 2.71 |
| 54 | 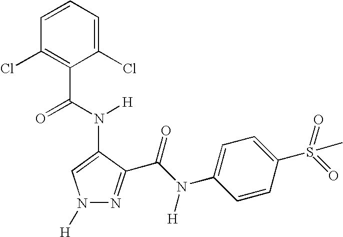 | General Procedure E, except productpurified by flash chromatography.4-(methanesulphonyl)aniline used asamine. | [M + H]⁺453.13Rₜ 2.83 |
| 55 | 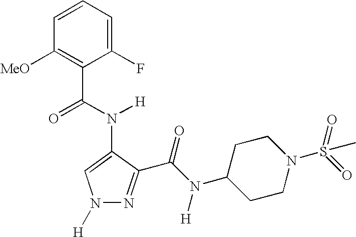 | As per Example 4B but using 5-fluoromethoxybenzoic acid instead of2-chloro-6-fluorobenzoic acid. | [M + H]⁺440.29Rₜ 2.44 |
| 56 | 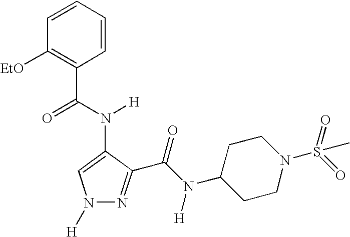 | As per Example 4B but using 2-ethoxybenzoic acid instead of 2-chloro-6-fluorobenzoic acid. | [M + H]⁺436.19Rₜ 2.72 |
| 57 | 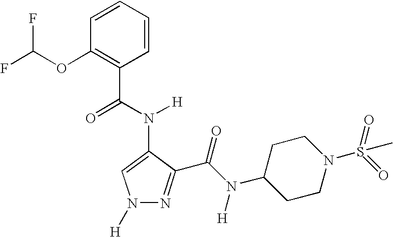 | As per Example 4B but using 2-(difluoromethoxy)benzoic acidinstead of 2-chloro-6-fluorobenzoicacid. | [M + H]⁺458.18Rₜ 2.71 |
| 58 | 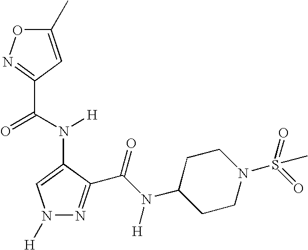 | As per Example 4B but using 5-methylisoxazole-3-carboxylic acidinstead of 2-chloro-6-fluorobenzoicacid. | [M + H]⁺397.25Rₜ 2.48 |
| 59 | 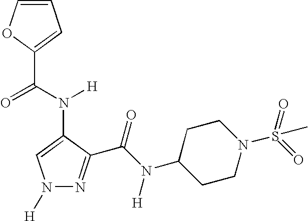 | As per Example 4B but using 2-furoic acid instead of 2-chloro-6-fluorobenzoic acid. | [M + H]⁺382.26Rₜ 2.29 |
| 60 | 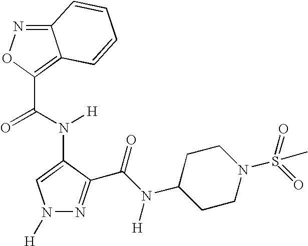 | As per Example 4B but using usingbenzo[c]isoxazole-3-carboxylic acidinstead of 2-chloro-6-fluorobenzoicacid. | [M + H]⁺433.25Rₜ 2.71 |
| 61 | 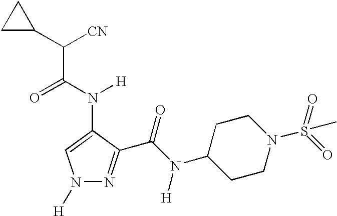 | As per Example 4B but using cyano-cyclopropyl-acetic acid instead of 2-chloro-6-fluorobenzoic acid. | [M + H]⁺395.28Rₜ 2.40 |
| 62 | 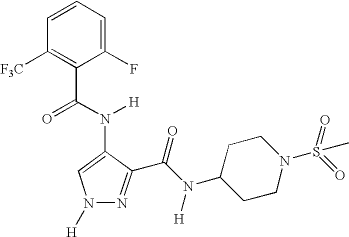 | As per Example 4B but using 2-fluoro-6-(trifluoromethyl)benzoylchloride and triethylamine instead of2-chloro-6-fluorobenzoic acid, EDCand HOBt | [M + H]⁺478.22Rₜ 2.66 |
| 63 | 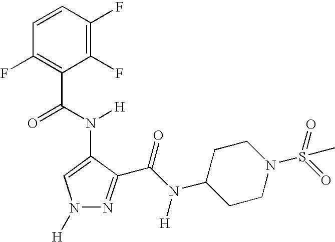 | As per Example 4B but using 2,3,6-trifluorobenzoyl chloride andtriethylamine instead of 2-chloro-6-fluorobenzoic acid, EDC and HOBt | [M + H]⁺446.23Rₜ 2.58 |
| 64 | 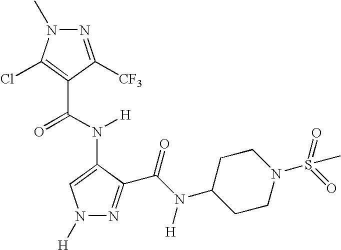 | As per Example 4B but using 5-chloro-1-methyl-3-(trifluoromethyl)-1H-pyrazole-4-carboxylic acidinstead of 2-chloro-6-fluorobenzoicacid | [M + H]⁺498.14Rₜ 2.58 |
| 65 | 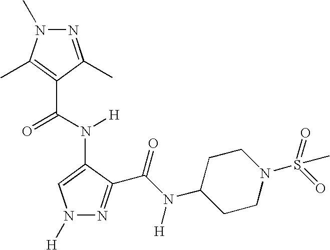 | As per Example 4B but using 1,3,5-trimethyl-1H-pyrazole-4-carboxylicacid instead of 2-chloro-6-fluorobenzoic acid | [M + H]⁺424.27Rₜ 2.13 |
| 66 | 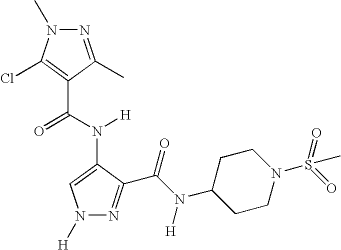 | As per Example 4B but using 5-chloro-1,3-dimethyl-1H-pyrazole-4-carboxylic acid instead of 2-chloro-6-fluorobenzoic acid | [M + H]⁺444.21Rₜ 2.26 |
| 67 | 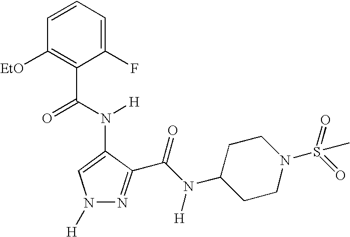 | As per Example 4B but using 2-ethoxy-6-fluorobenzoic acid insteadof 2-chloro-6-fluorobenzoic acid | [M + H]⁺454.27Rₜ 2.58 |
| 68 | 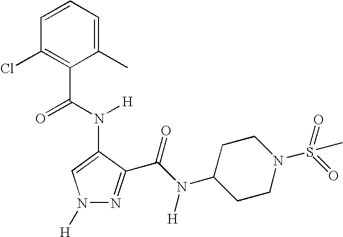 | As per Example 4B but using 2-chloro-6-methylbenzoic acid insteadof 2-chloro-6-fluorobenzoic acid | [M + H]⁺440.22Rₜ 2.63 |
| 69 | 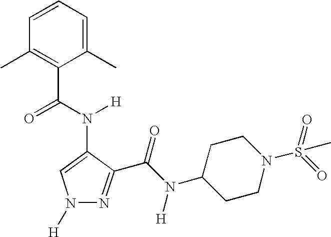 | As per Example 4B but using 2,6-dimethylbenzoyl chloride andtriethylamine instead of 2-chloro-6-fluorobenzoic acid, EDC and HOBt | [M + H]⁺420.29Rₜ 2.62 |
| 70 | 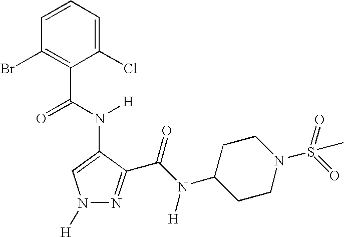 | As per Example 4B but using 2-bromo-6-chlorobenzoyl chloride andtriethylamine instead of 2-chloro-6-fluorobenzoic acid, EDC and HOBt | [M + H]⁺504.07Rₜ 2.64 |
| 71 | 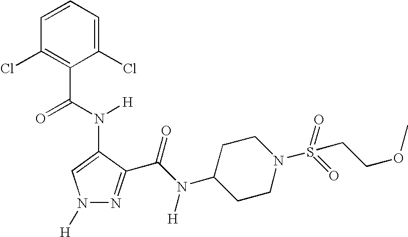 | General Procedure H using 2-chloroethyl methyl ether | [M + H]⁺504.16Rₜ 2.78 |
| 72 | 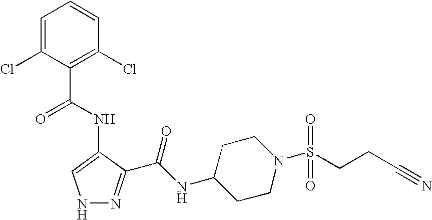 | General Procedure H using 2-chloropropionitrilePurified by preparative LC/MS | [M + H]⁺ 499Rₜ 2.75 |
| 73 | 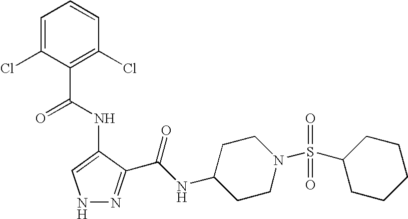 | General Procedure I usingcyclohexanethiol.Purified by preparative LC/MS | [M + H]⁺ 528Rₜ 3.25 |
| 74 | 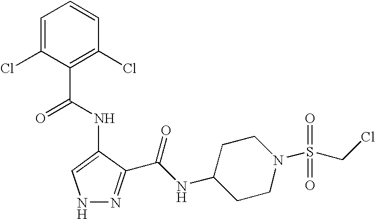 | General Procedure G usingchloromethanesulphonyl chloride.Purification by columnchromatography[P.E.-EtOAc (1:0-0:1)] | [M + H]⁺ 494Rₜ 3.11 |
| 76 | 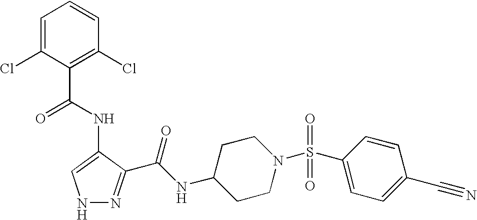 | General Procedure G using 4-cyanophenylsulphonyl chloride.Purification by preparative LC/MS | [M + H]⁺ 547Rₜ 3.26 |
| 77 | 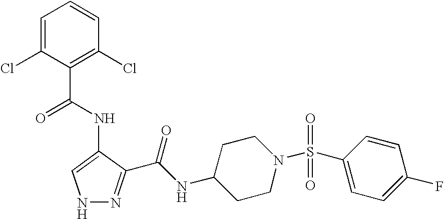 | General Procedure G using 4-fluorophenylsulphonyl chloride.Purification by columnchromatography[P.E.-EtOAc (1:0-0:1)] | [M + H]⁺ 540Rₜ 3.34 |
| 78 | 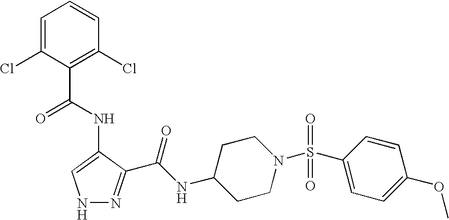 | General Procedure G using 4-methoxyphenylsulphonyl chloride.Purification by columnchromatography[P.E.-EtOAc (1:0-0:1)] | [M + H]⁺ 552Rₜ 3.31 |
| 79 | 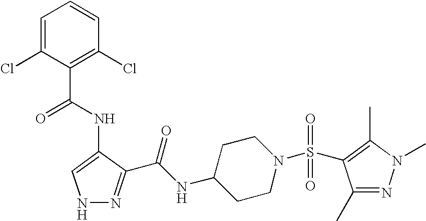 | General Procedure G using 1,3,5-trimethyl-1H-pyrazole-4-sulphonylchloride.Purified by precipitation from water. | [M + H]⁺ 554Rₜ 2.98 |
| 80 | 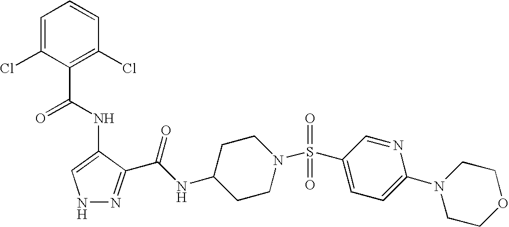 | General Procedure G using 6-morpholin-4-yl-pyridine-3-sulphonylchloride.Purified by precipitation from water. | [M + H]⁺ 608Rₜ 3.11 |
| 81 | 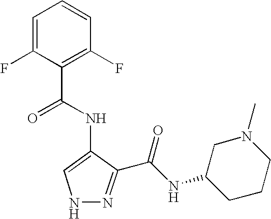 | General Procedure A using 1-methyl-piperidin-3-(S)-ylamine (PreparationIV). Purification by preparativeLC/MS | [M + H]⁺ 364Rₜ 2.54 |
| 83 | 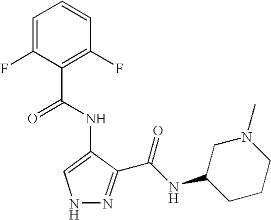 | General Procedure A using 1-methyl-piperidin-3-(R)-ylamine (PreparationV). Purification by preparativeLC/MS | [M + H]⁺ 364Rₜ 1.81 |
| 84 | 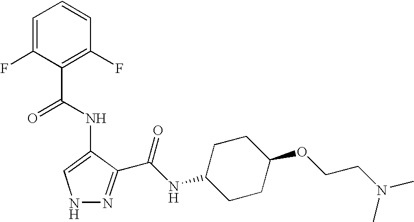 | General Procedure A using trans-4-(2-dimethylamino-ethoxy)-cyclohexylamine (Preparation VII).Purification by preparative LC/MS | [M + H]⁺ 436Rₜ 1.99 |
| 85 | 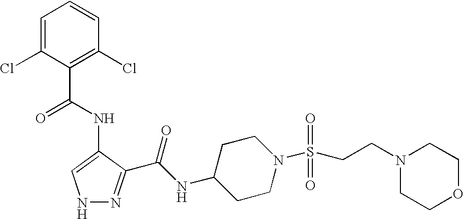 | As per Example 6, but usingmorpholine in step 6B instead ofdimethylamine | [M + H]⁺559.17Rₜ 2.19 |
| 86 | 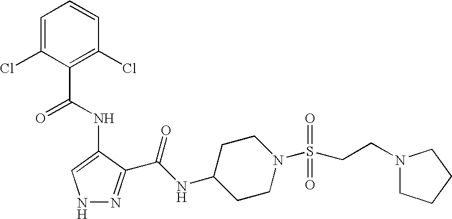 | As per Example 6, but usingpyrrolidine in step 6B instead ofdimethylamine | [M + H]⁺543.18Rₜ 2.24 |
| 87 | 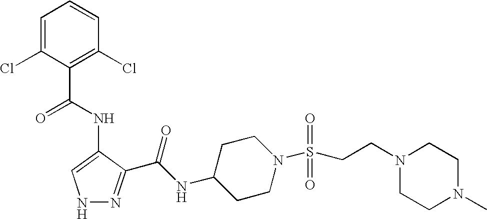 | As per Example 6, but using N-methyl piperazine in step 6B insteadof dimethylamine | [M + H]⁺572.28Rₜ 2.26 |
| 88 | 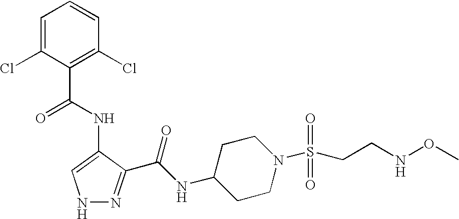 | As per Example 6, but usingmethoxyamine hydrochloride andtriethylamine in step 6B instead ofdimethylamine | [M + H]⁺519.19Rₜ 2.79 |
| 89 | 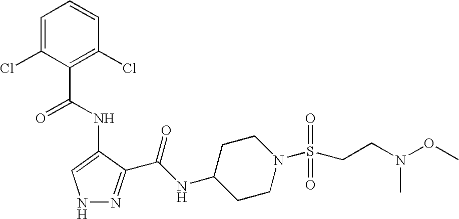 | As per Example 6, but using N,O-dimethylhydroxylaminehydrochlorie and triethylamine instep 6B instead of dimethylamine | [M + H]⁺533.28Rₜ 2.81 |
| 90 | 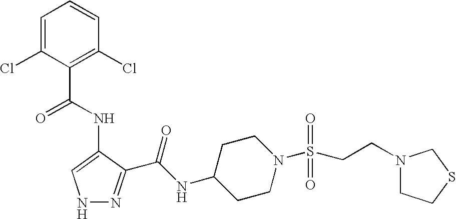 | As per Example 6, but usingthiazolidine in step 6B instead ofdimethylamine | [M + H]⁺561.16Rₜ 2.64 |
| 91 | 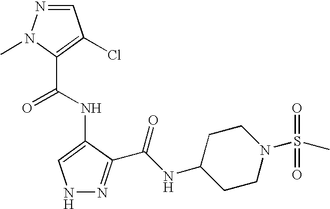 | As per Example 4 but using 4-chloro-2-methyl-2H-pyrazole-3-carboxylicacid instead of 2-chloro-6-fluorobenzoic acid | [M + H]⁺ 430Rₜ 2.44 |
| 92 | 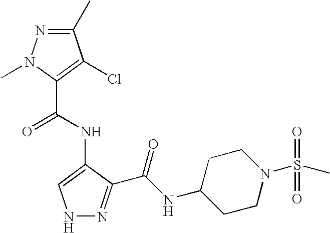 | As per Example 4 but using 4-chloro-2,5-dimethyl-2H-pyrazole-3-carboxylic acid instead of 2-chloro-6-fluorobenzoic acid | [M + H]⁺ 444Rₜ 2.54 |
| 93 | 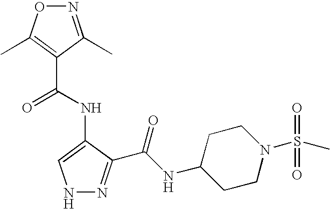 | As per Example 4 but using 3,5-dimethylisoxazole-4-carboxylic acidinstead of 2-chloro-6-fluorobenzoicacid | [M + H]⁺ 411Rₜ 2.35 |
| 94 | 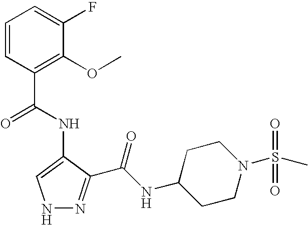 | As per Example 4 but using 3-fluoro-2-methoxybenzoic acid instead of 2-chloro-6-fluorobenzoic acid | [M + H]⁺ 440Rₜ 2.68 |
| 95 | 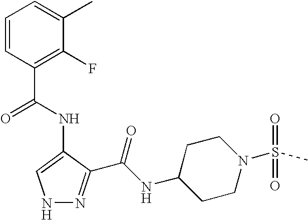 | As per Example 4 but using 2-fluoro-3-methylbenzoic acid instead of 2-chloro-6-fluorobenzoic acid | [M + H]⁺ 424Rₜ 2.70 |
| 96 | 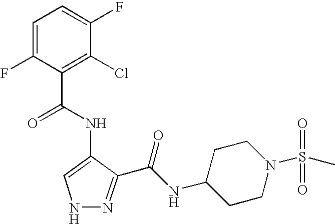 | As per Example 4 but using 2-chloro-3,6-difluorobenzoyl chloride andtriethylamine instead of 2-chloro-6-fluorobenzoic acid, HOBt and EDC | [M + H]⁺ 462Rₜ 2.66 |
| 97 | 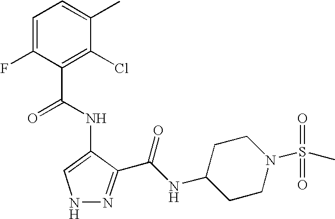 | As per Example 4 but using 2-chloro-6-fluoro-3-methylbenzoyl chlorideand triethylamine instead of 2-chloro-6-fluorobenzoic acid, HOBt and EDC | [M + H]⁺ 458Rₜ 2.73 |
| 98 | 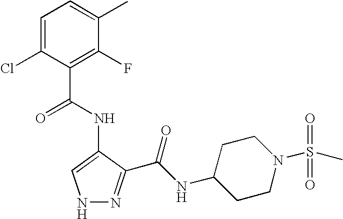 | As per Example 4 but using 6-chloro-2-fluoro-3-methylbenzoyl chlorideand triethylamine instead of 2-chloro-6-fluorobenzoic acid, HOBt and EDC | [M + H]⁺ 458Rₜ 2.73 |
| 99 | 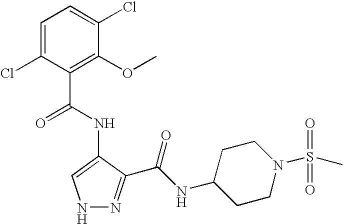 | As per Example 4 but using 3,6-dichloro-2-methoxybenzoic acidinstead of 2-chloro-6-fluorobenzoicacid | [M + H]⁺ 490Rₜ 2.79 |
| 100 | 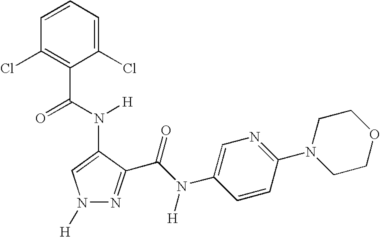 | As Preparation X, Step 1, except used6-morpholino-3-aminopyridineinstead of 4-amino-1-BOC-piperidine. | [M + H]+ 461/463Rt 2.34 |
| 101 | 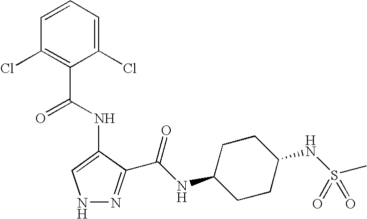 | As Preparation X, Step 1, except usedproduct of Preparation XII instead of4-amino-1-BOC-piperidine. | [M + H]+ 474/476Rt 2.54 |
| 102 | 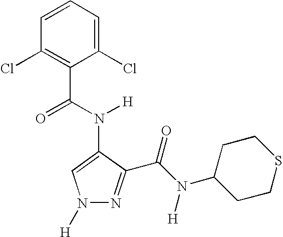 | As Preparation X, Step 1, exceptproduct purified by flashchromatography and used 4-aminotetrahydrothiopyran (WO03/082871) instead of 4-amino-1-BOC-piperidine | [M + H]+399.15Rt 2.94 |
| 103 | 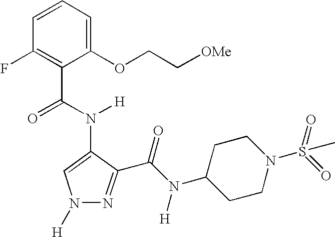 | As Example 4 except used 2-fluoro-6-(2-methoxy-ethoxy)-benzoic acidinstead of 2-chloro-6-fluorobenzoicacidSee Preparation XIII | [M + H]+484.31Rt 2.44 |
| 104 | 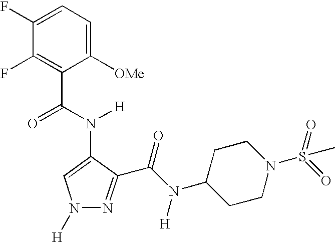 | As Example 4 except used 2,3-difluoro-6-methoxybenzoic acidinstead of 2-chloro-6-fluorobenzoicacidSee Preparation XIV | [M + H]+458.24Rt 2.53 |
| 105 | 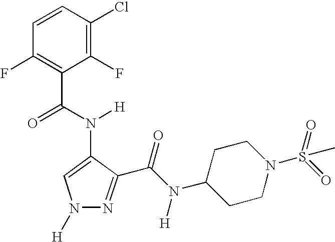 | As Example 4 except used 3-chloro-2,6-difluorobenzoyl chloride andtriethylamine instead of 2-chloro-6-fluorobenzoic acid, EDC and HOBt | [M + H]+462.23Rt 2.69 |
| 106 | 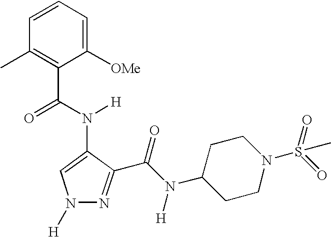 | As Example 4 except used 2-methoxy-6-methylbenzoic acidinstead of 2-chloro-6-fluorobenzoicacidSee Preparation XV | [M + H]+436.24Rt 2.55 |
| 107 | 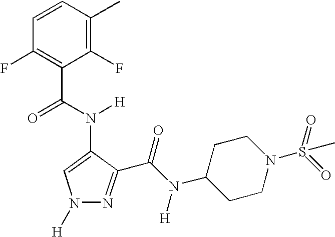 | As Example 4 except used 2,6-difluoro-3-methylbenozyl chlorideand triethylamine instead of 2-chloro-6-fluorobenzoic acid, EDC and HOBt | [M + H]+442.19Rt 2.68 |
| 108 | 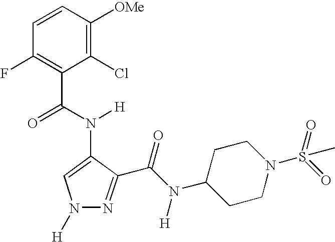 | As Example 4 except used 2-chloro-3-methoxy-6-fluorobenzoic acidinstead of 2-chloro-6-fluorobenzoicacidSee Preparation Example XVI | [M + H]+474.20Rt 2.56 |
| 109 | 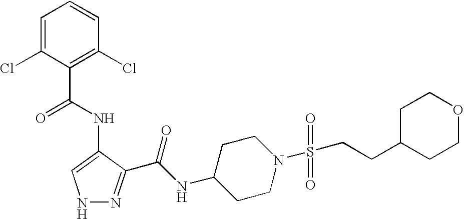 | General procedure HStarting material is 4-bromomethyltetrahydropyranPurified by column chromatography(EtOAc) | [M + H]+ 544Rt 2.79 |
| 110 | 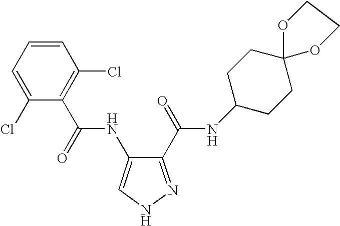 | General procedure A | [M + H]+ 439Rt 2.80 |
| 111 | 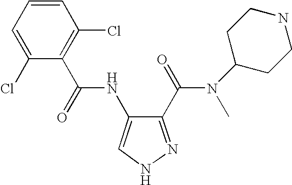 | General procedure A | [M + H]+ 396Rt 5.35 |

### EXAMPLE 112

### Synthesis of 4-(dichloro-benzoylamino)-1H-pyrazole-3-carboxylic acid 1,1-dioxo-hezahydro-llambda* 6*-thiopyran-4-yl)-amide

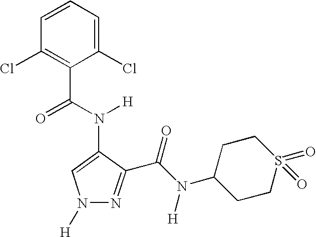

To a stirred solution of 4-(2,6-dichloro-benzoylamino)-1H-pyrazole-3-carboxylic acid (tetrahydro-thiopyran-4-yl)-amide (Example 102) (100 mg, 0.25 mmoles) in dichloromethane (10 ml) was added mCPBA (112 mg, 0.50 mmoles) and the resultant solution stirred at ambient temperature for 1 hour. The reaction mixture was diluted with ethyl acetate and washed with sequentially saturated sodium sulphite solution (twice), saturated sodium hydrogen carbonate solution (twice) and then brine solution. The organic portion was dried (MgSO₄), filtered and evaporated in vacuo. The residue was purified by flash chromatography (Biotage SP4, 25S, flow rate 25 ml/min, gradient 1:1 EtOAc/Petrol to EtOAc) to give 4-(2,6-dichloro-benzoylamino)-1H-pyrazole-3-carboxylic acid (1,1-dioxo-hezahydro-llambda*6*-thiopyran-4-yl)-amide as a white solid (47 mg, 44%). (LC/MS: Rₜ 2.44, [M+H]⁺ 431.14).

### EXAMPLE 113

### Preparation of trans-4-(2,6-dichloro-benzoylamino)-1H-pyrazole-3-carboxylic acid (4-isopropoxy-cyclohexyl)-amide

### 113A. Preparation of 4-isopropoxy-cyclohexylamine

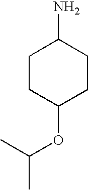

A mixture of 1-isopropoxy-4-nitrobenzene (500 mg, 2.76 mmol) and 5% Rh/alumina (400 mg) in EtOH (10 ml) and glacial AcOH (200 μl) was shaken under 50 psi of hydrogen at 60° C. for 4 hours. The mixture was filtered through a plug of Celite and reduced in vacuo to give the title compound as a mixture of isomers.

### 113B. Preparation of tmrφ-4-(2,6-dichloro-benzoylaminoV1H-pyrazole-3-carboxylic acid C4-isopropoxy-cyclohexyl)-amide

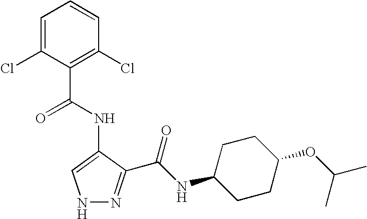

A mixture of 4-(2,6-dichloro-benzoylamino)-1H-pyrazole-3-carboxylic acid (600 mg), 4-isopropoxy-cyclohexylamine (400 mg), EDC (573 mg) and HOBt (405 mg) in DMF (20 ml) was stirred at ambient temperature for 18 hours. The mixture was reduced in vacuo and then partitioned between EtOAc and saturated aqueous NaHCO₃. The organic portion was washed with brine, dried (MgSO₄) and reduced in vacuo to give the title compound as a mixture of isomers. A portion of the residue was submitted to preparative LC/MS for purification and the desired trans-isomer isolated (1.4 mg). (LC/MS: Rₜ 3.09, [M+H]⁺ 439.24).

### EXAMPLE 114

### Synthesis of 4-[(2,6-dichloro-benzoyl)-methyl-amino]-1H-pyrazole-3-carboxylic acid piperidin-4-yl-amide

### 114A. Preparation of 4-[Y2,6-dichloro-benzoyl)-methyl-amino1-1-(tetrahydro-pyran-2-yl)-1H-pyrazole-3-carboxylic acid methy ester

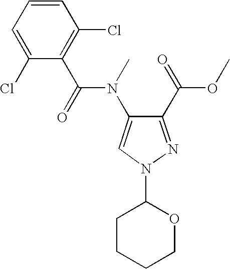

4-Amino-1-(tetrahydro-pyran-2-yl)-1H-pyrazole-3-carboxylic acid methyl ester (1 g, 4.4 mmol) was dissolved in ethanol (30 ml), triethyl orthoformate (5.3 mmol, 0.785 g) was added and mixture was refluxed for 15 hours, before slowly adding sodium borohydride (0.537 g, 14.2 mmol) at room temperature. The reaction mixture was refluxed for another hour and cooled down to room temperature before solvent was evaporated in vacuo. Crude purified by flash SiO₂ chromatography eluting Hexane:EtOAc (1:3) to give 4-Methyl amino-1-(tetrahydro-pyran-2-yl)-1H-pyrazole-3-carboxylic acid methyl ester as a white solid (0.238 g, 23% yield).

This compound was taken into the next reaction as starting material (0.238 g, 0.99 mmol), dissolved in DCM (10 ml), added triethyl amine (179 μl, 1.18 mmol) followed by addition of 2,6-dichloro-benzoyl chloride (228 μl, 1.08 mmol). The reaction mixture was stirred over 16 hours, and then the solvent was reduced in vacuo and the crude product partitioned between EtOAc and water. The organics were washed with saturated NaHCO₃ dried over MgSO₄, filtered and evaporated in vacuo to afford the title compound as an oil mixture. The crude product was taken into the next reaction.

### 114B. 4-{[(2,6-dichloro-benzoyl)-methyl-amino]-1-(tetrahydro-pyran-2-vD-1H-pyrazole-3-carbonyll-amino1-piperidine-1-carboxylic acid tert-butyl ester

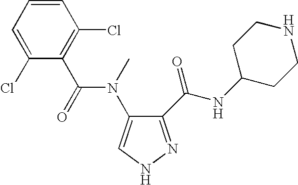

2 4-[(2,6-dichloro˜benzoyl)-methyl-amino]-1-(tetrahydro-pyran-2-yl)-1H-pyrazole-3-carboxylic acid methyl ester (0.513 g, 1.2 mmol) was dissolved in methanol (5 ml), 2N NaOH solution (5 ml) was added, and the reaction was stirred for 15 hours. The solvent was reduced in vacuo and then the crude product was partitioned between EtOAc and water. The water layers were neutralised with 2N HCl and extracted in EtOAc. The organics were dried over MgSO₄, filtered and evaporated in vacuo to afford 4-[(2,6-dichloro-benzoyl)-methyl-amino]-1-(tetrahydro-pyran-2-yl)-1H-pyrazole-3-carboxylic acid as a white solid.

The pyrazole acid (0.194 mg, 0.49 mmol) was the starting material for the next reaction which was carried out in a manner analogous to Example 113 but using N-Boc-4-amino piperidine (108 mg; 0.53 mmol) as the starting amine. The crude product was purified by flash SiO₂ chromatography eluting with Hexane:EtOAc (2:1) to afford 4-{[(2,6-dichloro-benzoyl)˜methyl-amino]-1-(tetrahydro-pyran-2-yl)-1H-pyrazole-3-carbonyl]-amino}-piperidine-1-carboxylic acid tert-butyl ester as a white solid. To this compound (30 mg, 0.05 mmol) was added HCl in ether (3 ml), the reaction mixture was stirred for 5 hours, and then the solvent was reduced in vacuo to afford the title compound as a hydrochloride salt, white solid (30 mg, 20%) (LC/MS: Rₜ 1.52, [M+H]⁺ 396).

### EXAMPLES 115-131

By using the methods set out above, the compounds of Examples 115 to 131 were prepared. In the Table below, the general synthetic route used in each case, together with any modifications (if any) to the reactants and conditions, are given for each example.

| Example | Structure | Method of Preparation | LCMS |
|---|---|---|---|
| 115 | 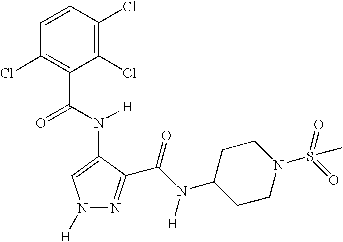 | As Example 4 except used 2,3,6-trichlorobenzoyl chloride (preparedfrom the corresponding acid andthionyl chloride as in Example 1) andtriethylamine instead of 2-chloro-6-fluorobenzoic acid, EDC and HOBt. | [M + H]+493Rt 2.83 |
| 116 | 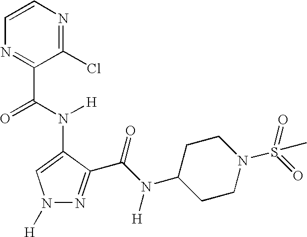 | As Example 4 except 3-chloro-pyrazine-2-carbonyl chloride(prepared from the correspondingacid and thionyl chloride as inExample 1) and triethylamine insteadof 2-chloro-6-fluorobenzoic acid,EDC and HOBt. | [M + H]+428Rt 2.28 |
| 117 | 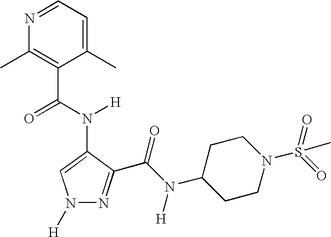 | As Example 4 except used 2,4-dimethyl-nicotinyl chloride(prepared from the correspondingacid and thionyl chloride as inExample 1) and triethylamineinstead of 2-chloro-6-fluorobenzoicacid, EDC and HOBt. | [M + H]+421Rt 1.58 |
| 118 | 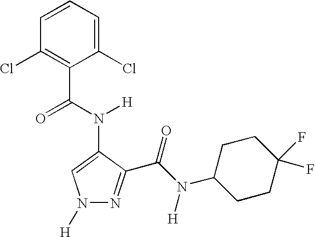 | As for General Procedure A using4,4-difluorocyclohexylaminehydrochloride. | [M + H]+417/419Rt 3.08 |
| 119 | 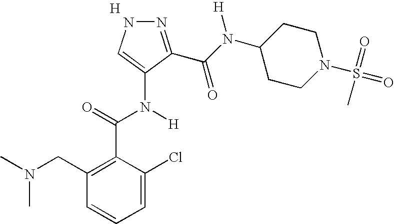 | As Example 4 except used 2-chloro-6-dimethylaminomethyl-benzoic acid(see Preparation XVII) | [M + H]+483.21Rt 1.86 |
| 120 | 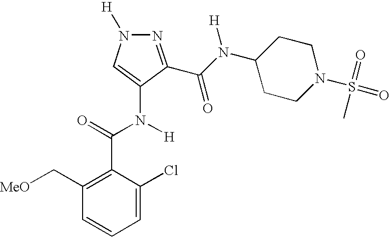 | As Example 4 except used 2-chloro-6-methoxymethyl-benzoic acidinstead of 2-chloro-6-fluorobenzoicacid (see Preparation XVIII) | [M + H]+470.23Rt 2.56 |
| 121 | 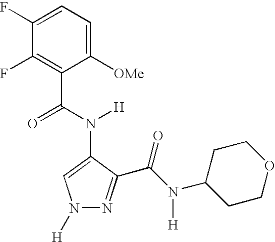 | General procedure J using in step J (i)4-amino tetrahydro pyrane.Partitioned crude between EtOAcand NaHCO₃Step J (ii), then General procedure Busing 2,3-difluoro-6-methoxybenzoic acid | [M + H]⁺381Rₜ 2.54 |
| 122 | 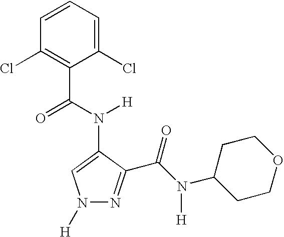 | General procedure AUsing 4-amino-tetrahydropyrane asstarting amine. Crude was purified byflash silica column chromatographyeluting with Hexane:EtOAc(1:1 to 100% EtOAc) | [M + H]⁺383Rₜ 2.26 |
| 123 | 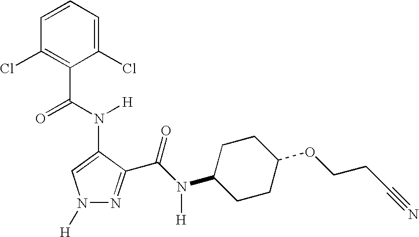 | Preparation I, except using 3-bromopropionitrile in DMF atambient temperature in step 2 thenGeneral Procedure A | [M + H]+450.16Rt 2.73 |
| 124 | 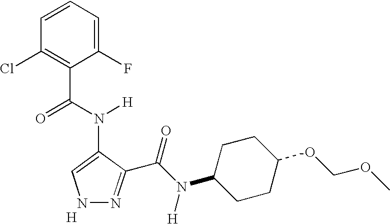 | Preparation XIX then Generalprocedure B using 2-chloro-6-fluorobenzoic acid | [M + H]+425.10Rt 2.79 |
| 125 | 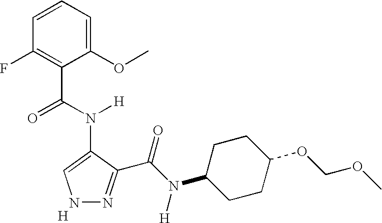 | Preparation XIX then Generalprocedure B using 2-fluoro-6-methoxybenzoic acid | [M + H]+421.17Rt 2.68 |
| 126 | 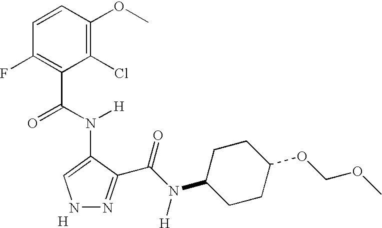 | Preparation XIX then Generalprocedure B using 2-chloro-6-fluoro-3-methoxy-benzoic acid(Preparation XVI) | [M + H]+455.15Rt 2.81 |
| 127 | 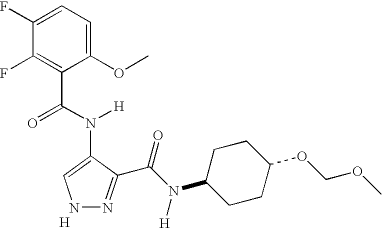 | Preparation XIX then Generalprocedure B using 2,3-difluoro-6-methoxy-benzoic acid(Preparation XIV) | [M + H]+439.18Rt 2.79 |
| 128 | 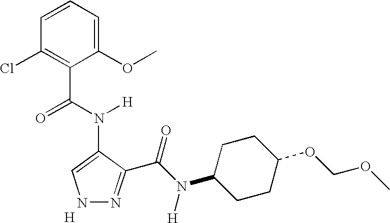 | Preparation XIX then Generalprocedure B using 2-chloro-6-methoxybenzoic acid (Synthesizedas in Example 5) | [M + H]+437.16Rt 2.76 |
| 129 | 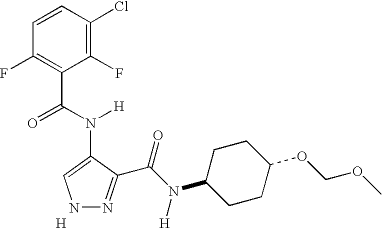 | Preparation XIX then Generalprocedure B using 3-chloro-2,6-difluorobenzoyl chloride and usingNEt₃ in place of HOBt and EDAC | [M + H]+443.10Rt 2.96 |
| 130 | 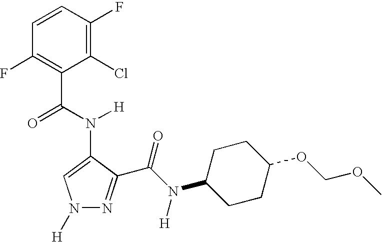 | Preparation XIX then Generalprocedure B using 2-chloro-3,6-difluorobenzoyl chloride and usingNEt3 in place of HOBt and EDAC | [M + H]+443.09Rt 2.94 |
| 131 | 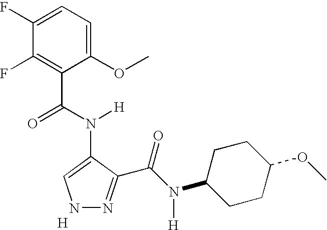 | Preparation IX, except using 2,3-difluoro-6-methoxy-benzoic acid(Preparation XIV), and preparation I,except using iodomethane in step 2,then General Procedure A | [M + H]+409.10Rt 2.71 |

### EXAMPLE 132

### Synthesis of 4-(2,6-difluoro-benzoylamino′)-1H-pyrazole-3-carboxylic acid d-Pyrimidin-2-yl-piperidin-4-vP)-amide

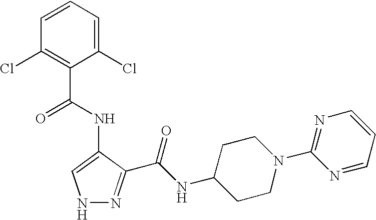

A mixture of 4-(2,6-dichlorobenzoylamino)-1H-pyrazole-3-carboxylic acid piperidin-4-yl-amide methanesulphonic acid salt (made in a manner analogous to Preparation X) (200 mg; 0.42 mmol) and 2-chloropyrimidine (55 mg; 0.46 mmol) in 5 ml of dioxane was treated with caesium carbonate (300 mg; 9.2 mmol) and a catalytic quantity of potassium iodide then heated at 95° C. overnight. The reaction was allowed to cool to room temperature, treated with water (20 ml) and the dioxane removed by evaporation under vacuum. The solid was collected by filtration, washed with water and dried. Purification by flash column chromatography (Eluant: 1:1 then 2:1 then 1:0 EtOAc/P.E.) gave 85 mg of 4-(2,6-difluoro-benzoylamino)-1H-pyrazole-3-carboxylic acid (1-pyrimidin-2-yl-piperidin-4-yl)-amide as a white solid. (LC/MS: Rₜ 2.78, [M+H]⁺ 460/462).

### EXAMPLES 133-137

By using the methods set out above, the compounds of Examples 133 to 137 were prepared. In the Table below, the general synthetic route used in each case, together with any modifications (if any) to the reactants and conditions, are given for each example.

| Example | Structure | Method of Preparation | LCMS |
|---|---|---|---|
| 133 | 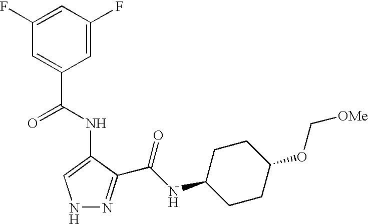 | Preparation I, except usingchloromethyl methyl ether in step 2then General procedure D (i) and (iii)then General Procedure B using 3,5-difluorobenzoic acid | [M + H]⁺409Rₜ 3.03 |
| 134 | 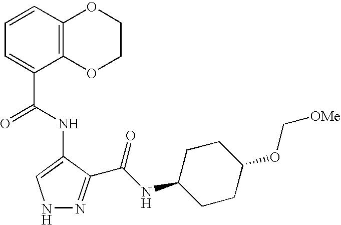 | Preparation I, except usingchloromethyl methyl ether in step 2then General procedure D (i) and (iii)then General Procedure B using 1,4-benzodioxan-5-carboxylic acid | [M + H]⁺431Rₜ 2.79 |
| 135 | 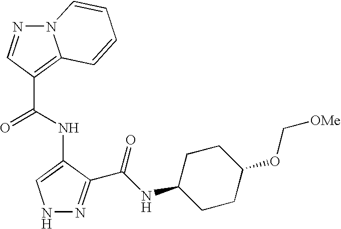 | Preparation I, except usingchloromethyl methyl ether in step 2then general procedure D (i) and (iii)then General Procedure B usingpyrazolo[1,5-a]pyridine-3-carboxylicacid | [M + H]⁺413Rₜ 2.53 |
| 136 | 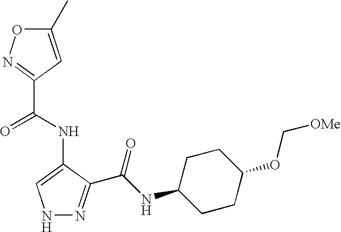 | Preparation I, except usingchloromethyl methyl ether in step 2then General procedure D (i) and (iii)then General Procedure B using 5-methylisoxazole-3-carboxylic acid | [M + H]⁺378Rₜ 2.69 |
| 137 | 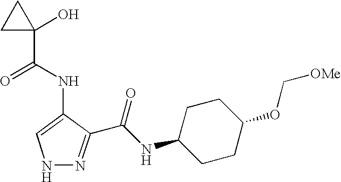 | Preparation I, except usingchloromethyl methyl ether in step 2then General procedure D (i) and (iii)then General Procedure B using 1-hydroxycyclopropane carboxylic acid | [M + H]+353Rt 2.14 |

### Biological Activity

### EXAMPLE 138

### Measurement of Activated CDK2/CyclinA Kinase Inhibitory Activity Assay f C₅₀)

Compounds of the invention were tested for kinase inhibitory activity using the following protocol.

Activated CDK2/CyclinA (Brown et al, Nat. Cell Biol., 1, pp 438-443, 1999; Lowe, E. D., et al Biochemistry, 41, pp 15625-15634, 2002) is diluted to 125 pM in 2.5× strength assay buffer (5O mM MOPS pH 7.2, 62.5 mM β-glycerophosphate, 12.5 mM EDTA, 37.5 mM MgCl₂, 112.5 mM ATP, 2.5 mM DTT, 2.5 mM sodium orthovanadate, 0.25 mg/ml bovine serum albumin), and 10 μl mixed with 10 μl of histone substrate mix (60 μl bovine histone H1 (Upstate Biotechnology, 5 mg/ml), 940 μl H₂O, 35 μCi γ³³P-ATP) and added to 96 well plates along with 5 μl of various dilutions of the test compound in DMSO (up to 2.5%). The reaction is allowed to proceed for 2 to 4 hours before being stopped with an excess of ortho-phosphoric acid (5 μl at 2%). γ³³P-ATP which remains unincorporated into the histone H1 is separated from phosphorylated histone H1 on a Millipore MAPH filter plate. The wells of the MAPH plate are wetted with 0.5% orthophosphoric acid, and then the results of the reaction are filtered with a Millipore vacuum filtration unit through the wells. Following filtration, the residue is washed twice with 200 μl of 0.5% orthophosphoric acid. Once the filters have dried, 20 μl of Microscint 20 scintillant is added, and then counted on a Packard Topcount for 30 seconds.

The % inhibition of the CDK2 activity is calculated and plotted in order to determine the concentration of test compound required to inhibit 50% of the CDK2 activity (IC₅₀).

### EXAMPLE 139

### Measurement of Activated CDK1/CyclinB Kinase Inhibitory Activity Assay (ICsn)

CDK1/CyclinB assay is identical to the CDK2/CyclinA above except that CDK1/CyclinB (Upstate Discovery) is used and the enzyme is diluted to 6.25 nM.

Compounds of the invention have IC₅₀ values less than 20 μM or provide at least 50% inhibition of the CDK2 activity at a concentration of 10 μM. Preferred compounds of the invention have IC₅₀ values of less than 1 μM in the CDK2 or CDK1 assay.

### EXAMPLE 140

### GSK3-B Kinase Inhibitory Activity Assay

GSK3-γ (Upstate Discovery) are diluted to 7.5 nM in 25 mM MOPS, pH 7.00, mg/ml BSA, 0.0025% Brij-35, 1.25% glycerol, 0.5 mM EDTA, 25 mM MgCl₂, 0.025% β-mercaptoethanol, 37.5 mM ATP and 10 μl mixed with 10 μl of substrate mix. The substrate mix for GSK3-β is 12.5 μM phospho-glycogen synthase peptide-2 (Upstate Discovery) in 1ml of water with 35 μCi γ³³P-ATP. Enzyme and substrate are added to 96 well plates along with 5 μl of various dilutions of the test compound in DMSO (up to 2.5%). The reaction is allowed to proceed for 3 hours (GSK3-β) before being stopped with an excess of ortho-phosphoric acid (5 μl at 2%). The filtration procedure is as for Activated CDK2/CyclinA assay above.

### EXAMPLE 141

### Anti-Proliferative Activity

The anti-proliferative activities of compounds of the invention can be determined by measuring the ability of the compounds to inhibition of cell growth in a number of cell lines. Inhibition of cell growth is measured using the Alamar Blue assay (Nociari, M. M, Shalev, A., Benias, P., Russo, C. Journal of Immunological Methods 1998, 213, 157-167). The method is based on the ability of viable cells to reduce resazurin to its fluorescent product resorufin. For each proliferation assay cells are plated onto 96 well plates and allowed to recover for 16 hours prior to the addition of inhibitor compounds for a further 72 hours. At the end of the incubation period 10% (v/v) Alamar Blue is added and incubated for a further 6 hours prior to determination of fluorescent product at 535 nM ex/59 OnM em. In the case of the non-proliferating cell assay cells are maintained at confluence for 96 hour prior to the addition of inhibitor compounds for a further 72 hours. The number of viable cells is determined by Alamar Blue assay as before. Cell lines can be obtained from the ECACC (European Collection of cell Cultures).

In particular, compounds of the invention were tested against the HCT-1 16 cell line (ECACC Reference: 91091005) derived from human colon carcinoma.

Many compounds of the invention were found to have IC₅₀ values of less than 20 μM in this assay and preferred compounds have IC₅₀ values of less than 1 μM.

### EXAMPLE 142

### Determination of Oral Bioavailability

The oral bioavailability of the compounds of formula (I) may be determined as follows.

The test compound is administered as a solution both LV. and orally to balb/c mice at the following dose level and dose formulations;
1 mg/kg IV formulated in 110% DMSO/90% (2-hydroxypropyl)-β-cyclodextrin (25% w/v); and
5 mg/kg PO formulated in 10% DMSO/20% water/70% PEG200.

At various time points after dosing, blood samples are taken in heparinised tubes and the plasma fraction is collected for analysis. The analysis is undertaken by LC-MS/MS after protein precipitation and the samples are quantified by comparison with a standard calibration line constructed for the test compound. The area under the curve (AUC) is calculated from the plasma level vs time profile by standard methods. The oral bioavailability as a percentage is calculated from the following equation:

$$\frac{AUCpo}{AUCiv} \times \frac{doseIV}{dosePO} \times 100$$

### Pharmaceutical Formulations

### EXAMPLE 143

### (T) Tablet Formulation

A tablet composition containing a compound of the formula (I) is prepared by mixing 50 mg of the compound with 197 mg of lactose (BP) as diluent, and 3 mg magnesium stearate as a lubricant and compressing to form a tablet in known manner.

### (ip Capsule Formulation

A capsule formulation is prepared by mixing 100 mg of a compound of the formula (I) with 100 mg lactose and filling the resulting mixture into standard opaque hard gelatin capsules.

(iii) Injectable Formulation I

A parenteral composition for administration by injection can be prepared by dissolving a compound of the formula (I) (e.g. in a salt form) in water containing 10% propylene glycol to give a concentration of active compound of 1.5% by weight. The solution is then sterilised by filtration, filled into an ampoule and sealed.

### (W) Injectable Formulation II

A parenteral composition for injection is prepared by dissolving in water a compound of the formula (I) (e.g. in salt form) (2 mg/ml) and mannitol (50 mg/ml), sterile filtering the solution and filling into sealable 1 ml vials or ampoules.

### v) Injectable Formulation III

A formulation for i.v. delivery by injection or infusion can be prepared by dissolving the compound of formula (I) (e.g. in a salt form) in water at 20 mg/ml. The vial is then sealed and sterilised by autoclaving.

### vp Injectable Formulation IV

A formulation for i.v. delivery by injection or infusion can be prepared by dissolving the compound of formula (I) (e.g. in a salt form) in water containing a buffer (e.g. 0.2 M acetate pH 4.6) at 20 mg/ml. The vial is then sealed and sterilised by autoclaving.

(vii) Subcutaneous Injection Formulation

A composition for sub-cutaneous administration is prepared by mixing a compound of the formula (I) with pharmaceutical grade corn oil to give a concentration of 5 mg/ml. The composition is sterilised and filled into a suitable container.

viii) Lyophilised Formulation

Aliquots of formulated compound of formula (I) are put into 50 mL vials and lyophilized. During lyophilisation, the compositions are frozen using a one-step freezing protocol at (−45° C.). The temperature is raised to −10° C. for annealing, then lowered to freezing at −45° C., followed by primary drying at +25° C. for approximately 3400 minutes, followed by a secondary drying with increased steps if temperature to 50° C. The pressure during primary and secondary drying is set at 80 millitor.

### (ix) Solid Solution Formulation

The compound of formula (I) is dissolved in dichloromethane/ethanol (1:1) at a concentration of 5 to 50% (for example 16 or 20%) and the solution is spray dried using conditions corresponding to those set out in the table below. The data given in the table include the concentration of the compound of Formula (I), and the inlet and outlet temperatures of the spray drier.

| conc. sol. w/vol | temperature of inlet | temperature of outlet |
|---|---|---|
| 16% | 140° C. | 80° C. |
| 16% | 180° C. | 80° C. |
| 20% | 160° C. | 80° C. |
| 20% | 180° C. | 100° C. |

A solid solution of the compound of formula (I) and PVP can either be filled directly into hard gelatin or HPMC (hydroxypropylmethyl cellulose) capsules, or be mixed with pharmaceutically acceptable excipients such as bulking agents, glidants or dispersants. The capsules could contain the compound of formula (I) in amounts of between 2 mg and 200 mg, for example 10, 20 and 80 mg.

### EXAMPLE 144

### Determination of Antifungal Activity

The antifungal activity of the compounds of the formula (I) can be determined using the following protocol.

The compounds are tested against a panel of fungi including Candida parpsilosis, Candida tropicalis, Candida albicans-ATCC 36082 and Cryptococcus neoformans.

The test organisms are maintained on Sabourahd Dextrose Agar slants at 4° C. Singlet suspensions of each organism are prepared by growing the yeast overnight at 27° C. on a rotating drum in yeast-nitrogen base broth (YNB) with amino acids (Difco, Detroit, Mich.), pH 7.0 with 0.05 M morpholine propanesulphonic acid (MOPS). The suspension is then centrifuged and washed twice with 0.85% NaCl before sonicating the washed cell suspension for 4 seconds (Branson Sonifier, model 350, Danbury, Conn.). The singlet blastospores are counted in a haemocytometer and adjusted to the desired concentration in 0.85% NaCl.

The activity of the test compounds is determined using a modification of a broth microdilution technique. Test compounds are diluted in DMSO to a 1.0 mg/ml ratio then diluted to 64 μg/ml in YNB broth, pH 7.0 with MOPS (Fluconazole is used as the control) to provide a working solution of each compound. Using a 96-well plate, wells 1 and 3 through 12 are prepared with YNB broth, ten fold dilutions of the compound solution are made in wells 2 to 11 (concentration ranges are 64 to 0.125 μg/ml). Well 1 serves as a sterility control and blank for the spectrophotometric assays. Well 12 serves as a growth control. The microtitre plates are inoculated with 10 μl in each of well 2 to 11 (final inoculum size is 10⁴ organisms/ml). Inoculated plates are incubated for 48 hours at 35° C. The IC50 values are determined spectrophotometrically by measuring the absorbance at 420 nm (Automatic Microplate Reader, DuPont Instruments, Wilmington, Del.) after agitation of the plates for 2 minutes with a vortex-mixer (Vorte-Genie 2 Mixer, Scientific Industries, Inc., Bolemia, N.Y.). The IC50 endpoint is defined as the lowest drug concentration exhibiting approximately 50% (or more) reduction of the growth compared with the control well. With the turbidity assay this is defined as the lowest drug concentration at which turbidity in the well is <50% of the control (IC50). Minimal Cytolytic Concentrations (MCC) are determined by sub-culturing all wells from the 96-well plate onto a Sabourahd Dextrose Agar (SDA) plate, incubating for 1 to 2 days at 35° C. and then checking viability.

### EXAMPLE 145

### Protocol for the Biological Evaluation of Control of In Vivo Whole Plant Fungal Infection

Compounds of the formula (I) are dissolved in acetone, with subsequent serial dilutions in acetone to obtain a range of desired concentrations. Final treatment volumes are obtained by adding 9 volumes of 0.05% aqueous Tween-20™ or 0.01% Triton X-100™, depending upon the pathogen.

The compositions are then used to test the activity of the compounds of the invention against tomato blight (Phytophthora infestans) using the following protocol. Tomatoes (cultivar Rutgers) are grown from seed in a soil-less peat-based potting mixture until the seedlings are 10-20 cm tall. The plants are then sprayed to run-off with the test compound at a rate of 100 ppm. After 24 hours the test plants are inoculated by spraying with an aqueous sporangia suspension of Phytophthora infestans, and kept in a dew chamber overnight. The plants are then transferred to the greenhouse until disease develops on the untreated control plants.

Similar protocols are also used to test the activity of the compounds of the invention in combatting Brown Rust of Wheat (Puccinia), Powdery Mildew of Wheat (Ervsiphe vraminis), Wheat (cultivar Monon), Leaf Blotch of Wheat (Septoria tritici), and Glume Blotch of Wheat (Leptosphaeria nodorum).

### Equivalents

The foregoing examples are presented for the purpose of illustrating the invention and should not be construed as imposing any limitation on the scope of the invention. It will readily be apparent that numerous modifications and alterations may be made to the specific embodiments of the invention described above and illustrated in the examples without departing from the principles underlying the invention. All such modifications and alterations are intended to be embraced by this application.

1. A compound of the formula (I): 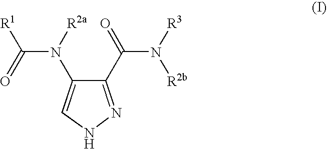
or a salt, tautomer, N-oxide or solvate thereof
wherein:
R¹ is selected from:
(a) 2,6-dichlorophenyl;
(b) 2,6-difluorophenyl;
(c) a 2,3,6-trisubstituted phenyl group wherein the substituents for the phenyl group are selected from fluorine, chlorine, methyl and methoxy;
(d) a group R⁰;
(e) a group R^{1a};
(f) a group R^{1b};
(g) a group R^{1c};
(h) a group R^{1d}; and
(j) 2,6-difluorophenylamino;
R⁰ is a carbocyclic or heterocyclic group having from 3 to 12 ring members; or a C₁₋₈ hydrocarbyl group optionally substituted by one or more substituents selected from fluorine, hydroxy, cyano; C₁₋₄ hydrocarbyloxy, amino, mono- or di-C₁₋₄ hydrocarbylamino, and carbocyclic or heterocyclic groups having from 3 to 12 ring members, and wherein 1 or 2 of the carbon atoms of the hydrocarbyl group may optionally be replaced by an atom or group selected from O, S, NH, SO, SO₂;
R^{1a} is selected from cyclopropyl-cyano-methyl; furyl; benzoisoxazolyl; methylisoxazolyl; 2-monosubstituted phenyl and 2,6-disubstituted phenyl wherein the substituents on the phenyl moiety are selected from methoxy, ethoxy, fluorine, chlorine, and difluoromethoxy; provided that R^{1a} is not 2,6-difluorophenyl or 2,6-dichlorophenyl;
R^{1b} is selected from tetrahydrofuryl; and mono-substituted and disubstituted phenyl wherein the substituents on the phenyl moiety are selected from fluorine; chlorine; methoxy; ethoxy and methylsulphonyl;
R^{1c} is selected from; benzoisoxazoyl; five membered heteroaryl rings containing one or two heteroatoms selected from O and N and six-membered heteroaryl rings containing one or two nitrogen heteroatom ring members, the heteroaryl rings in each case being optionally substituted by methyl, fluorine, chlorine or trifluoromethyl; and phenyl substituted by one, two or three substituents selected from bromine, chlorine, fluorine, methyl, trifluoromethyl, ethoxy, methoxy, methoxyethoxy, methoxymethyl, dimethylaminomethyl and difluoromethoxy; provided that R^{1a} is not 2,6-difluorophenyl;
R^{1d} is a group R^{1e}—CH(CN)— where R^{1e} is a carbocylic or heterocyclic group having from 3 to 12 ring members;
R^{2a} and R^{2b} are each hydrogen or methyl;
and wherein:
A. when R¹ is (a) 2,6-dichlorophenyl and R^{2a} and R^{2b} are both hydrogen; then R³ can be selected from:
(i) a group 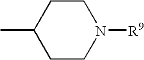
where R⁹ is selected from C(O)NR⁵R⁶, where R⁵ and R⁶ are selected from hydrogen and C₁₋₄ alkyl, C₁₋₂ alkoxy and C₁₋₂ alkoxy-C₁₋₄ alkyl, provided that no more than one of R⁵ and R⁶ is C₁₋₂alkoxy, or NR⁵R⁶ forms a five or six membered saturated heterocyclic ring containing one or two heteroatom ring members selected from O, N and S, the heterocyclic ring being optionally substituted by one or more methyl groups; C(O)—R¹⁰ where R¹⁰ is a C₁₋₄ alkyl group optionally substituted by one or more substituents chosen from fluorine, chlorine, cyano and methoxy; 2-pyrimidinyl; and R¹¹ where R¹¹ is a C₁₋₄ alkyl group substituted by one or more substituents chosen from fluorine, chlorine and cyano;
(ii) a group 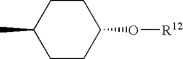
where R¹² is C₂₋₄ alkyl;
(iii) a group 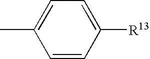
wherein R¹³ is selected from methylsulphonyl, 4-morpholino, 4-thiomorpholino, 1-piperidino, 1-methyl-4-piperazino and 1-pyrrolidino;
(iv) a substituted 3-pyridyl or 4-pyridyl group of the formula 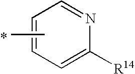
wherein the group R¹⁴ is meta or para with respect to the bond labelled with an asterisk and is selected from methyl, methylsulphonyl, 4-morpholino, 4-thiomorpholino, 1-piperidino, 1-methyl-4-piperazino, 1-pyrrolidino, 4-piperidinyloxy, 1-C₁₋₄alkoxycarbonyl-piperidin-4-yloxy, 2-hydroxyethoxy and 2-methoxyethoxy; and
(v) a group selected from 2-pyrazinyl, 5-pyrimidinyl, cyclohexyl, 1,4-dioxa-spiro[4.5]decan-8-yl (4-cyclohexanone ethylene glycol ketal), 4-methylsulphonylamino-cyclohexyl, tetrahydrothiopyran-4-yl, 1,1-dioxo-tetrahydrothiopyran-4-yl, tetrahydropyran-4-yl, 4,4-difluorocyclohexyl and 3,5-dimethylisoxazol-4-yl; and
B. when R¹ is (b) 2,6-difluorophenyl and R^{2a} and R^{2b} are both hydrogen; then R³ can be selected from:
(vi) 1-methyl-piperidin-3-yl; 4-(2-dimethylaminoethoxy)-cyclohexyl; and an N-substituted 4-piperidinyl group wherein the N-substituent is selected from cyanomethyl and cyanoethyl; and
(vii) a group 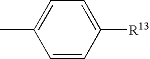
wherein R¹³ is as hereinbefore defined; and
C. when R¹ is (c) a 2,3,6-trisubstituted phenyl group wherein the substituents for the phenyl group are selected from fluorine, chlorine, methyl and methoxy; and
R^{2a} and R^{2b} are both hydrogen; then R³ can be selected from groups (ii), (xi), (xii) and (xiii) as defined herein; and
(viii) 4-piperidinyl and 1-methyl-4-piperidinyl;
(ix) tetrahydropyran-4-yl; and
(x) a group: 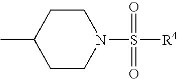
where R⁴ is C₁₋₄ alkyl;
D. when R¹ is (d), a group R⁰, where R⁰ is a carbocyclic or heterocyclic group having from 3 to 12 ring members; or a C₁₋₈ hydrocarbyl group optionally substituted by one or more substituents selected from fluorine, hydroxy, cyano; C₁₋₄ hydrocarbyloxy, amino, mono- or di-C₁₋₄ hydrocarbylamino, and carbocyclic or heterocyclic groups having from 3 to 12 ring members, and wherein 1 or 2 of the carbon atoms of the hydrocarbyl group may optionally be replaced by an atom or group selected from O, S, NH, SO, SO₂; then R³ can be selected from:
(xi) a group: 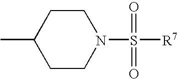
where R⁷ is:
unsubstituted hydrocarbyl other than C₁₋₄ alkyl;
substituted C₁₋₄ hydrocarbyl bearing one or more substituents chosen from fluorine, chlorine, hydroxy, methylsulphonyl, cyano, methoxy, NR⁵R⁶, and 4 to 7 membered saturated carbocyclic or heterocyclic rings containing up to two heteroatom ring members selected from O, N and S;
a group NR⁵R⁶ where R⁵ and R⁶ are selected from hydrogen and C₁₋₄ alkyl, C₁₋₂ alkoxy and C₁₋₂ alkoxy-C₁₋₄ alkyl, provided that no more than one of R⁵ and R⁶ is C₁₋₂ alkoxy, or NR⁵R⁶ forms a five or six membered saturated heterocyclic ring containing one or two heteroatom ring members selected from O, N and S, the heterocyclic ring being optionally substituted by one or more methyl groups;
a five or six membered heteroaryl group containing one or two heteroatom ring members selected from N, S and O and being optionally substituted by methyl, methoxy, fluorine, chlorine, or a group NR⁵R⁶;
a phenyl group optionally substituted by methyl, methoxy, fluorine, chlorine, cyano or a group NR⁵R⁶;
C₃₋₆ cycloalkyl; and
a five or six membered saturated heterocyclic ring containing one or two heteroatom ring members selected from O, N and S, the heterocyclic ring being optionally substituted by one or more methyl groups; and
(xii) a group: 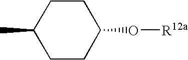
where R^{12a} is C₁₋₄ alkyl substituted by one or more substituents chosen from fluorine, chlorine, C₃₋₆ cycloalkyl, oxa-C₄₋₆ cycloalkyl, cyano, methoxy and NR⁵R⁶, provided that there are at least two carbon atoms between the oxygen atom to which R¹² is attached and a group NR⁵R⁶ when present; and
E. when R¹ is (e) a group R^{1a} and R^{2a} and R^{2b} are both hydrogen, then R³ can be (xiii) a group 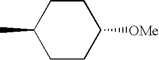
and
F. when R¹ is (f) a group R^{1b}, and R^{2a} and R^{2b} are both hydrogen, then R³ can be (xiv) a methyl group; and
G. when R¹ is (g) a group R^{1c} and R^{2a} and R^{2b} are both hydrogen, then R³ can be (xv) a group 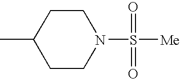
and:
H. when R¹ is (h), a group R^{1d}, then R³ is a group -Y—R^{3a} where Y is a bond or an alkylene chain of 1, 2 or 3 carbon atoms in length and R^{3a} is selected from hydrogen and carbocyclic and heterocyclic groups having from 3 to 12 ring members;
J. when R¹ is (j), 2,6-difluorophenylamino, and R^{2a} and R^{2b} are both hydrogen; then R³ can be methyl; and
K. when R¹ is 2,6-dichlorophenyl and either (k) R^{2a} is methyl and R^{2b} is hydrogen, or (1) R^{2a} is hydrogen and R^{2b} is methyl; then R³ can be a 4-piperidine group;
or salts, tautomers, solvates and N-oxides thereof.

2. A compound according to claim 1 wherein R¹ is 2,6-dichlorophenyl, R^{2a} and R^{2b} are both hydrogen and R³ is (i) a group: 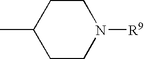
where R⁹ is selected from C(O)NR⁵R⁶; C(O)—R¹⁰ where R¹⁰ is a C₁₋₄ alkyl group optionally substituted by one or more substituents chosen from fluorine, chlorine, cyano and methoxy; and R¹¹ where R¹¹ is a C₁₋₄ alkyl group substituted by one or more substituents chosen from fluorine, chlorine and cyano.

3. A compound according to claim 2 wherein R⁹ is C(O)NR⁵R⁶ and NR⁵R⁶ is selected from dimethylamino, morpholine, piperidine, piperazine, N-methylpiperazine, pyrrolidine and thiazolidine.

4. A compound according to claim 2 wherein R⁹ is C(O)—R¹⁰ and R¹⁰ is selected from methyl, trifluoromethyl and methoxymethyl.

5. A compound according to claim 2 wherein R⁹ is a group R¹¹ and R¹¹ is selected from substituted methyl groups and 2-substituted ethyl groups.

6. A compound according to claim 1 wherein R¹ is 2,6-dichlorophenyl, R^{2a} and R^{2b} are both hydrogen and R³ is (ii) a group: 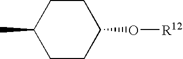
where R¹² is C₂₋₄ alkyl.

7. A compound according to claim 1 wherein R¹ is 2,6-dichlorophenyl, R^{2a} and R^{1b} are both hydrogen and R³ is (iii) a group: 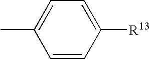
wherein R¹³ is selected from methylsulphonyl, 4-morpholino, 4-thiomorpholino, 1-piperidino, 1-methyl-4-piperazino and 1-pyrrolidino.

8. A compound according to claim 1 wherein R¹ is 2,6-dichlorophenyl, R^{2a} and R^{2b} are both hydrogen and R³ is (iv) a substituted 3-pyridyl or 4-pyridyl group of the formula 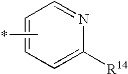
wherein the group R¹⁴ is meta or para with respect to the bond labelled with an asterisk and is selected from methyl, methylsulphonyl, 4-morpholino, 4-thiomorpholino, 1-piperidino, 1-methyl-4-piperazino, 1-pyrrolidino, 4-piperidinyloxy, 1-C₁₋₄alkoxycarbonyl-piperidin-4-yloxy, 2-hydroxyethoxy and 2-methoxyethoxy.

9. A compound according to claim 1 wherein R¹ is 2,6-dichlorophenyl, R^{2a} and R^{2b} are both hydrogen and R³ is (v) a group selected from 2-pyrazinyl, 5-pyrimidinyl, cyclohexyl, 1,4-dioxa-spiro[4.5]decan-8-yl (4-cyclohexanone ethylene glycol ketal), 4-methylsulphonylamino-cyclohexyl, tetrahydrothiopyran-4-yl, 1,1-dioxo-tetrahydrothiopyran-4-yl, tetrahydropyran-4-yl, 4,4-difluorocyclohexyl and 3,5-dimethylisoxazol-4-yl.

10. A compound according to claim 1 wherein R¹ is (b) 2,6-difluorophenyl, R^{2a} and R^{2b} are both hydrogen and R³ is selected from:
(vi) 1-methyl-piperidin-3-yl; 4-(2-dimethylaminoethoxy)-cyclohexyl; and an N-substituted 4-piperidinyl group wherein the N-substituent is selected from cyanomethyl and cyanoethyl; and
(vii) a group: 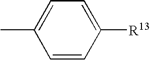
wherein R¹³ is as defined in claim 1.

11. A compound according to claim 10 wherein R¹ is 2,6-difluorophenyl, R^{2a} and R^{2b} are both hydrogen and R³ is selected from 1-methyl-piperidin-3-yl; 4-(2-dimethylaminoethoxy)-cyclohexyl; and an N-substituted 4-piperidinyl group wherein the N-substituent is selected from cyanomethyl and cyanoethyl.

12. A compound according to claim 10 wherein R¹ is 2,6-difluorophenyl, R^{2a} and R^{2b} are both hydrogen and R³ is (vii) a group: 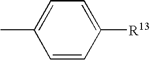
wherein R¹³ is selected from 4-morpholino, 4-thiomorpholino, 1-piperidino, 1-methyl-4-piperazino and 1-pyrrolidino.

13. A compound according to claim 1 wherein R¹ is a 2,3,6-trisubstituted phenyl group wherein the substituents for the phenyl group are selected from fluorine, chlorine, methyl and methoxy; and R^{2a} and R^{2b} are both hydrogen; and R³ is selected from (viii) 4-piperidinyl and 1-methyl-4-piperidinyl, (ix) tetrahydropyran-4-yl, and groups (ii), (x), (xi), (xii) and (xiii) as defined in claim 1.

14. A compound according to claim 13 wherein the 2,3,6-trisubstituted phenyl group has a fluorine, chlorine, methyl or methoxy group in the 2-position.

15. A compound according to claim 14 wherein the 2,3,6-trisubstituted phenyl group has at least two substituents present that are chosen from fluorine and chlorine.

16. A compound according to claim 13 wherein the 2,3,6-trisubstituted phenyl group is selected from are 2,3,6-trichlorophenyl, 2,3,6-trifluorophenyl, 2,3,difluoro-6-chlorophenyl, 2,3-difluoro-6-methylphenyl, 3-chloro-2,6-difluorophenyl, 2-chloro-3,6-difluorophenyl, 2-chloro-3-methoxy-6-fluorophenyl and 2-methoxy-3-fluoro-6-chlorophenyl groups.

17. A compound according to claim 13 wherein R³ is a 4-piperidinyl or 1-methyl-4-piperidinyl group.

18. A compound according to claim 13 wherein R³ is (x) a group: 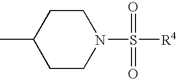
where R⁴ is as defined in claim 1.

19. A compound according to claim 13 wherein R³ is (ii) a group: 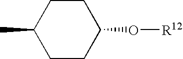
where R¹² is as defined in claim 1.

20. A compound according to claim 13 wherein R³ is (xi) a group: 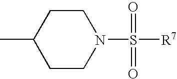
where R⁷ is as defined in claim 1.

21. A compound according to claim 13 wherein R³ is (xii) a group: 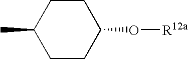
where R^{12a} is as defined in claim 1.

22. A compound according to claim 1 wherein R¹ is a group R^{1a}, R^{2a} and R^{2b} are both hydrogen, and R³ is (xiii) a group

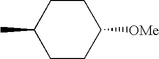

23. A compound according to claim 1 wherein R¹ is a group R^{1b}, R^{2a} and R^{2b} are both hydrogen, and R³ is (xiv) a methyl group.

24. A compound according to claim 1 wherein R¹ is a group R^{1c}, R^{2a} and R^{2b} are both hydrogen, and R³ is (xv) a group

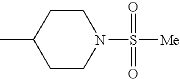

25. A compound according to claim 1 wherein R¹ is (j), 2,6-difluorophenylamino, R^{2a} and R^{2b} are both hydrogen; and R³ is methyl.

26. A compound according to claim 1 wherein R¹ is 2,6-dichlorophenyl, R³ is a 4-piperidine group and either (k) R^{2a} is methyl and R^{2b} is hydrogen, or (l) R^{2a} is hydrogen and R^{2b} is methyl.

27. A compound according to claim 1 wherein R¹ is (d), a group R⁰, where R⁰ is a carbocyclic or heterocyclic group having from 3 to 12 ring members; or a C₁₋₈ hydrocarbyl group optionally substituted by one or more substituents selected from fluorine, hydroxy, cyano; C₁₋₄ hydrocarbyloxy, amino, mono- or di-C₁₋₄ hydrocarbylamino, and carbocyclic or heterocyclic groups having from 3 to 12 ring members, and wherein 1 or 2 of the carbon atoms of the hydrocarbyl group may optionally be replaced by an atom or group selected from O, S, NH, SO, SO₂; and R³ is selected from:
(xi) a group: 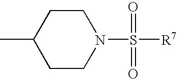
(xii) a group: 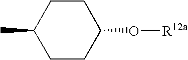
where R⁷, R^{7a} and R^{12a} are as defined herein.

28. A compound according to claim 1 selected from:
4-(2,6-dichloro-benzoylamino)-1H-pyrazole-3-carboxylic acid (4-methoxy methoxy-cyclohexyl)-amide;
4-(2,3-difluoro-6-methoxy-benzoylamino)-1H-pyrazole-3-carboxylic acid (1-methanesulphonyl-piperidin-4-yl)-amide;
4-(3-chloro-2,6-difluoro-benzoylamino)-1H-pyrazole-3-carboxylic acid (1-methanesulphonyl-piperidin-4-yl)-amide; and
4-(2-chloro-3,6-difluoro-benzoylamino)-1H-pyrazole-3-carboxylic acid (1-methanesulphonyl-piperidin-4-yl)-amide; and salts, solvates, tautomers and N-oxides thereof.

29. A compound according to claim 1 in the form of a salt, solvate or N-oxide.

30. (canceled)

31. (canceled)

32. (canceled)

33. A method for treating a disease or condition comprising or arising from abnormal cell growth in a mammal, which method comprises administering to the mammal a compound according to claim 1 in an amount effective in inhibiting abnormal cell growth.

34. (canceled)

35. (canceled)

36. (canceled)

37. (canceled)

38. A method of modulating a cellular process which method comprises contacting a cell with a compound according to claim 1.

39. (canceled)

40. (canceled)

41. A pharmaceutical composition comprising a compound according to claim 1 and a pharmaceutically acceptable carrier.

42. A pharmaceutical composition according to claim 41 wherein said pharmaceutically acceptable carrier is in a form suitable for oral administration.

43. (canceled)

44. (canceled)

45. A method for the diagnosis and treatment of a disease state or condition mediated by a cyclin dependent kinase, which method comprises (i) screening a patient to determine whether a disease or condition from which the patient is or may be suffering is one which would be susceptible to treatment with a compound having activity against cyclin dependent kinases; and (ii) where it is indicated that the disease or condition from which the patient is thus susceptible, thereafter administering to the patient a compound according to claim 1.

46. (canceled)

47. (canceled)

48. (canceled)

49. A method of inhibiting tumour growth in a mammal which method comprises administering to the mammal an effective tumour growth-inhibiting amount of a compound according to claim 1.

50. A method of inhibiting the growth of tumour cells, which method comprises
contacting the tumour cells with an effective tumour cell growth-inhibiting amount of a compound according to claim 1.

51. A method of claim 33 wherein the disease state or condition is a cancer.

52. A method according to claim 51 wherein the disease state or condition is a cancer which is selected from a carcinoma of the bladder, breast, colon, kidney, epidermis, liver, lung, oesophagus, gall bladder, ovary, pancreas, stomach, cervix, thyroid, prostate, or skin; a hematopoietic tumour of lymphoid lineage; a hematopoietic tumour of myeloid lineage; thyroid follicular cancer; a tumour of mesenchymal origin; a tumour of the central or peripheral nervous system; melanoma; seminoma; teratocarcinoma; osteosarcoma; xeroderma pigmentosum; keratoctanthoma; thyroid follicular cancer; or Kaposi's sarcoma.
